(19) United States
(12) Patent Application Publication
(10) Pub. No.: US 2023/0383149 A1
(43) Pub. Date: Nov. 30, 2023

(54) COORDINATION COMPOUND AND LIGHT ABSORBER COMPRISING SAME

(71) Applicant: LG CHEM, LTD., Seoul (KR)

(72) Inventors: Seonkyoung Son, Daejeon (KR); Duy Hieu Le, Daejeon (KR); Junhyun An, Daejeon (KR); Hoyong Lee, Daejeon (KR)

(21) Appl. No.: 18/031,597

(22) PCT Filed: Oct. 22, 2021
(86) PCT No.: PCT/KR2021/014871

§ 371 Date: Apr. 12, 2023

(30) Foreign Application Priority Data
Oct. 22, 2020 (KR) 10-2020-0137608

(51) Int. Cl. C09J 11/06; C07F 15/06; C07F 11/00; C09B 57/10; C09J 7/38; C09J 7/22; G02B 5/20; G02B 1/04

(52) U.S. Cl. CPC C09J 11/06; C07F 15/065; C07F 11/005; C09B 57/10; C09J 7/38; C09J 7/22; G02B 5/208; G02B 1/04; C09J 2203/326; C09J 2301/408

(57) ABSTRACT

A coordination compound, and a light absorber including the same.

### CROSS-REFERENCE TO RELATED APPLICATION

This application is a US national phase of international Application No. PCT/KR2021/014871, filed on Oct. 22, 2021, and claims priority to and the benefits of Korean Patent Application No. 10-2020-0137608, filed on Oct. 22, 2020, the entire contents of which are incorporated herein by reference.

### TECHNICAL FIELD

The present disclosure relates to a coordination compound, and a light absorber including the same.

### BACKGROUND

A display apparatus included in an electronic device normally expresses an image using a combination of light having specific wavelengths of R (red), G (green) and B (blue). Visibility of the display apparatus is favorable indoors because there is no external light having high energy.

However, strong light reflection occurs on the apparatus surface, an electrode in the apparatus or the like when outdoors due to external light such as sunlight having high energy, and the amount of light emitted by the display decreases, which causes problems of significantly reducing contrast ratio and visibility of the display.

In order to resolve the problems of reducing contrast ratio and visibility of the display caused by the external light reflection, attempts have been made to reduce reflected light by design to include a region absorbing external light inside the display. For example, an organic light emitting device (OLED) includes a polarizing plate in the device to reduce reflected light caused by the external light. The polarizing plate included in the electronic device improves surface reflection, electrode reflection and image emission brightness by controlling the amount of absorbed external light.

However, using a polarizing plate in an electronic device to control the amount of external light has problems of not flexibly adjusting color tones of the emitted color, increasing material costs, and not flexibly designing a structure of the device.

### Patent Document

(Patent Document 1) Publication of Japanese Patent Application Laid-Open No. 2012-211305

### SUMMARY

The present disclosure provides a novel coordination compound.

The present disclosure provides a light absorber including the coordination compound.

The present disclosure provides an adhesive film including the light absorber.

The present disclosure provides an optical film including the light absorber.

The present disclosure provides an electronic device including the light absorber.

One embodiment of the present disclosure provides a coordination compound, wherein, in a compound represented by Aⁿ⁻, A is represented by the following Chemical Formula A, and n is 1 or 2:

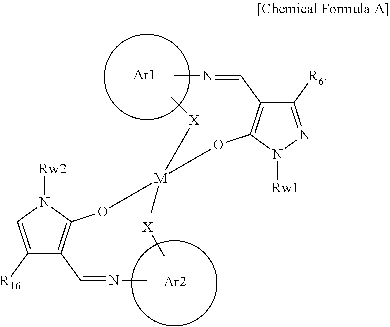

In Chemical Formula A,

M is a Cr ion or a Co ion,

X is O or O—C═O,

Rw1 and Rw2 are the same as or different from each other, and each independently may be a substituted or unsubstituted alkyl group; a substituted or unsubstituted alkenyl group; a substituted or unsubstituted cycloalkyl group; a substituted or unsubstituted aryl group; or a substituted or unsubstituted heteroaryl group,

Ar1 and Ar2 are the same as or different from each other, and each independently may be a substituted or unsubstituted arylene group; or a substituted or unsubstituted heteroarylene group,

R₆ and R₁₆ are the same as or different from each other, and each independently may be hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; a substituted or unsubstituted styryl group; —OR₁₀₀; —CO₂R₁₀₁; —COR₁₀₂; —OCOR₁₀₃; —CONR₁₀₄R₁₀₅; or —SO₂R₁₀₆, and

R₁₀₀ to R₁₀₆ are the same as or different from each other, and each independently may be hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

Another embodiment of the present disclosure provides a light absorber including the coordination compound.

Still another embodiment of the present disclosure provides an adhesive film including the light absorber.

Still another embodiment of the present disclosure provides an optical film including the light absorber.

Still another embodiment of the present disclosure provides an electronic device including the light absorber.

A coordination compound according to one embodiment of the present disclosure is used as a light absorber.

A coordination compound according to one embodiment of the present disclosure or a light absorber including the same is used in a film to enhance blue luminance transmittance.

A coordination compound according to one embodiment of the present disclosure or a light absorber including the same improves reflected color.

### BRIEF DESCRIPTION OF DRAWINGS

FIG. 1 illustrates an adhesive film according to one exemplary embodiment of the present disclosure.

FIG. 2 illustrates an optical film according to one exemplary embodiment of the present disclosure.

### DETAILED DESCRIPTION

Hereinafter, the present disclosure will be described in more detail.

In the present disclosure, a description of a certain part “including” certain constituents means capable of further including other constituents, and does not exclude other constituents unless particularly stated on the contrary.

In the present disclosure, a description of a certain member (layer) being placed “on” another member (layer) includes not only a case of the certain member (layer) being in contact with the another member but a case of still another member (layer) being present between the two members (layers).

In the present disclosure, the “layer” has a meaning compatible with a ‘film’ mainly used in the art, and means a coating that covers a target area. The size of the “layer” is not limited, and each “layer” may have the same or a different size. According to one embodiment, the size of the “layer” may be the same as the whole device, may correspond to the size of a specific functional area, or may be as small as a single sub-pixel.

Unless defined otherwise in the present disclosure, all technological and scientific terms used in the present disclosure have the same meanings as terms commonly understood by those skilled in the art. Although methods and materials similar or equivalent to those described in the present disclosure may be used in implementing or experimenting embodiments of the present disclosure, suitable methods and materials are described later. All publications, patent applications, patents and other reference documents mentioned in the present disclosure are incorporated by reference in the present disclosure as a whole, and when conflicting, the present disclosure including definitions herein has priority unless specific passage is mentioned. Furthermore, materials, methods and examples are for illustrative purposes only, and not to limit the present disclosure.

One exemplary embodiment of the present disclosure provides a coordination compound represented by Aⁿ⁻, wherein A is represented by Chemical Formula A, and n is 1 or 2:

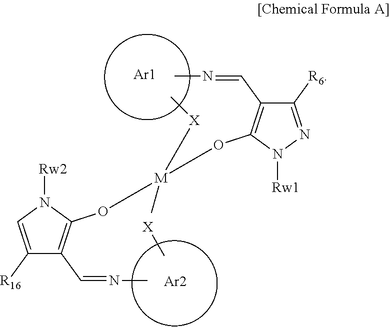

In Chemical Formula A,

M is a Cr ion or a Co ion,

X is O or O—C═O,

Rw1 and Rw2 are the same as or different from each other, and each independently may be a substituted or unsubstituted alkyl group; a substituted or unsubstituted alkenyl group; a substituted or unsubstituted cycloalkyl group; a substituted or unsubstituted aryl group; or a substituted or unsubstituted heteroaryl group,

Ar¹ and Ar² are the same as or different from each other, and each independently may be a substituted or unsubstituted arylene group; or a substituted or unsubstituted heteroarylene group,

R₆ and R₁₆ are the same as or different from each other, and each independently may be hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; a substituted or unsubstituted styryl group; —OR₁₀₀; —CO₂R₁₀₁; —COR₁₀₂; —OCOR₁₀₃; —CONR₁₀₄R₁₀₅; or —SO₂R₁₀₆, and

R₁₀₀ to R₁₀₆ are the same as or different from each other, and each independently may be hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

An organic light emitting device (OLED) panel generally has high reflectivity because a reflective electrode is included as an essential constitution. One way to reduce reflectivity of an OLED panel is using a light absorber that absorbs visible light in an optical film. The coordination compound according to the present disclosure absorbs visible light and is used as a light absorber. In addition, a light absorber including the coordination compound according to the present disclosure is used in a film to achieve an advantage of enhancing blue luminance transmittance or improving reflected color.

Specifically, when using the coordination compound of the present disclosure or the light absorber of the present disclosure, in an OLED, external light reflection may be efficiently suppressed without using a circular polarizing plate. Advantages of not using a circular polarizing plate in an OLED includes saving material costs and properly maintaining flexibility of the OLED.

In addition, because the structure represented by Chemical Formula A includes an imine bond (C═N) instead of an azo bond (N═N), a maximum absorption wavelength present in a region of 450 nm or greater may be shortened, and adjusted to have a target maximum absorption wavelength between 380 nm and 450 nm.

In a coordination compound of Chemical Formula where M bonds to N and O instead of bonding to O or O—C═O of X as in the coordination compound of this application, and particularly when M is a Co ion bonded to N and O, a maximum absorption wavelength is expressed in a short wavelength region of 320 nm to 340 nm, and when M is a Cu ion bonded to N and O, a maximum absorption wavelength is expressed between 500 nm to 560 nm with a very wide shape, and an actual effect as a blue-cut dye of such coordination compounds is insignificant.

In the present disclosure, a “complex” also refers to a coordination compound or a complex compound, and means a compound formed through a coordination bond of ligand lone pair electrons to a central metal ion having many empty orbitals.

Examples of substituents in the present disclosure are described below, however, the substituents are not limited thereto.

In the present disclosure, the term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when the compound includes two or more substituents, the two or more substituents may be the same as or different from each other.

In the present disclosure, the term “substituted or unsubstituted” means being substituted with one, two or more substituents selected from the group consisting of deuterium; a halogen group; a nitrile group (—CN); a nitro group (—NO₂); a hydroxyl group; —COOH; an imide group; an amide group; an alkylthioxy group; an arylthioxy group; an alkylsulfoxy group; an arylsulfoxy group; an alkenyl group; a silyl group; a boron group; an arylphosphine group; a phosphine oxide group; an alkoxy group; an aryloxy group; —CO₂R; —COR; —OCOR; —SO₂R; an alkyl group; a cycloalkyl group; an aryl group; a heterocyclic group; an amine group; a styryl group; and a heteroaryl group, or being substituted with a substituent linking two or more substituents among the substituents illustrated above, or having no substituents. Herein, R is hydrogen; an alkyl group; a cycloalkyl group; an aryl group; or a heteroaryl group.

Examples of the substituents are described below, however, the substituents are not limited thereto.

In the present disclosure, “deuterium” refers to a stable isotope of hydrogen having a mass approximately twice that of a most common isotope, that is, a mass of approximately 2 atomic mass units.

In the present disclosure, examples of the halogen group may include fluorine, chlorine, bromine or iodine.

In the present disclosure, the alkyl group may be linear or branched, and although not particularly limited thereto, the number of carbon atoms is preferably from 1 to 30; 1 to 20; 1 to 10; or 1 to 5. Specific examples thereof may include methyl, ethyl, propyl, n-propyl, isopropyl, butyl, n-butyl, isobutyl, t-butyl, sec-butyl, 1-methylbutyl, 1-ethylbutyl, pentyl, n-pentyl, isopentyl, neopentyl, t-pentyl, hexyl, n-hexyl, 1-methylpentyl, 2-methylpentyl, 3,3-dimethylbutyl, 2-ethylbutyl, heptyl, n-heptyl, 1-methylhexyl, cyclopentylmethyl, cyclohexylmethyl, octyl, n-octyl, t-octyl, 1-methylheptyl, 2-ethylhexyl, 2-propylpentyl, n-nonyl, 2,2-dimethylheptyl, 1-ethylpropyl, 1,1-dimethylpropyl, isohexyl, 4-methylhexyl, 5-methylhexyl and the like, but are not limited thereto. For reference, in the present disclosure, the propyl group includes an isopropyl group. In addition, in the present disclosure, the butyl group includes a tert-butyl group.

In the present disclosure, the descriptions on the alkyl group provided above are applied to the alkylene group except that the alkylene group is a divalent group.

In the present disclosure, the cycloalkyl group is not particularly limited, and although not particularly limited thereto, the number of carbon atoms is preferably from 3 to 60; 3 to 30; or 3 to 20. Specific examples of the cycloalkyl group may include a cyclopropyl group, a cyclobutyl group, a cyclopentyl group, a cyclohexyl group, a cycloheptyl group, a cyclooctyl group and the like, but are not limited thereto.

In the present disclosure, the descriptions of the cycloalkyl group provided above are applied to the cycloalkylene group except that the cycloalkylene group is a divalent group.

In the present disclosure, the aryl group means a monovalent aromatic hydrocarbon or a monovalent group of an aromatic hydrocarbon derivative. In the present disclosure, the aromatic hydrocarbon means a compound including a planar ring in which pi electrons are fully conjugated, and the group derived from aromatic hydrocarbon means a structure in which aromatic hydrocarbon or cyclic aliphatic hydrocarbon is fused to aromatic hydrocarbon. In addition, in the present disclosure, the aryl group includes a monovalent group in which two or more aromatic hydrocarbons or aromatic hydrocarbon derivatives are linked to each other. The aryl group is not particularly limited, but preferably has 6 to 60; 6 to 50; 6 to 30; 6 to 25; 6 to 20; 6 to 18; 6 to 15; 6 to 13; or 6 to 12 carbon atoms, and may be a monocyclic aryl group or a polycyclic aryl group.

The monocyclic aryl group is not particularly limited, but preferably has 6 to 60; 6 to 54; 6 to 48; 6 to 42; 6 to 36; 6 to 30; 6 to 24; 6 to 18; or 6 to 12 carbon atoms. Specific examples thereof may include a phenyl group, a biphenyl group, a terphenyl group and the like, but are not limited thereto.

The polycyclic aryl group is not particularly limited, but preferably has 6 to 60 carbon atoms; 6 to 45 carbon atoms; 6 to 30 carbon atoms; 6 to 25; 6 to 22; 6 to 20; 6 to 18; 6 to 16; 6 to 15; 6 to 14; 6 to 13; 6 to 12; or 6 to 10 carbon atoms. Examples thereof may include a naphthyl group, an anthracenyl group, a phenanthrenyl group, a pyrenyl group, a perylenyl group, a triphenylenyl group, a chrysenyl group, a fluorenyl group and the like, but are not limited thereto.

In the present disclosure, the heteroaryl group means a monovalent aromatic heteroring. Herein, the aromatic heteroring means a monovalent group of an aromatic ring or aromatic ring derivative, and means a group including one or more selected from the group consisting of N, O, P, S, Si and Se as a heteroatom. The aromatic ring derivative includes all structures in which an aromatic ring or aliphatic ring is fused to an aromatic ring. In addition, in the present disclosure, the heteroaryl group includes a monovalent group in which two or more aromatic rings including a heteroatom or aromatic ring derivatives including a heteroatom are linked to each other. The heteroaryl group preferably has 2 to 60; 2 to 50; 2 to 30; 2 to 20; 2 to 18; or 2 to 13 carbon atoms. Examples of the heteroaryl group may include a thiophene group, a furanyl group, a pyrrole group, an imidazole group, a thiazole group, an oxazole group, a pyridine group, a pyrimidine group, a triazine group, a triazole group, an acridine group, a pyridazine group, a pyrazine group, a quinoline group, a quinazoline group, a quinoxaline group, an isoquinoline group, an indole group, a carbazole group, a benzoxazole group, a benzimidazole group, a benzothiazole group, a benzocarbazole group, a benzothiophene group, a dibenzothiophene group, a benzofuran group, a phenanthrolinyl group, a dibenzofuran group and the like, but are not limited thereto.

In the present disclosure, the heteroaryl group may be monocyclic or polycyclic, and may be aromatic, aliphatic or a fused ring of aromatic and aliphatic groups.

In the present disclosure, the heterocyclic group is a monovalent group of an aliphatic ring or an aliphatic ring derivative, and means a group including one or more selected form the group consisting of N, O, P, S, Si and Se as a heteroatom.

In the present disclosure, the aliphatic ring is a hydrocarbon ring that is not aromatic or an aliphatic heteroring, and the examples of the cycloalkyl group of the present disclosure may be included as examples of the hydrocarbon ring, and morpholine may be included as examples of the aliphatic heteroring, however, the hydrocarbon ring and the aliphatic heteroring are not limited thereto.

In the present disclosure, the descriptions of the aryl group or the heteroaryl group may be applied to the aromatic ring.

In the present disclosure, the imide group may be represented by —C(O) NR^{x}C (O) R^{y}. Specifically, R^{x} and R^{y} may be each independently hydrogen, or a substituted or unsubstituted alkyl group, cycloalkyl group, alkenyl group, heterocyclic group, aryl group or heteroaryl group as defined in the present disclosure. Specifically, the number of carbon atoms of the imide group is not particularly limited, but is preferably from 1 to 25.

In the present disclosure, the amide group may be represented by —CONRoRp, and Ro and Rp and may be each independently hydrogen; an alkyl group; a cycloalkyl group; an aryl group; or a heteroaryl group as defined in the present disclosure.

In the present disclosure, the alkylthioxy group may be represented by —ORs1, and Rs1 may be an alkyl group as defined in the present disclosure.

In the present disclosure, the arylthioxy group may be represented by —ORs2, and Rs2 may be an aryl group as defined in the present disclosure.

In the present disclosure, the alkoxy group may be represented by —ORx, and Rx may be an alkyl group as defined in the present disclosure.

In the present disclosure, the aryloxy group may be represented by —ORy, and Ry may be an aryl group as defined in the present disclosure.

In the present disclosure, the alkylsulfoxy group may be represented by —SORs3, and Rs3 may be an alkyl group as defined in the present disclosure.

In the present disclosure, the arylsulfoxy group may be represented by —SORs4, and Rs4 may be an aryl group as defined in the present disclosure.

In the present disclosure, the alkenyl group refers to linear or branched unsaturated hydrocarbon including one or more double bonds. Specifically, the alkenyl group may be linear or branched, and although not particularly limited thereto, the number of carbon atoms is preferably from 2 to 40. According to one embodiment, the number of carbon atoms of the alkenyl group is from 2 to 20. According to another embodiment, the number of carbon atoms of the alkenyl group is from 2 to 10. According to another embodiment, the number of carbon atoms of the alkenyl group is from 2 to 6. Specific examples thereof may include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group and the like, but are not limited thereto.

In the present disclosure, the silyl group may be represented by —Si(Ri)₃, and Ri may be an alkyl group; an alkenyl group; or an aryl group as defined in the present disclosure. Examples thereof may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, the boron group may be represented by —B(Rb)₃, and Rb may be an alkyl group or an aryl group as defined in the present disclosure. Specific examples thereof may include a trimethylboron group, a triethylboron group, a t-butyldimethylboron group, a triphenylboron group, a phenylboron group and the like, but are not limited thereto.

In the present disclosure, the arylphosphine group may be represented by —PR^{a}R^{b}R^{c}. R^{a}, R^{b} and R^{c} may be each independently hydrogen, or an alkyl group, a cycloalkyl group, an alkenyl group, a heterocyclic group, an aryl group or a heteroaryl group as defined in the present disclosure.

In the present disclosure, the phosphine oxide group may be represented by —P (═O) R^{a}R^{b}R^{c}. R^{a}, R^{b} and R^{c} may be each independently hydrogen, or an alkyl group, a cycloalkyl group, an alkenyl group, a heterocyclic group, an aryl group or a heteroaryl group as defined in the present disclosure.

In the present disclosure, the amine group means a structure of —N(Rn)₂. Rn may be hydrogen; an alkyl group; a cycloalkyl group; an aryl group; or a heteroaryl group, as defined in the present disclosure.

In the present disclosure, the styryl group means that any one or more hydrogens of styrene (CHCH═CH₂) function as a bonding position.

In the present disclosure, an “adjacent group” may mean, for example, a first substituent substituted on a first atom that is directly linked to a second atom that is substituted with a second substituent, a first substituent sterically most closely positioned to the second substituent, or another substituent substituting the same atom that is substituted by the first or second substituent. For example, two substituents substituting ortho positions in a benzene ring, and two substituents substituting the same carbon in an aliphatic ring may be interpreted as groups “adjacent” to each other.

In the present disclosure, “adjacent substituents bonding to each other to form an aromatic ring” may mean adjacent substituents bonded to each other to form the aryl group or the heteroaryl group described in the present disclosure.

In the present disclosure, “adjacent substituents bonding to each other to form an aliphatic ring” may mean two adjacent substituents bonded to each other to form the cycloalkyl group or the heterocyclic group described above.

In one embodiment of the present disclosure, Rw1 and Rw2 are the same as or different from each other, and each independently may be a substituted or unsubstituted alkyl group having 1 to 30 carbon atoms; a substituted or unsubstituted alkenyl group having 2 to 30 carbon atoms; a substituted or unsubstituted cycloalkyl group having 3 to 30 carbon atoms; a substituted or unsubstituted aryl group having 6 to 30 carbon atoms; or a substituted or unsubstituted heteroaryl group having 3 to 30 carbon atoms.

In one embodiment of the present disclosure, Rw1 and Rw2 are the same as or different from each other, and each independently may be a substituted or unsubstituted alkyl group having 1 to 20 carbon atoms; a substituted or unsubstituted alkenyl group having 2 to 20 carbon atoms; a substituted or unsubstituted cycloalkyl group having 3 to 20 carbon atoms; a substituted or unsubstituted aryl group having 6 to 20 carbon atoms; or a substituted or unsubstituted heteroaryl group having 3 to 20 carbon atoms.

In one embodiment of the present disclosure, Rw1 and Rw2 are the same as or different from each other, and each independently may be a substituted or unsubstituted alkyl group having 1 to 10 carbon atoms; a substituted or unsubstituted alkenyl group having 2 to 10 carbon atoms; a substituted or unsubstituted cycloalkyl group having 3 to 10 carbon atoms; a substituted or unsubstituted aryl group having 6 to 10 carbon atoms; or a substituted or unsubstituted heteroaryl group having 3 to 10 carbon atoms.

In one embodiment of the present disclosure, Rw1 and Rw2 are the same as or different from each other, and each independently may be a substituted or unsubstituted phenyl group.

In one embodiment of the present disclosure, Rw1 and Rw2 are the same as or different from each other, and each independently may be an unsubstituted phenyl or a phenyl group substituted with a nitro group, a nitrile group, a sulfonic acid group, —COOH, chlorine, fluorine, a methyl group, a trifluoromethyl group, an ethyl group, an isopropyl group, a tert-butyl group, a methoxy group, an ethoxy group, —OCF₃, —COOCH₂CH₃, —COOCH₃, a cyclohexyl group, a phenyl group, a phenyl group substituted with an n-propyl group, a pyridine group or a thiazole group.

In one embodiment of the present disclosure, Ar1 and Ar2 are the same as or different from each other, and each independently may be a substituted or unsubstituted arylene group having 6 to 30 carbon atoms; or a substituted or unsubstituted heteroarylene group having 2 to 30 carbon atoms.

In one embodiment of the present disclosure, Ar1 and Ar2 are the same as or different from each other, and each independently may be a substituted or unsubstituted arylene group having 6 to 20 carbon atoms; or a substituted or unsubstituted heteroarylene group having 2 to 20 carbon atoms.

In one embodiment of the present disclosure, Ar1 and Ar2 are the same as or different from each other, and each independently may be a substituted or unsubstituted arylene group having 6 to 10 carbon atoms; or a substituted or unsubstituted heteroarylene group having 2 to 10 carbon atoms.

In one embodiment of the present disclosure, Ar1 and Ar2 are the same as or different from each other, and each independently may be a substituted or unsubstituted phenylene group; or a substituted or unsubstituted divalent pyridine group.

In one embodiment of the present disclosure, Ar1 and Ar2 are the same as or different from each other, and each independently may be a phenylene group substituted with fluorine, chlorine, bromine, a nitro group, a nitrile group, a sulfonic acid group, a methyl group, a trifluoromethyl group, a tert-butyl group, a methoxy group, a cyclohexyl group, a phenyl group, a phenyl group substituted with a methyl group, a phenyl group substituted with a methoxy group, a thiophene group, a pyridine group, —COCH₃, —COOCH₃ or —SO₂CH₃, a phenylene group having adjacent substituents bonded to each other to form a benzene ring; an unsubstituted phenylene group; or an unsubstituted divalent pyridine group.

In one embodiment of the present disclosure, M is Co²⁺; Co³⁺; Cr²⁺; or Cr³⁺.

In one embodiment of the present disclosure, M is Co²⁺.

In one embodiment of the present disclosure, M is Co³⁺.

In one embodiment of the present disclosure, M is Cr²⁺.

In one embodiment of the present disclosure, M is Cr³⁺.

In one embodiment of the present disclosure, X is O.

In one embodiment of the present disclosure, X is O—C═O.

In one embodiment of the present disclosure, when X is O—C═O, X bonds with M in Chemical Formula A in the order of M-O—(C═O)—Ar1 or M-O—(C═O)—Ar2.

According to one embodiment of the present disclosure, Chemical Formula A is represented by Chemical Formula A-1:

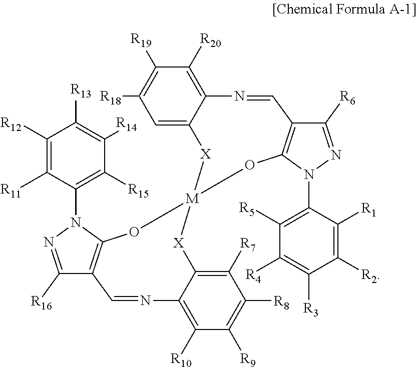

In Chemical Formula A-1,

M, X, R₆ and R₁₆ have the same definitions as in Chemical Formula A,

R₁ to R₅, R₇ to R₁₅ and R₁₇ to R₂₀ are the same as or different from each other, and each independently may be hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; —OR₁₀₀; —CO₂R₁₀₁; —COR₁₀₂; —OCOR₁₀₃; —CONR₁₀₄R₁₀₅; or —SO₂R₁₀₆, or adjacent substituents bonded to each other to form a substituted or unsubstituted aromatic ring or a substituted or unsubstituted aliphatic ring, and

R₁₀₀ to R₁₀₆ are the same as or different from each other, and each independently may be hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

In one embodiment of the present disclosure, R₁ to R₂₀ are the same as or different from each other, and each independently may be hydrogen; deuterium; a nitrile group; a nitro group; a hydroxyl group; —COOH; a halogen group; an imide group; a substituted or unsubstituted alkyl group having 1 to 30 carbon atoms; a substituted or unsubstituted cycloalkyl group having 3 to 30 carbon atoms; a substituted or unsubstituted heterocyclic group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted alkylthioxy group having 1 to 30 carbon atoms; a substituted or unsubstituted arylthioxy group having 6 to 30 carbon atoms; a substituted or unsubstituted alkylsulfoxy group having 1 to 30 carbon atoms; a substituted or unsubstituted arylsulfoxy group having 6 to 30 carbon atoms; a substituted or unsubstituted alkenyl group having 2 to 30 carbon atoms; a substituted or unsubstituted silyl group having 1 to 30 carbon atoms; a substituted or unsubstituted boron group having 1 to 30 carbon atoms; a substituted or unsubstituted arylphosphine group having 6 to 30 carbon atoms; a substituted or unsubstituted phosphine oxide group having 1 to 30 carbon atoms; —OR₁₀₀; —CO₂R₁₀₁; —COR₁₀₂; —OCOR₁₀₃; —CONR₁₀₄R₁₀₅; or —SO₂R₁₀₆, wherein R₁₀₀ and R₁₀₆ are as defined in the present disclosure, or adjacent substituents bonded to each other to form a substituted or unsubstituted aromatic ring having 6 to 30 carbon atoms or a substituted or unsubstituted aliphatic ring having 2 to 30 carbon atoms, and R₁₀₀ to R₁₀₆ are the same as or different from each other and each independently may be hydrogen; a substituted or unsubstituted alkyl group having 1 to 30 carbon atoms; a substituted or unsubstituted cycloalkyl group having 3 to 30 carbon atoms; a substituted or unsubstituted aryl group having 6 to 30 carbon atoms; a substituted or unsubstituted heteroaryl group having 2 to 30 carbon atoms; or a substituted or unsubstituted amine group.

In one embodiment of the present disclosure, R₁ to R₂₀ are the same as or different from each other, and each independently may be hydrogen; deuterium; a nitrile group; a nitro group; a hydroxyl group; —COOH; a halogen group; an imide group; a substituted or unsubstituted alkyl group having 1 to 20 carbon atoms; a substituted or unsubstituted cycloalkyl group having 3 to 20 carbon atoms; a substituted or unsubstituted heterocyclic group having 2 to 20 carbon atoms; a substituted or unsubstituted aryl group having 6 to 20 carbon atoms; a substituted or unsubstituted heteroaryl group having 2 to 20 carbon atoms; a substituted or unsubstituted alkylthioxy group having 1 to 20 carbon atoms; a substituted or unsubstituted arylthioxy group having 6 to 20 carbon atoms; a substituted or unsubstituted alkylsulfoxy group having 1 to 20 carbon atoms; a substituted or unsubstituted arylsulfoxy group having 6 to 20 carbon atoms; a substituted or unsubstituted alkenyl group having 2 to 20 carbon atoms; a substituted or unsubstituted silyl group having 1 to 20 carbon atoms; a substituted or unsubstituted boron group having 1 to 20 carbon atoms; a substituted or unsubstituted arylphosphine group having 6 to 20 carbon atoms; a substituted or unsubstituted phosphine oxide group having 1 to 20 carbon atoms; —OR₁₀₀; —CO₂R₁₀₁; —COR₁₀₂; —OCOR₁₀₃; —CONR₁₀₄R₁₀₅; or —SO₂R₁₀₆, wherein R₁₀₀ and R₁₀₆ are as defined in the present disclosure, or adjacent substituents bonded to each other to form a substituted or unsubstituted aromatic ring having 6 to 20 carbon atoms or a substituted or unsubstituted aliphatic ring having 2 to 20 carbon atoms, and R₁₀₀ to R₁₀₆ are the same as or different from each other and each may be independently hydrogen; a substituted or unsubstituted alkyl group having 1 to 20 carbon atoms; a substituted or unsubstituted cycloalkyl group having 3 to 20 carbon atoms; a substituted or unsubstituted aryl group having 6 to 20 carbon atoms; a substituted or unsubstituted heteroaryl group having 2 to 20 carbon atoms; or a substituted or unsubstituted amine group.

In one embodiment of the present disclosure, R₁ to R₂₀ are the same as or different from each other, and each independently may be hydrogen; deuterium; a nitrile group; a nitro group; a hydroxyl group; —COOH; a halogen group; an imide group; a substituted or unsubstituted alkyl group having 1 to 10 carbon atoms; a substituted or unsubstituted cycloalkyl group having 3 to 10 carbon atoms; a substituted or unsubstituted heterocyclic group having 2 to 10 carbon atoms; a substituted or unsubstituted aryl group having 6 to 10 carbon atoms; a substituted or unsubstituted heteroaryl group having 2 to 10 carbon atoms; a substituted or unsubstituted alkylthioxy group having 1 to 10 carbon atoms; a substituted or unsubstituted arylthioxy group having 6 to 10 carbon atoms; a substituted or unsubstituted alkylsulfoxy group having 1 to 10 carbon atoms; a substituted or unsubstituted arylsulfoxy group having 6 to 10 carbon atoms; a substituted or unsubstituted alkenyl group having 2 to 10 carbon atoms; a substituted or unsubstituted silyl group having 1 to 10 carbon atoms; a substituted or unsubstituted boron group having 1 to 10 carbon atoms; a substituted or unsubstituted arylphosphine group having 6 to 10 carbon atoms; a substituted or unsubstituted phosphine oxide group having 1 to 10 carbon atoms; —OR₁₀₀; —CO₂R₁₀₁; —COR₁₀₂; —OCOR₁₀₃; —CONR₁₀₄R₁₀₅; or —SO₂R₁₀₆, wherein R₁₀₀ and R₁₀₆ are as defined in the present disclosure, or adjacent substituents bonded to each other to form a substituted or unsubstituted aromatic ring having 6 to 10 carbon atoms or a substituted or unsubstituted aliphatic ring having 2 to 10 carbon atoms, and R₁₀₀ to R₁₀₆ are the same as or different from each other and each independently may be hydrogen; a substituted or unsubstituted alkyl group having 1 to 10 carbon atoms; a substituted or unsubstituted cycloalkyl group having 3 to 10 carbon atoms; a substituted or unsubstituted aryl group having 6 to 10 carbon atoms; a substituted or unsubstituted heteroaryl group having 2 to 10 carbon atoms; or a substituted or unsubstituted amine group.

In one embodiment of the present disclosure, R₁ to R₂₀ are the same as or different from each other, and each independently may be hydrogen; deuterium; a nitrile group; a nitro group; a hydroxyl group; —COOH; a halogen group; a substituted or unsubstituted alkyl group; a substituted or unsubstituted cycloalkyl group; a substituted or unsubstituted alkenyl group; a substituted or unsubstituted aryl group; a substituted or unsubstituted heteroaryl group; —OR₁₀₀; —CO₂R₁₀₁; —COR₁₀₂; or —SO₂R₁₀₆, wherein R₁₀₀ and R₁₀₂ and R₁₀₆ are as defined in the present disclosure, or adjacent substituents bonded to each other to form a substituted or unsubstituted aromatic ring.

In one embodiment of the present disclosure, R₁ to R₂₀ are the same as or different from each other, and each independently may be hydrogen; deuterium; a nitrile group; a nitro group; a hydroxyl group; —COOH; a halogen group; a substituted or unsubstituted alkyl group having 1 to 30 carbon atoms; a substituted or unsubstituted cycloalkyl group having 3 to 30 carbon atoms; a substituted or unsubstituted alkenyl group having 2 to 30 carbon atoms; a substituted or unsubstituted aryl group having 6 to 30 carbon atoms; a substituted or unsubstituted heteroaryl group having 2 to 30 carbon atoms; —OR₁₀₀; —CO₂R₁₀₁; —COR₁₀₂; or —SO₂R₁₀₆, wherein R₁₀₀ to R₁₀₂ and R₁₀₆ are as defined in the present disclosure, or adjacent substituents bonded to each other to form a substituted or unsubstituted aromatic ring having 6 to 30 carbon atoms.

In one embodiment of the present disclosure, R₁ to R₂₀ are the same as or different from each other, and each independently may be hydrogen; deuterium; a nitrile group; a nitro group; a hydroxyl group; —COOH; a halogen group; a substituted or unsubstituted alkyl group having 1 to 20 carbon atoms; a substituted or unsubstituted cycloalkyl group having 3 to 20 carbon atoms; a substituted or unsubstituted alkenyl group having 2 to 20 carbon atoms; a substituted or unsubstituted aryl group having 6 to 20 carbon atoms; a substituted or unsubstituted heteroaryl group having 2 to 20 carbon atoms; —OR₁₀₀; —CO₂R₁₀₁; —COR₁₀₂; or —SO₂R₁₀₆, wherein R₁₀₀ to R₁₀₂ and R₁₀₆ are as defined in the present disclosure, or adjacent substituents bonded to each other to form a substituted or unsubstituted aromatic ring having 6 to 20 carbon atoms.

In one embodiment of the present disclosure, R₁ to R₂₀ are the same as or different from each other, and each independently may be hydrogen; deuterium; a nitrile group; a nitro group; a hydroxyl group; —COOH; a halogen group; a substituted or unsubstituted alkyl group having 1 to 10 carbon atoms; a substituted or unsubstituted cycloalkyl group having 3 to 10 carbon atoms; a substituted or unsubstituted alkenyl group having 2 to 10 carbon atoms; a substituted or unsubstituted aryl group having 6 to 10 carbon atoms; a substituted or unsubstituted heteroaryl group having 2 to 10 carbon atoms; —OR₁₀₀; —CO₂R₁₀₁; —COR₁₀₂; or —SO₂R₁₀₆, wherein R₁₀₀ to R₁₀₂ and R₁₀₆ are as defined in the present disclosure, or adjacent substituents bonded to each other to form a substituted or unsubstituted aromatic ring having 6 to 10 carbon atoms.

In one embodiment of the present disclosure, R₁ to R₂₀ are the same as or different from each other, and each independently may be hydrogen; deuterium; a nitrile group; a nitro group; a hydroxyl group; —COOH; chlorine; fluorine; bromine; a substituted or unsubstituted methyl group; a substituted or unsubstituted ethyl group; a substituted or unsubstituted propyl group; a substituted or unsubstituted butyl group; a substituted or unsubstituted cyclohexyl group; a substituted or unsubstituted ethenyl group; a substituted or unsubstituted phenyl group; a substituted or unsubstituted biphenyl group; a substituted or unsubstituted benzoxazole group; a substituted or unsubstituted pyridine group; a substituted or unsubstituted thiophene group; a substituted or unsubstituted furan group; a substituted or unsubstituted thiazole group; —OR₁₀₀; —CO₂R₁₀₁; —COR₁₀₂; or —SO₂R₁₀₆, wherein R₁₀₀ to R₁₀₂ and R₁₀₆ are as defined in the present disclosure, or adjacent substituents bonded to each other to form a substituted or unsubstituted benzene ring.

In one embodiment of the present disclosure, R₁ to R₂₀ are the same as or different from each other, and each independently may be hydrogen; deuterium; a nitrile group; a nitro group; a hydroxyl group; —COOH; chlorine; fluorine; bromine; a methyl group; a trifluoromethyl group; an ethyl group; an isopropyl group; a tert-butyl group; a cyclohexyl group; an ethenyl group substituted with a phenyl group substituted with a nitro group, an isopropyl group or a tert-butyl group; a phenyl group substituted with a nitro group, a nitrile group, —COOH, fluorine, a methoxy group, —OCF₃, —COOCH₂CH₃, a methyl group, an n-propyl group, a cyclohexyl group or a pyridine group; a biphenyl group; a benzoxazole group; a pyridine group; a thiophene group; a furan group; a thiazole group; —OR₁₀₀; —CO₂R₁₀₁; —COR₁₀₂; or —SO₂R₁₀₆, wherein R₁₀₀ to R₁₀₂ and R₁₀₆ are as defined in the present disclosure, or adjacent substituents bonded to each other to form a benzene ring.

In one embodiment of the present disclosure, R₁ to R₂₀ are the same as or different from each other, and each independently may be hydrogen; chlorine; fluorine; a nitro group; a methyl group; a trifluoromethyl group; an isopropyl group; a tert-butyl group; a phenyl group; —OR₁₀₀; —CO₂R₁₀₁; or —SO₂R₁₀₆, wherein R₁₀₀ to R₁₀₂ and R₁₀₆ are as defined in the present disclosure, or adjacent substituents bonded to each other to form a benzene ring.

In one embodiment of the present disclosure, R₈ and R₉, R₁₉ and R₁₈, R₉ and R₁₀ or R₁₉ and R₂₀ are bonded to each other to form a substituted or unsubstituted benzene ring.

In one embodiment of the present disclosure, R₈ and R₉, R₁₉ and R₁₈, R₉ and R₁₀ or R₁₉ and R₂₀ are bonded to each other to form a benzene ring.

In one embodiment of the present disclosure, R₁₀₀ to R₁₀₆ are the same as or different from each other, and each independently may be a substituted or unsubstituted alkyl group; or a substituted or unsubstituted amine group.

In one embodiment of the present disclosure, R₁₀₀ to R₁₀₆ are the same as or different from each other, and each independently may be a substituted or unsubstituted alkyl group having 1 to 30 carbon atoms; or a substituted or unsubstituted amine group.

In one embodiment of the present disclosure, R₁₀₀ to R₁₀₆ are the same as or different from each other, and each independently may be a substituted or unsubstituted alkyl group having 1 to 20 carbon atoms; or a substituted or unsubstituted amine group.

In one embodiment of the present disclosure, R₁₀₀ to R₁₀₆ are the same as or different from each other, and each independently may be a substituted or unsubstituted alkyl group having 1 to 10 carbon atoms; or a substituted or unsubstituted amine group.

In one embodiment of the present disclosure, R₁₀₀ to R₁₀₆ are the same as or different from each other, and each independently may be a substituted or unsubstituted methyl group; a substituted or unsubstituted ethyl group; or —NH₂.

In one embodiment of the present disclosure, R₁₀₀ to R₁₀₆ are the same as or different from each other, and each independently may be a methyl group; a fluoromethyl group; an ethyl group; or —NH₂.

In one embodiment of the present disclosure, R₁₀₀ is a methyl group; a trifluoromethyl group; or an ethyl group.

In one embodiment of the present disclosure, R₁₀₁ is a methyl group; or an ethyl group.

In one embodiment of the present disclosure, R₁₀₂ is a methyl group.

In one embodiment of the present disclosure, R₁₀₆ is a methyl group; or —NH₂.

According to one embodiment of the present disclosure, R₁ to R₅ and R₁₁ to R₁₅ are the same as or different from each other, and each independently may be hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; —OR₁₀₀; —CO₂R₁₀₁; —COR₁₀₂; —OCOR₁₀₃; —CONR₁₀₄R₁₀₅; or —SO₂R₁₀₆, wherein R₁₀₀ to R₁₀₆ are as defined in the present disclosure, or adjacent substituents may be bonded to each other to form a substituted or unsubstituted aromatic ring or a substituted or unsubstituted aliphatic ring, and R₁₀₀ to R₁₀₆ are the same as or different from each other and each independently may be hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

According to one embodiment of the present disclosure, R₁ to R₅ and R₁₁ to R₁₅ are the same as or different from each other, and each independently may be hydrogen; deuterium; a halogen group; a nitrile group; a nitro group; —COOH; a substituted or unsubstituted alkyl group having 1 to 30 carbon atoms; a substituted or unsubstituted cycloalkyl group having 3 to 30 carbon atoms; an unsubstituted aryl group having 6 to 30 carbon atoms or an aryl group having 6 to 30 carbon atoms substituted with an alkyl group; a substituted or unsubstituted heteroaryl group having 2 to 30 carbon atoms; —OR₁₀₀; or —CO₂R₁₀₁, and R₁₀₀ and R₁₀₁ are the same as or different from each other and each independently may be a substituted or unsubstituted alkyl group having 1 to 30 carbon atoms.

According to one embodiment of the present disclosure, R₁ to R₅ and R₁₁ to R₁₅ are the same as or different from each other, and each independently may be hydrogen; deuterium; a halogen group; a nitrile group; a nitro group; —COOH; a substituted or unsubstituted alkyl group having 1 to 20 carbon atoms; a substituted or unsubstituted cycloalkyl group having 3 to 20 carbon atoms; an unsubstituted aryl group having 6 to 20 carbon atoms or an aryl group having 6 to 20 carbon atoms substituted with an alkyl group; a substituted or unsubstituted heteroaryl group having 2 to 20 carbon atoms; —OR₁₀₀; or —CO₂R₁₀₁, and R₁₀₀ and R₁₀₁ are the same as or different from each other and each independently may be a substituted or unsubstituted alkyl group having 1 to 20 carbon atoms.

According to one embodiment of the present disclosure, R₁ to R₅ and R₁₁ to R₁₅ are the same as or different from each other, and each independently may be hydrogen; deuterium; a halogen group; a nitrile group; a nitro group; —COOH; a substituted or unsubstituted alkyl group having 1 to 10 carbon atoms; a substituted or unsubstituted cycloalkyl group having 3 to 10 carbon atoms; an unsubstituted aryl group having 6 to 10 carbon atoms or an aryl group having 6 to 10 carbon atoms substituted with an alkyl group; a substituted or unsubstituted heteroaryl group having 2 to 10 carbon atoms; —OR₁₀₀; or —CO₂R₁₀₁, and R₁₀₀ and R₁₀₁ are the same as or different from each other and each independently may be a substituted or unsubstituted alkyl group having 1 to 10 carbon atoms.

According to one embodiment of the present disclosure, R₁ to R₅ and R₁₁ to R₁₅ are the same as or different from each other, and each independently may be hydrogen; deuterium; a fluoro group; a chloro group; a nitrile group; a nitro group; —COOH; a substituted or unsubstituted methyl group; a substituted or unsubstituted ethyl group; a substituted or unsubstituted propyl group; a substituted or unsubstituted tert-butyl group; a substituted or unsubstituted cyclohexyl group; a substituted or unsubstituted phenyl group; a substituted or unsubstituted pyridine group; a substituted or unsubstituted thiazole group; —OR₁₀₀; or —CO₂R₁₀₁, and R₁₀₀ and R₁₀₁ are the same as or different from each other and each independently may be a substituted or unsubstituted methyl group; or a substituted or unsubstituted ethyl group.

According to one embodiment of the present disclosure, R₁ to R₅ and R₁₁ to R₁₅ are the same as or different from each other, and each independently may be hydrogen; deuterium; a fluoro group; a chloro group; a nitrile group; a nitro group; —COOH; an unsubstituted methyl group or a methyl group substituted with a halogen group; an ethyl group; a propyl group; a tert-butyl group; a cyclohexyl group; an unsubstituted phenyl group or a phenyl group substituted with an alkyl group; a pyridine group; a thiazole group; —OR₁₀₀; or —CO₂R₁₀₁, and R₁₀₀ and R₁₀₁ are the same as or different from each other and each independently may be an unsubstituted methyl group or a methyl group substituted with a halogen group; or an ethyl group.

According to one embodiment of the present disclosure, R1 to R₅ and R₁₁ to R₁₅ are the same as or different from each other, and each independently may be hydrogen; deuterium; a fluoro group; a chloro group; a nitrile group; a nitro group; —COOH; an unsubstituted methyl group or a methyl group substituted with a chloro group; an ethyl group; a propyl group; a tert-butyl group; a cyclohexyl group; an unsubstituted phenyl group or a phenyl group substituted with a propyl group; a pyridine group; a thiazole group; —OR₁₀₀; or —CO₂R₁₀₁, and R₁₀₀ and R₁₀₁ are the same as or different from each other and each independently may be a methyl group unsubstituted or substituted with a chloro group; or an ethyl group.

According to one embodiment of the present disclosure, R₁ to R₅ and R₁₁ to R₁₅ are the same as or different from each other, and each independently may be hydrogen; deuterium; a fluoro group; a chloro group; a nitrile group; a nitro group; —COOH; an unsubstituted methyl group or a methyl group substituted with a chloro group; an ethyl group; an isopropyl group; a tert-butyl group; a cyclohexyl group; an unsubstituted phenyl group or a phenyl group substituted with a propyl group; a pyridine group; a thiazole group; —OCH₃; —OCF₃; —OC₂H₅; —CO₂CH₃; or —CO₂C₂H₅.

According to one embodiment of the present disclosure, R₇ to R₁₀ and R₁₇ to R₂₀ are the same as or different from each other, and each independently may be hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; —OR₁₀₀; —CO₂R₁₀₁; —COR₁₀₂; —OCOR₁₀₃; —CONR₁₀₄R₁₀₅; or —SO₂R₁₀₆, or adjacent substituents are bonded to each other to form a substituted or unsubstituted aromatic ring or a substituted or unsubstituted aliphatic ring, and R₁₀₀ to R₁₀₆ are the same as or different from each other and each independently may be hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

According to one embodiment of the present disclosure, R₇ to R₁₀ and R₁₇ to R₂₀ are the same as or different from each other, and each independently may be hydrogen; deuterium; a halogen group; a nitrile group; a nitro group; a substituted or unsubstituted alkyl group having 1 to 30 carbon atoms; a substituted or unsubstituted cycloalkyl group having 3 to 30 carbon atoms; an unsubstituted aryl group having 6 to 30 carbon atoms or an aryl group having 6 to 30 carbon atoms substituted with —OR₁₀₀ or an alkyl group; a substituted or unsubstituted heteroaryl group having 2 to 30 carbon atoms; —OR₁₀₀; —CO₂R₁₀₁; —COR₁₀₂; or —SO₂R₁₀₆, or adjacent substituents bonded to each other to form a substituted or unsubstituted aromatic ring, R₁₀₀, R₁₀₁ and R₁₀₂ are the same as or different from each other and each independently may be a substituted or unsubstituted alkyl group having 1 to 30 carbon atoms, and R₁₀₆ is a substituted or unsubstituted alkyl group having 1 to 30 carbon atoms; or —NH₂.

According to one embodiment of the present disclosure, R₇ to R₁₀ and R₁₇ to R₂₀ are the same as or different from each other, and each independently may be hydrogen; deuterium; a halogen group; a nitrile group; a nitro group; a substituted or unsubstituted alkyl group having 1 to 20 carbon atoms; a substituted or unsubstituted cycloalkyl group having 3 to 20 carbon atoms; an unsubstituted aryl group having 6 to 20 carbon atoms or an aryl group having 6 to 20 carbon atoms substituted with —OR₁₀₀ or an alkyl group; a substituted or unsubstituted heteroaryl group having 2 to 20 carbon atoms; —OR₁₀₀; —CO₂R₁₀₁; —COR₁₀₂; or —SO₂R₁₀₆, or adjacent substituents bonded to each other to form a substituted or unsubstituted aromatic ring, R₁₀₀, R₁₀₁ and R₁₀₂ are the same as or different from each other and each independently may be a substituted or unsubstituted alkyl group having 1 to 20 carbon atoms, and R₁₀₆ is a substituted or unsubstituted alkyl group having 1 to 20 carbon atoms; or —NH₂.

According to one embodiment of the present disclosure, R₇ to R₁₀ and R₁₇ to R₂₀ are the same as or different from each other, and each independently may be hydrogen; deuterium; a halogen group; a nitrile group; a nitro group; a substituted or unsubstituted alkyl group having 1 to 10 carbon atoms; a substituted or unsubstituted cycloalkyl group having 3 to 10 carbon atoms; an unsubstituted aryl group having 6 to 10 carbon atoms or an aryl group having 6 to 10 carbon atoms substituted with —OR₁₀₀ or an alkyl group; a substituted or unsubstituted heteroaryl group having 2 to 10 carbon atoms; —OR₁₀₀; —CO₂R₁₀₁; —COR₁₀₂; or —SO₂R₁₀₆, or adjacent substituents bonded to each other to form a substituted or unsubstituted aromatic ring, R₁₀₀, R₁₀₁ and R₁₀₂ are the same as or different from each other and each independently may be a substituted or unsubstituted alkyl group having 1 to 10 carbon atoms, and R₁₀₆ is a substituted or unsubstituted alkyl group having 1 to 10 carbon atoms; or —NH₂.

According to one embodiment of the present disclosure, R₇ to R₁₀ and R₁₇ to R₂₀ are the same as or different from each other, and each independently may be hydrogen; deuterium; a fluoro group; a chloro group; a bromo group; a nitrile group; a nitro group; a substituted or unsubstituted methyl group; a substituted or unsubstituted propyl group; a substituted or unsubstituted tert-butyl group; a substituted or unsubstituted cyclohexyl group; a substituted or unsubstituted phenyl group; a substituted or unsubstituted thiophene group; a substituted or unsubstituted pyridine group; —OR₁₀₀; —CO₂R₁₀₁; —COR₁₀₂; or —SO₂R₁₀₆, or adjacent substituents bonded to each other to form a substituted or unsubstituted benzene ring, R₁₀₀, R₁₀₁ and R₁₀₂ are the same as or different from each other and each independently may be a substituted or unsubstituted methyl group; or a substituted or unsubstituted ethyl group, and R₁₀₆ is a substituted or unsubstituted methyl group; or —NH₂.

According to one embodiment of the present disclosure, R₇ to R₁₀ and R₁₇ to R₂₀ are the same as or different from each other, and each independently may be hydrogen; deuterium; a fluoro group; a chloro group; a bromo group; a nitrile group; a nitro group; an unsubstituted methyl group or a methyl group substituted with a fluoro group; an isopropyl group; a tert-butyl group; a cyclohexyl group; an unsubstituted phenyl group or a phenyl group substituted with —OCH₃ or an alkyl group; a thiophene group; a pyridine group; —OCH₃; —CO₂CH₃; —CO₂C₂H₅; —SO₂CH₃ or —SO₂NH₂, or adjacent substituents bonded to each other to form a benzene ring.

According to one embodiment of the present disclosure, R₆ and R₁₆ are the same as or different from each other, and each independently may be hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; a substituted or unsubstituted styryl group; —OR₁₀₀; —CO₂R₁₀₁; —COR₁₀₂; —OCOR₁₀₃; —CONR₁₀₄R₁₀₅; or —SO₂R₁₀₆, and R₁₀₀ to R₁₀₆ are the same as or different from each other and each independently may be hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

In one embodiment of the present disclosure, R₆ and R₁₆ are the same as or different from each other, and each independently may be a nitrile group; a substituted or unsubstituted alkyl group; a substituted or unsubstituted alkenyl group; a substituted or unsubstituted aryl group; a substituted or unsubstituted heteroaryl group; —OR₁₀₀; or —CO₂R₁₀₁, wherein R₁₀₀ and R₁₀₁ are as defined in the present disclosure.

In one embodiment of the present disclosure, R₆ and R₁₆ are the same as or different from each other, and each independently may be a nitrile group; a substituted or unsubstituted alkyl group having 1 to 30 carbon atoms; a substituted or unsubstituted alkenyl group having 2 to 30 carbon atoms; a substituted or unsubstituted aryl group having 6 to 30 carbon atoms; a substituted or unsubstituted heteroaryl group having 2 to 30 carbon atoms; —OR₁₀₀; or —CO₂R₁₀₁, and wherein R₁₀₀ and R₁₀₁ are as defined in the present disclosure.

In one embodiment of the present disclosure, R₆ and R₁₆ are the same as or different from each other, and each independently may be a nitrile group; a substituted or unsubstituted alkyl group having 1 to 20 carbon atoms; a substituted or unsubstituted alkenyl group having 2 to 20 carbon atoms; a substituted or unsubstituted aryl group having 6 to 20 carbon atoms; a substituted or unsubstituted heteroaryl group having 2 to 20 carbon atoms; —OR₁₀₀; or —CO₂R₁₀₁, wherein R₁₀₀ and R₁₀₁ are as defined in the present disclosure.

In one embodiment of the present disclosure, R₆ and R₁₆ are the same as or different from each other, and each independently may be a nitrile group; a substituted or unsubstituted alkyl group having 1 to 10 carbon atoms; a substituted or unsubstituted alkenyl group having 2 to 10 carbon atoms; a substituted or unsubstituted aryl group having 6 to 10 carbon atoms; a substituted or unsubstituted heteroaryl group having 2 to 10 carbon atoms; —OR₁₀₀; or —CO₂R₁₀₁, wherein R₁₀₀ and R₁₀₁ are as defined in the present disclosure.

In one embodiment of the present disclosure, R₆ and R₁₆ are the same as or different from each other, and each independently may be a nitrile group; a substituted or unsubstituted methyl group; a substituted or unsubstituted ethyl group; a substituted or unsubstituted propyl group; a substituted or unsubstituted ethenyl group; a substituted or unsubstituted phenyl group; a substituted or unsubstituted pyridine group; a substituted or unsubstituted benzoxazole group; —OR₁₀₀; or —CO₂R₁₀₁, wherein R₁₀₀ and R₁₀₁ are as defined in the present disclosure.

According to one embodiment of the present disclosure, R₆ and R₁₆ are the same as or different from each other, and each independently may be a nitrile group; a methyl group unsubstituted or substituted with a fluoro group; a substituted or unsubstituted ethyl group; a substituted or unsubstituted isopropyl group; an unsubstituted phenyl group or a phenyl group substituted with one or more substituents selected from the group consisting of a halogen group, a nitrile group, a nitro group, —COOH, an alkyl group, —OR₁₀₀ and —CO₂R₁₀₁; a substituted or unsubstituted benzoxazole group; a substituted or unsubstituted pyridine group; an unsubstituted styryl group or a styryl group substituted with an alkyl group or a nitro group; —OR₁₀₀; or —CO₂R₁₀₁, and R₁₀₁ and R₁₀₂ are the same as or different from each other and each independently may be a substituted or unsubstituted methyl group; or a substituted or unsubstituted ethyl group.

In one embodiment of the present disclosure, R₆ and R₁₆ are the same as or different from each other, and each independently a nitrile group; a methyl group; an ethyl group; an isopropyl group; an ethenyl group substituted with a phenyl group substituted with a nitro group, an isopropyl group or a tert-butyl group; a phenyl group unsubstituted or substituted with a nitro group, fluorine, a nitrile group; —COOH, a methyl group, a methoxy group, —OCF₃ or —COOCH₂CH₃; a pyridine group; a benzoxazole group; —OR₁₀₀; or —CO₂R₁₀₁, and R₁₀₁ and R₁₀₂ are the same as or different from each other and each independently may be a substituted or unsubstituted methyl group; or a substituted or unsubstituted ethyl group.

In one embodiment of the present disclosure, Chemical Formula A is any one selected from the group consisting of the following materials:

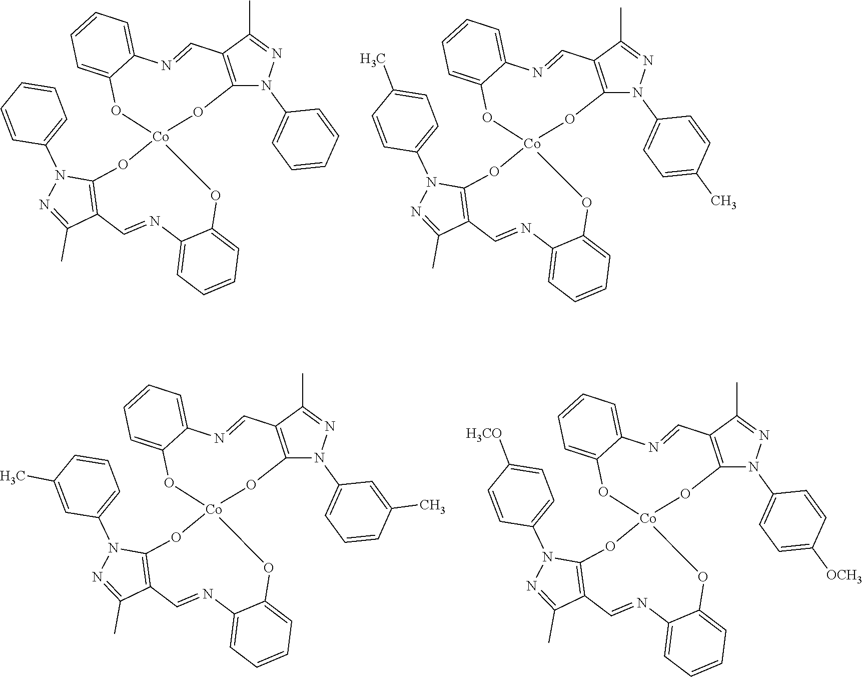

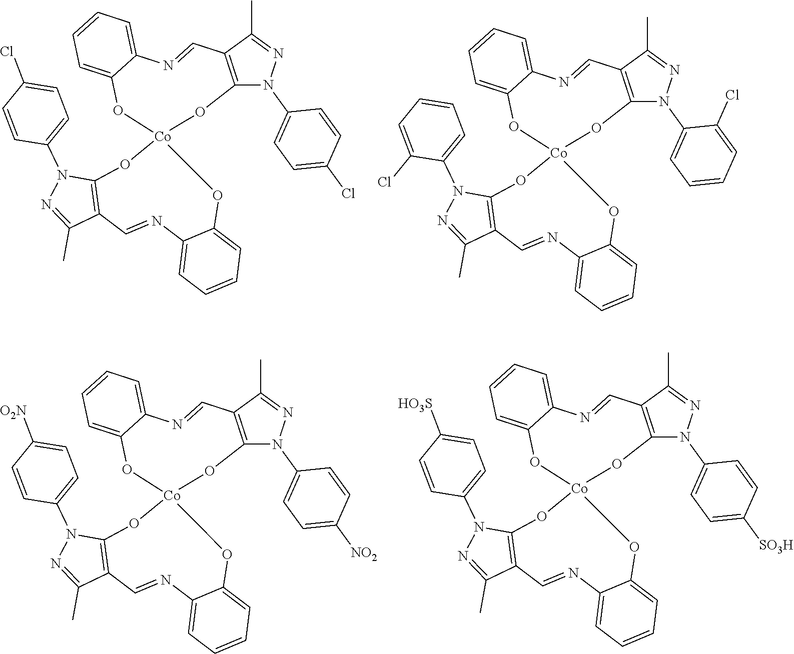

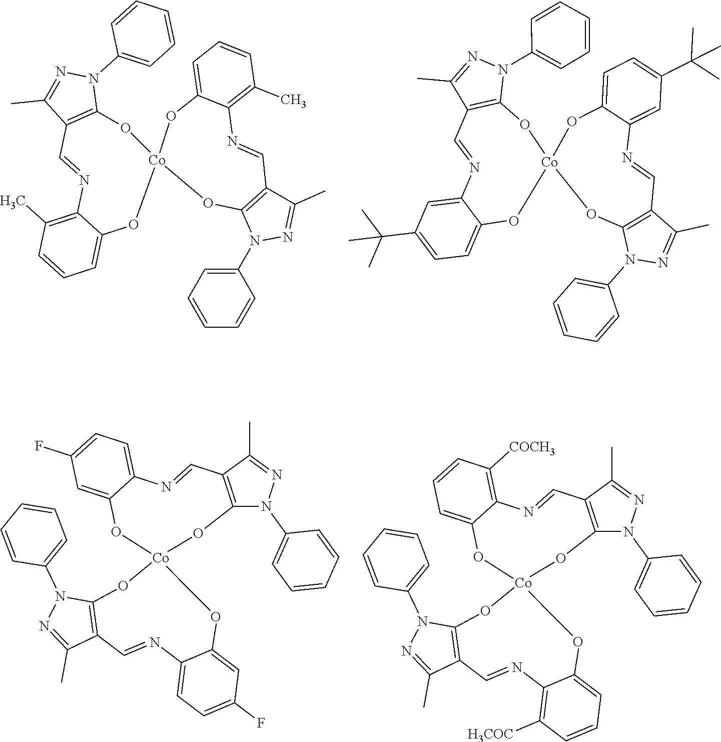

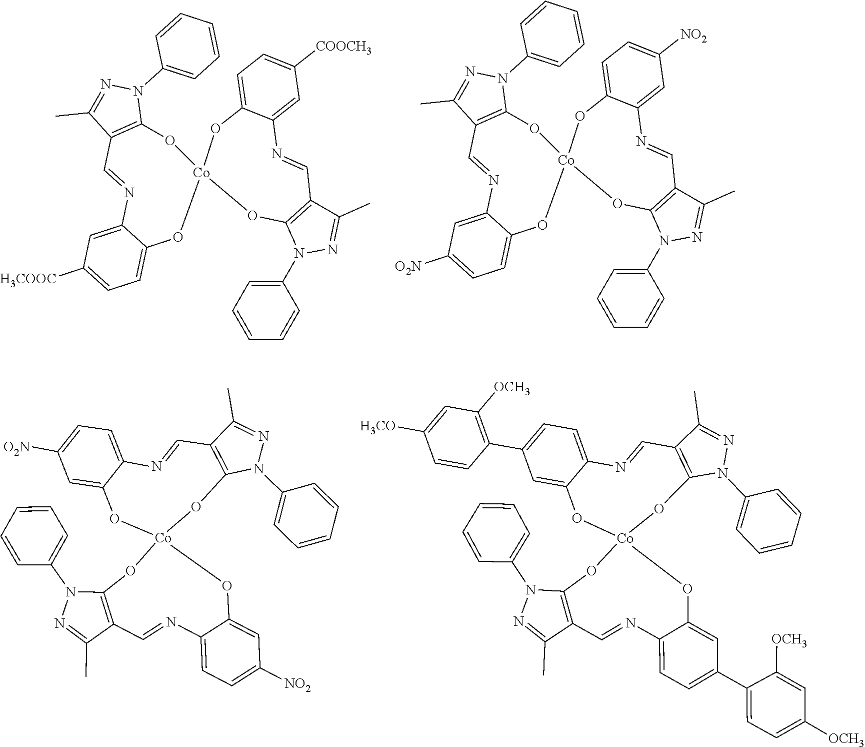

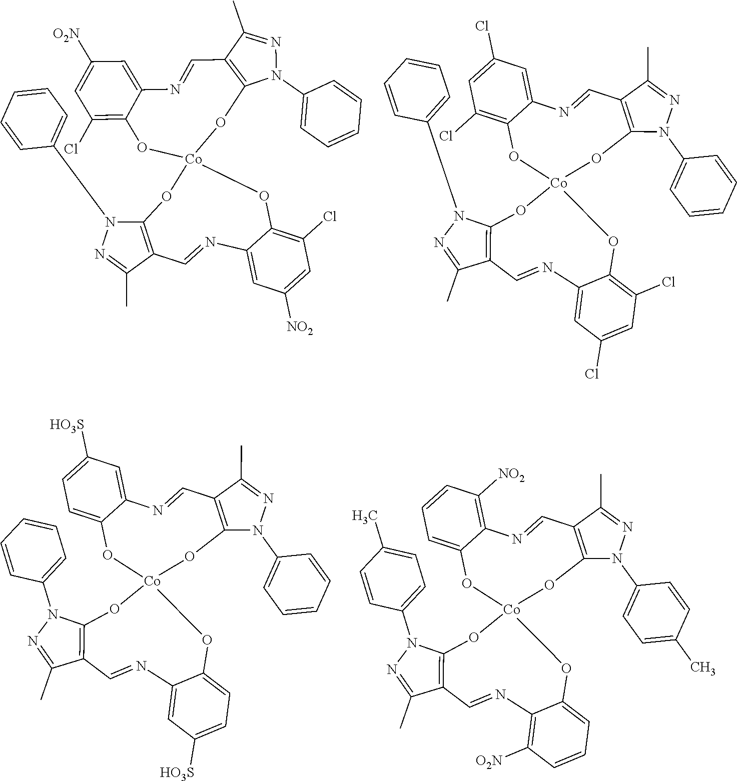

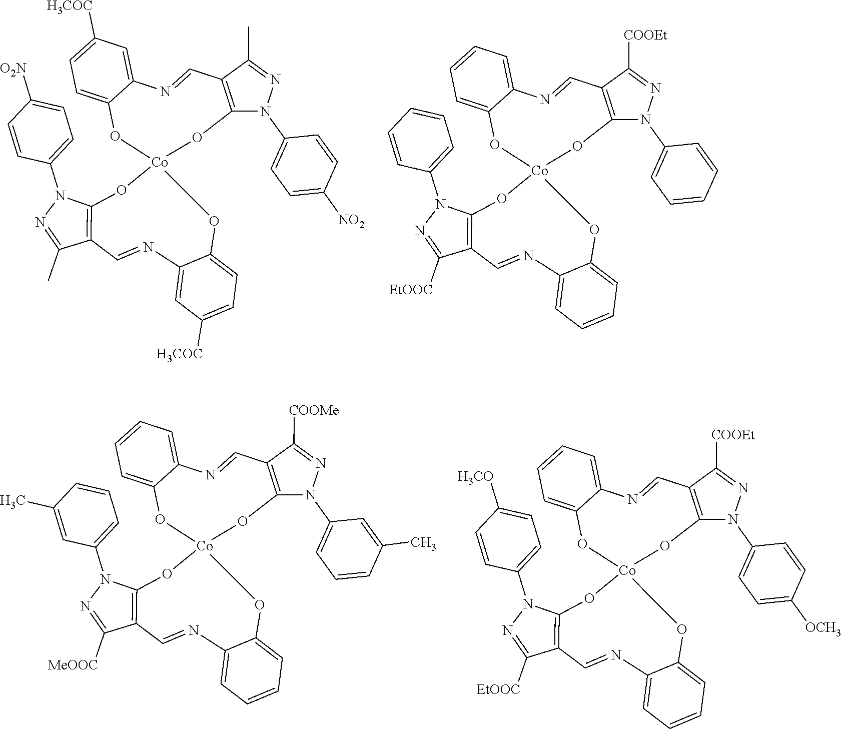

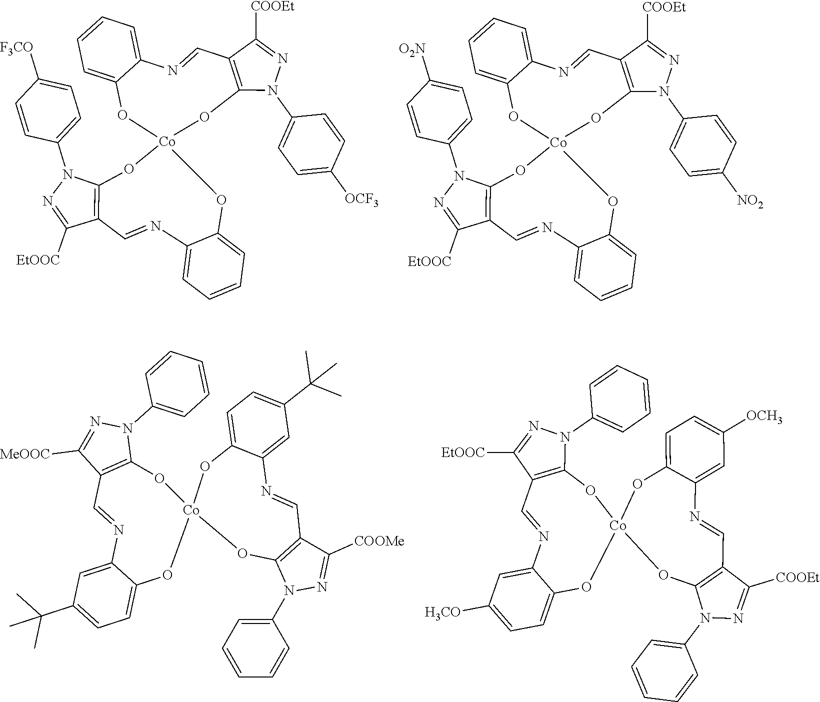

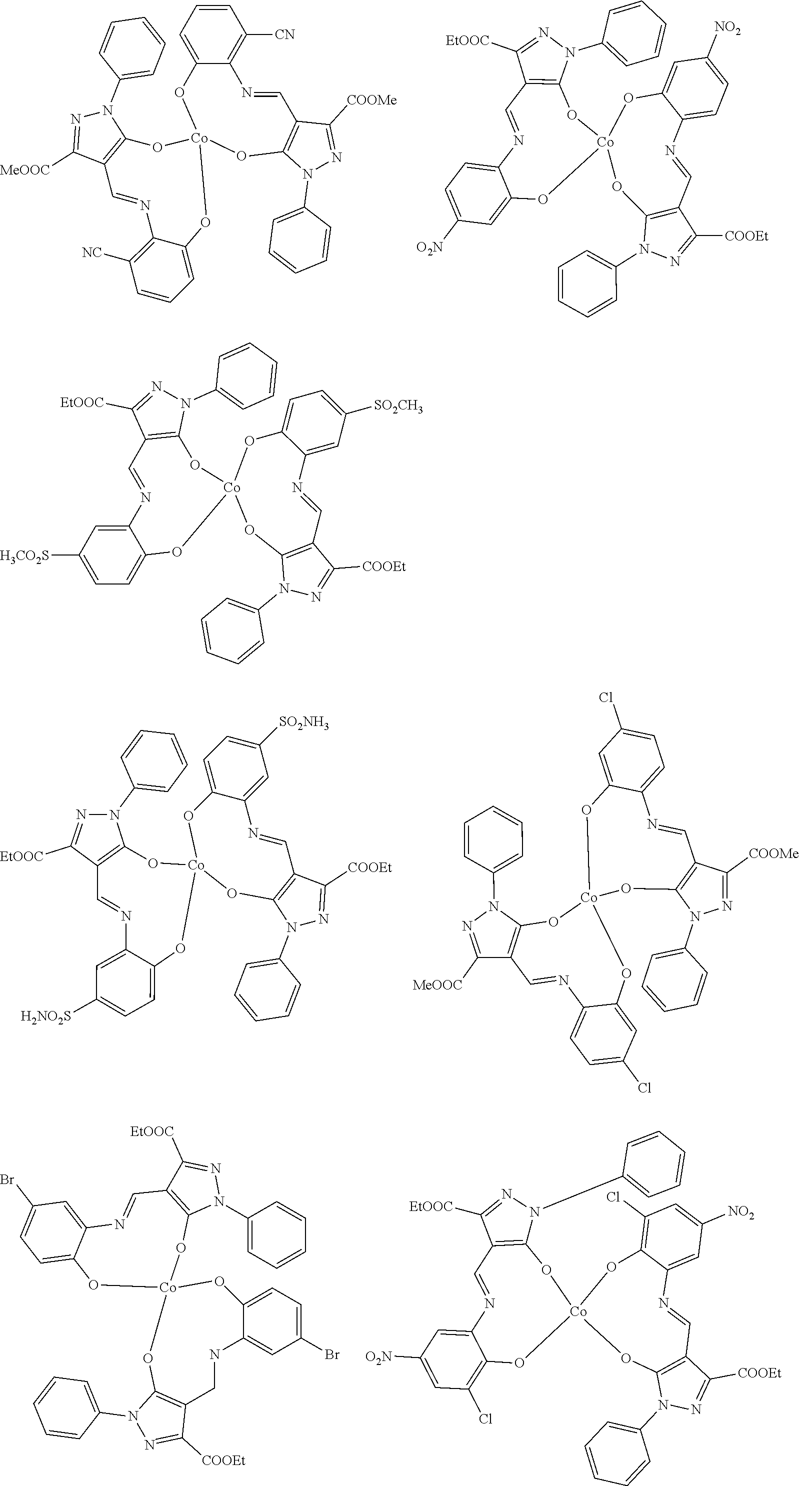

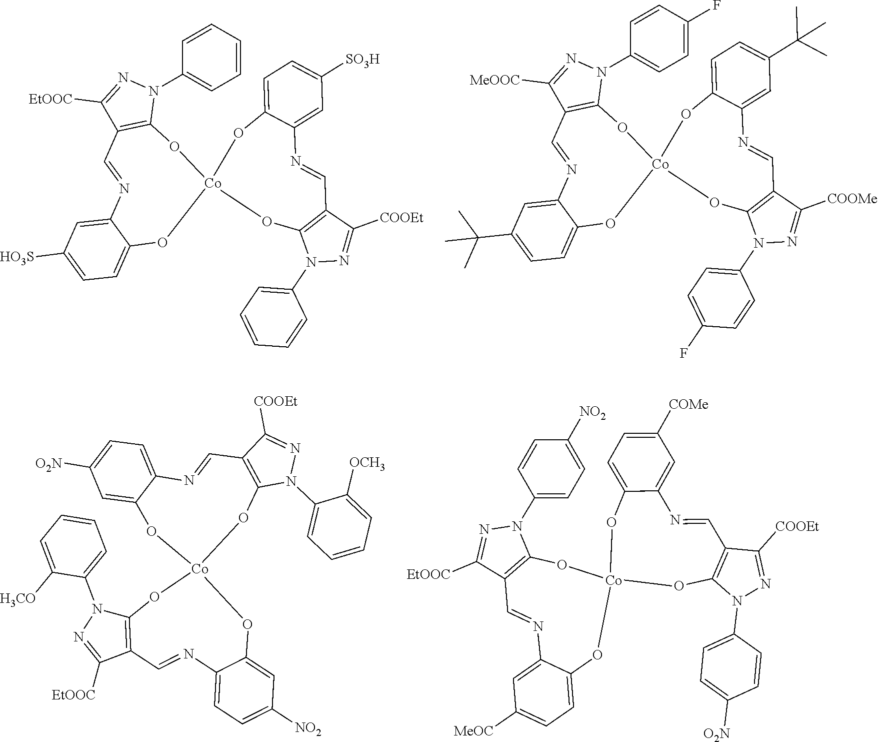

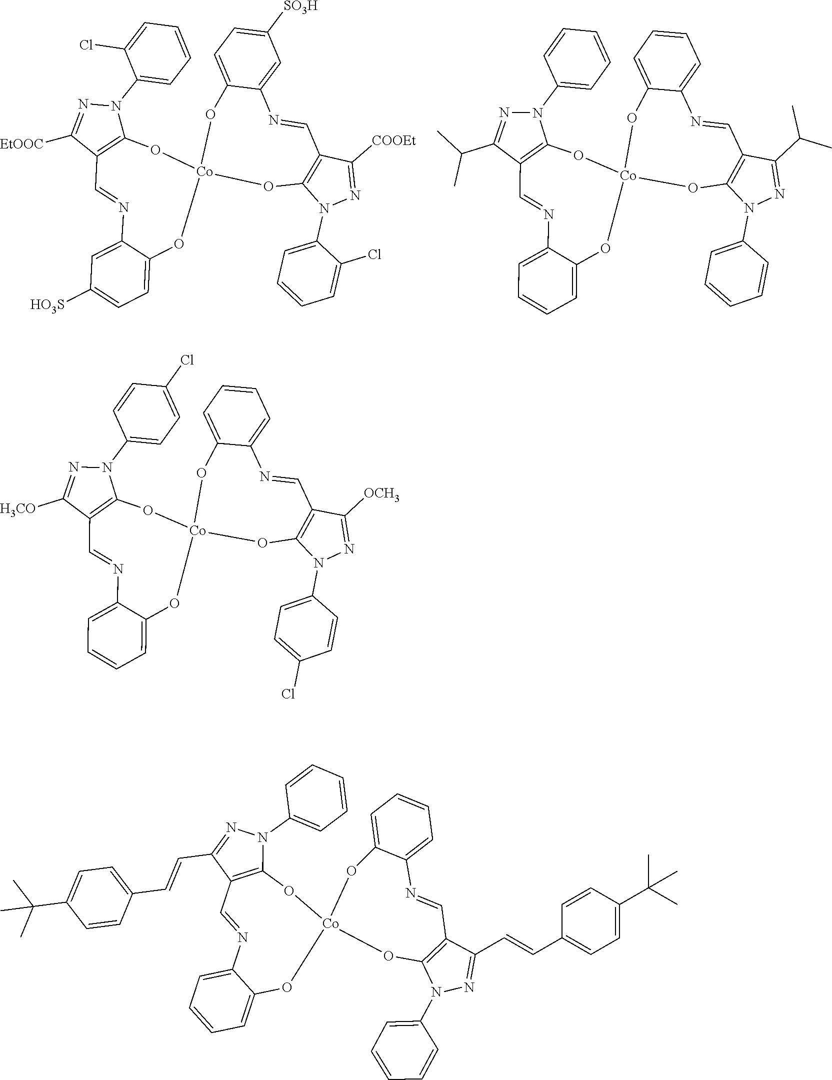

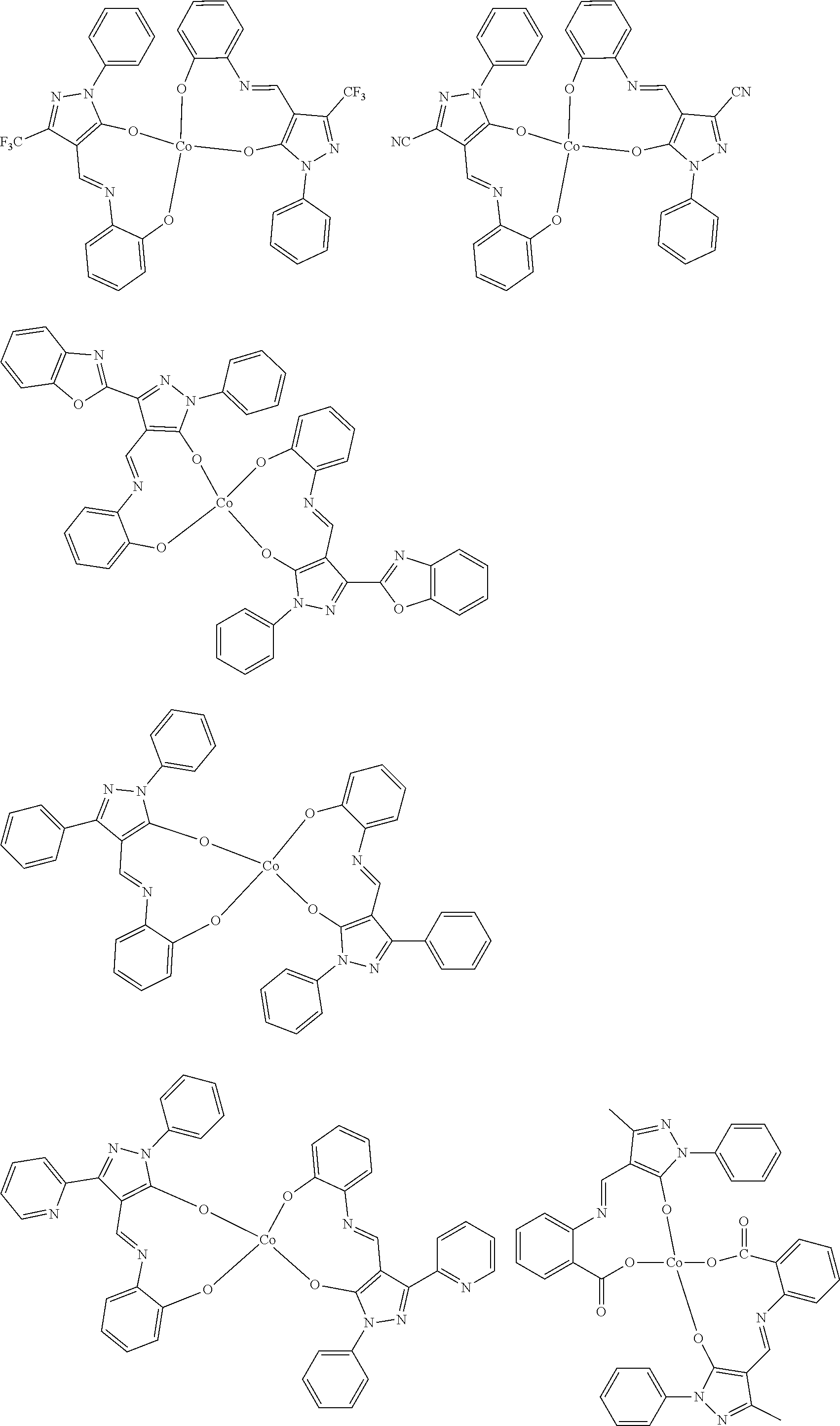

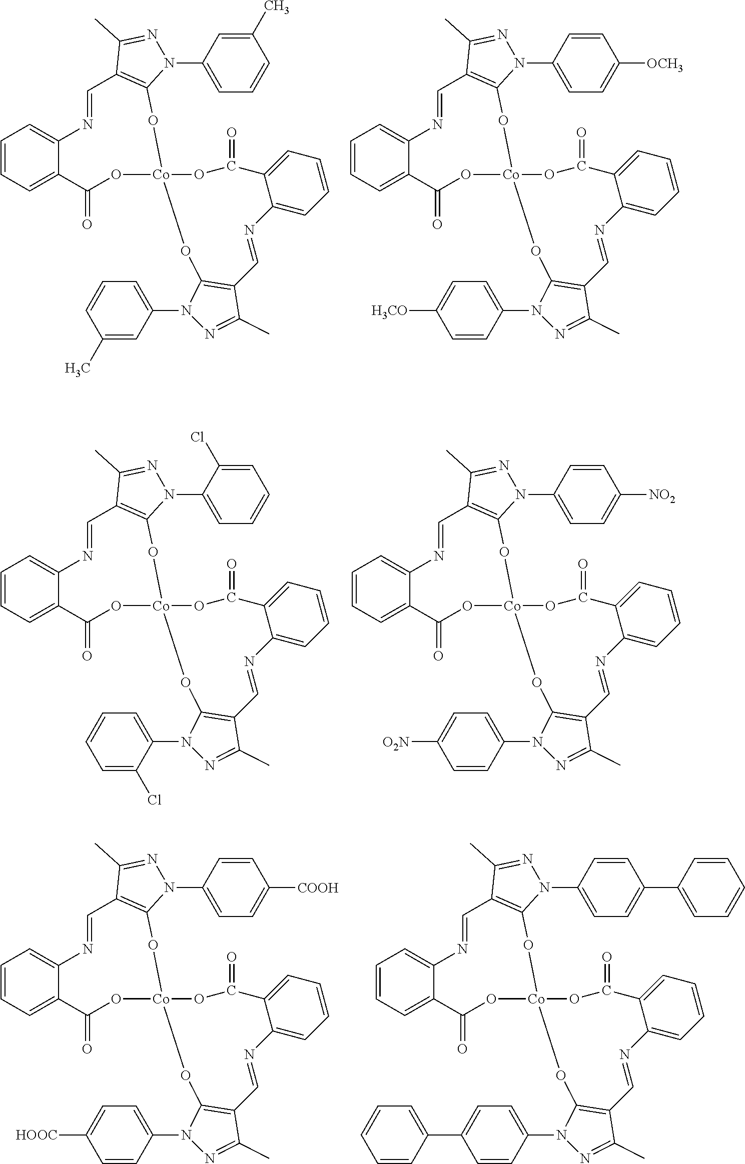

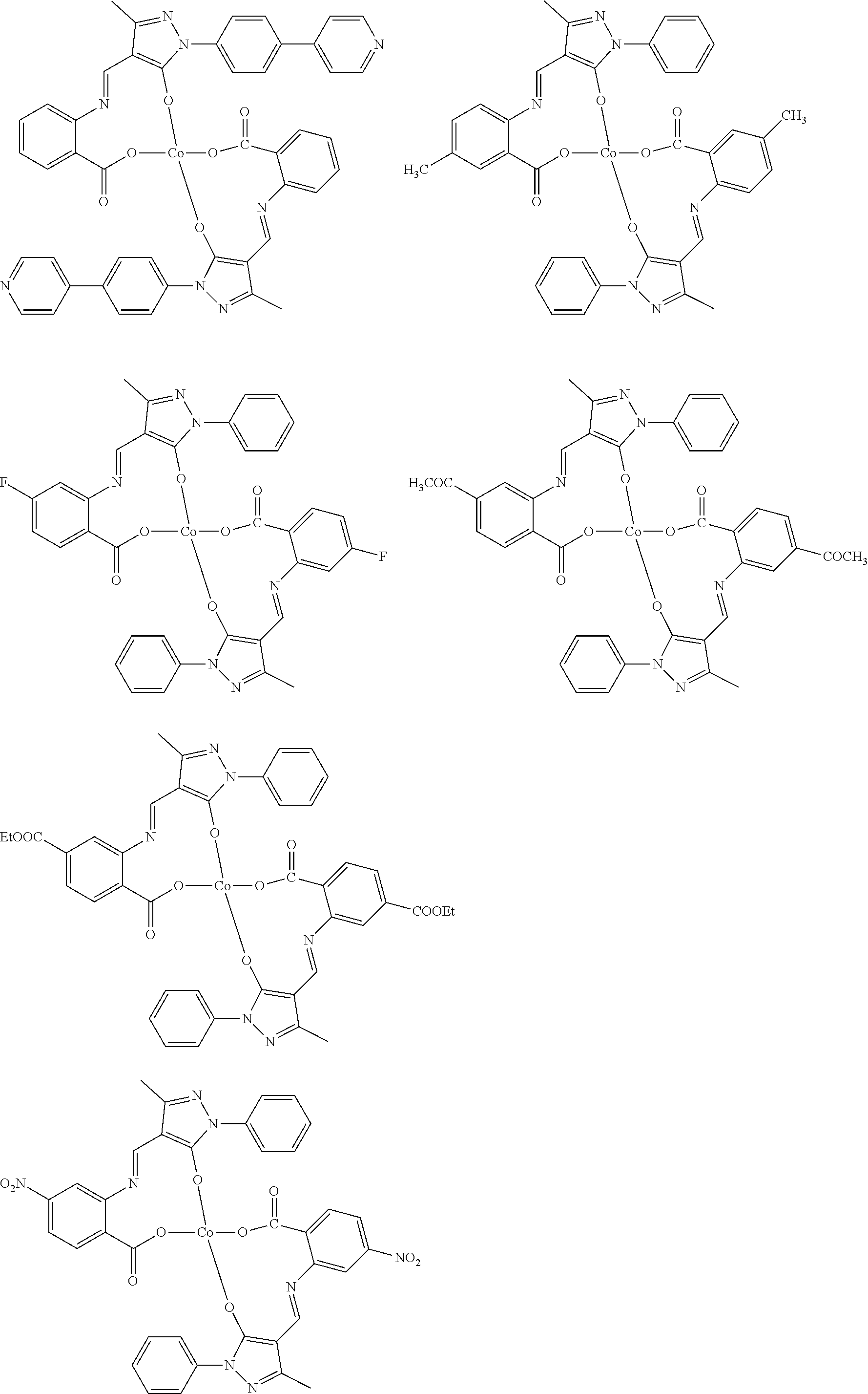

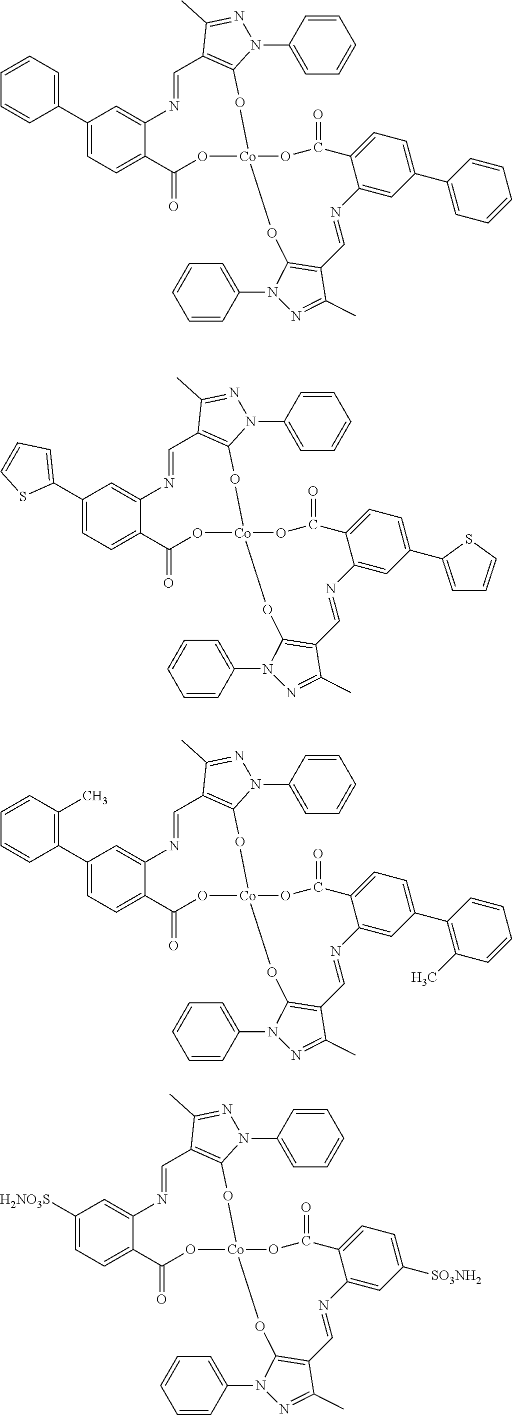

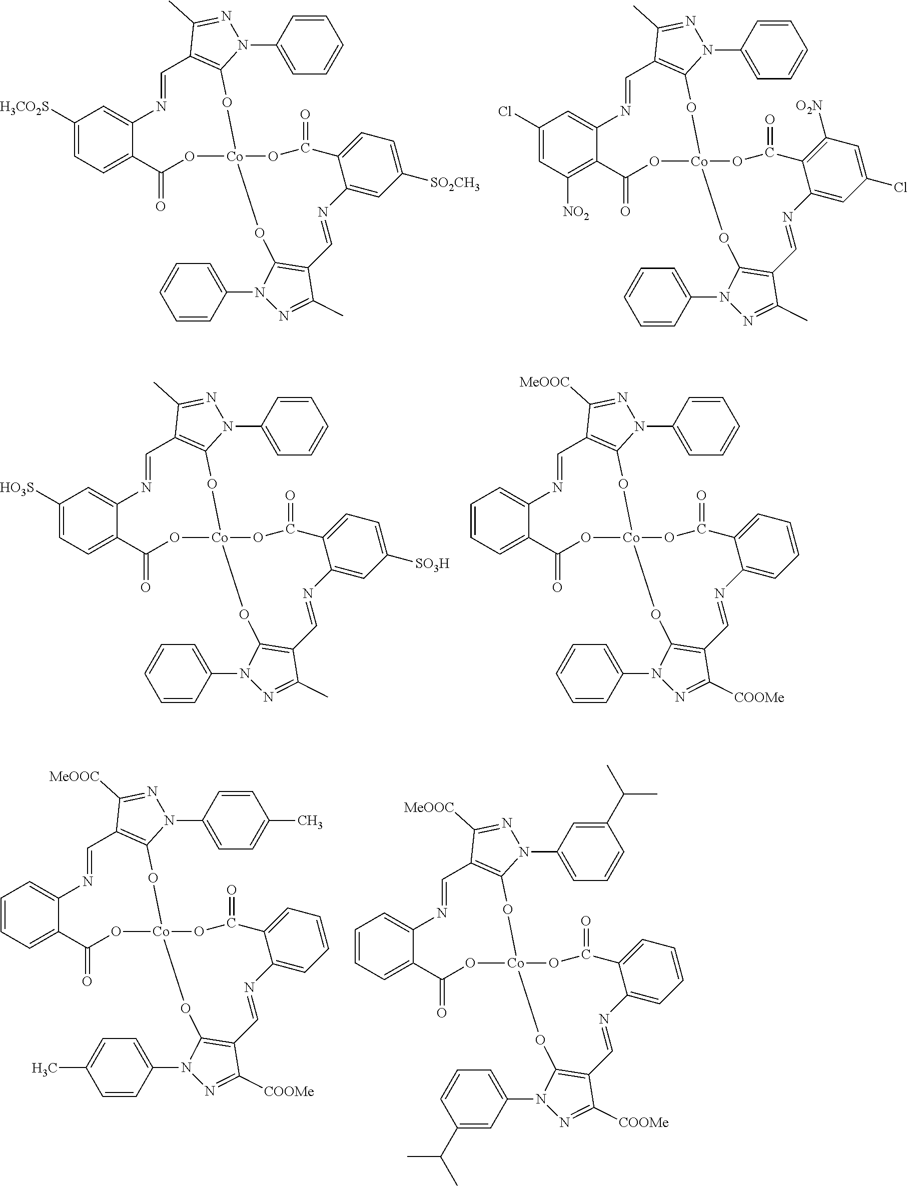

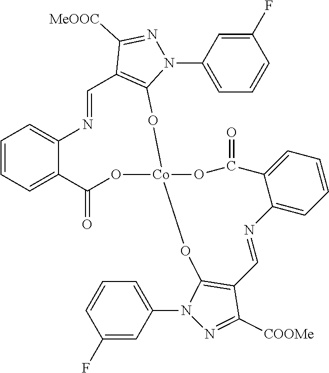

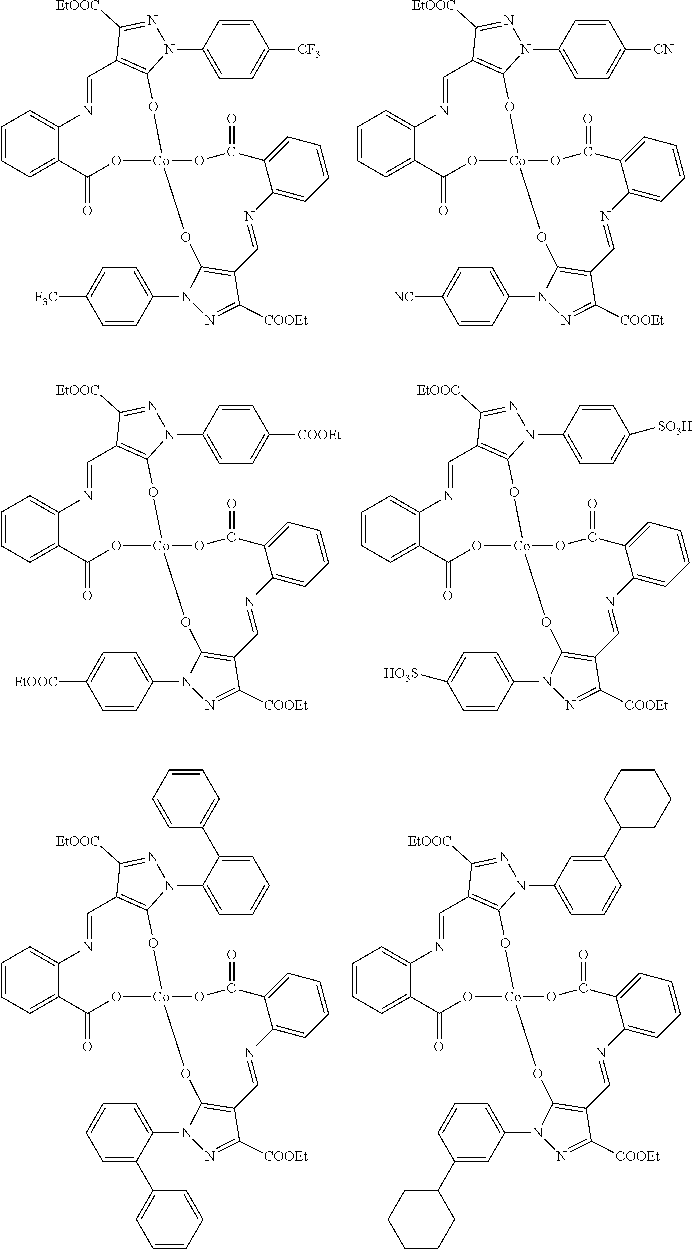

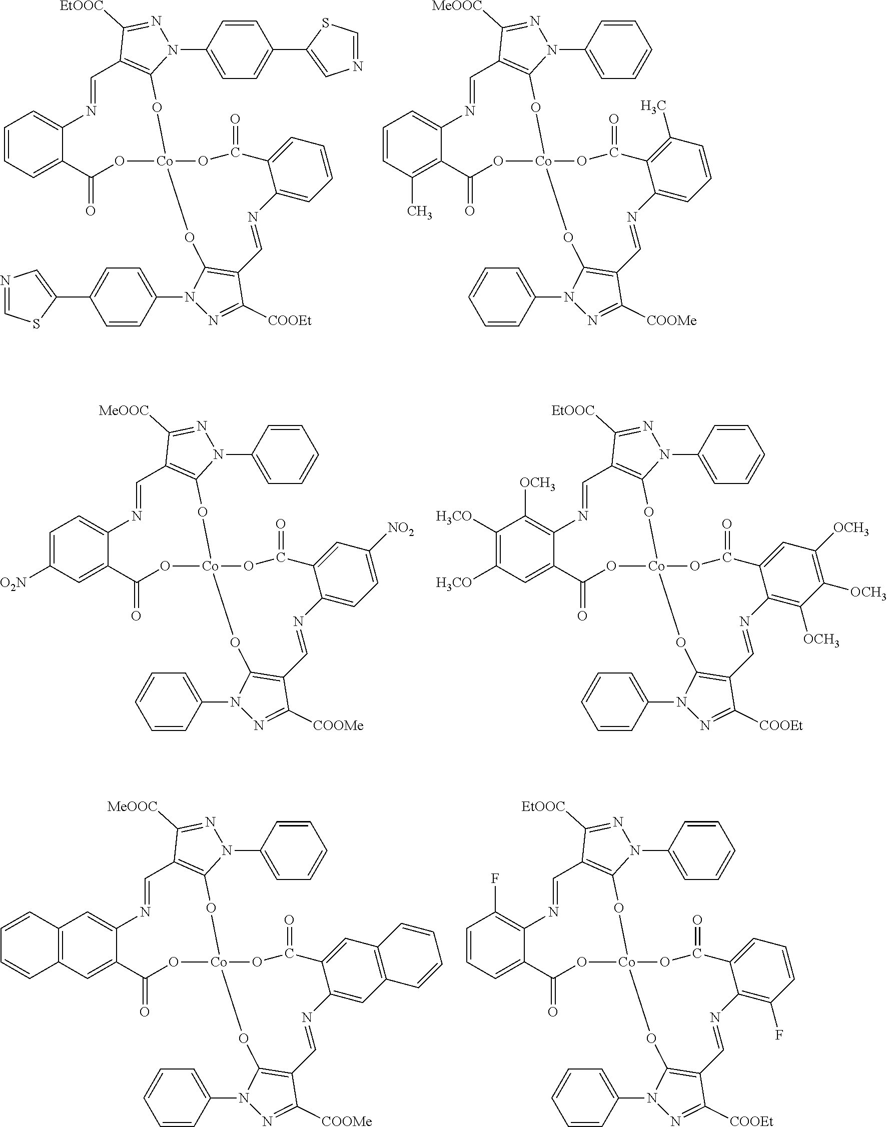

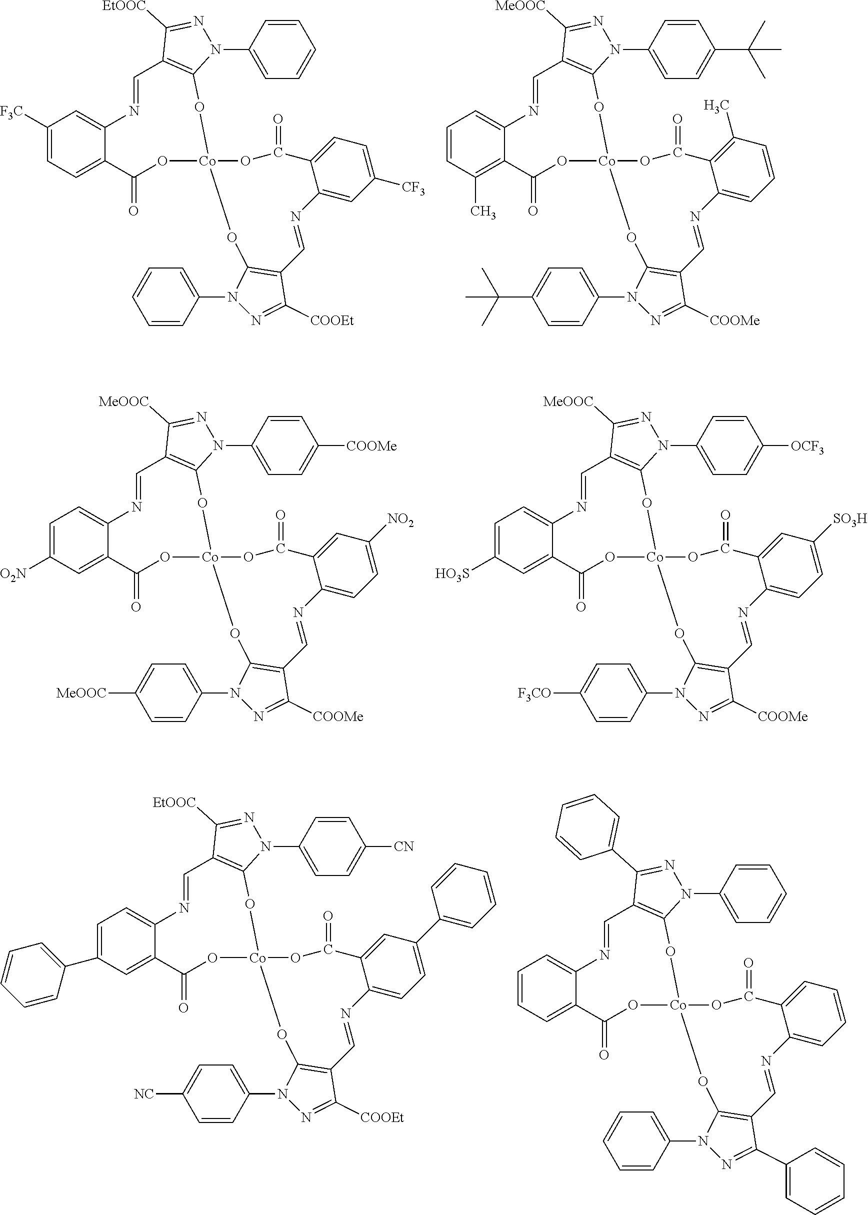

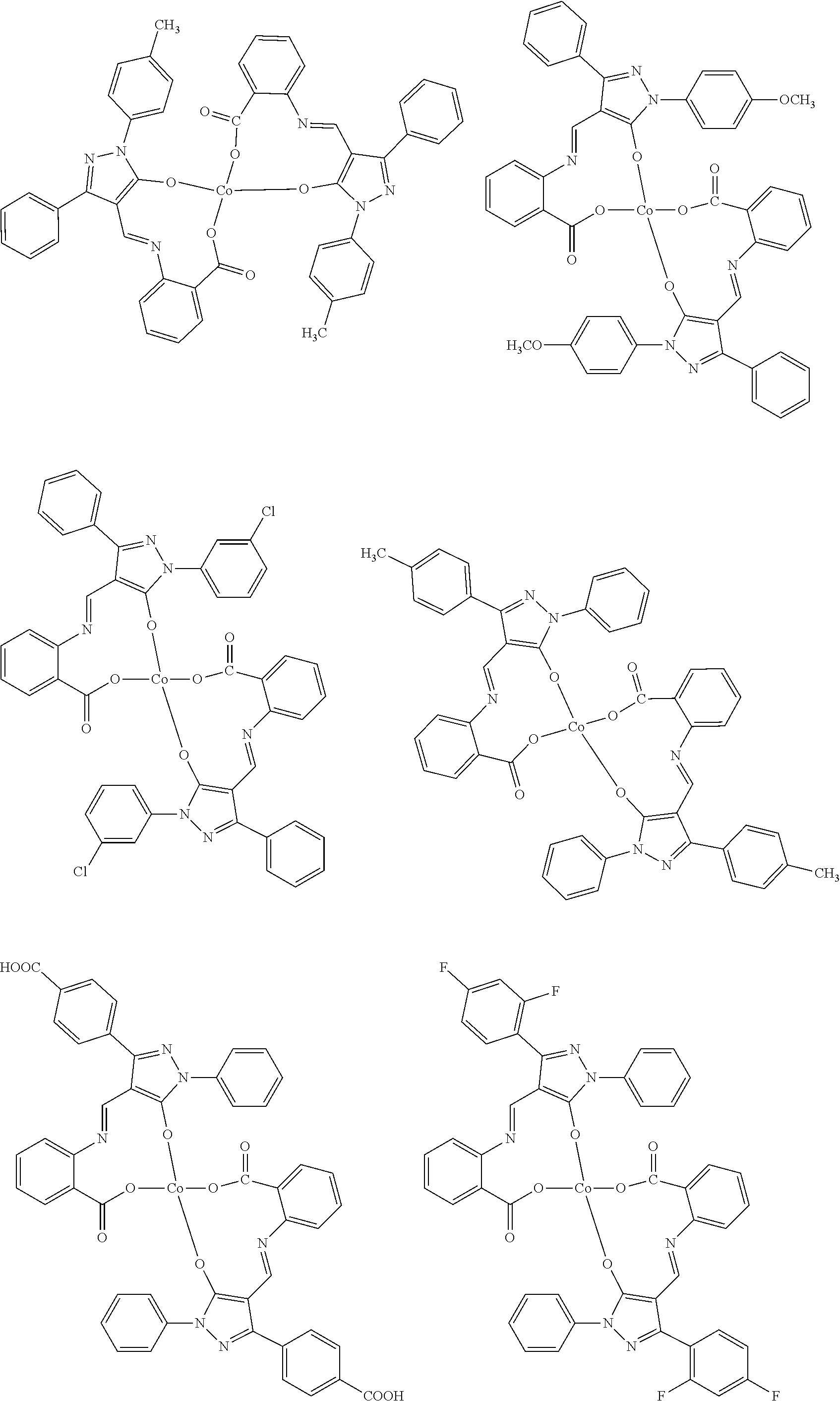

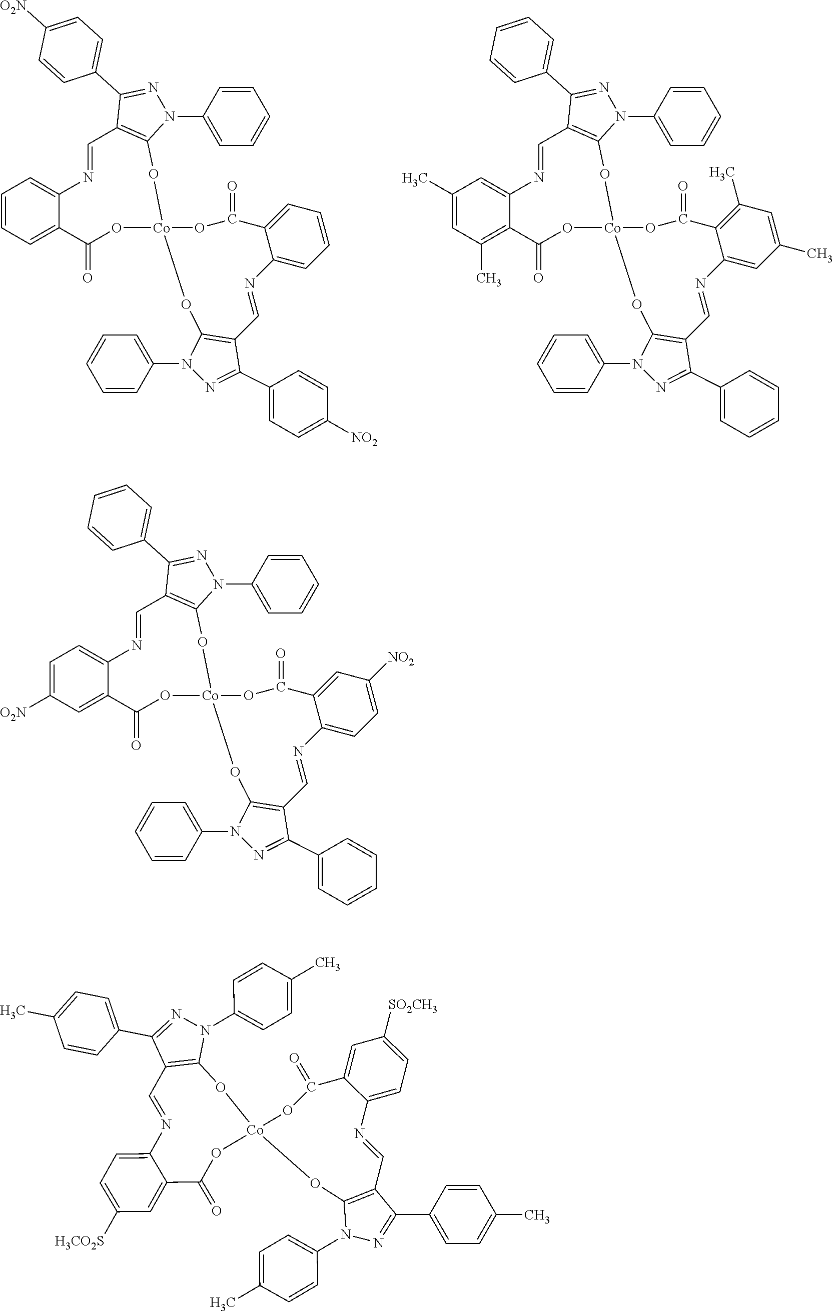

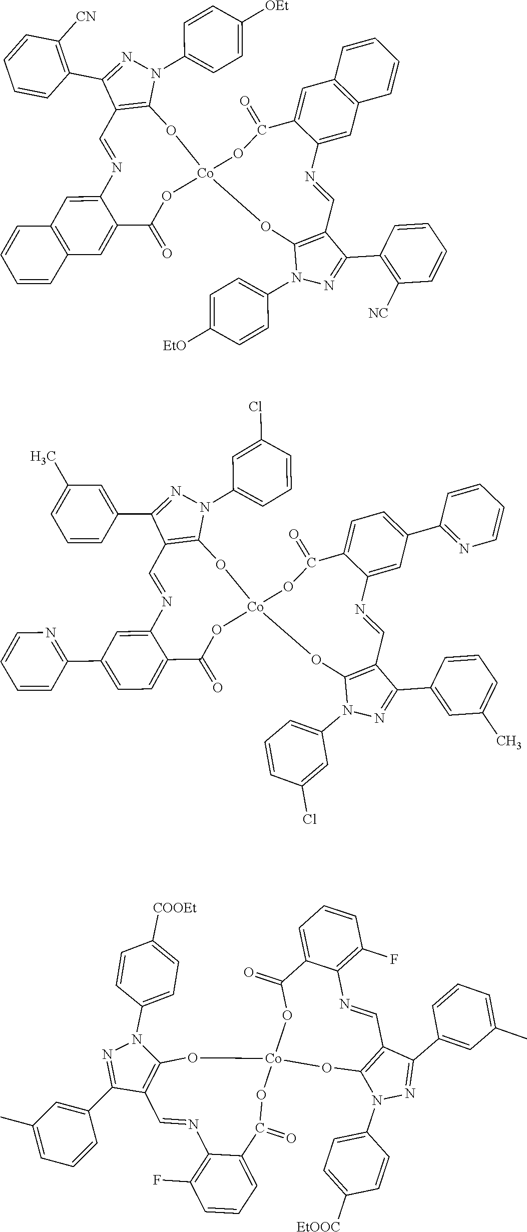

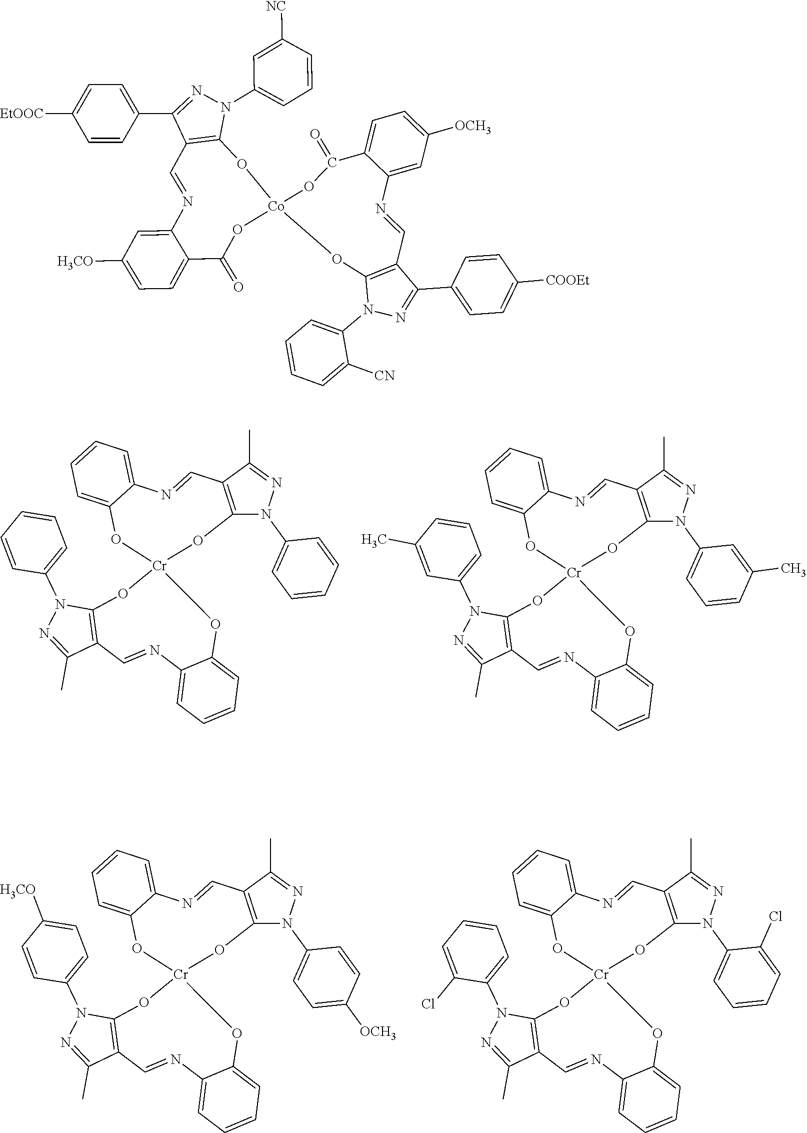

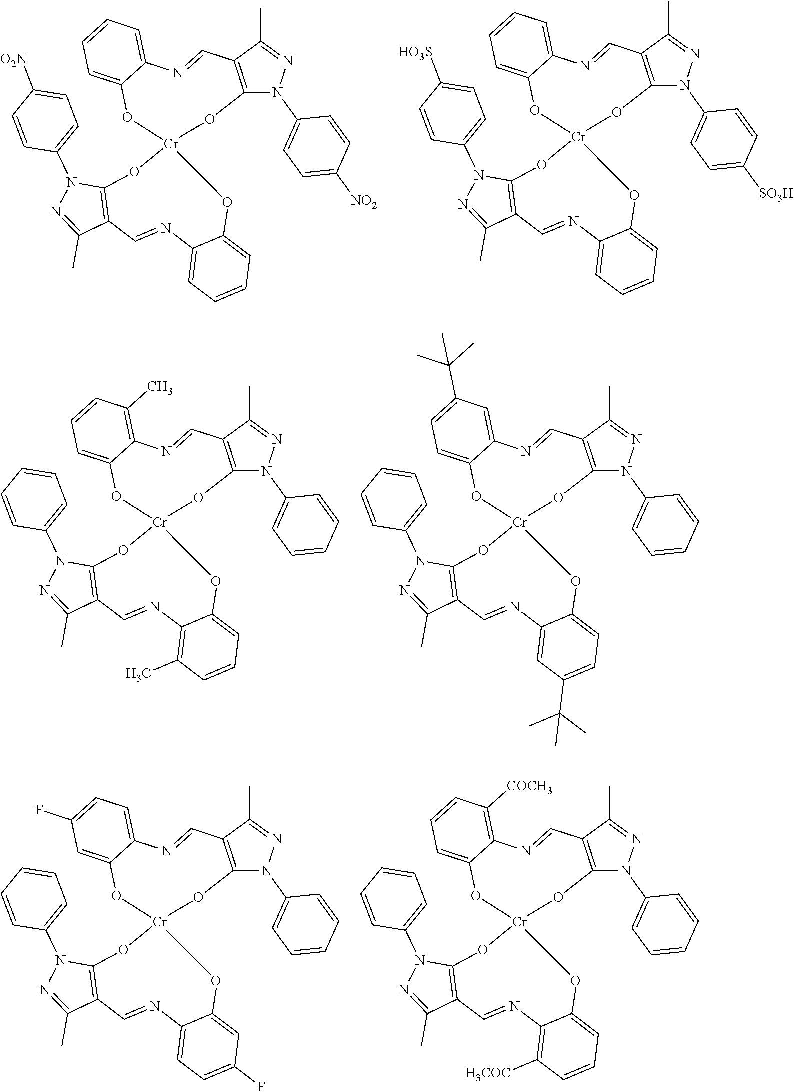

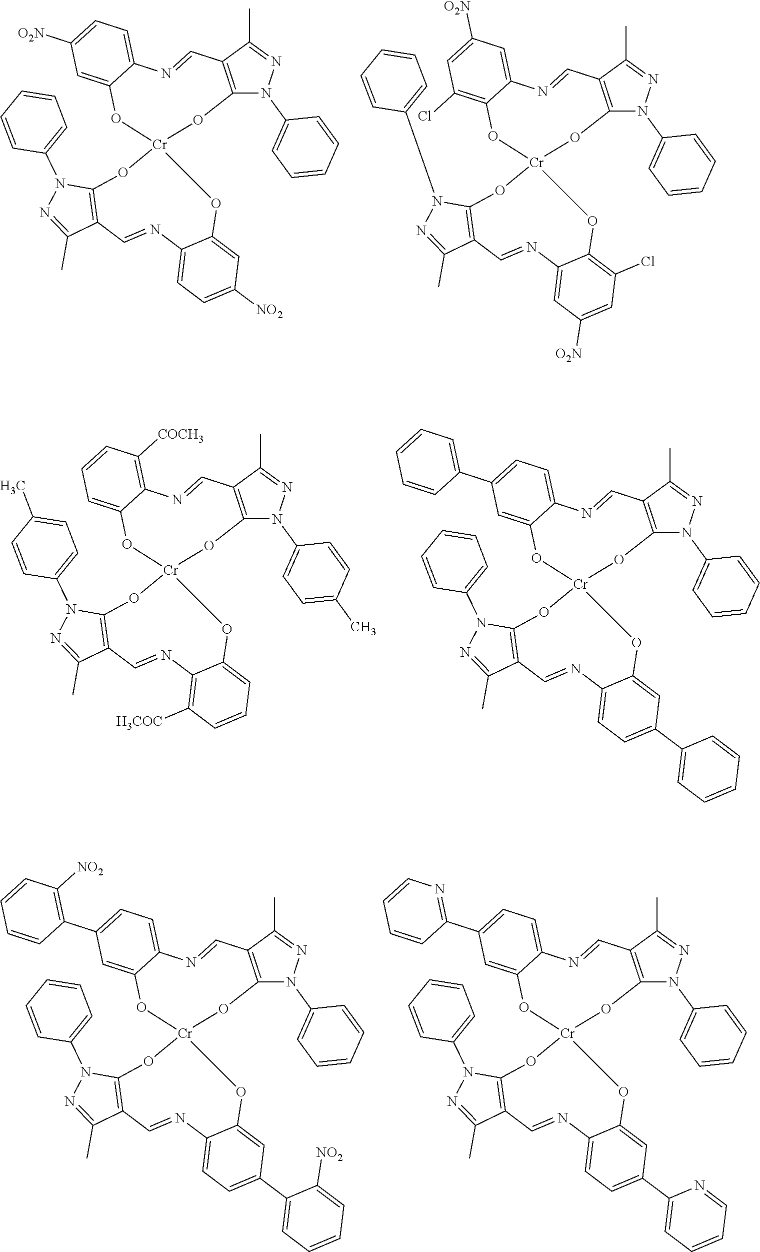

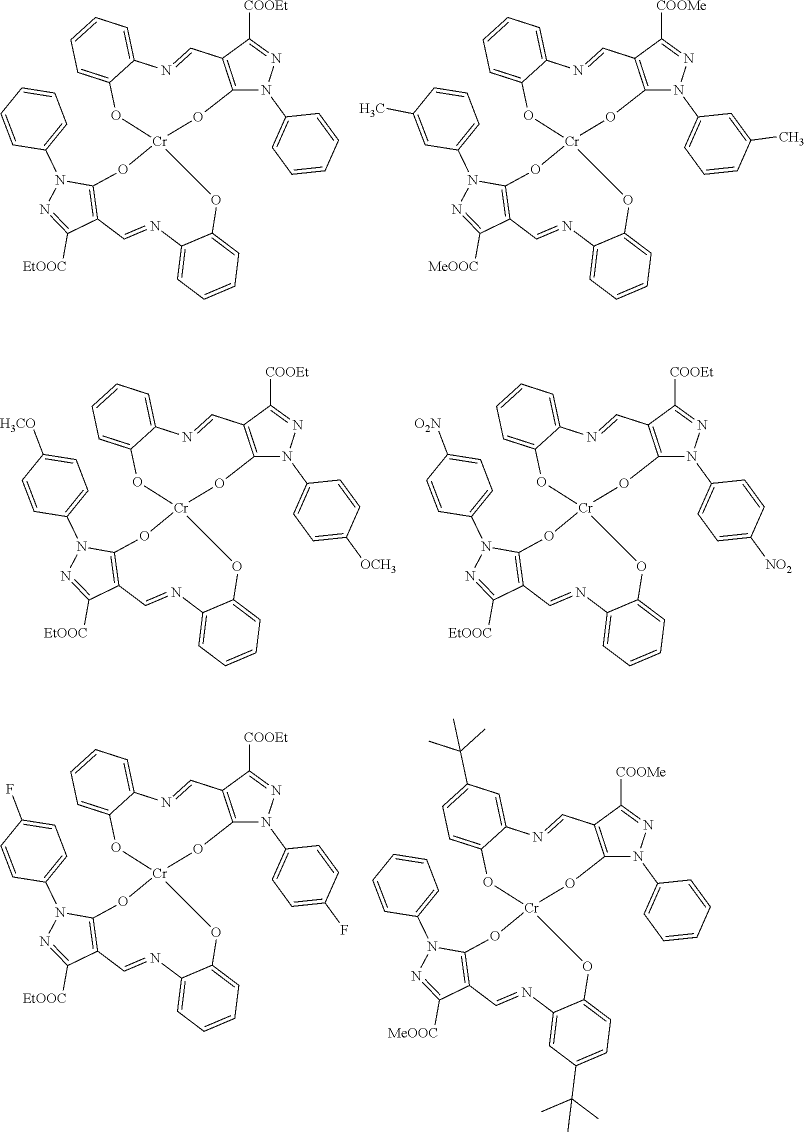

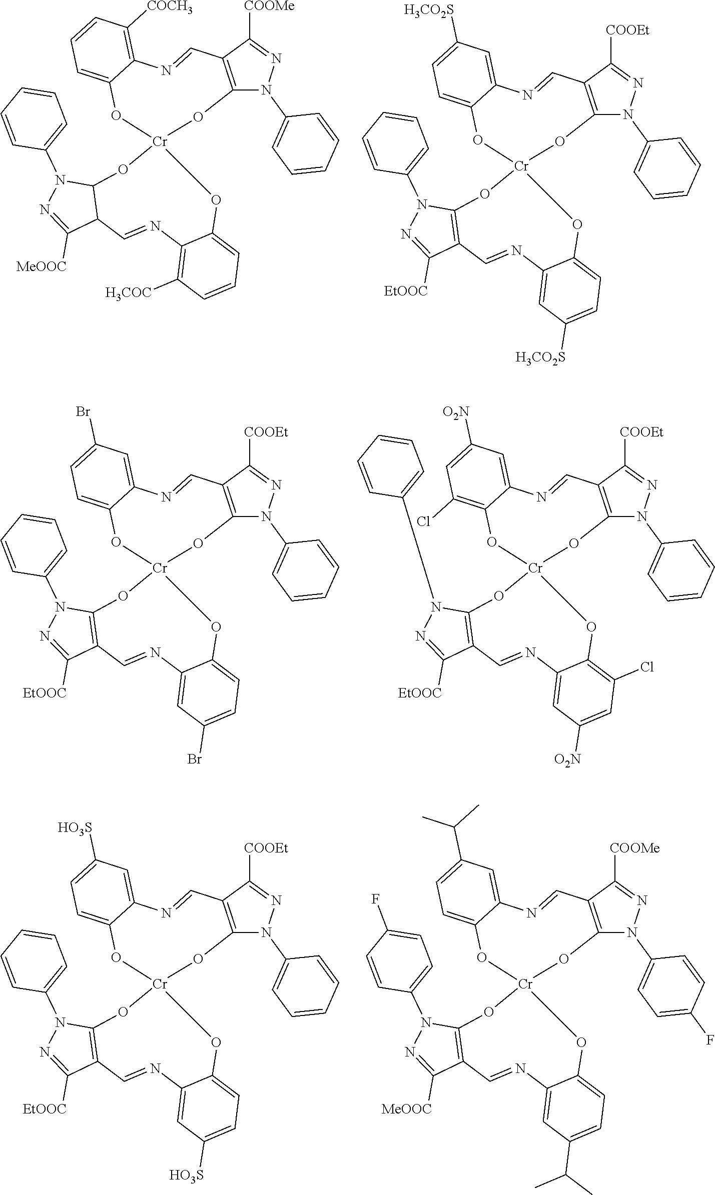

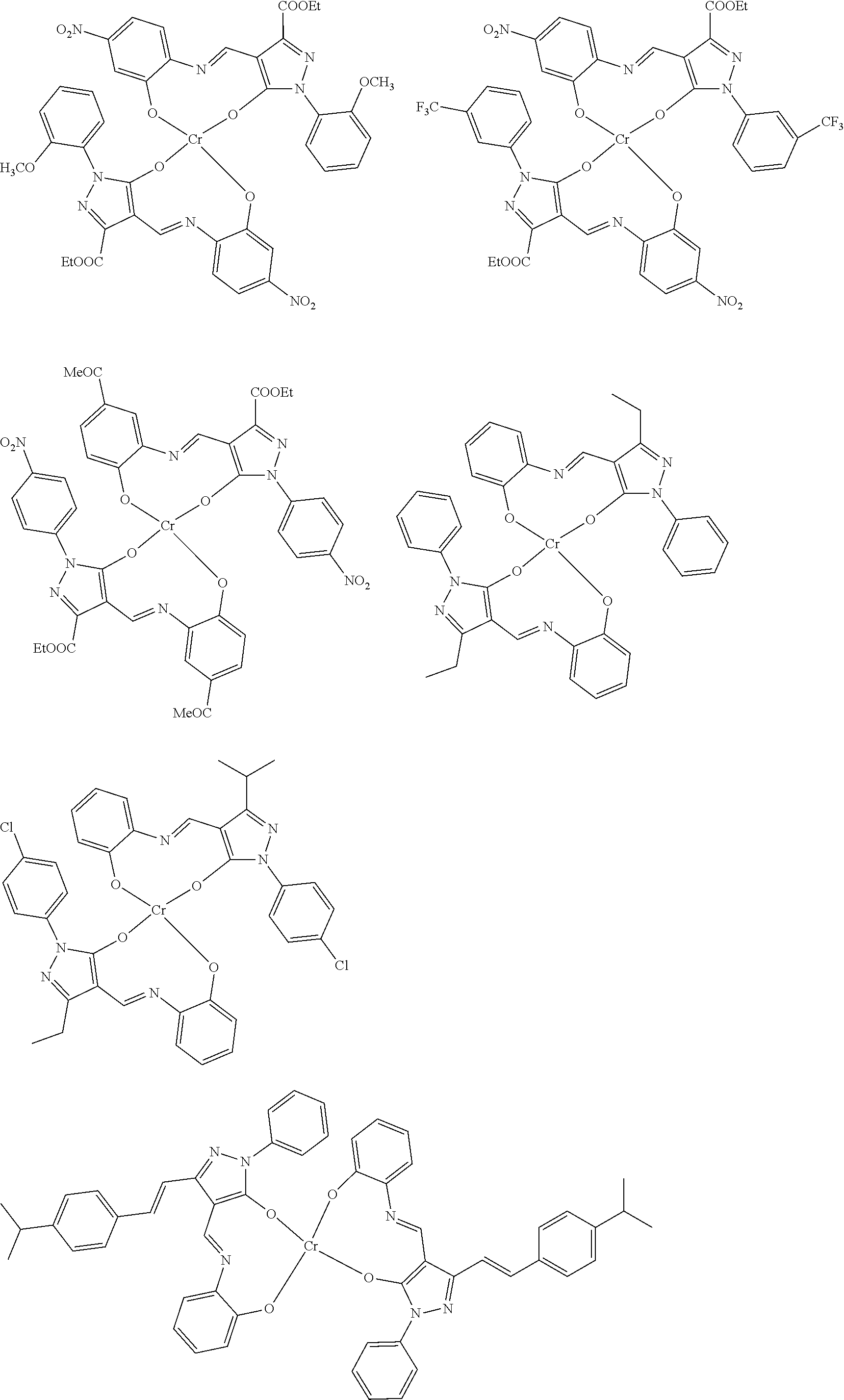

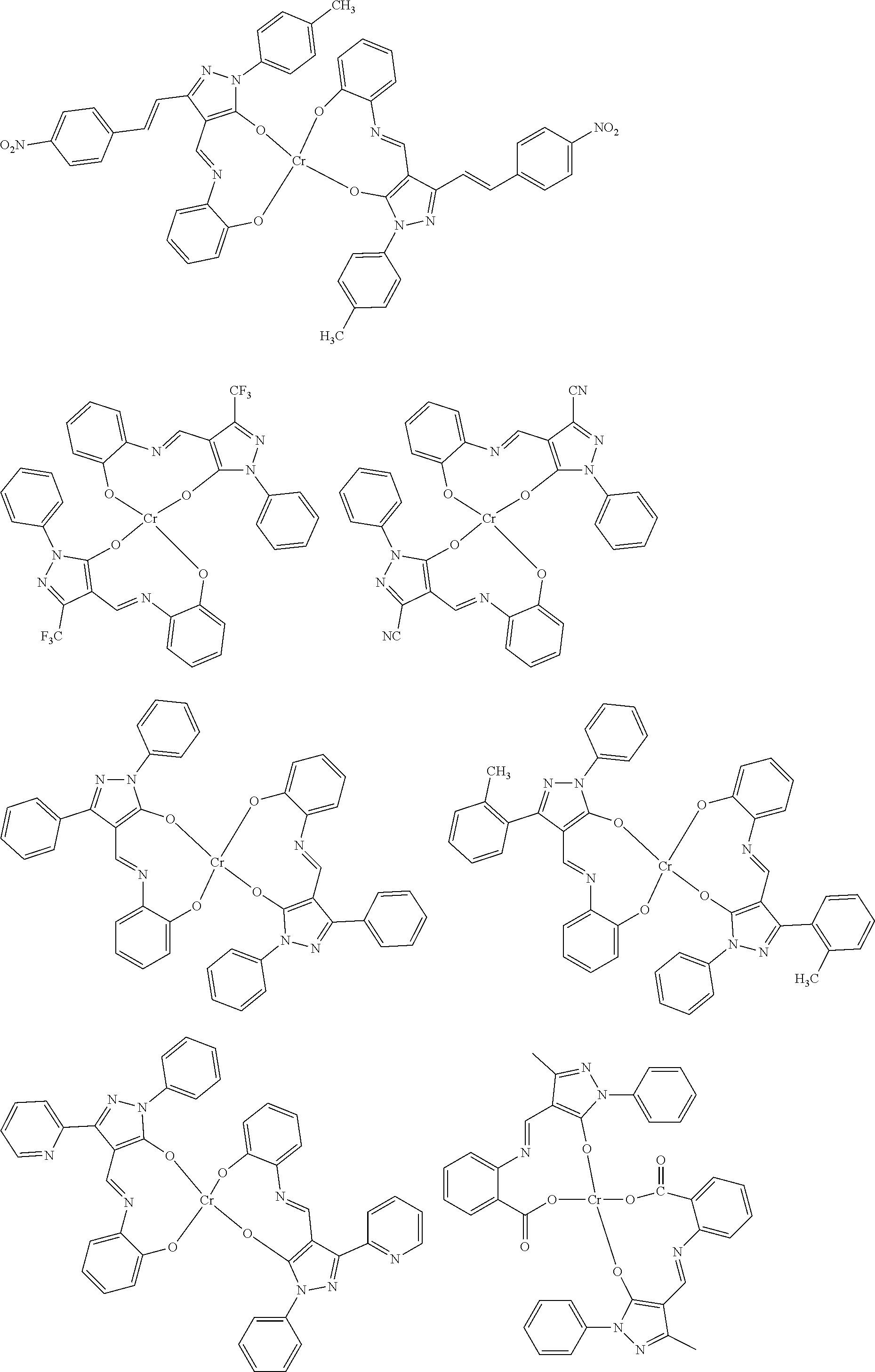

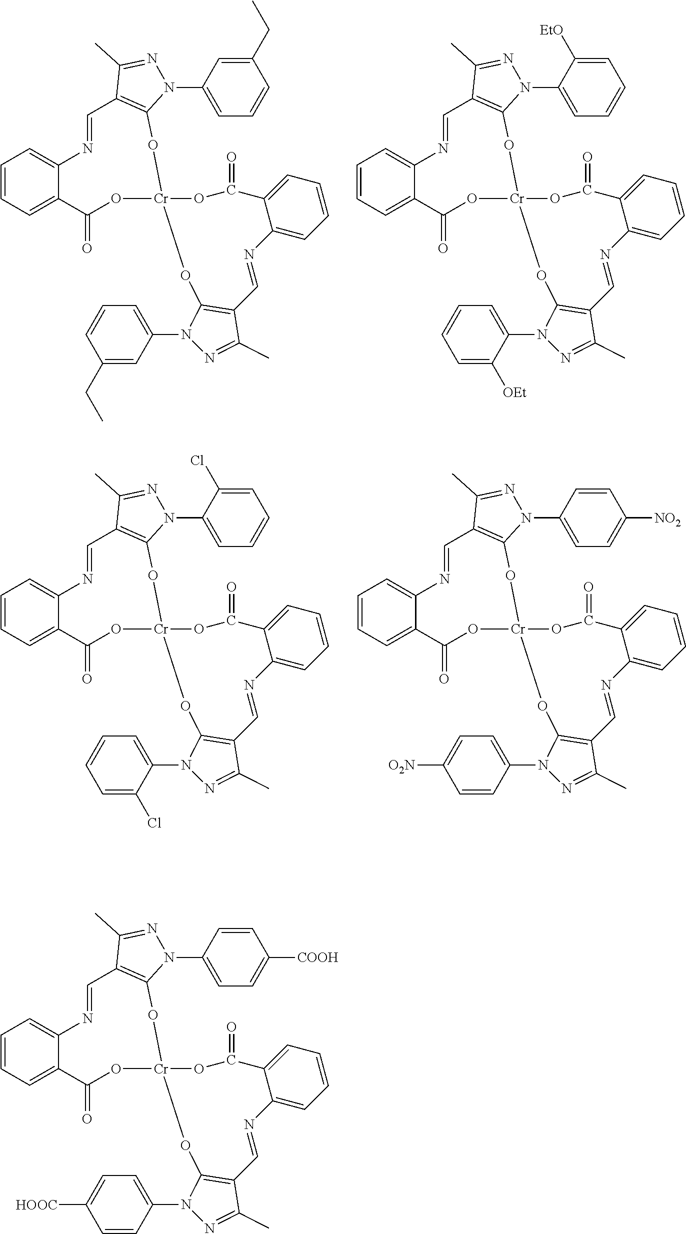

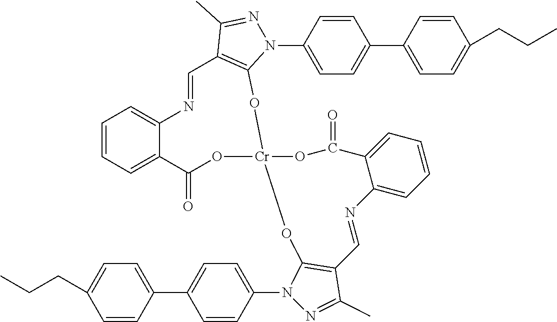

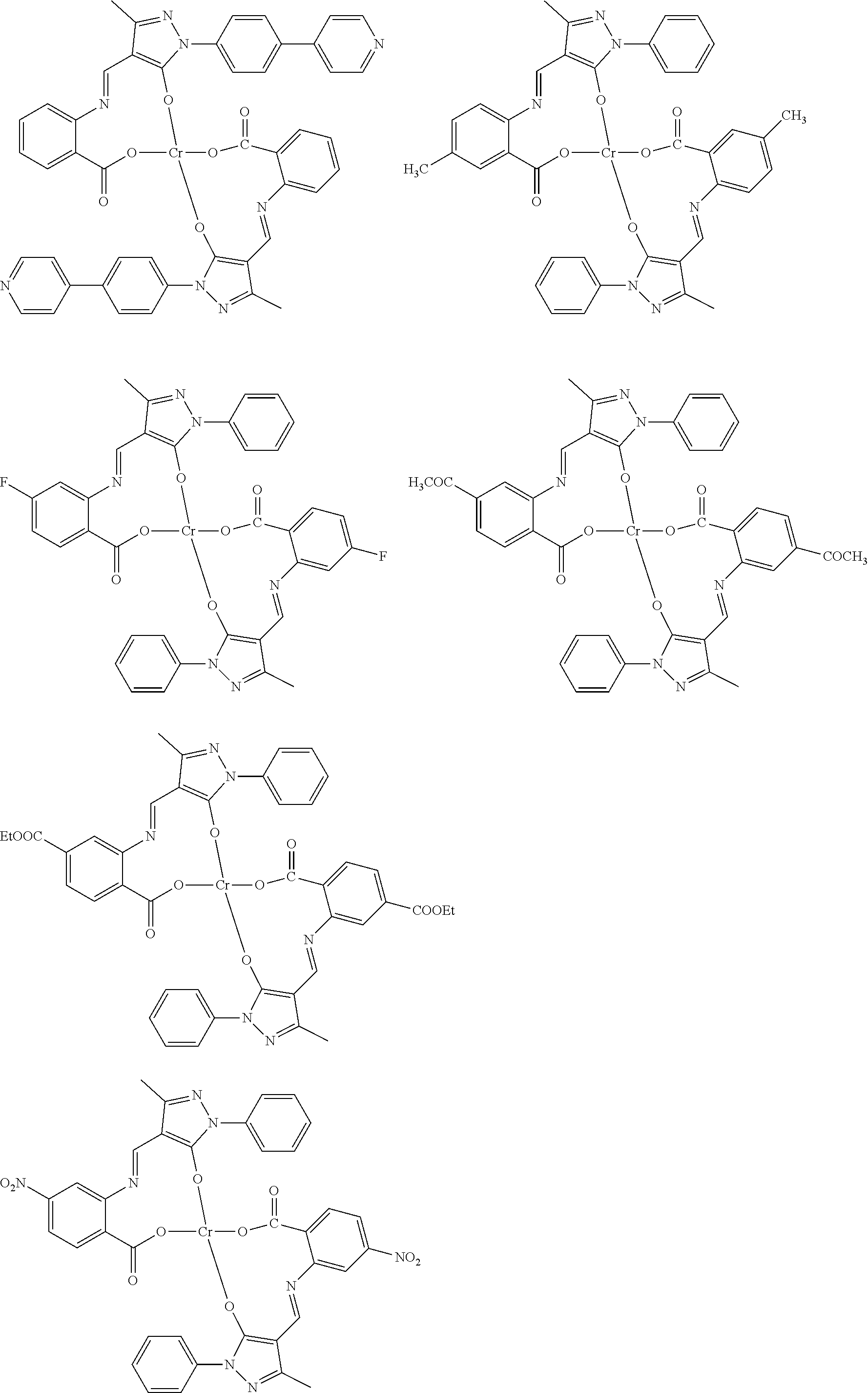

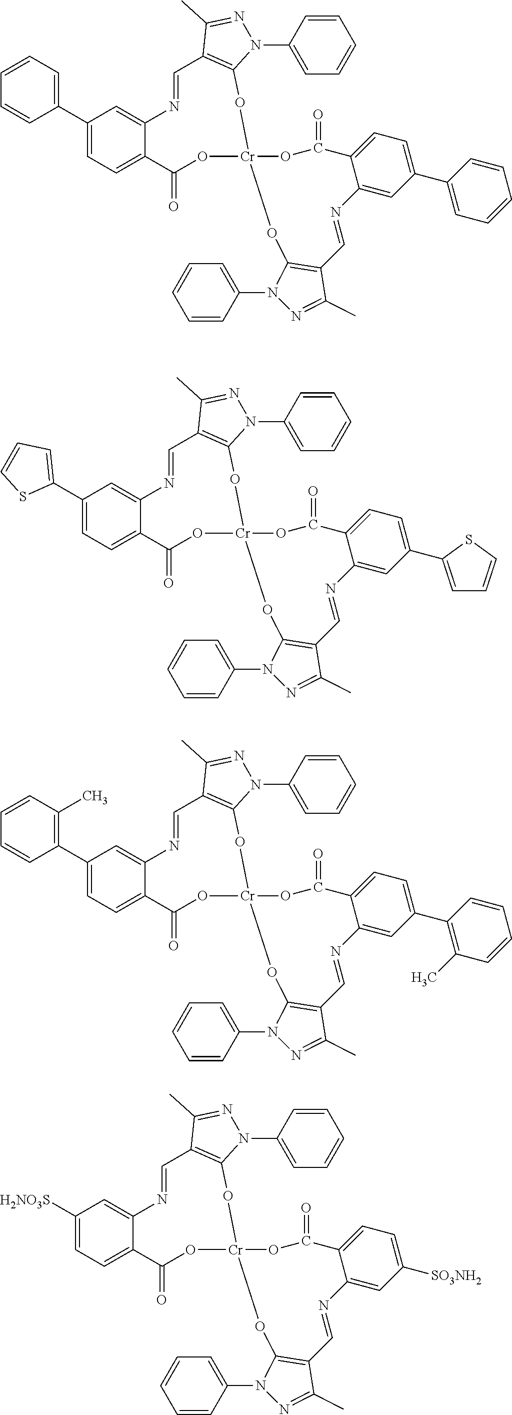

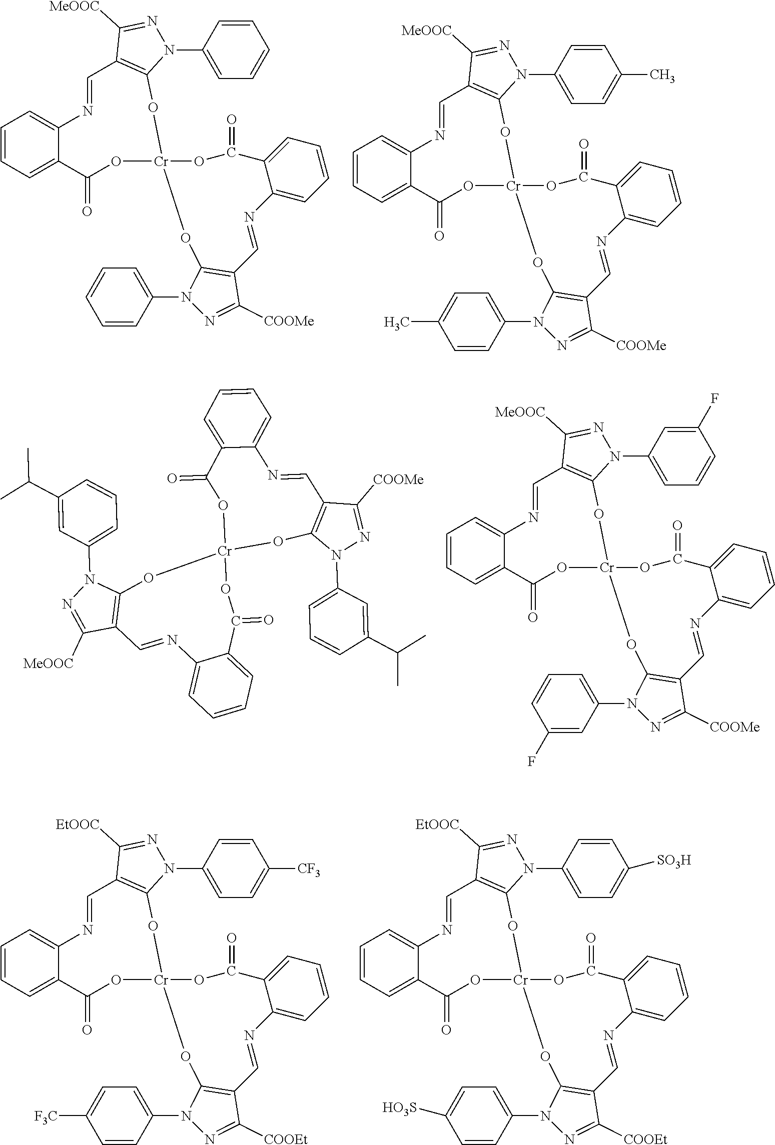

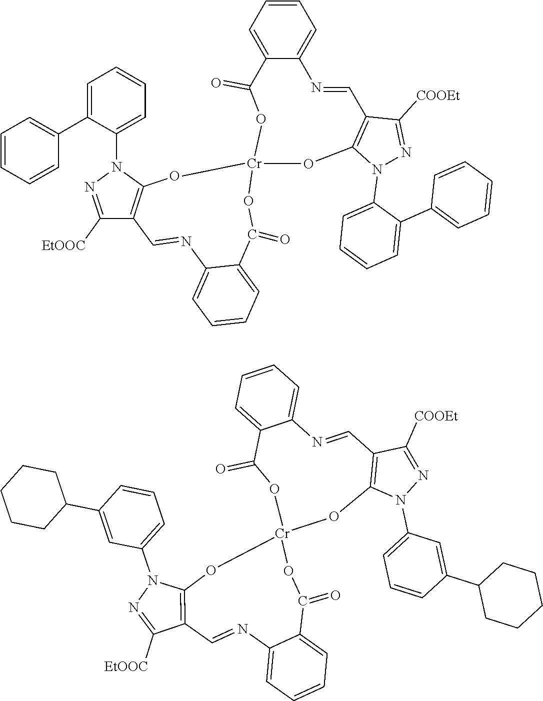

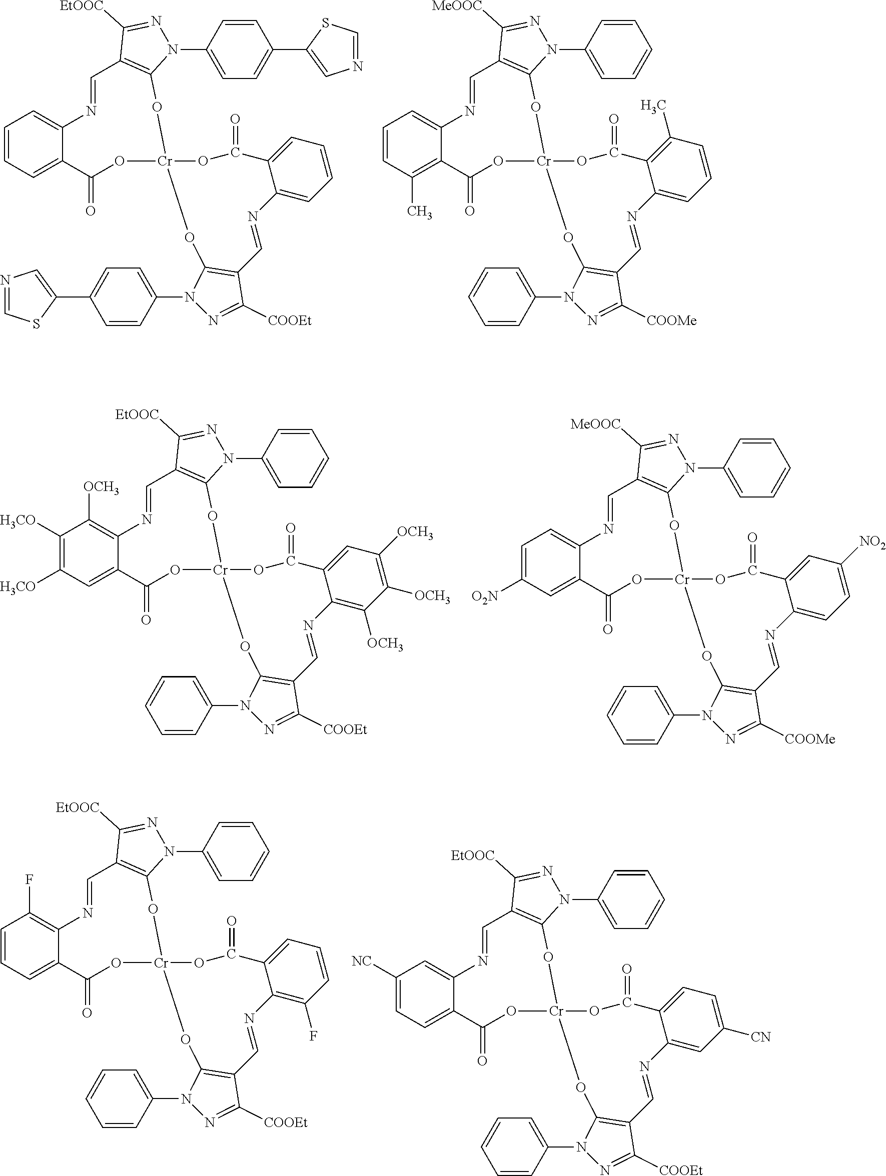

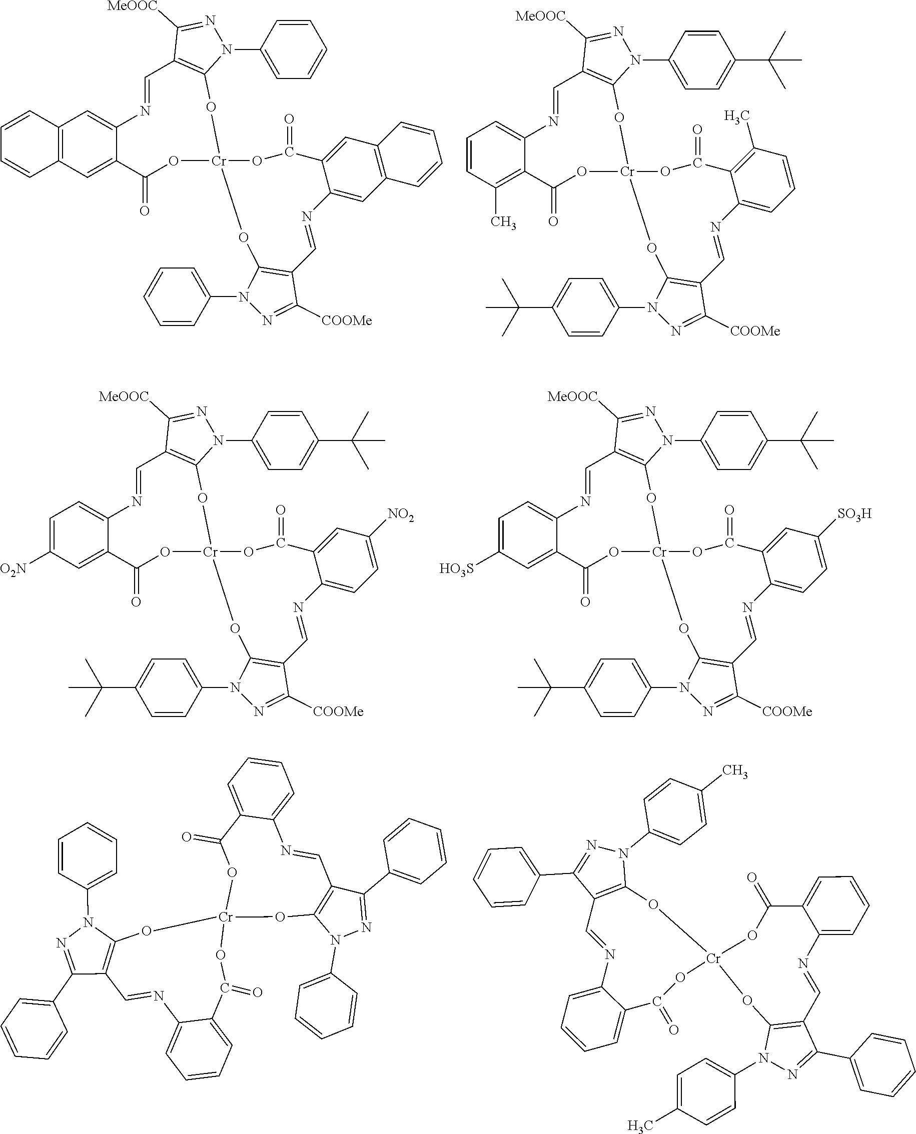

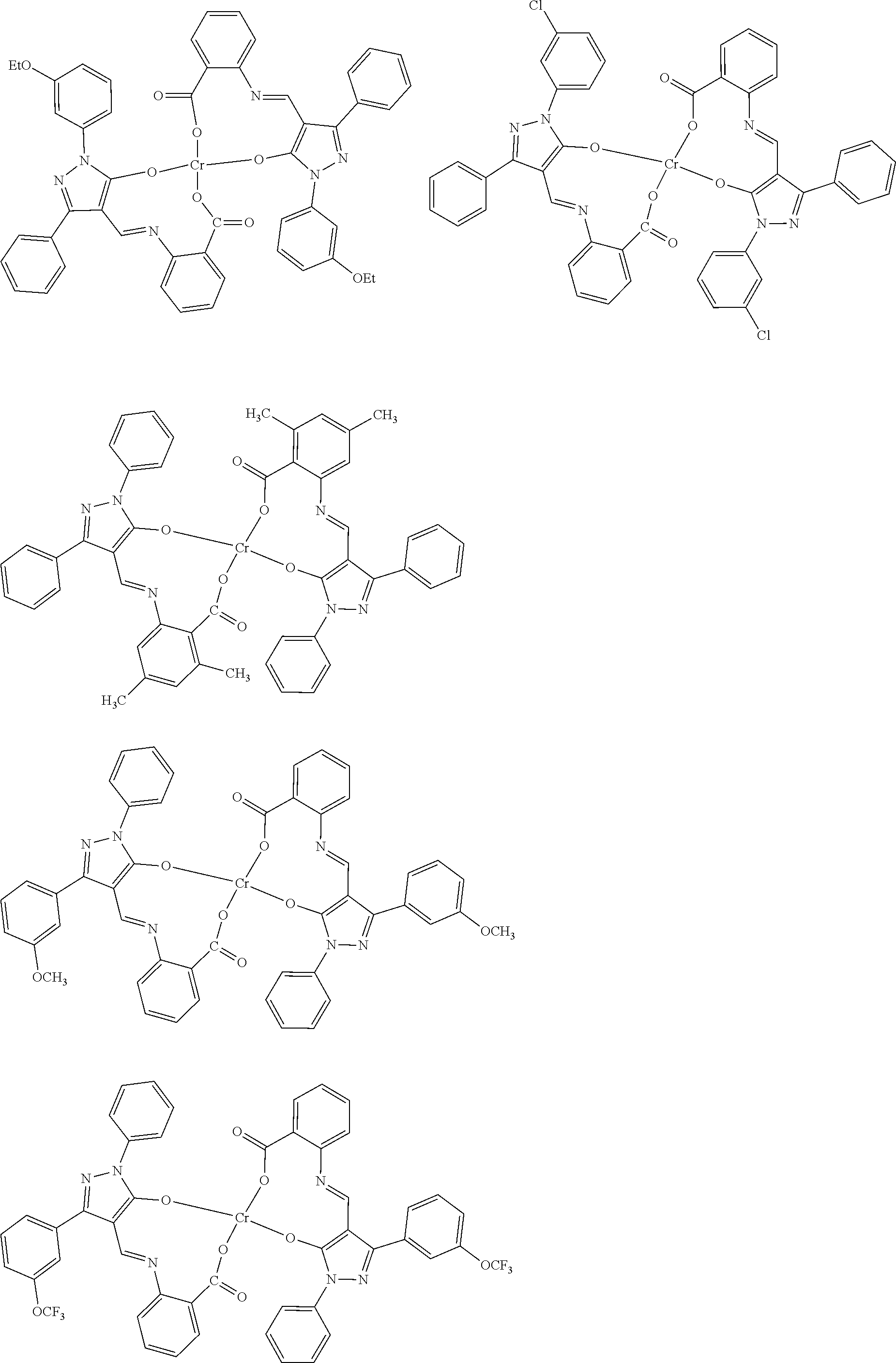

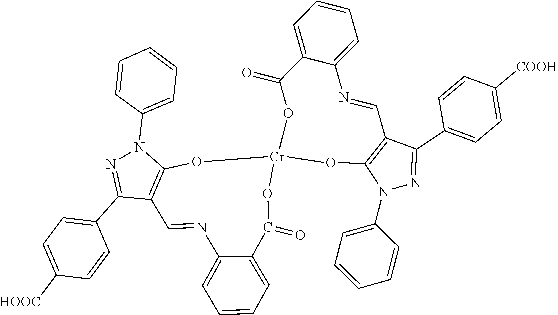

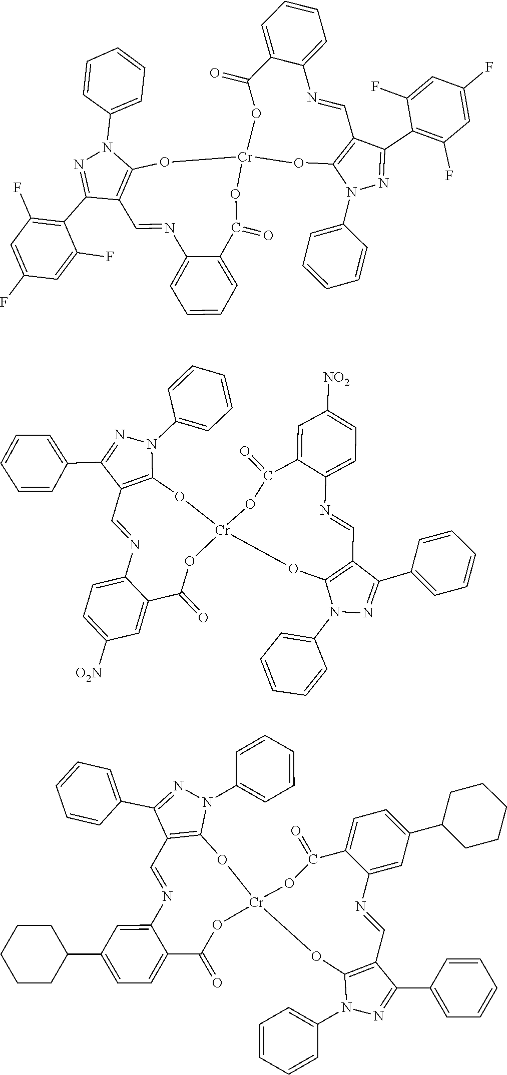

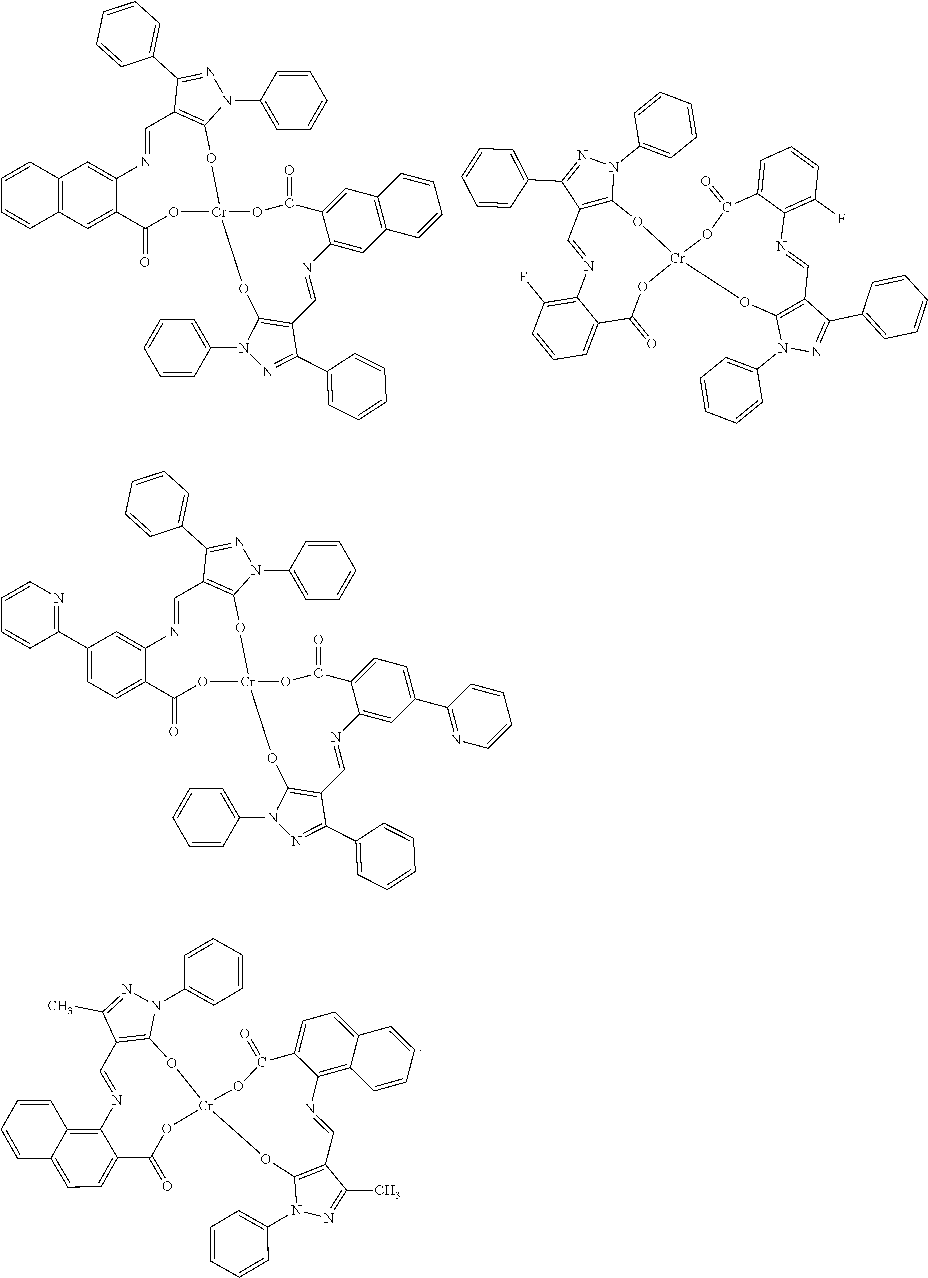

In these materials, Me means a methyl group and Et means an ethyl group.

According to one embodiment of the present disclosure, the coordination compound further includes (B⁺)m.

According to one embodiment of the present disclosure, B⁺ is H⁺; Na⁺; K⁺; P⁺ReRfRgRh; N⁺RiRjRkRl; S⁺RmRnRo; or a cationic heteroring, and Re to Ro are the same as or different from each other and each independently may be hydrogen; a substituted or unsubstituted alkyl group; or a substituted or unsubstituted cycloalkyl group.

According to one embodiment of the present disclosure, the cationic heteroring is any one selected from the group consisting of the following Structural Formulae B1 to B4:

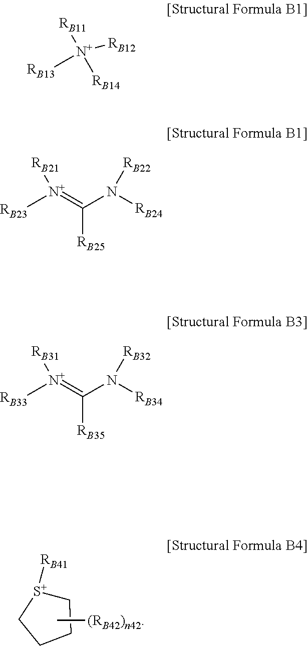

In Structural Formulae B1 to B4,

R_{B11} and R_{B12} bond to each other to form a substituted or unsubstituted heteroring having 4 to 30 carbon atoms, and R_{B13} and R_{B14} are the same as or different from each other and each independently may be hydrogen; a hydrocarbon group having 1 to 30 carbon atoms; or a substituent including one, two or more heteroatoms selected from the group consisting of N, O, P, S, Si and Se, R_{B}21 and R_{B}22 bond to each other to form a substituted or unsubstituted heteroring having 2 to 30 carbon atoms, and R_{B23} to R_{B25} are the same as or different from each other and each independently may be hydrogen; a hydrocarbon group having 1 to 30 carbon atoms; or a substituent including one, two or more heteroatoms selected from the group consisting of N, O, P, S, Si and Se,

R_{B31} and R_{B32} bond to each other to form a substituted or unsubstituted heteroring having 2 to 30 carbon atoms, and R_{B33} to R_{B35} are the same as or different from each other and each independently may be hydrogen; a hydrocarbon group having 1 to 30 carbon atoms; or a substituent including one, two or more heteroatoms selected from the group consisting of N, O, P, S, Si and Se, and

R_{B41} is hydrogen; a hydrocarbon group having 1 to 30 carbon atoms; or a substituent including one, two or more heteroatoms selected from the group consisting of N, O, P, S, Si and Se, R_{B42} is hydrogen; a hydrocarbon group having 1 to 30 carbon atoms; or a substituent including one, two or more heteroatoms selected from the group consisting of N, O, P, S, Si and Se, and n42 is an integer of 1 to 8.

According to another embodiment of the present disclosure, B⁺ is H⁺; Na⁺; K⁺; a phosphonium cation; an ammonium cation; a sulfonium cation; or a cationic heteroring, and m is an integer of 1 or 2.

In another embodiment of the present disclosure, B is an alkali metal; NR₄; or PR′₄, and R and R′ are the same as or different from each other and each independently may be hydrogen; a substituted or unsubstituted alkyl group having 1 to 10 carbon atoms; or a substituted or unsubstituted cycloalkyl group having 3 to 10 carbon atoms, or two or more Rs bonded to each other to form a substituted or unsubstituted aromatic ring having 2 to 10 carbon atoms or a substituted or unsubstituted aliphatic ring having 2 to 10 carbon atoms.

In one embodiment of the present disclosure, B is hydrogen.

In one embodiment of the present disclosure, B is an alkali metal.

The alkali metal means a chemical element in Group 1 of the periodic table other than hydrogen, and examples thereof may include lithium, sodium, potassium, rubidium, cesium or francium.

In one embodiment of the present disclosure, B is Na or K.

In one embodiment of the present disclosure, B is NR₄.

In one embodiment of the present disclosure, B is PR′₄.

In one embodiment of the present disclosure, R and R′ are the same as or different from each other, and each independently may be hydrogen; a substituted or unsubstituted alkyl group having 1 to 30 carbon atoms; or a substituted or unsubstituted cycloalkyl group having 3 to 30 carbon atoms, or two or more Rs may bond to each other to form a substituted or unsubstituted aromatic heteroring having 6 to 30 carbon atoms or a substituted or unsubstituted aliphatic heteroring having 1 to 30 carbon atoms.

In one embodiment of the present disclosure, R and R′ are the same as or different from each other, and each independently may be hydrogen; a substituted or unsubstituted alkyl group having 1 to 20 carbon atoms; or a substituted or unsubstituted cycloalkyl group having 3 to 20 carbon atoms, or two or more Rs may bond to each other to form a substituted or unsubstituted aromatic heteroring having 6 to 20 carbon atoms or a substituted or unsubstituted aliphatic heteroring having 1 to 20 carbon atoms.

In one embodiment of the present disclosure, R and R′ are the same as or different from each other, and each independently may be hydrogen; a substituted or unsubstituted alkyl group having 1 to 10 carbon atoms; or a substituted or unsubstituted cycloalkyl group having 3 to 10 carbon atoms, or two or more Rs may bond to each other to form a substituted or unsubstituted aromatic heteroring having 6 to 12 carbon atoms or a substituted or unsubstituted aliphatic heteroring having 1 to 10 carbon atoms.

In one embodiment of the present disclosure, B is NR₄; or BR′₄, and R and R′ are the same as or different from each other and each independently may be hydrogen; a substituted or unsubstituted alkyl group having 1 to 10 carbon atoms; or a substituted or unsubstituted cycloalkyl group having 3 to 10 carbon atoms, or two or more Rs may bond to each other to form a substituted or unsubstituted aromatic ring having 2 to 10 carbon atoms or a substituted or unsubstituted aliphatic ring having 2 to 10 carbon atoms.

In one embodiment of the present disclosure, B is an alkali metal; NR₄; or PR′₄, and R and R′ are the same as or different from each other and each independently may be hydrogen; a substituted or unsubstituted alkyl group having 1 to 10 carbon atoms; or a substituted or unsubstituted cycloalkyl group having 3 to 10 carbon atoms, or two or more Rs may bond to each other to form a substituted or unsubstituted aromatic ring having 2 to 10 carbon atoms or a substituted or unsubstituted aliphatic ring having 2 to 10 carbon atoms.

In one embodiment of the present disclosure, R is hydrogen; a substituted or unsubstituted butyl group; a substituted or unsubstituted hexyl group; a substituted or unsubstituted decyl group; a substituted or unsubstituted dodecyl group; or a substituted or unsubstituted cyclohexyl group, or adjacent R groups may bond to each other to form a compound represented by the following Chemical Formula N-1; or a compound represented by the following Chemical Formula N-2:

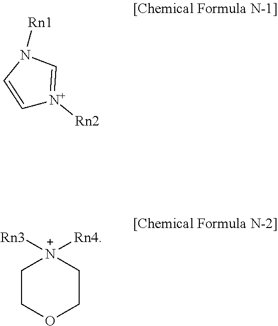

In Chemical Formulae N-1 and N-2,

Rn1 to Rn4 are the same as or different from each other, and each independently may be hydrogen; or a substituted or unsubstituted alkyl group.

In one embodiment of the present disclosure, R is hydrogen; an n-butyl group; an n-hexyl group; a hexyl group substituted with an ethyl group; an n-decyl group; an n-dodecyl group; or a cyclohexyl group, or adjacent R groups may bond to each other to form the compound represented by Chemical Formula N-1; or the compound represented by Chemical Formula N-2.

In one embodiment of the present disclosure, R is hydrogen; an n-butyl group; an n-hexyl group; or a hexyl group substituted with an ethyl group, or adjacent R groups may bond to each other to form the compound represented by Chemical Formula N-1; or the compound represented by Chemical Formula N-2.

The hexyl group substituted with an ethyl group may be a 2-ethylhexyl group.

In one embodiment of the present disclosure, R′ is a substituted or unsubstituted butyl group.

In one embodiment of the present disclosure, R′ is an n-butyl group.

In one embodiment of the present disclosure, B is NR₄, NR₄ is NH₄; NHR″₃; NH₂R″₂; NR″₄; a compound represented by the following Chemical Formula N-1; or a compound represented by the following Chemical Formula N-2, and R″ is a substituted or unsubstituted alkyl group having 1 to 20 carbon atoms; or a substituted or unsubstituted cycloalkyl group having 3 to 20 carbon atoms:

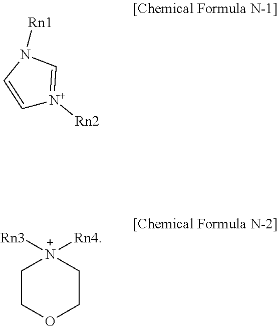

In Chemical Formulae N-1 and N-2,

Rn1 to Rn4 are the same as or different from each other, and each independently may be hydrogen; or a substituted or unsubstituted alkyl group.

In one embodiment of the present disclosure, Rn1 to Rn4 are the same as or different from each other, and each independently may be hydrogen; or a substituted or unsubstituted alkyl group having 1 to 30 carbon atoms.

In one embodiment of the present disclosure, Rn1 to Rn4 are the same as or different from each other, and each independently may be hydrogen; or a substituted or unsubstituted alkyl group having 1 to 20 carbon atoms.

In one embodiment of the present disclosure, Rn1 to Rn4 are the same as or different from each other, and each independently may be hydrogen; or a substituted or unsubstituted alkyl group having 1 to 10 carbon atoms.

In one embodiment of the present disclosure, Rn1 to Rn4 are the same as or different from each other, and each independently may be hydrogen; a substituted or unsubstituted methyl group; a substituted or unsubstituted ethyl group; or a substituted or unsubstituted butyl group.

In one embodiment of the present disclosure, Rn1 to Rn4 are the same as or different from each other, and each independently may be hydrogen; a methyl group; an ethyl group; or an n-butyl group.

In one embodiment of the present disclosure, Rn1 is an n-butyl group.

In one embodiment of the present disclosure, Rn2 is a methyl group.

In one embodiment of the present disclosure, Rn3 and Rn4 are hydrogen; or an ethyl group.

According to one embodiment of the present disclosure, the compound further including (B⁺)m is any one selected from the group consisting of the following compounds:

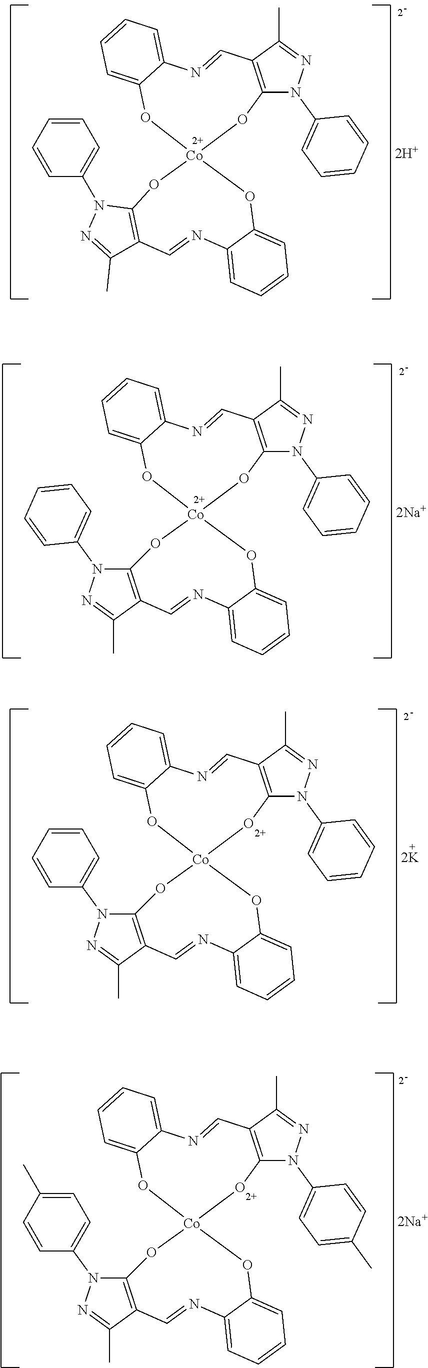

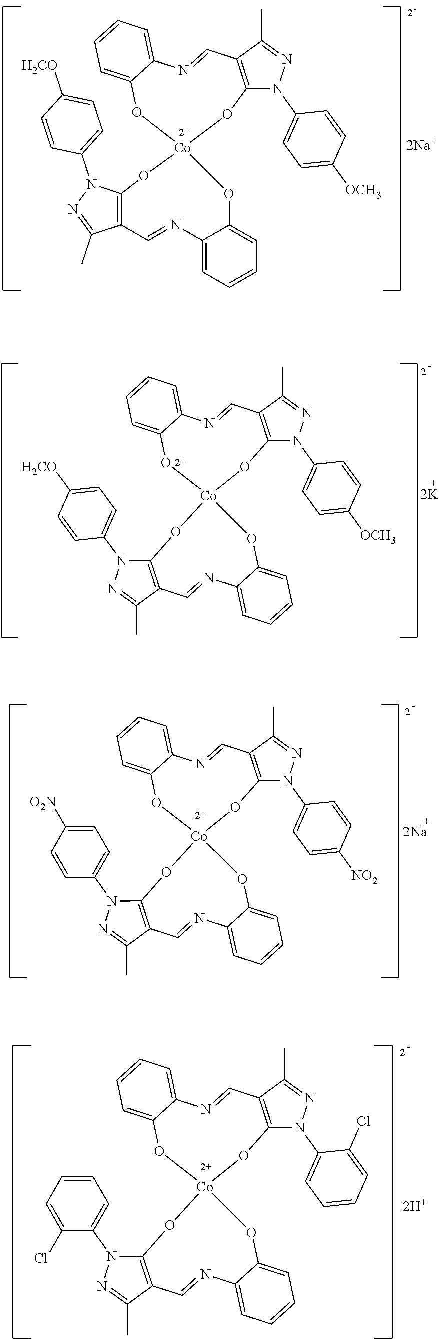

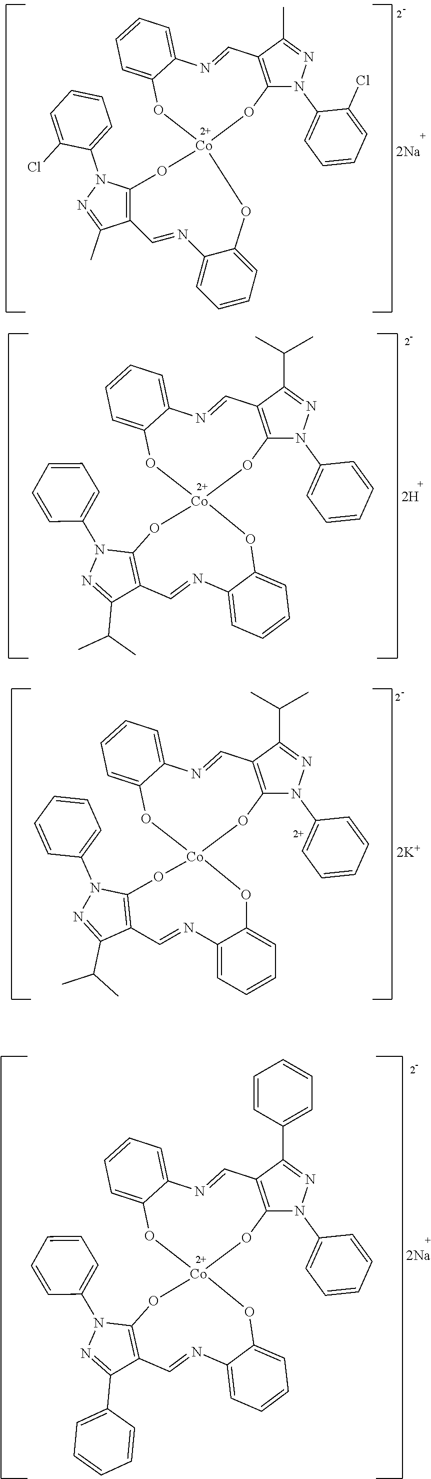

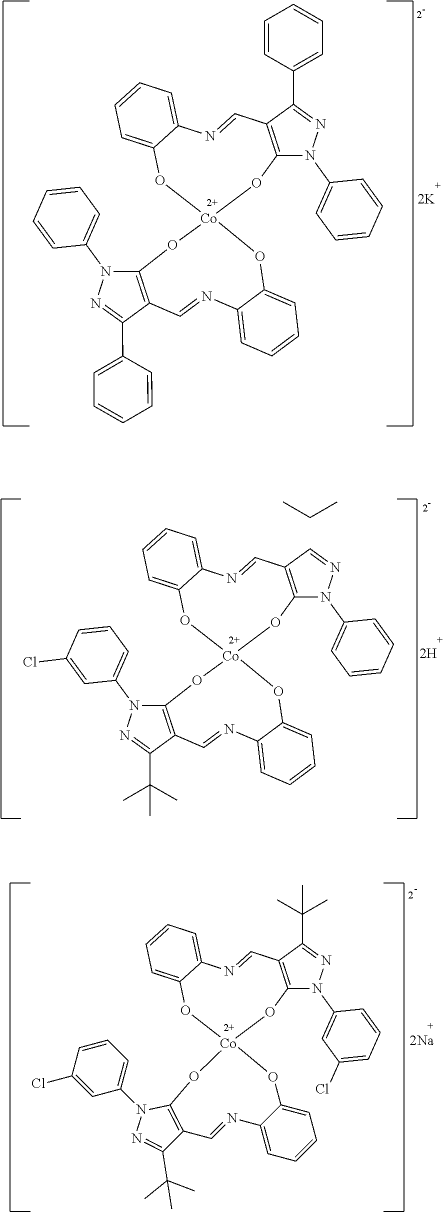

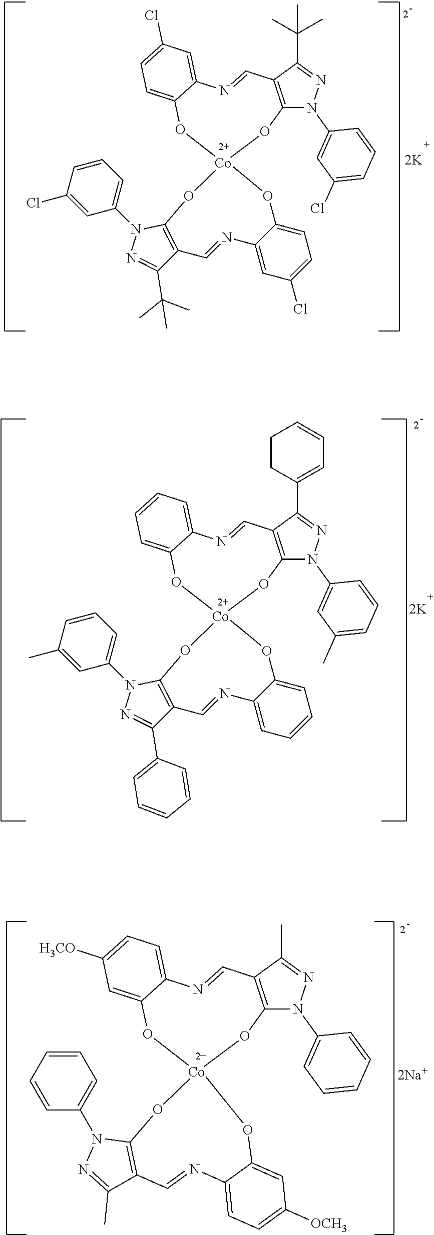

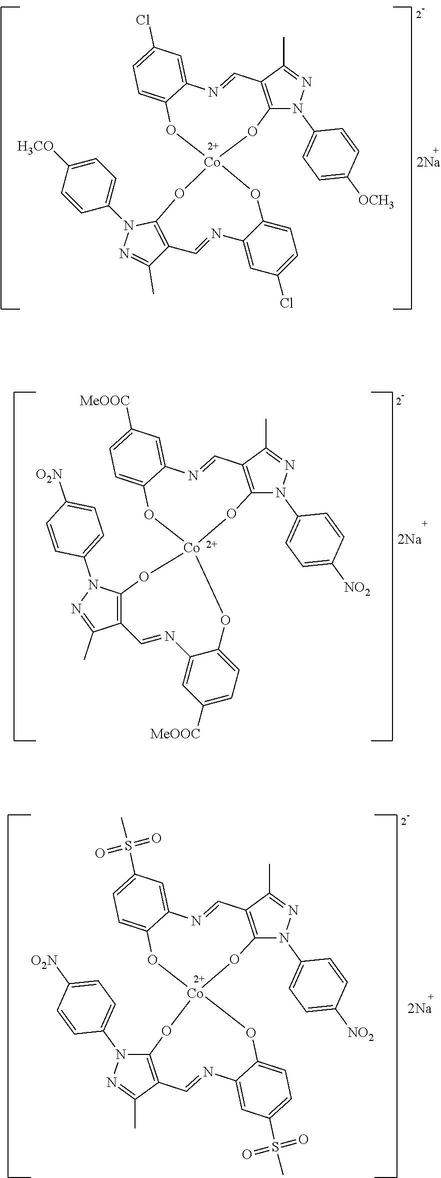

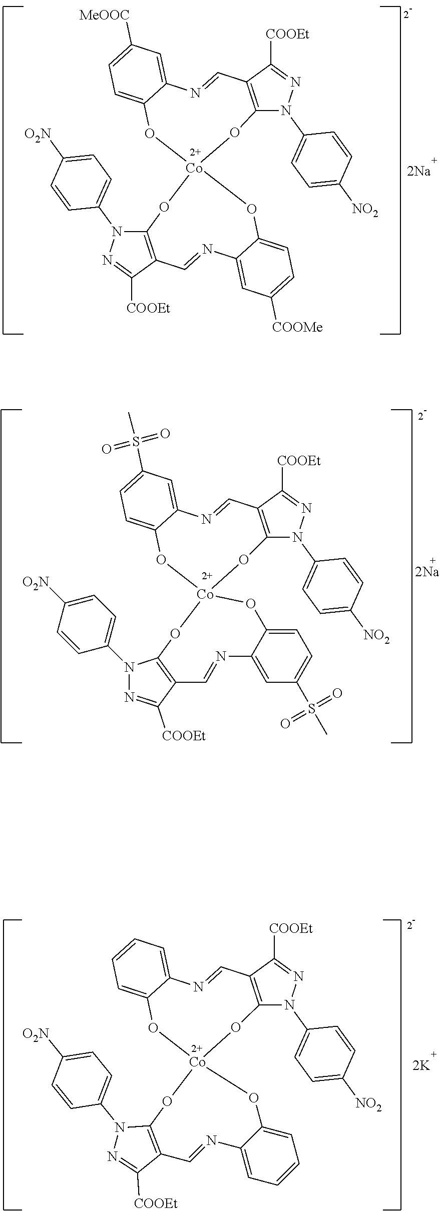

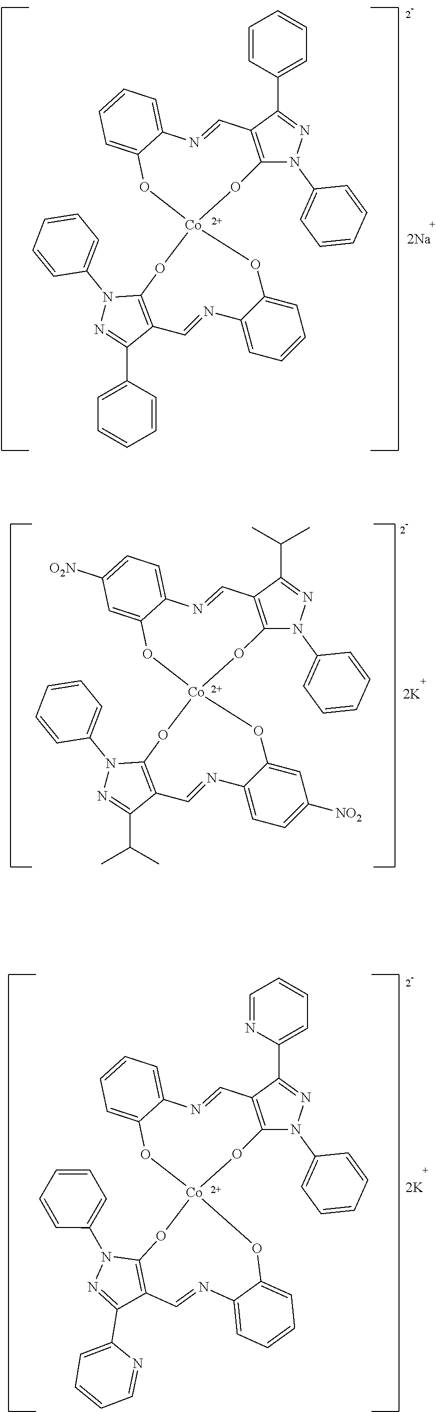

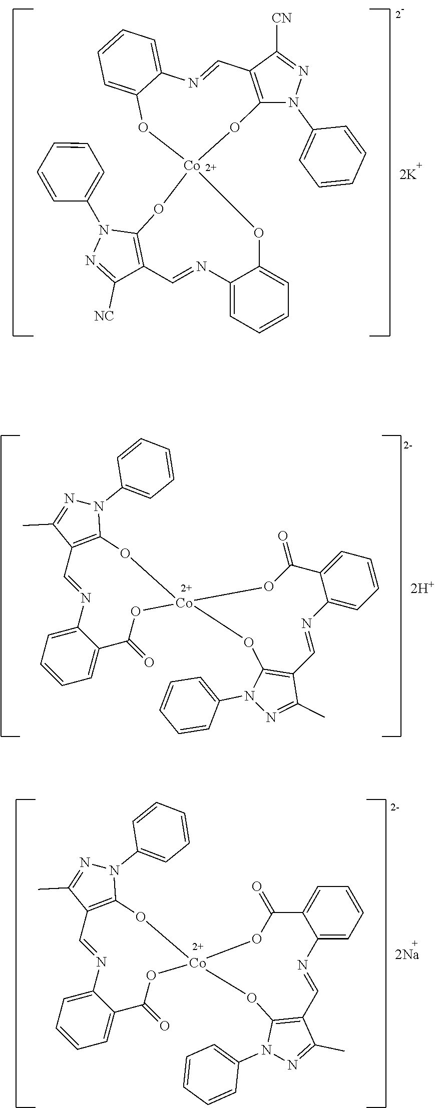

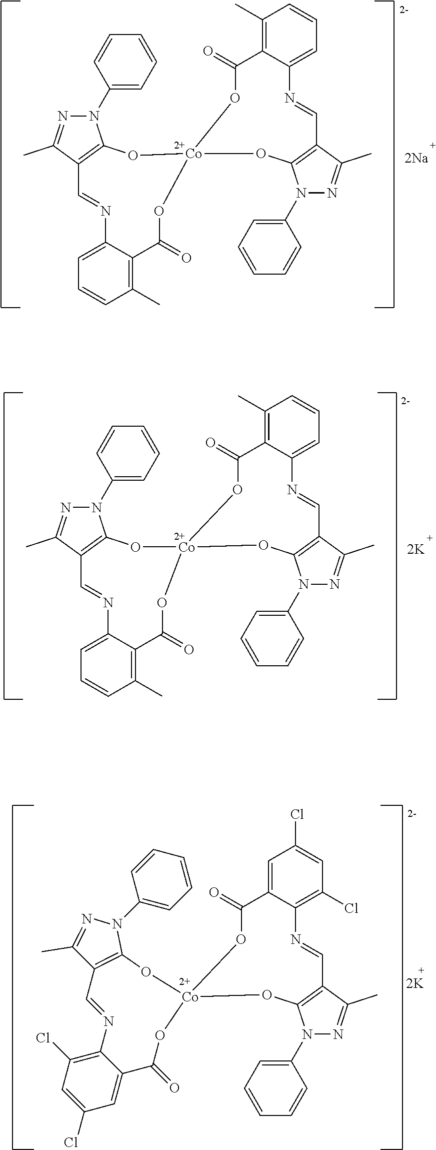

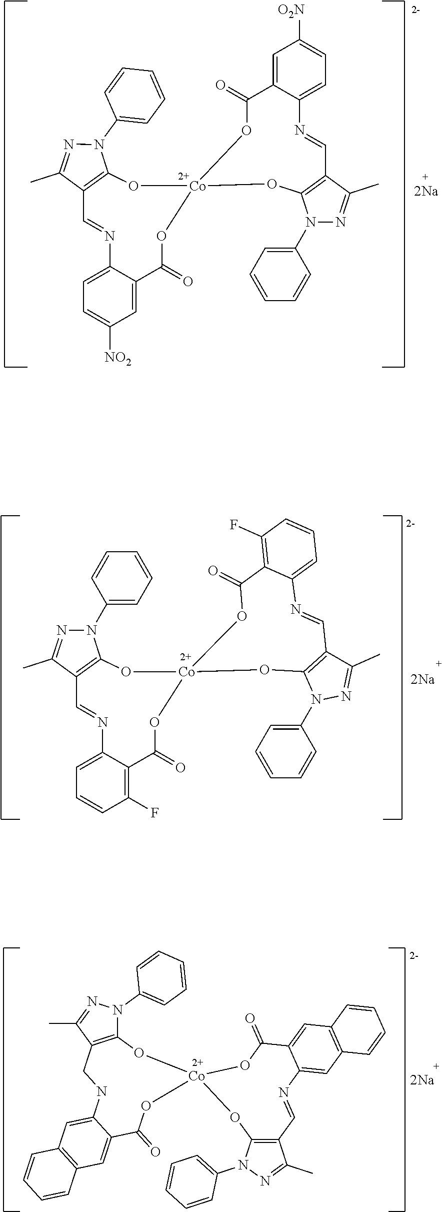

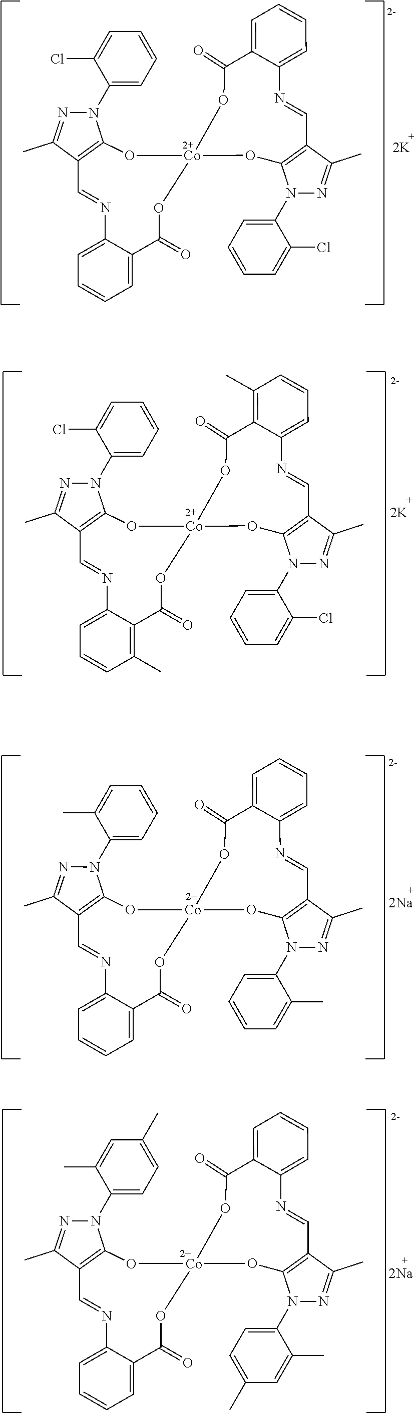

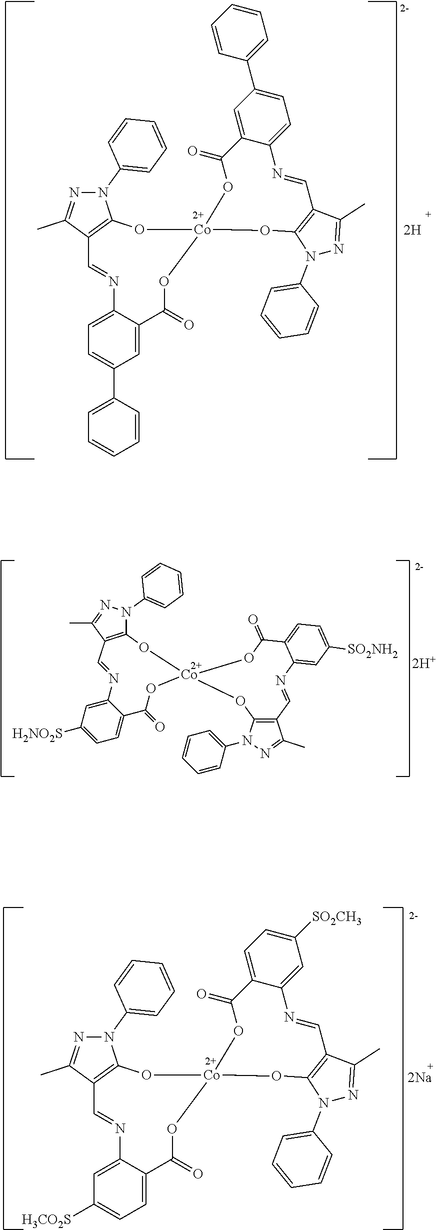

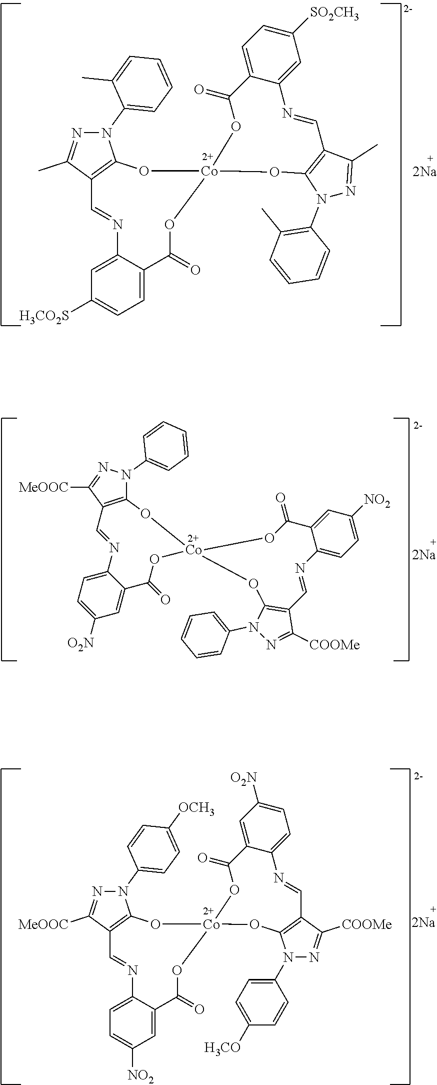

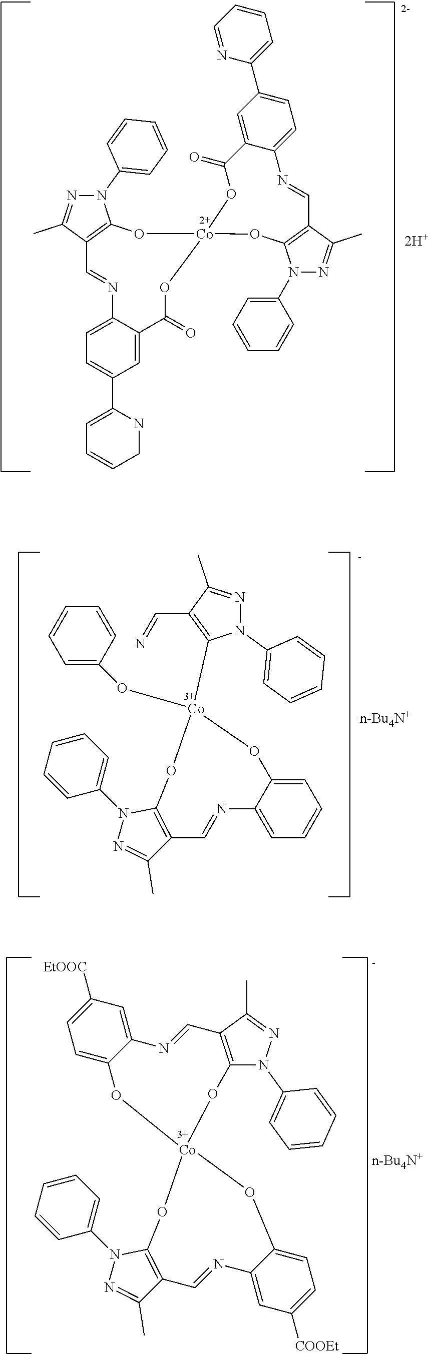

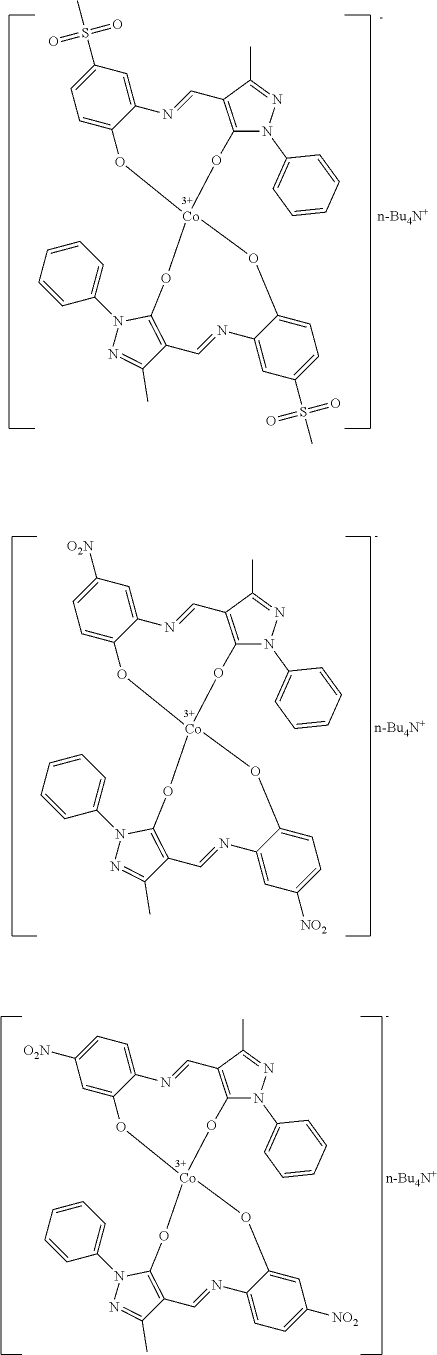

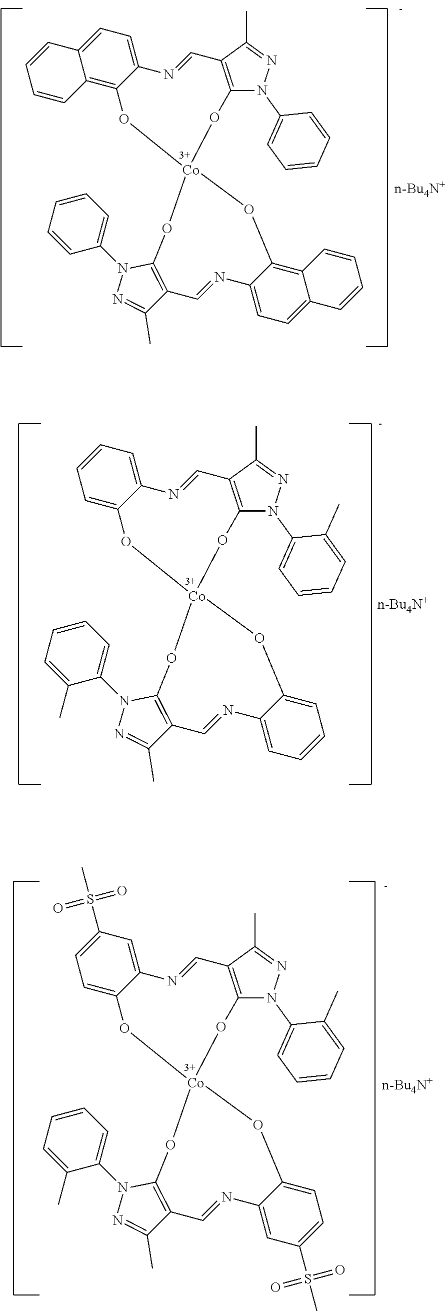

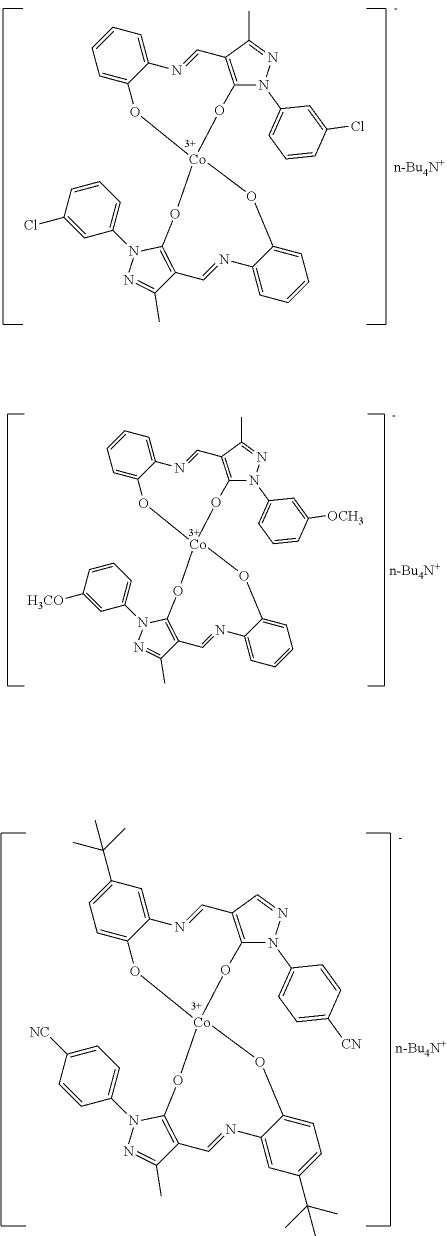

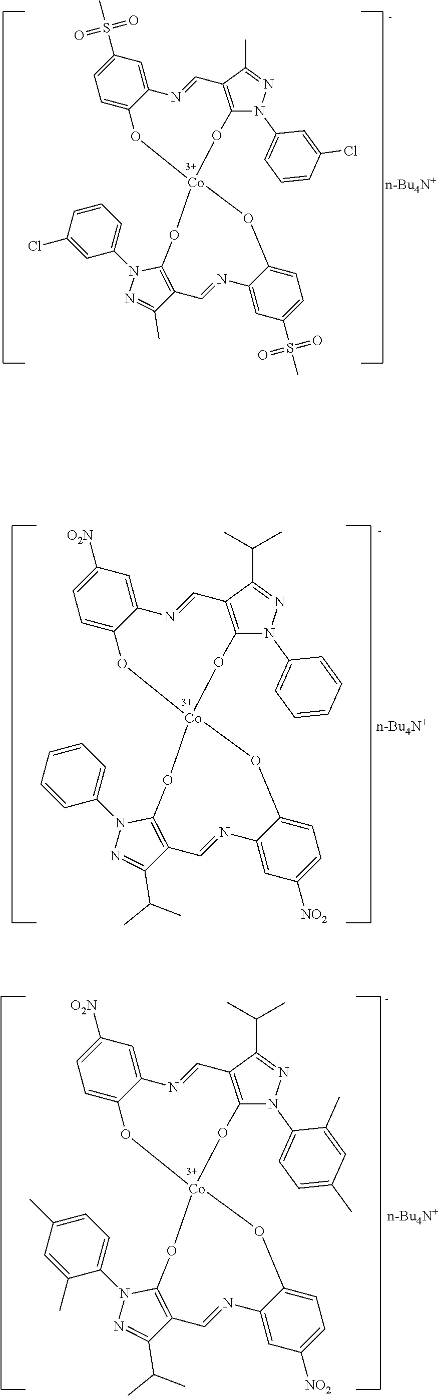

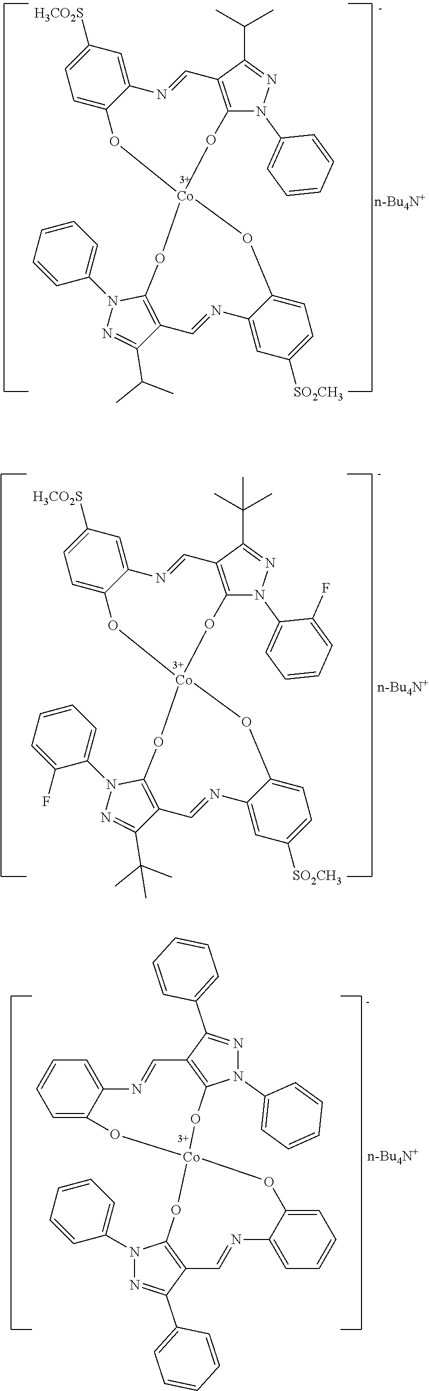

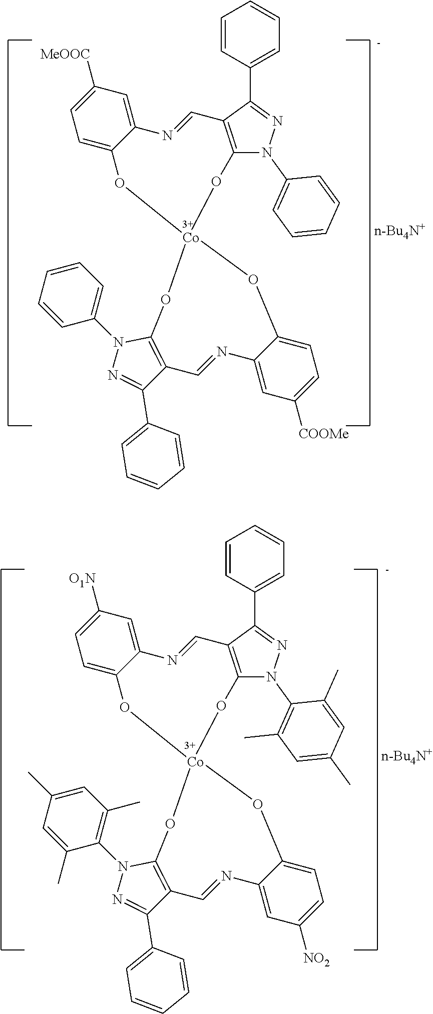

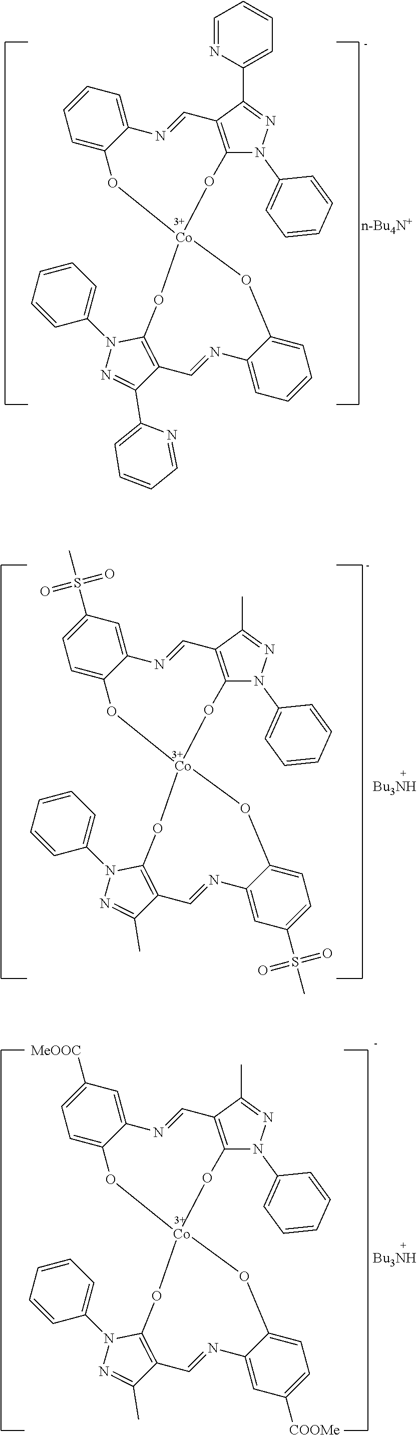

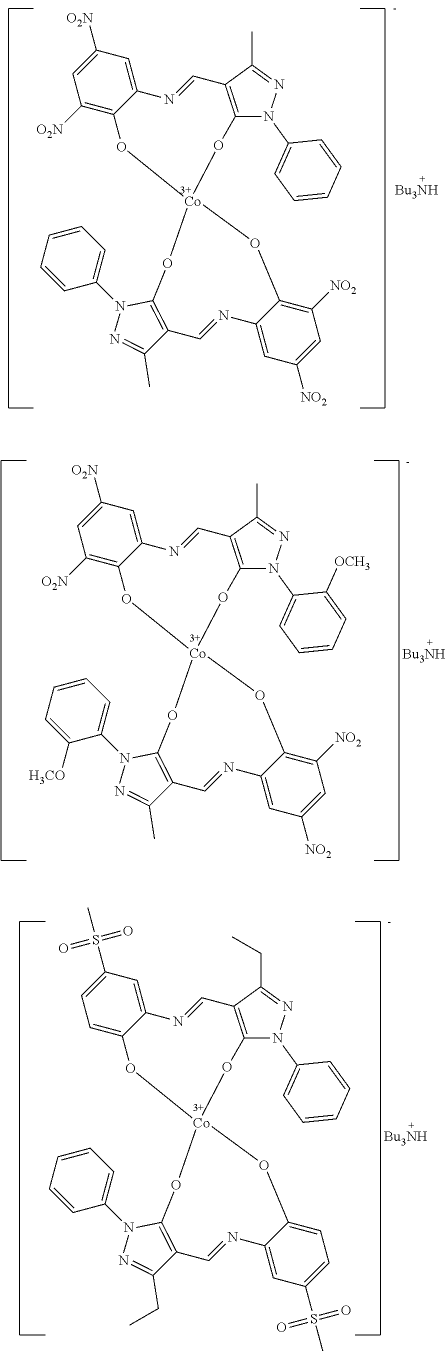

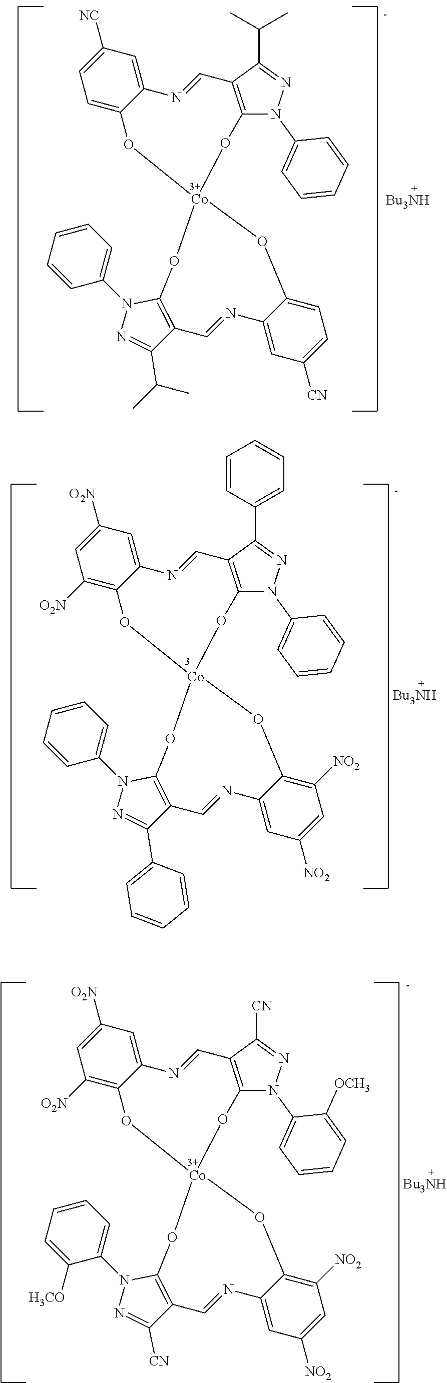

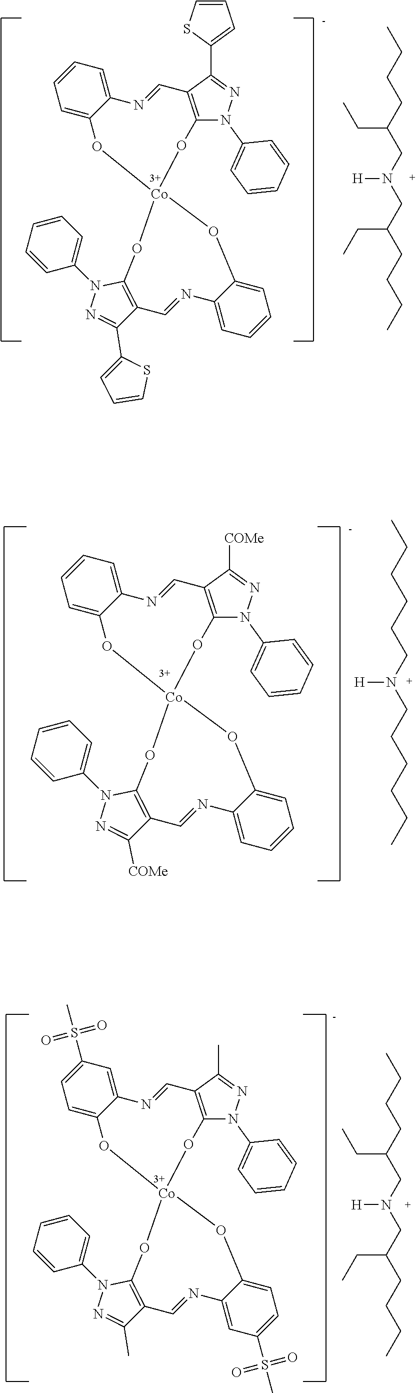

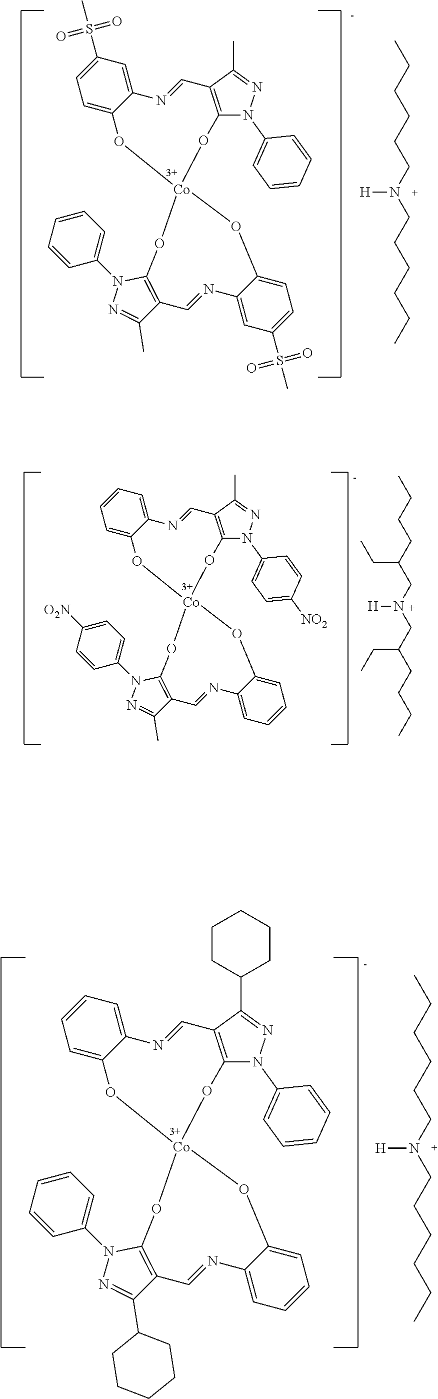

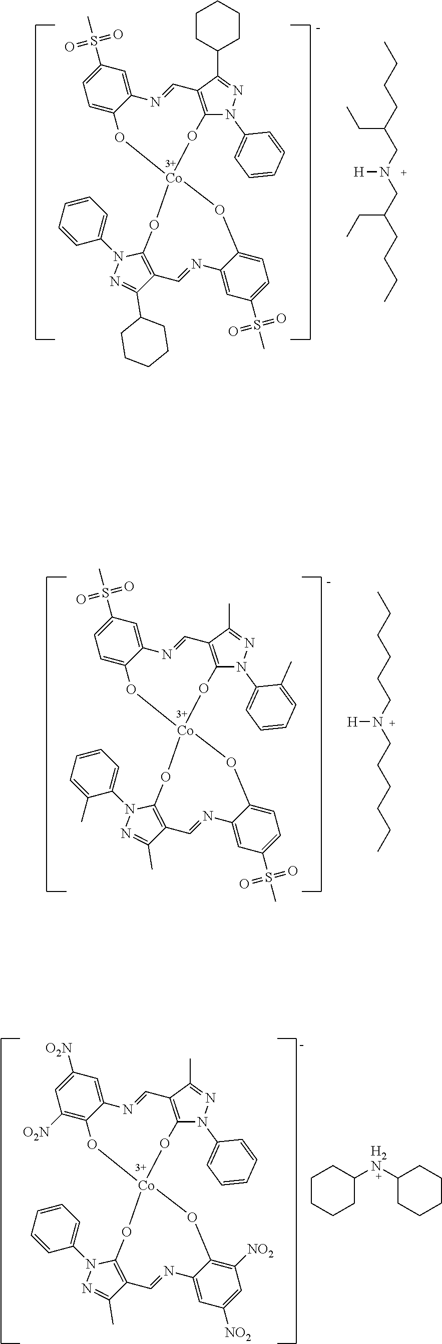

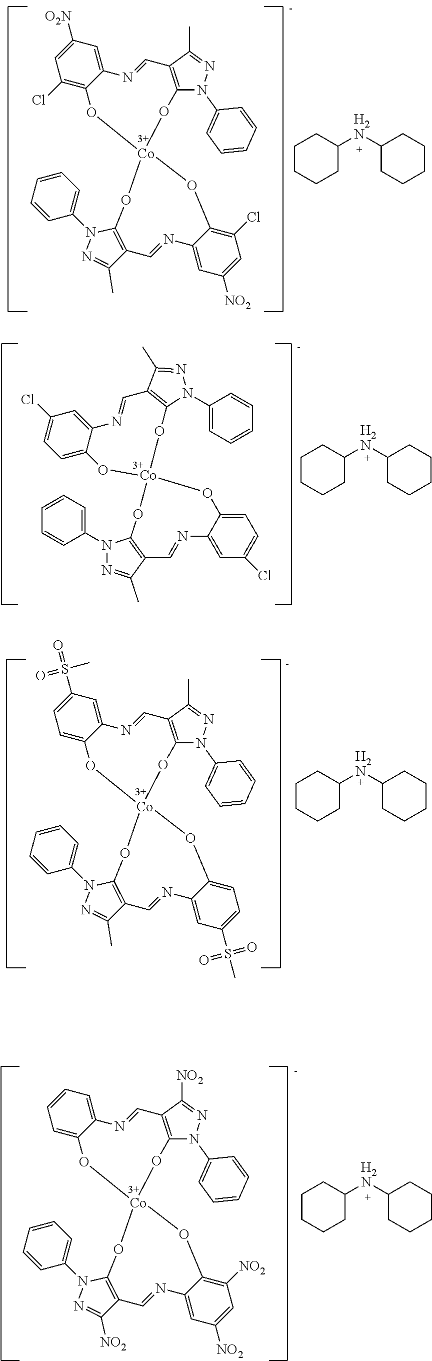

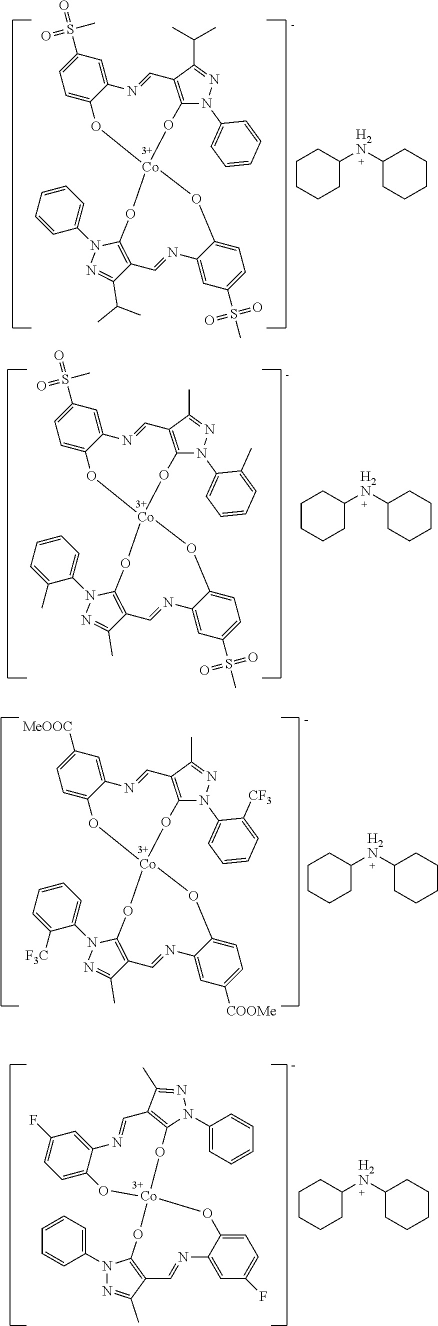

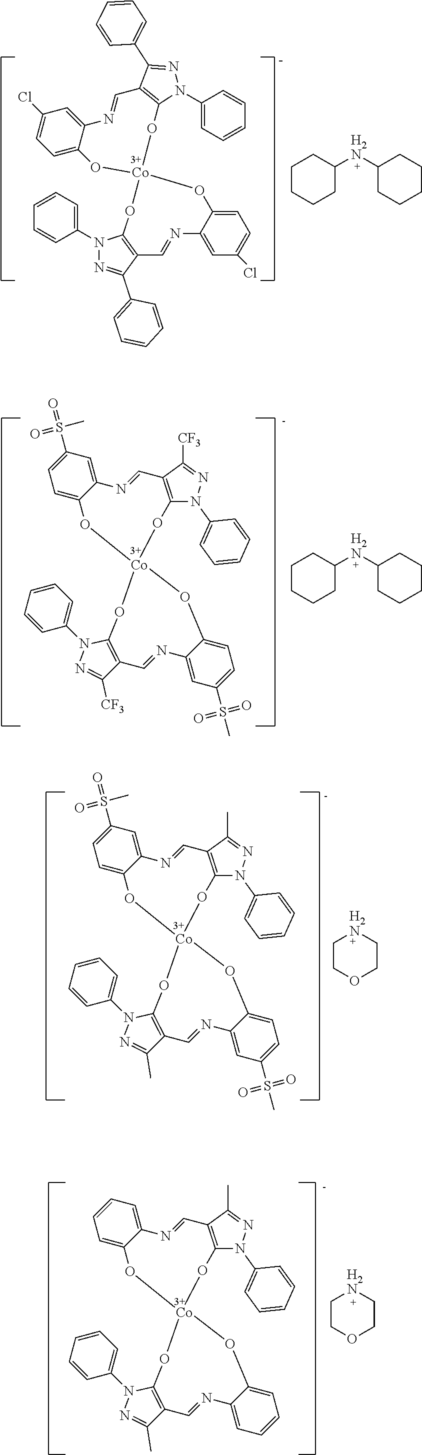

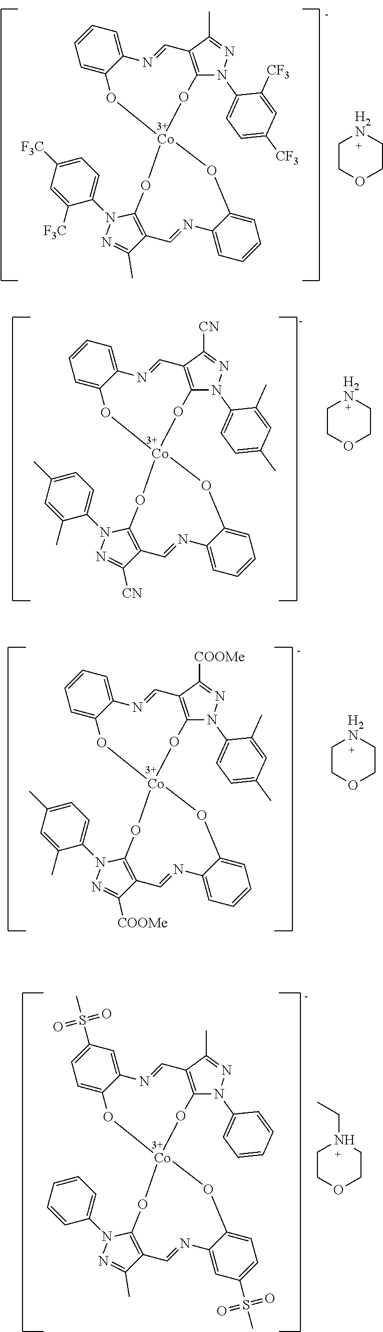

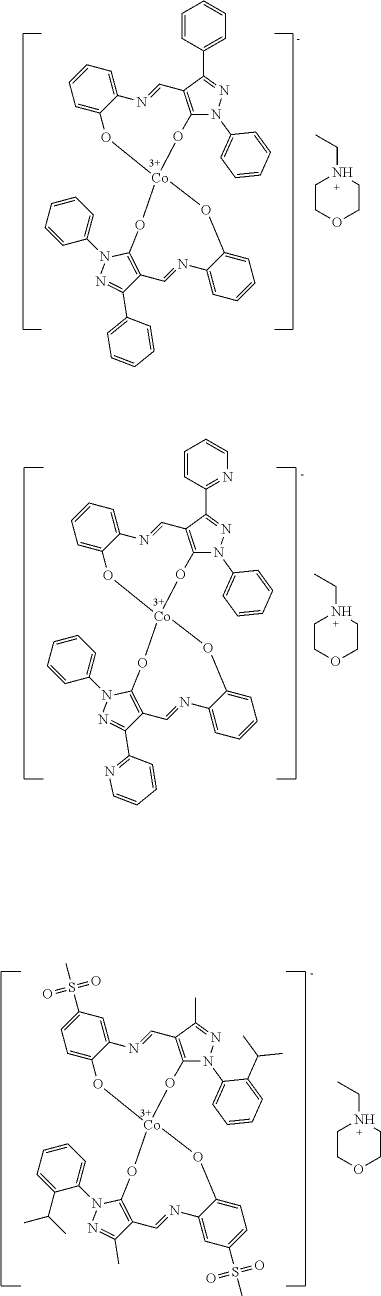

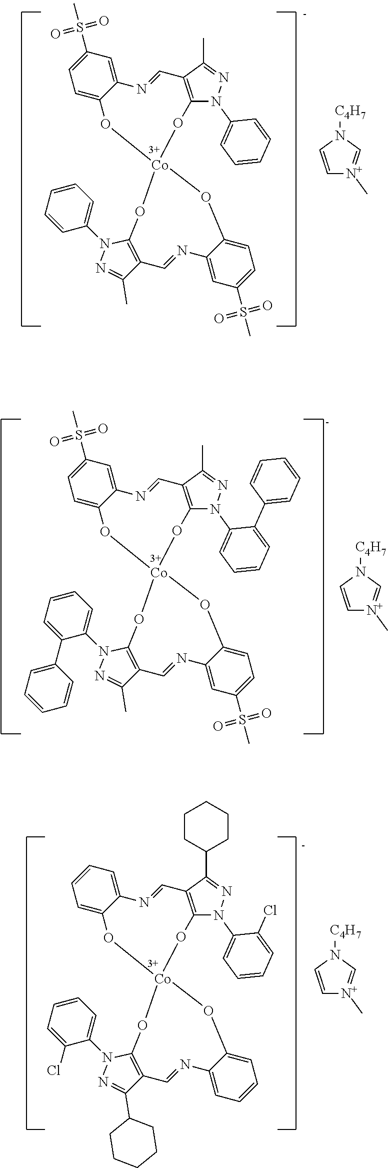

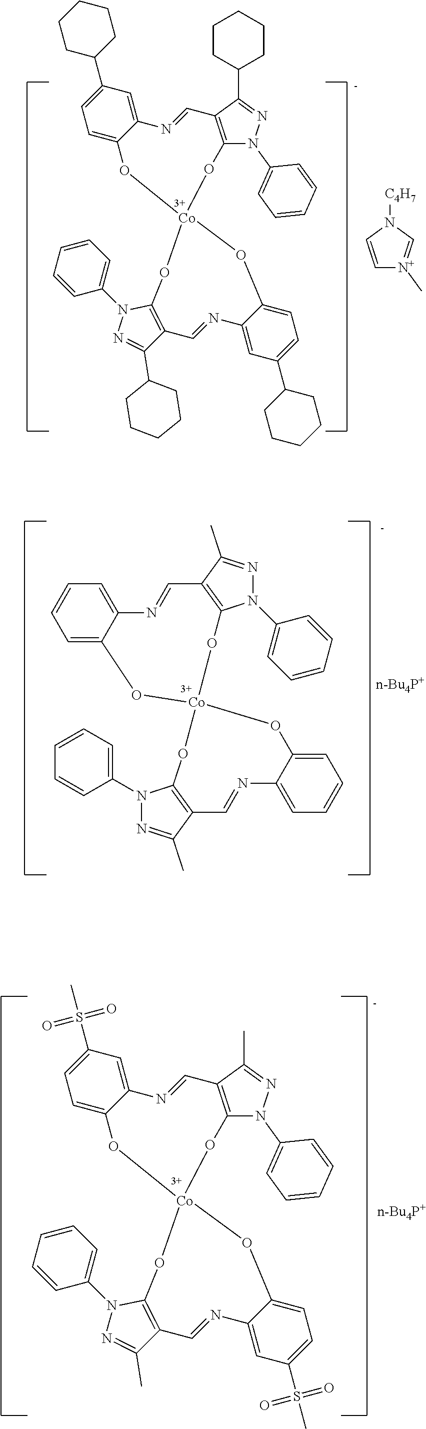

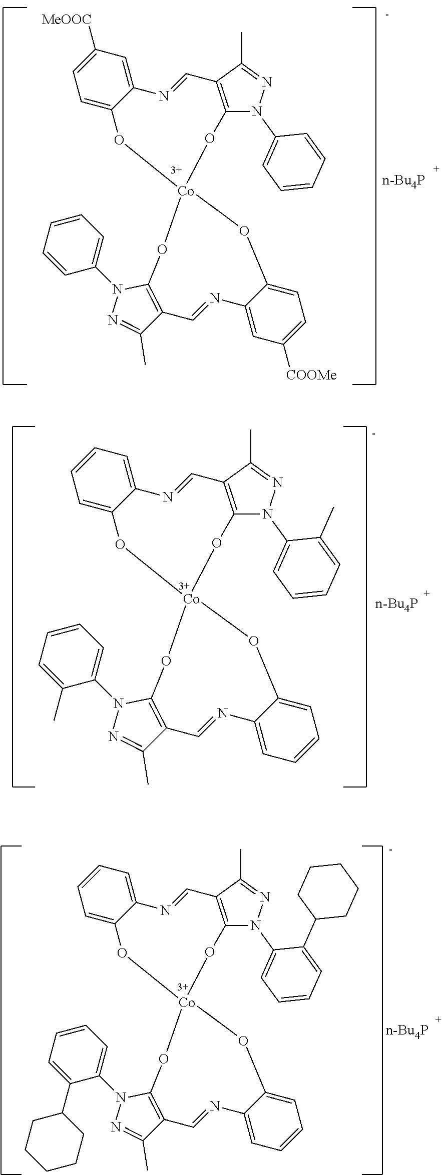

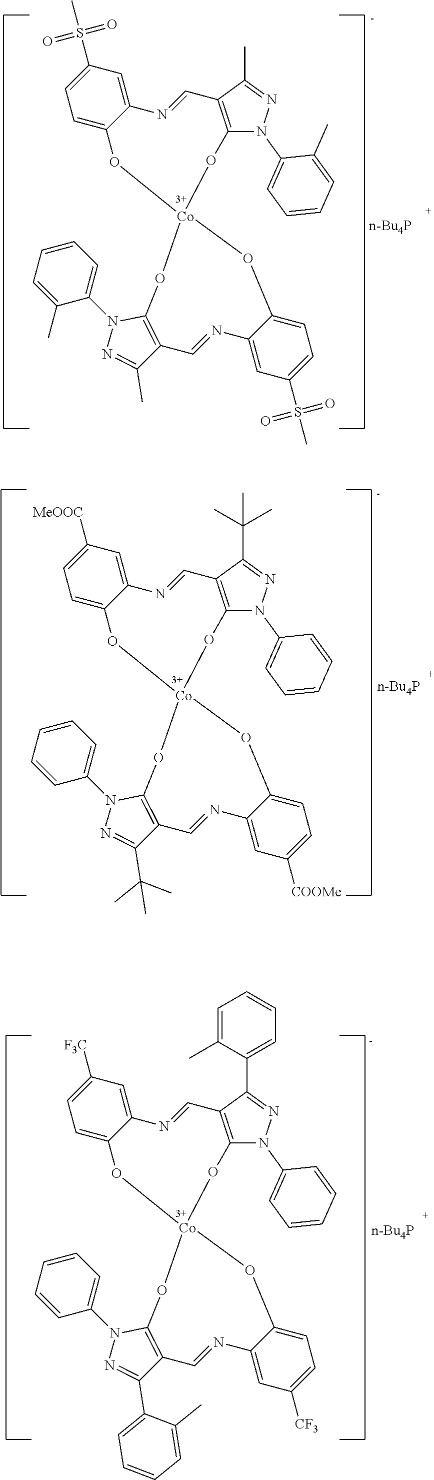

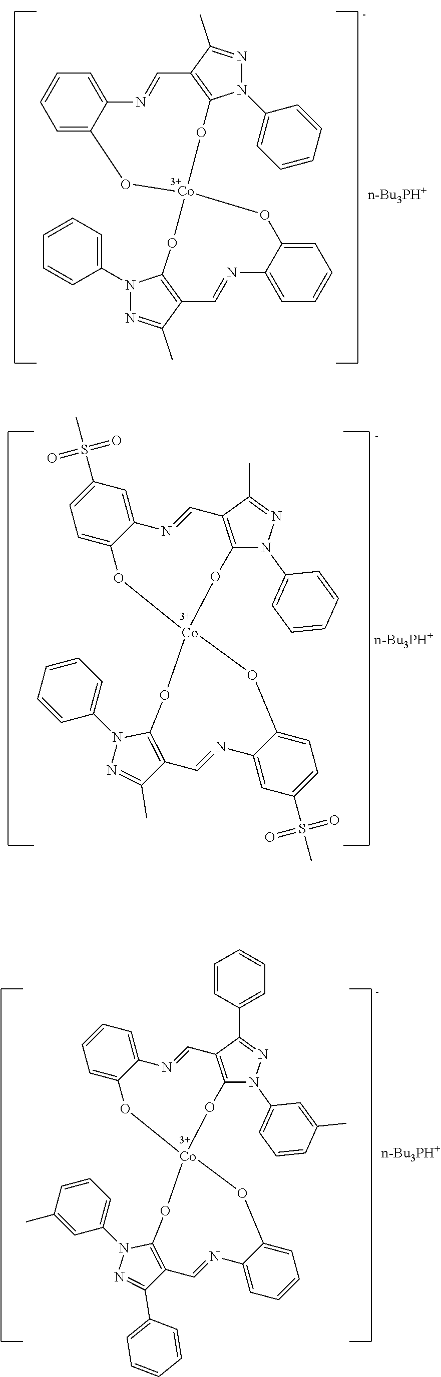

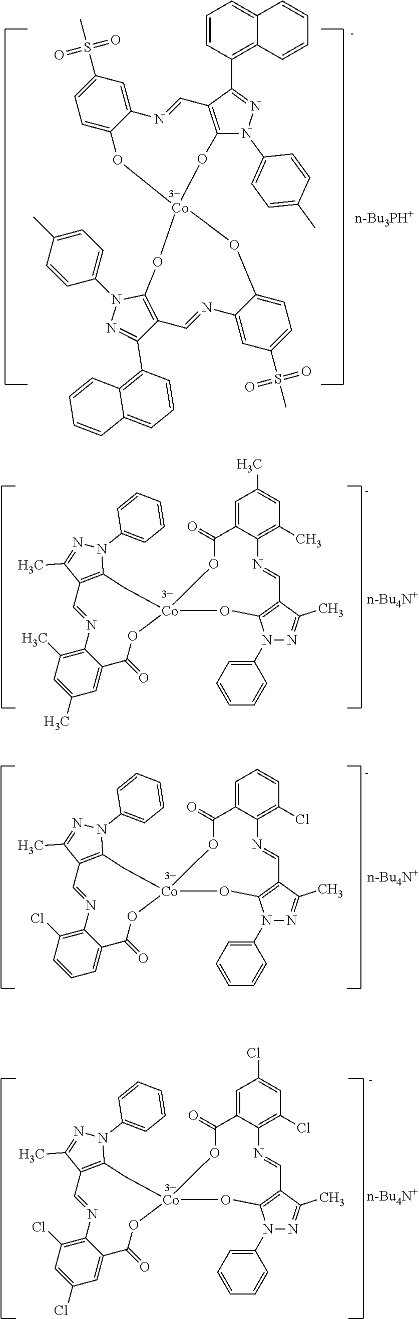

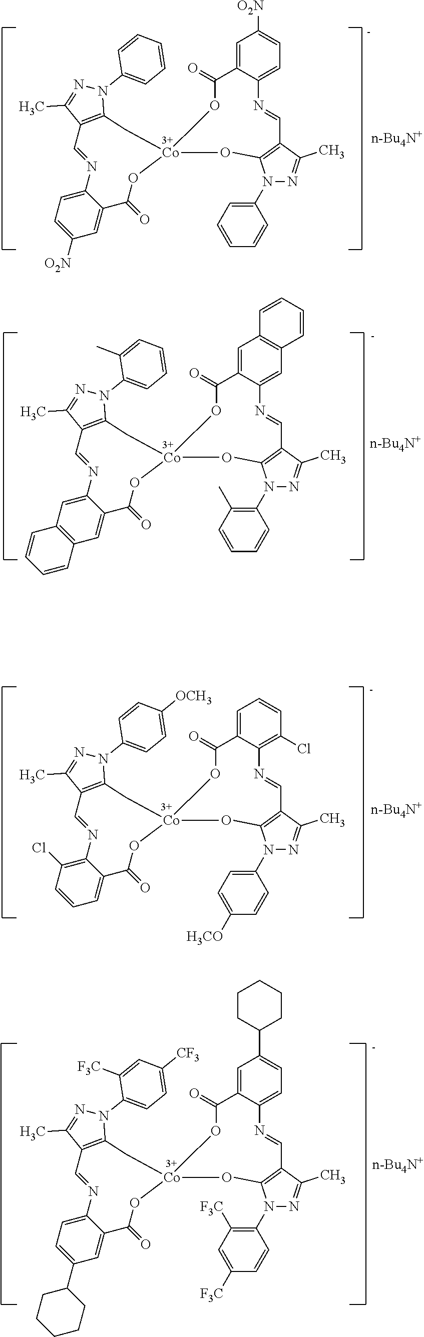

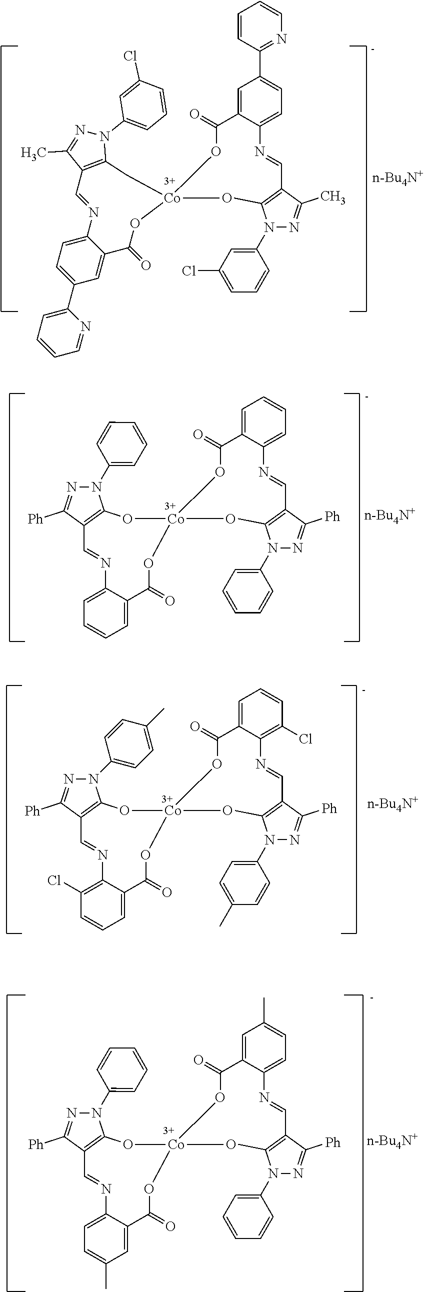

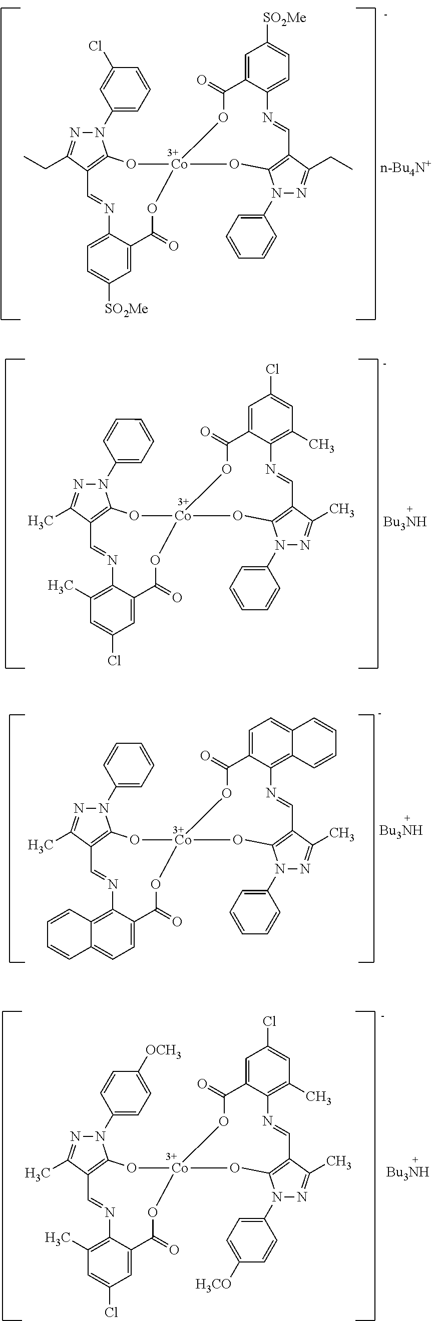

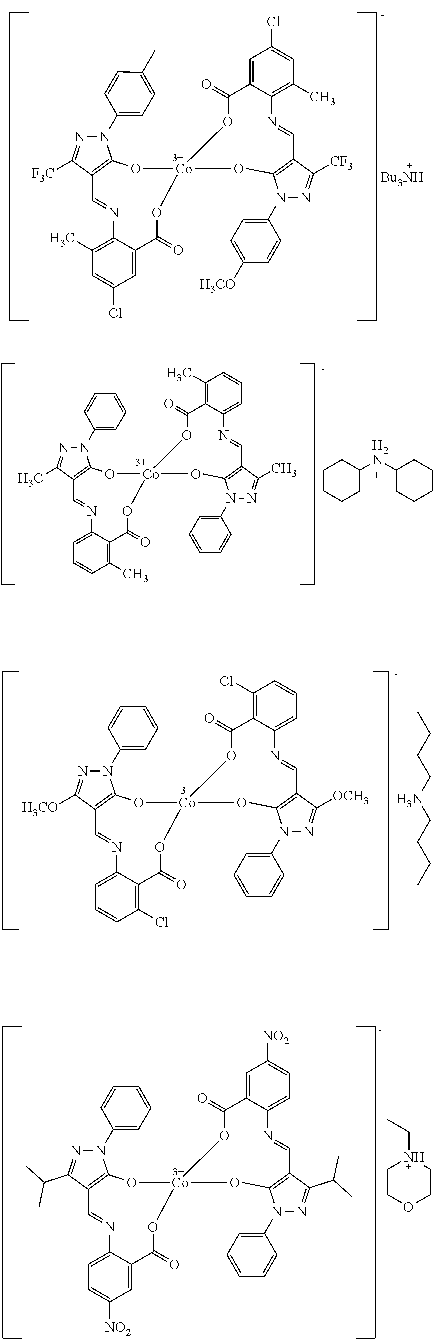

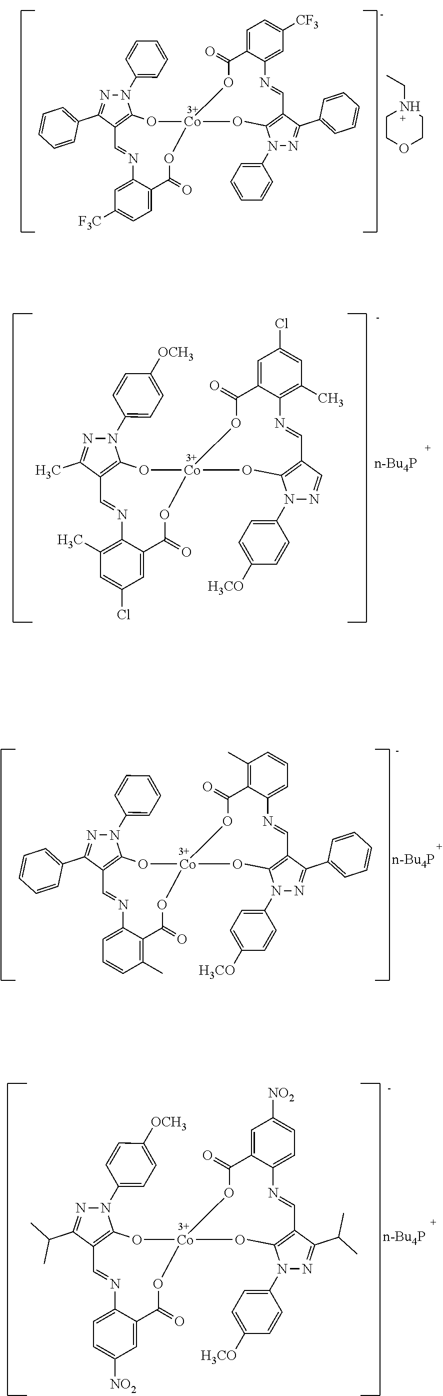

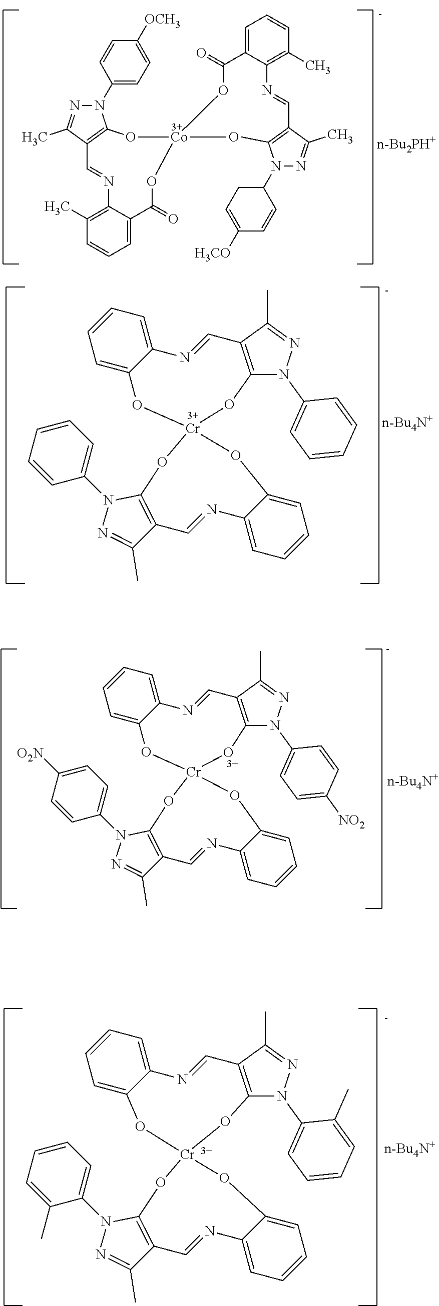

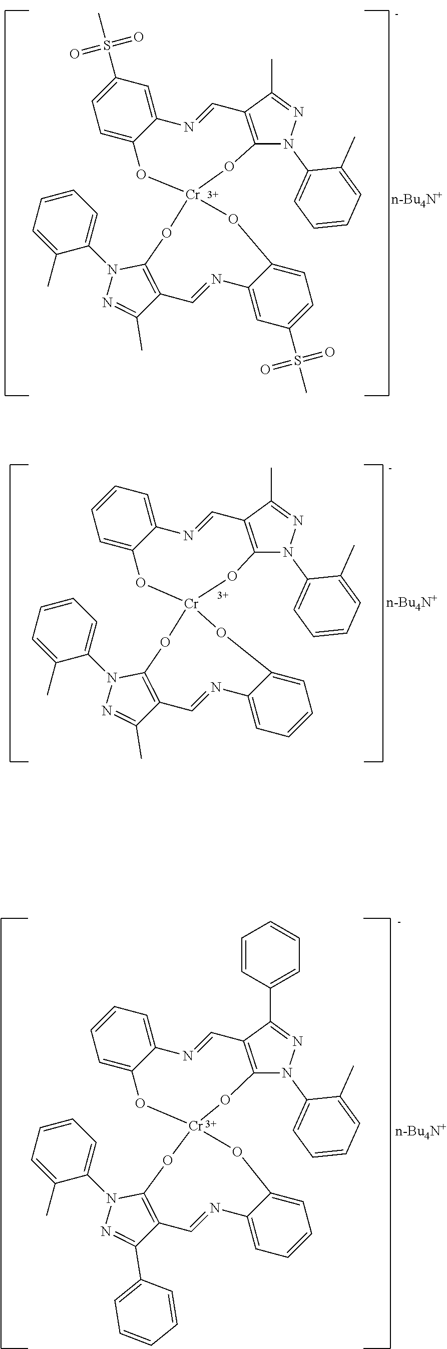

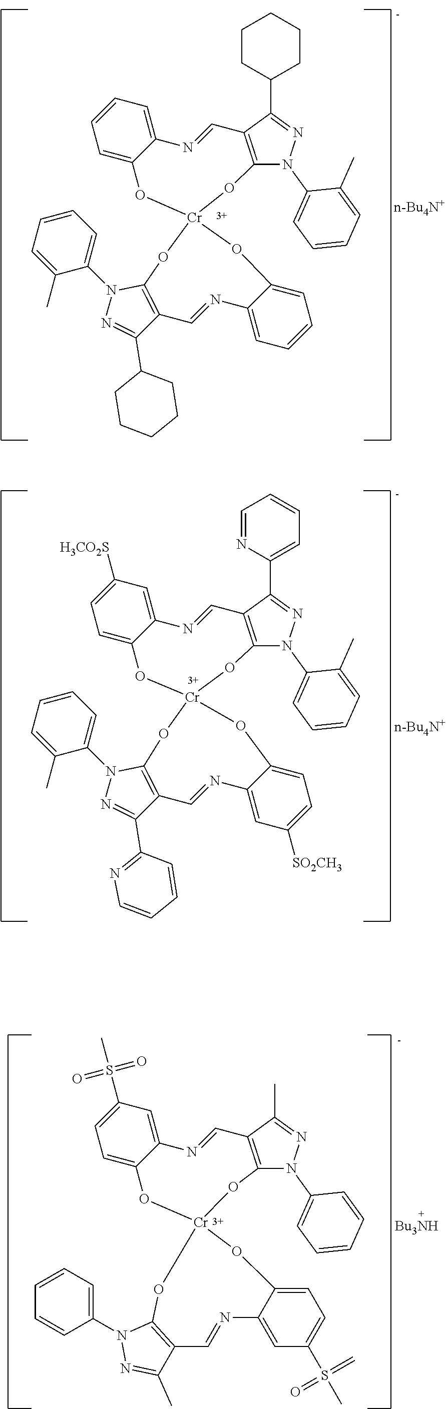

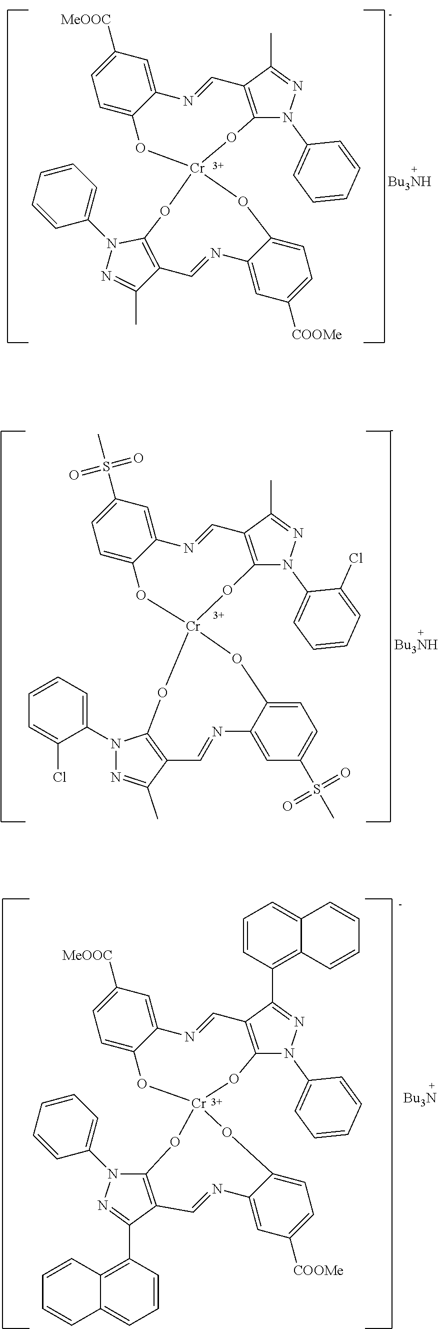

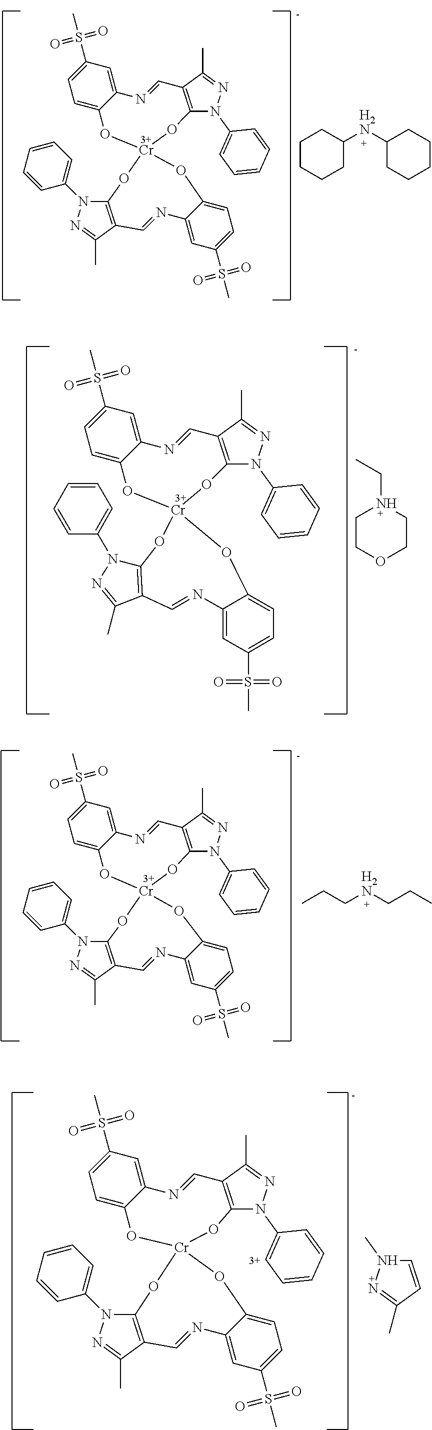

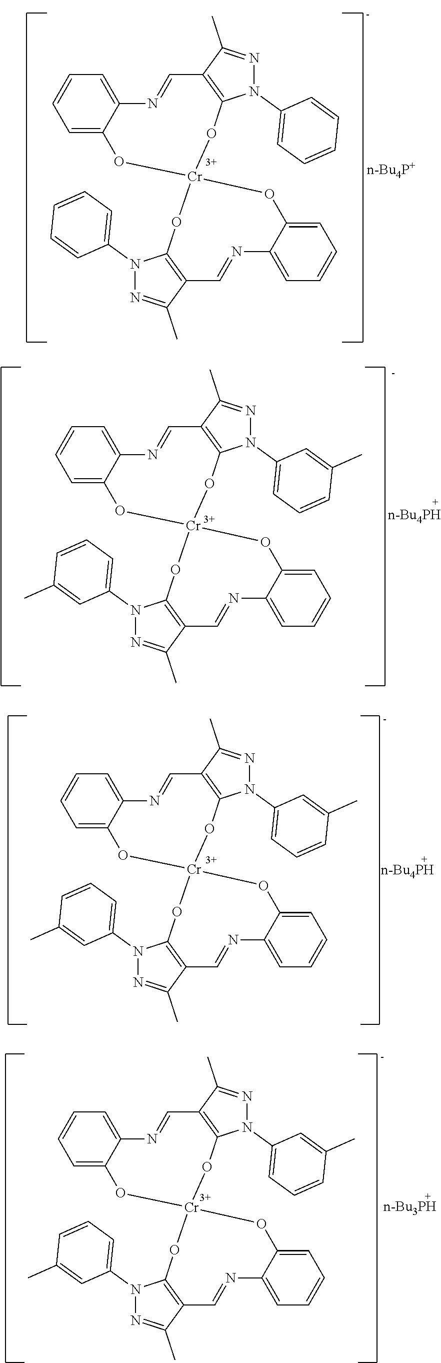

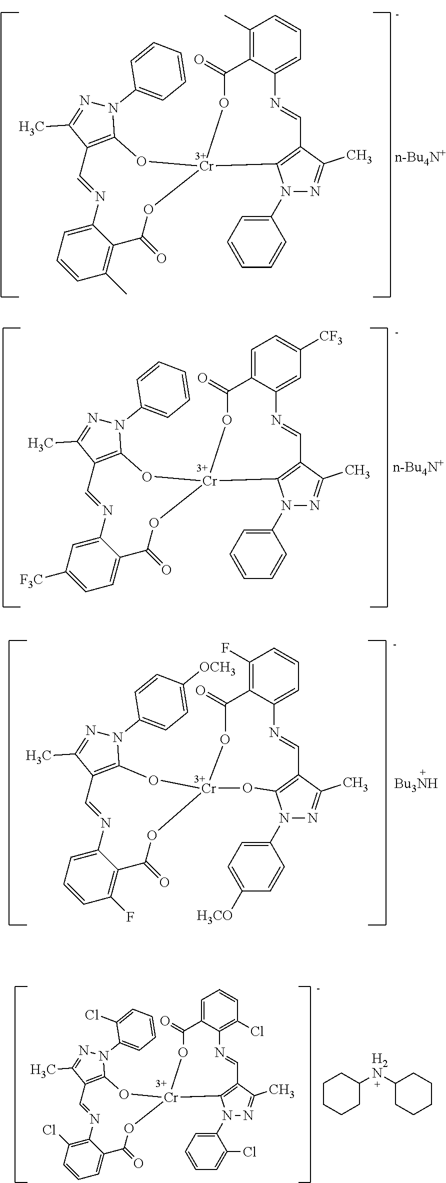

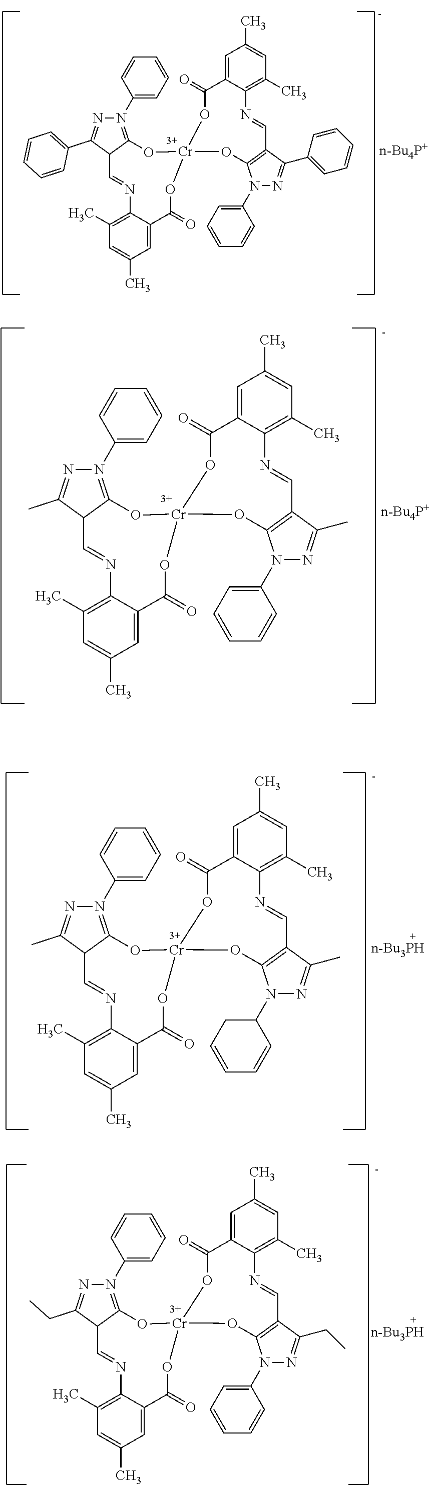

According to one embodiment of the present disclosure, the coordination compound described above is used as a light absorber.

Another embodiment of the present disclosure provides a light absorber including the coordination compound described above.

According to one embodiment of the present disclosure, the light absorber may further include a solvent.

As the solvent, materials known to enable formation of a light absorber from the coordination compound described above in the art relating to the present disclosure may be used without particular limit. For example, the solvent may be one or more types of compounds selected from the group consisting of esters, ethers, ketones, aromatic hydrocarbons and sulfoxides.

Examples of the ester-based solvent may include ethyl acetate, n-butyl acetate, isobutyl acetate, amyl formate, isoamyl acetate, isobutyl acetate, butyl propionate, isopropyl butyrate, ethyl butyrate, butyl butyrate, methyl lactate, ethyl lactate, gamma-butyrolactone, epsilon-caprolactone, delta-valerolactone, alkyl oxyacetate (for example, methyl oxyacetate, ethyl oxyacetate, butyl oxyacetate or the like), alkyl methoxyacetate (for example, ethyl methoxyacetate, butyl methoxyacetate, methyl ethoxyacetate, ethyl methoxyacetate or the like)), alkyl 3-oxypropionates (for example, methyl 3-oxypropionate, ethyl 3-oxypropionate or the like), alkyl 3-methoxypropionates (for example, methyl 3-methoxypropionate, ethyl 3-methoxypropionate, methyl 3-ethoxypropionate, ethyl 3-ethoxypropionate or the like), alkyl 2-oxypropionates (for example, methyl 2-oxypropionate, ethyl 2-oxypropionate, propyl 2-oxypropionate or the like), alkyl 2-methoxypropionates (for example, methyl 2-methoxypropionate, ethyl 2-methoxypropionate, propyl 2-methoxypropionate, methyl 2-ethoxypropionate, ethyl 2-ethoxypropionate), methyl 2-oxy-2-alkylpropionate and ethyl 2-oxy-2-alkylpropionate, (for example, methyl 2-methoxy-2-methylpropionate, ethyl 2-ethoxy-2-methylpropionate or the like), methyl pyruvate, ethyl pyruvate, propyl pyruvate, methyl acetoacetate, ethyl acetoacetate, methyl 2-oxobutanoate, 2-ethyl 2-oxobutanoate and the like.

Examples of the ether-based solvent may include diethylene glycol dimethyl ether, tetrahydrofuran, ethylene glycol monomethyl ether, ethylene glycol monoethyl ether, methyl cellosolve acetate, ethyl cellosolve acetate, diethylene glycol monomethyl ether, diethylene glycol monoethyl ether, diethylene glycol monobutyl ether, propylene glycol monomethyl ether, propylene glycol monomethyl ether acetate, propylene glycol monoethyl ether acetate, propylene glycol monopropyl ether acetate and the like.

Examples of the ketone-based solvent may include methyl ethyl ketone, cyclohexanone, cyclopentanone, 2-heptanone, 3-heptanone, N-methyl-2-pyrrolidone and the like.

Examples of the aromatic hydrocarbon-based solvent may include toluene, xylene, anisole, limonene and the like.

Examples of the sulfoxide-based solvent may include dimethyl sulfoxide and the like.

The solvent is exemplified as above, however, the solvent is not limited to the solvents exemplified above, and any solvent capable of dissolving or dispersing the coordination compound represented by Chemical Formula A of the present disclosure may be used. In addition, the solvent may be used either alone as one type, or as a mixture of two or more types of solvents.

According to one embodiment of the present disclosure, the coordination compound represented by Chemical Formula A has an organic metal complex structure, and the light absorber employing the same has an advantage of enhancing blue luminance transmittance or improving reflected color.

According to one embodiment of the present disclosure, the light absorber may absorb one or more wavelengths selected from among 350 nm to 450 nm and more preferably 380 nm to 450 nm, however, the wavelength is not limited thereto.

One embodiment of the present disclosure provides an adhesive composition including the coordination compound described above. As one example, the adhesive composition is the light absorber described above. As another example, the adhesive composition includes the light absorber described above and other additives.

One embodiment of the present disclosure provides an adhesive composition including at least one selected from the group consisting of a binder resin, a crosslinking agent, a coupling agent, an antioxidant, an antistatic agent, a light stabilizer and a catalyst.

According to one embodiment of the present disclosure, the crosslinking agent induces a crosslinking reaction between an alkali-soluble polyimide resin or other additive components to increase heat resistance and chemical resistance of a produced film. Herein, compounds including a functional group such as an acrylic group or an isocyanate group may be used as the crosslinking agent. In addition, the crosslinking agent may be, for example, a thermal crosslinking agent, compounds including a thermally reactive functional group such as a methylol group or an epoxy group may be used as such a thermal crosslinking agent. Examples of crosslinking agents generally used in the art include, for example, DML-PC, DML-PEP, DML-OC, DML-OEP, DML-34X, DML-PTBP, DML-PCHP, DML-OCHP, DML-PFP, DML-PSBP, DML-POP, DML-MBOC, DML-MBPC, DML-MTrisPC, DML-BisOC-Z, DML-BisOCHP-Z, DML-BPC, DML-BisOC-P, DMOM-PC, DMOM-PTBP, DMOM-MBPC, TriML-P, TriML-35XL, TML-HQ, TML-BP, TML-pp-BPF, TML-BPE, TML-BPA, TML-BPAF, TML-BPAP, TMOM-BP, TMOM-BPE, TMOM-BPA, TMOM-BPAF, TMOM-BPAP, HML-TPPHBA, HML-TPHAP, HMOM-TPPHBA, HMOM-TPHAP (hereinbefore, trade names, manufactured by Honshu Chemical Industry Co., Ltd.), “NIKALAC” (registered trademark) MX-290, “NIKALAC” (registered trademark) MX-280, “NIKALAC” (registered trademark) MX-270, “NIKALAC” (registered trademark) MX-279, “NIKALAC” (registered trademark) MW-100LM, “NIKALAC” (registered trademark) MX-750LM (hereinbefore, trade names, manufactured by Sanwa Chemical Co., Ltd.) and T39M (Soken Chemical & Engineering Co., Ltd.).

According to one embodiment of the present disclosure, the coupling agent may be a silane-based coupling agent, but is not limited thereto, and those known in the art may be properly employed.

According to one embodiment of the present disclosure, the antioxidant may perform a role of preventing a chain reaction that generates radicals during polymer film formation. Herein, a phenol-based antioxidant or the like may be included as the antioxidant, and an antioxidant generally used in the art, such as 2,2-thiobis(4-methyl-6-t-butylphenol), 2,6-g,t-butylphenol or the like, may be used. However, the antioxidant is not limited thereto. According to a preferred embodiment of the present disclosure, Kinox-80 (Hannong Chemicals Inc.) may be used as the antioxidant, however, the antioxidant is not limited thereto.

According to one embodiment of the present disclosure, the antistatic agent is included in the adhesive composition to perform a role of providing antistatic performance, and known antistatic agents may all be used. Examples of the antistatic agent may include ionic compounds. Examples of the ionic compound may include metal salts or organic salts. Examples of the metal salt ionic compound may include alkali metal cations or alkaline-earth metal cations and anions. Examples of the cation included in the metal salts may include one, two or more types of a lithium ion (Li⁺), a sodium ion (Na⁺), a potassium ion (K⁺), a rubidium ion (Rb⁺), a cesium ion (Cs⁺), a beryllium ion (Be²⁺), a magnesium ion (Mg²⁺), a calcium ion (Ca²⁺), a strontium ion (Sr²⁺) and a barium ion (Ba²⁺), and for example, one, or more types of a lithium ion, a sodium ion, a potassium ion, a magnesium ion, a calcium ion and a barium ion may be used, or considering ion stability and mobility, a lithium ion may be used. Examples of the anion included in the metal salts may include PF₆⁻, AsF⁻, NO₂⁻, fluoride (F⁻), chloride (Cl⁻), bromide (Br⁻), iodide (I⁻), perchlorate (ClO₄⁻), hydroxide (OH⁻), carbonate (CO₃²⁻), nitrate (NO₃⁻), trifluoromethanesulfonate (CF₃SO₃⁻), sulfonate (SO₄⁻), hexafluorophosphate (PF₆⁻), methylbenzenesulfonate (CH₃ (C₆H₄) SO₃), p-toluenesulfonate (CH₃C₆H₄SO₃⁻), tetraborate (B₄O₇²⁻), carboxybenzenesulfonate (COOH (C₆H₄) SO₃⁻), trifluoromethanesulfonate (CF₃SO₂⁻), benzonate (C₆H₅COO⁻), acetate (CH₃COO⁻), trifluoroacetate (CF₃COO⁻), tetrafluoroborate (BF₄⁻), tetrabenzylborate (B(C₆H₅)₄⁻), trispentafluoroethyl trifluorophosphate (P(C₂F₅)₃F₃⁻) or the like. According to a preferred embodiment of the present disclosure, FC-4400 (3M Corporation) may be used as the antistatic agent, however, the antistatic agent is not limited thereto.

According to one embodiment of the present disclosure, the light stabilizer is a material that does not agglomerate even when the adhesive is left unattended at a high temperature, and thereby significantly improves storage stability of the adhesive composition by preventing the decomposition of the bonding site by heat and generating radicals, and without causing a phenomenon of increasing a concentration of the antistatic agent. Known light stabilizers may be used. According to preferred one embodiment of the present disclosure, hindered amine compounds may be used as the light stabilizer, and specifically, Tinuvin 123 (BASF Corporation) may be used, however, the light stabilizer is not limited thereto.

According to one embodiment of the present disclosure, the catalyst is a material controlling a rate of the reaction to prepare the adhesive composition according to one embodiment of the present disclosure. According to one embodiment of the present disclosure, dibutyltin dilaurate (Sigma-Aldrich) may be used as the catalyst, however, the catalyst is not limited thereto.

In one embodiment of the present disclosure, the coordination compound represented by Chemical Formula A is included in 0.1 parts by weight to 0.5 parts by weight based on 100 parts by weight of the total weight of the adhesive composition solid content.

Preferably, the coordination compound represented by Chemical Formula A is included in 0.2 parts by weight to 0.4 parts by weight based on 100 parts by weight of the total weight of the adhesive composition solid content. More preferably, the coordination compound represented by Chemical Formula A is included in 0.25 parts by weight to 0.35 parts by weight based on 100 parts by weight of the total weight of the adhesive composition solid content.

When the coordination compound represented by Chemical Formula A is included in the above-described content in the adhesive composition, excellent light resistance, heat resistance and moisture resistance are obtained. When the coordination compound represented by Chemical Formula A is included in less than 0.2 parts by weight, a sufficient effect of light resistance may not be achieved, and including the coordination compound represented by Chemical Formula A in greater than 0.4 parts by weight may cause undesirable precipitation of the dye on the thin film.

In one embodiment of the present disclosure, the binder resin is included in 90 parts by weight to 99 parts by weight, the crosslinking agent is included in 0.05 parts by weight to 0.5 parts by weight, the coupling agent is included in 0.05 parts by weight to 0.5 parts by weight, the antioxidant is included in 0.2 parts by weight to 0.8 parts by weight, the antistatic agent is included in 0.5 parts by weight to 2.5 parts by weight, the light stabilizer is included in 1 parts by weight to 4 parts by weight and the catalyst is included in 0.001 parts by weight to 0.02 parts by weight, based on 100 parts by weight of the total weight of the adhesive composition solid content.

In one embodiment of the present disclosure, the binder resin is included in 93 parts by weight to 97 parts by weight based on 100 parts by weight of the total weight of the adhesive composition solid content.

In one embodiment of the present disclosure, the crosslinking agent is included in 0.3 parts by weight to 0.4 parts by weight based on 100 parts by weight of the total weight of the adhesive composition solid content.

In one embodiment of the present disclosure, the coupling agent is included in 0.1 parts by weight to 0.3 parts by weight based on 100 parts by weight of the total weight of the adhesive composition solid content.

In one embodiment of the present disclosure, the antioxidant is included in 0.4 parts by weight to 0.6 parts by weight based on 100 parts by weight of the total weight of the adhesive composition solid content.

In one embodiment of the present disclosure, the antistatic agent is included in 0.1 parts by weight to 2 parts by weight based on 100 parts by weight of the total weight of the adhesive composition solid content.

In one embodiment of the present disclosure, the light stabilizer is included in 1.5 parts by weight to 3.5 parts by weight based on 100 parts by weight of the total weight of the adhesive composition solid content.

In one embodiment of the present disclosure, the catalyst is included in 0.008 parts by weight to 0.02 parts by weight based on 100 parts by weight of the total weight of the adhesive composition solid content.

When the binder resin, the crosslinking agent, the coupling agent, the antioxidant, the antistatic agent, the light stabilizer or the catalyst is included in the above-described content in the adhesive composition, a film having a uniform thickness may be prepared using the adhesive composition.

According to one embodiment of the present disclosure, the adhesive composition may further include a solvent. As the solvent, the solvent described above in reference to the light absorber may be used.

The solvent is not limited to the solvents exemplified above, and any solvent capable of dissolving or dispersing the coordination compound represented by Chemical Formula A of present disclosure may be used.

According to preferred one embodiment of the present disclosure, the solvent included in the adhesive composition is ethyl acetate or methyl ethyl ketone.

In addition, the solvent may be used either alone as one type, or as a mixture of two or more types of solvents. For example, according to one embodiment of the present disclosure, the solvent included in the adhesive composition is a mixed solution of ethyl acetate or methyl ethyl ketone. Herein, the solvents of the mixed solution may be mixed and used in a ratio of 1:3 to 3:1, however, this is just an example, and the mixing ratio is not limited to the example.

According to preferred one embodiment of the present disclosure, methyl ethyl ketone (MEK) may be used as the solvent, however, the solvent is not limited thereto.

According to one embodiment of the present disclosure, the solvent may be included in 10 parts by weight to 50 parts by weight or 20 parts by weight to 40 parts by weight based on 100 parts by weight of the total weight of the adhesive composition. According to a preferred embodiment of the present disclosure, the solvent may be included in 20 parts by weight to 30 parts by weight based on the total parts by weight of the adhesive composition, however, the content is not limited thereto.

When the solvent is included in the adhesive composition in the above-described range, there is no concern for precipitation of a dye or the like, and performance of the adhesive film including the adhesive composition may be readily identified.

In one embodiment of the present disclosure, the adhesive composition may further include a binder resin.

Known materials commonly used in the art may be used as the binder resin as long as it is a material providing adhesiveness and usable in a film for a display. Examples of the binder resin may include an acryl-based polymer, an acrylate-based copolymer, a silicone-based polymer, polyester, polyurethane, polyether, an epoxy resin and the like, but are not limited thereto.

According to one embodiment of the present disclosure, the binder resin may be an acrylate-based resin. The acrylate-based resin may be an acrylate-based polymer or an acrylate-based copolymer. Examples of the acrylate-based copolymer may include butyl acrylate/hydroxyethyl acrylate, but are not limited thereto.

According to a preferred embodiment of the present disclosure, AD-701 of LG Chem. may be used as the binder resin, however, the binder resin is not limited thereto.

According to one embodiment of the present disclosure, the binder resin may be included in 93 parts by weight to 97 parts by weight; or 94 parts by weight to 96 parts by weight based on 100 parts by weight of the total weight of the adhesive composition solid content. According to a preferred embodiment of the present disclosure, the binder resin may be included in 95 parts by weight to 96 parts by weight based on 100 parts by weight of the total weight of the adhesive composition solid content.

When the binder resin is included in the adhesive composition in the above-mentioned range, adhesiveness of the adhesive film including the adhesive composition is maintained, and an adhesive film having a uniform thickness may be prepared from the adhesive composition.

In the present disclosure, the ‘adhesive composition solid content’ means excluding the solvent from the adhesive composition and including the coordination compound represented by Chemical Formula A.

According to another embodiment of the present disclosure, the coordination compound of Chemical Formula A may be used together with other types of dyes. Dyes commonly used in the art may be used together with the coordination compound of Chemical Formula A. As a specific example of the dye that may be used together with the coordination compound of Chemical Formula A, one or more types of compounds may be selected from the group consisting of metal-complex-based compounds; azo-based compounds; metal azo-based compounds; quinophthalone-based compounds; isoindoline-based compounds; methine-based compounds; phthalocyanine-based compounds; metal phthalocyanine-based compounds; porphyrin-based compounds; metal porphyrin-based compounds; tetra-azaporphyrin-based compounds; metal tetra-azaporphyrin-based compounds; cyanine-based compounds; xanthene-based compounds; metal dipyrromethane-based compounds; boron dipyrromethane-based compounds; metal dipyrromethane-based compounds; anthraquinone-based compounds; diketopyrrolopyrrole-based compounds; triarylmethane-based compounds; and perylene-based compounds. However, the dye is not limited thereto.

One embodiment of the present disclosure provides an adhesive film including the light absorber.

According to one embodiment of the present disclosure, the adhesive film including the light absorber may include the light absorber as it is.

According to another embodiment of the present disclosure, the adhesive film including the light absorber may include the light absorber after the solvent has been removed by drying.

Another embodiment of the present disclosure provides an adhesive film including the adhesive composition.

According to one embodiment of the present disclosure, the adhesive film may be used as an optical film used in a display, an electronic device or the like. The optical film may be used as a retardation film, an anti-reflection film, an anti-glare film, an ultraviolet absorption film, an infrared absorption film, an optical compensation film, a brightness enhancement film or the like.

According to one embodiment of the present disclosure, the adhesive film including the adhesive composition may include the adhesive composition as it is.

According to another embodiment of the present disclosure, the adhesive film including the adhesive composition may include the adhesive composition after the solvent has been removed by drying.

According to still another embodiment of the present disclosure, the adhesive film including the adhesive composition may include a cured material of the adhesive composition.

In one embodiment of the present disclosure, a maximum absorption wavelength of the adhesive film is in a range of 380 nm to 450 nm.

In still another embodiment of the present disclosure, a maximum absorption wavelength of the adhesive film is in a range of 380 nm to 440 nm.

When the adhesive film has a maximum absorption wavelength in a range of 380 nm to 450 nm, a decrease in the blue luminance may be minimized by absorbing a longer wavelength region compared to an existing blue light blocking film, and efficiency may be maximized.

One embodiment of the present disclosure provides an optical film including the light absorber.

According to one embodiment of the present disclosure, the optical film includes an adhesive film and a binder resin film.

According to one embodiment of the present disclosure, the adhesive film includes the light absorber.

According to one embodiment of the present disclosure, the optical film includes an adhesive film and a binder resin film, and the adhesive film includes the light absorber.

Another embodiment of the present disclosure provides an optical film including the adhesive film and other films.

Examples of the other films may include a release film, an anti-reflection film, a binder resin film and the like.

Specifically, one embodiment of the present disclosure provides an optical film including an adhesive film and a binder resin film.

The optical film may further include a base as necessary.

FIG. 2 illustrates an optical film according to one embodiment of the present disclosure in which a base (1); an adhesive film (2); and a binder resin film (3) are consecutively laminated.

In the present disclosure, transmittance means transmittance for light.

In the present disclosure, a change in the transmittance for light is obtained by measuring transmittance of the optical film including the coordination compound represented by Chemical Formula A, using the transmittance of an optical film that does not include the coordination compound represented by Chemical Formula A as 100%, and measuring the changed value.

Specifically, the changed value may be calculated by the following formula:

ΔT(%)={(transmittance measured immediately after preparing the optical film-transmittance remeasured after exposing the optical film to each condition of light resistance, heat resistance and moisture resistance)/transmittance measured immediately after preparing the optical film}×100.

According to one embodiment of the present disclosure, the adhesive film is used in a display, an electronic device or the like instead of a polarizing plate, which more effectively reduces a change in the transmittance for each wavelength compared to an optical film including a polarizing plate. In addition, the adhesive film includes an adhesive composition including the coordination compound of Chemical Formula A, which more effectively reduces a change in the transmittance for each wavelength compared to an optical film including other compositions. Accordingly, color tones of the emitted color may be flexibly adjusted.

According to one embodiment of the present disclosure, the adhesive film including the adhesive composition may have a form in which an adhesive composition is laminated and/or coated on one surface of a base.

According to one embodiment of the present disclosure, the material of the base may be selected from the group consisting of PET (polyethylene terephthalate), polyester, PC (polycarbonate), PI (polyimide), PEN (polyethylene naphthalate), PEEK (polyether ether ketone), PAR (polyarylate), PCO (polycyclicolefin), polynorbornene, PES (polyethersulphone) and COP (cycloolefin polymer.

In addition, the base is preferably transparent. The base being transparent referred herein means that light transmittance of visible light having a wavelength range of 400 nm to 700 nm is 80% or greater. When the base has light transmittance in the above-mentioned range, the laminated adhesive film may be a thin film.

According to one embodiment of the present disclosure, the adhesive film including the adhesive composition has a thickness of 10 μm to 40 μm; 15 μm to 30 μm; or 20 μm to 25 μm. According to a preferred embodiment of the present disclosure, the adhesive film including the adhesive composition has a thickness of 22 μm to 23 μm.

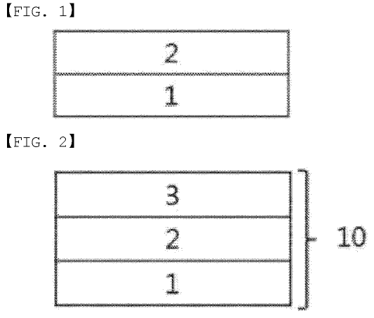

According to one embodiment of the present disclosure, the adhesive film may be prepared by coating the adhesive composition on the base using a bar coater. As a specific example, FIG. 1 illustrates a case of the base (1) and the adhesive film (2) being consecutively laminated according to one embodiment of the present disclosure.

According to one embodiment of the present disclosure, the release film may be included on another surface other than the one surface of the base on which the adhesive composition is laminated and/or coated.

A hydrophobic film may be used as the release film. The release film refers to, as a film for protecting a very thin adhesive film, a transparent layer attached to one surface of the adhesive film, and films having excellent mechanical strength, thermal stability, moisture shielding properties, isotropy and the like may be used. For example, acetate-based such as triacetyl cellulose (TAC), polyester-based, polyethersulphone-based, polycarbonate-based, polyamide-based, polyimide-based, polyolefin-based, cycloolefin-based, polyurethane-based and acryl-based resin films, and the like may be used, and commercially available silicone-treated release films may be used. However, the release film is not limited to these examples.

According to one embodiment of the present disclosure, the anti-reflection film improves reflected color sense by adjusting the degree of light reflection generated from an external light source.

According to one embodiment of the present disclosure, the anti-reflection film has a lowest reflection wavelength of 550 nm or less; or 530 nm or less. According to a preferred embodiment of the present disclosure, the anti-reflection film has a lowest reflection wavelength of 500 nm or less.

A material of the anti-reflection film may be properly selected to satisfy properties of the lowest reflection wavelength. For example, the anti-reflection film may include a low refractive index layer. For example, the lowest reflection wavelength of the anti-reflection film tends to shift to a longer wavelength as a thickness of the low refractive index layer increases, and tends to shift to a shorter wavelength as a thickness of the low refractive index layer decreases. For example, the lowest reflectivity of the anti-reflection film tends to decrease as a refractive index of a low refractive material decreases.

The low refractive index layer may include a low refractive material. In one example, the low refractive material may be a low refractive inorganic particle. In another example, the low refractive inorganic particle may be a silica-based particle. Examples of the silica-based particle may include hollow silica, mesoporous silica and the like, but are not limited thereto. In another example, magnesium fluoride (MgF₂) may be used as the low refractive inorganic particle.

According to one embodiment of the present disclosure, the binder resin film may include a TAC (triacetate cellulose) film or the like, but is not limited thereto, and known binder resin films commonly used in the art may be used.

The binder resin film has a thickness of 20 μm to 60 μm; 30 μm to 50 μm; or 35 μm to 45 μm.

In one embodiment of the present disclosure, the adhesive film may be provided on the base, and the binder resin film may be further provided on a surface opposite to the surface on which the adhesive film and the base are in contact with each other.

One embodiment of the present disclosure provides an electronic device including the light absorber described above.

One embodiment of the present disclosure provides an electronic device including the adhesive film.

According to one embodiment of the present disclosure, the electronic device includes an adhesive film, and the adhesive film includes the light absorber.

The electronic device may include all of an interlayer insulating film of a semiconductor device, a color filter, a black matrix, an overcoat, a column spacer, a passivation film, a buffer coat film, an insulating film for a multilayer print substrate, a cover coat of a flexible copper clad plate, a buffer coat film, an insulating film solder resist film for a multilayer print substrate, an insulating film of an OLED, a protective film of a thin film transistor of a liquid crystal display apparatus, an electrode protective film of an organic EL device and a semiconductor protective film, an OLED insulating film, an LCD insulating film, a semiconductor insulating film, a display apparatus and the like, but is not limited thereto.

One embodiment of the present disclosure provides a display apparatus including the light absorber.

Another embodiment of the present disclosure provides a display apparatus including the adhesive film.

One embodiment of the present disclosure provides an organic light emitting display apparatus including the light absorber.

Another embodiment of the present disclosure provides an organic light emitting display apparatus including the adhesive film.

One embodiment of the present disclosure provides an organic light emitting display apparatus including an adhesive film including the adhesive composition described above; a substrate provided on one surface of the adhesive film; and an organic light emitting layer provided on a surface of the substrate opposite to the surface in contact with the adhesive film.

Hereinafter, the present disclosure will be described in detail with reference to examples in order to specifically describe the present disclosure. However, the examples according to the present disclosure may be modified to various different forms, and the scope of the present disclosure is not to be construed as being limited to the examples described below. Examples of the present disclosure are provided in order to more fully describe the present disclosure to those having average knowledge in the art.

### Synthesis Example

The coordination compound represented by Chemical Formula A may be prepared using, for example, a preparation method as shown in Reaction Formula 1. The preparation method may be more specified in the preparation examples described below.

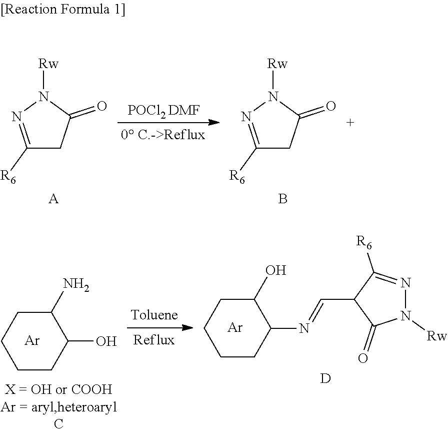

### 1) Synthesis of Compound B

Phosphoryl chloride (1.2 equivalents) was diluted in an excess dimethylformamide (DMF) solvent, and stirred for 30 minutes under nitrogen while maintaining a temperature at 0° C. When the temperature was sufficiently stabilized, Compound A (1 equivalent) was slowly added thereto, and after raising the reaction temperature to 80° C., the mixture was sufficiently reacted under nitrogen. After identifying the completion of the reaction through thin layer chromatography (TLC), the reaction temperature was lowered to room temperature, and the result was introduced to prepared ice water and sufficiently stirred. After that, a produced solid compound was secured through vacuum filtration, and extracted using chloroform and water. Anhydrous magnesium sulfate was introduced to the extracted organic layer to remove water, and the result was concentrated and recrystallized with chloroform and ethanol to obtain a product.

2) Synthesis of Compound D

Secured Compound B (1 equivalent) was diluted in a toluene solution together with synthesized or commercially available Compound C (1.2 equivalents), and the mixture was stirred under nitrogen and reacted while heating to a temperature of 120° C. After the reaction was completed, the temperature was lowered to room temperature, and a formed solid product was vacuum filtered while sufficiently washing with ethanol and hexane to obtain a product. The solvent was sufficiently removed from Secured Compound D through oven drying.

### PREPARATION EXAMPLE: PREPARATION OF COMPOUNDS

### Preparation Example 1: Synthesis of Compound P1

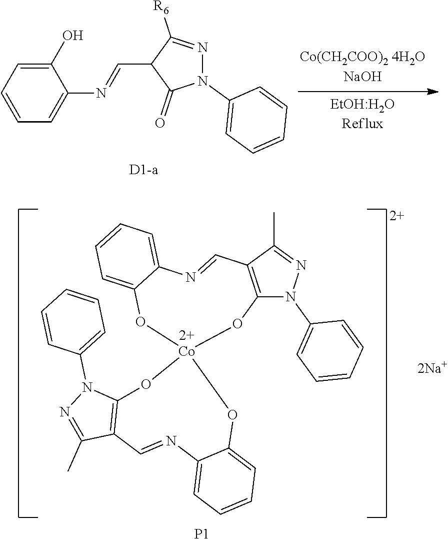

Compound D1-a (100 mg, 1 equivalent) synthesized using the method of Reaction Formula 1 was diluted in ethanol (5 mL), and stirred while heating under nitrogen. After that, cobalt acetate tetrahydrate (42.5 mg, 0.5 equivalents) dissolved in water (5 mL) was added thereto, and sodium hydroxide (10 mg, 2 equivalents) was further introduced thereto an reacted. After the reaction was finished, the result was concentrated, and extracted using chloroform and water. From the organic layer secured through extraction, water was removed using anhydrous magnesium sulfate, and the result was concentrated through vacuum distillation. The concentrated product was recrystallized using chloroform and ethanol to obtain Compound P1 (86 mg, yield: 72%).

HR LC/MS/MS m/z calculated for C₃₄H₂₆CoN₆Na₂O₄ (M+): 687.1143; found: 687.1143.

### Preparation Example 2: Synthesis of Compound P2

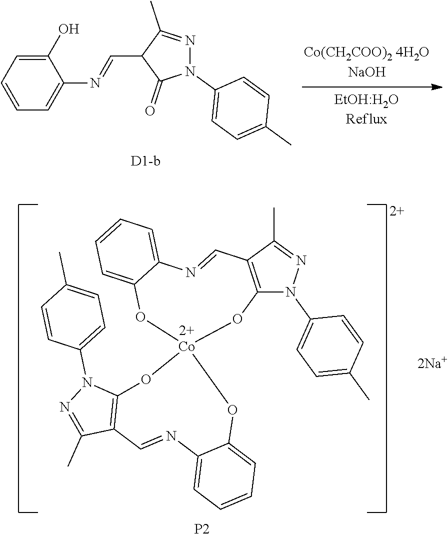

Compound P2 (156 mg, yield: 67%) was prepared by conducting a reaction in the same manner as in Preparation Example 1 except that D1-b (200 mg, 1 equivalent) was used instead of D1-a.

HR LC/MS/MS m/z calculated for C₃₆H₃₀CoN₆Na₂O₄ (M+): 715.1456; found: 715.1455.

### Preparation Example 3: Synthesis of Compound P3

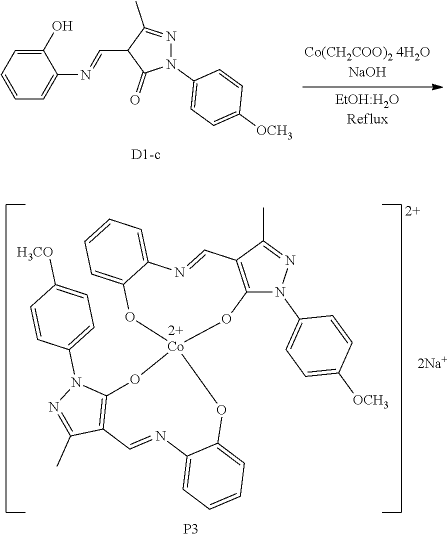

Compound P3 (100 mg, yield: 58%) was prepared by conducting a reaction in the same manner as in Preparation Example 1 except that D1-c (150 mg, 1 equivalent) was used instead of D1-a.

HR LC/MS/MS m/z calculated for C₃₆H₃₀CoN₆Na₂O₄ (M+): 747.1354; found: 747.1354.

### Preparation Example 4: Synthesis of Compound P4

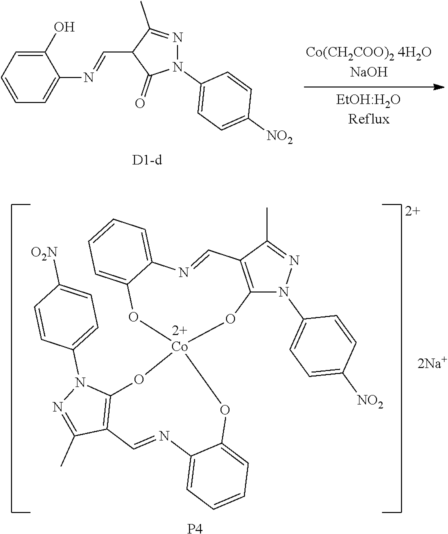

Compound P4 (122 mg, yield: 71%) was prepared by conducting a reaction in the same manner as in Preparation Example 1 except that D1-d (150 mg, 1 equivalent) was used instead of D1-a.

HR LC/MS/MS m/z calculated for C₃₄H₂₄CoN₈Na₂O₈(M+): 777.0844; found: 777.0844.

### Preparation Example 5: Synthesis of Compound P5

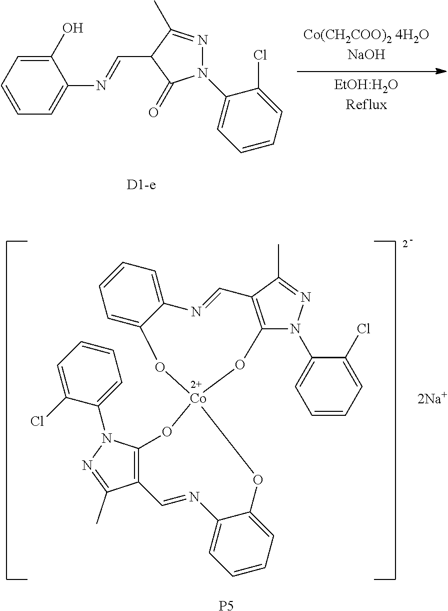

Compound P5 (103 mg, yield: 45%) was prepared by conducting a reaction in the same manner as in Preparation Example 1 except that D1-e (200 mg, 1 equivalent) was used instead of D1-a.

HR LC/MS/MS m/z calculated for C₃₄H₂₄ClCoN₈Na₂O₄ (M+): 755.0358; found: 755.0358.

### Preparation Example 6: Synthesis of Compound P6

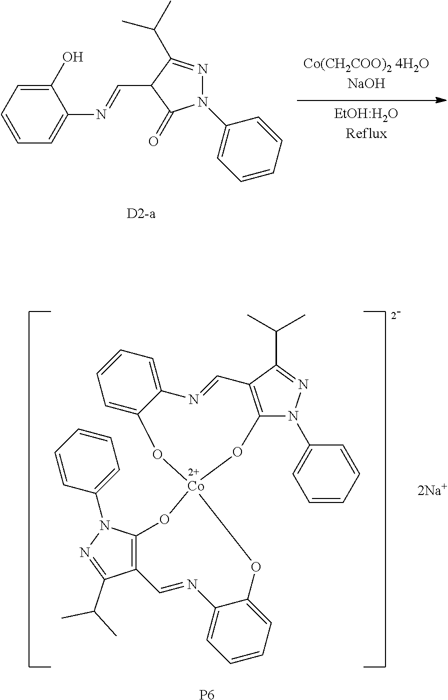

Compound P6 (142 mg, yield: 59%) was prepared by conducting a reaction in the same manner as in Preparation Example 1 except that D2-a (200 mg, 1 equivalent) was used instead of D1-a, and potassium hydroxide (69.8 mg, 2 equivalents) was used instead of sodium hydroxide.

HR LC/MS/MS m/z calculated for C₃₈H₃₄CoK₂N₆O₄ (M+): 775.1248; found: 775.1248.

### Preparation Example 7: Synthesis of Compound P7

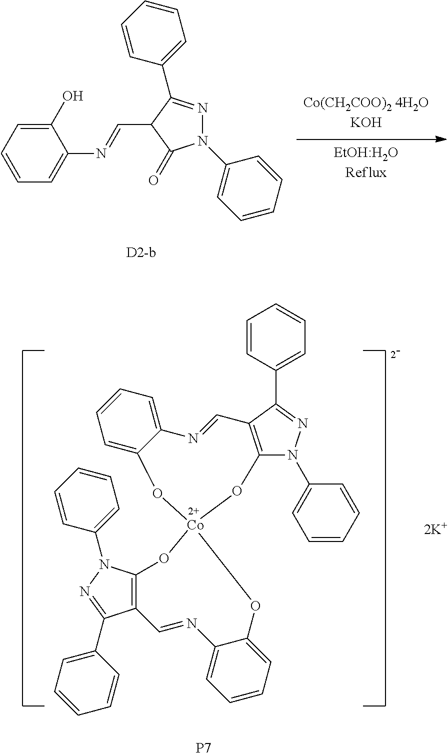

Compound P7 (171 mg, yield: 72%) was prepared by conducting a reaction in the same manner as in Preparation Example 1 except that D2-b (200 mg, 1 equivalent) was used instead of D1-a, and potassium hydroxide (69.8 mg, 2 equivalents) was used instead of sodium hydroxide.

HR LC/MS/MS m/z calculated for C₄₄H₃₀CoK₂N₆O₄ (M+): 843.8918; found: 843.8918.

### Preparation Example 8: Synthesis of Compound P8

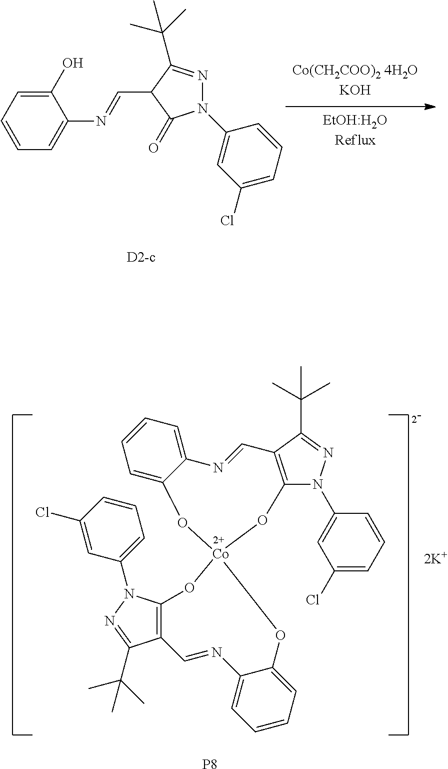

Compound P8 (146 mg, yield: 62%) was prepared by conducting a reaction in the same manner as in Preparation Example 1 except that D2-c (200 mg, 1 equivalent) was used instead of D1-a, and potassium hydroxide (69.8 mg, 2 equivalents) was used instead of sodium hydroxide.

HR LC/MS/MS m/z calculated for C₄₀H₃₆Cl₂CoK₂N₆O₄ (M+): 871.0781; found: 871.0781.

### Preparation Example 9: Synthesis of Compound P9

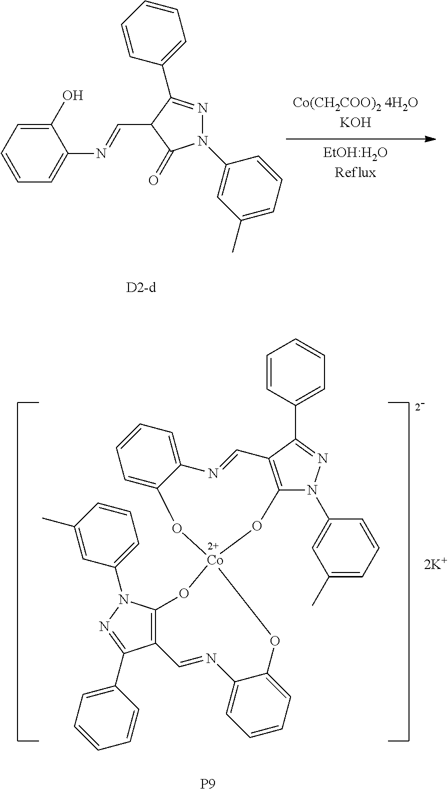

Compound P9 (137 mg, yield: 58%) was prepared by conducting a reaction in the same manner as in Preparation Example 1 except that D2-d (200 mg, 1 equivalent) was used instead of D1-a, and potassium hydroxide (69.8 mg, 2 equivalents) was used instead of sodium hydroxide.

HR LC/MS/MS m/z calculated for C₄₆H₃₄CoK₂N₆O₄ (M+): 871.1248; found: 871.1248.

### Preparation Example 10: Synthesis of Compound P10

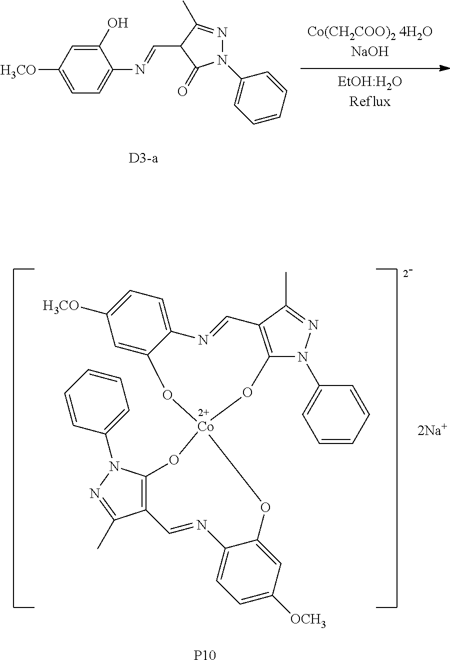

Compound P10 (146 mg, yield: 42%) was prepared by conducting a reaction in the same manner as in Preparation Example 1 except that D3-a (300 mg, 1 equivalent) was used instead of D1-a.

HR LC/MS/MS m/z calculated for C₃₆H₃₀CoN₆Na₂O₆(M+): 747.5847; found: 747.5846.

### Preparation Example 11: Synthesis of Compound P11

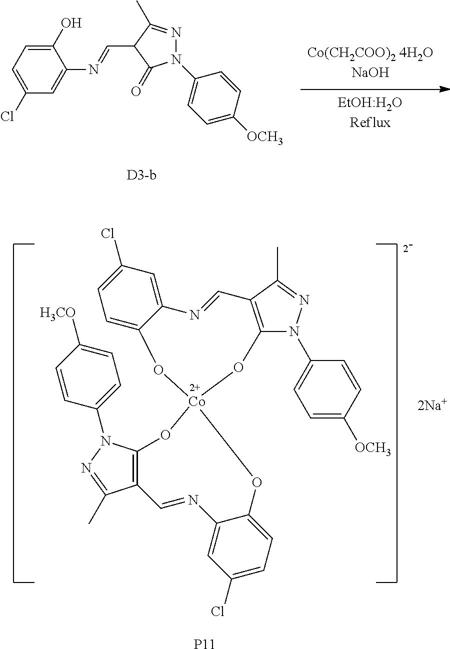

Compound P11 (110 mg, yield: 48%) was prepared by conducting a reaction in the same manner as in Preparation Example 1 except that D3-b (200 mg, 1 equivalent) was used instead of D1-a.

HR LC/MS/MS m/z calculated for C₃₆H₂₈Cl₂CoN₆Na₂O₆(M+): 815.4687; found: 815.4687.

### Preparation Example 12: Synthesis of Compound P12

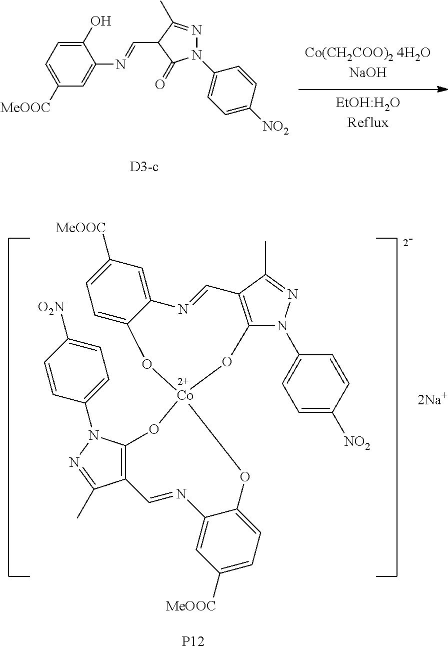

Compound P12 (189 mg, yield: 67%) was prepared by conducting a reaction in the same manner as in Preparation Example 1 except that D3-c (250 mg, 1 equivalent) was used instead of D1-a.

HR LC/MS/MS m/z calculated for C₃₈H₂₈CoN₈Na₂O₁₂ (M+): 893.09542; found: 893.0954.

### Preparation Example 13: Synthesis of Compound P13

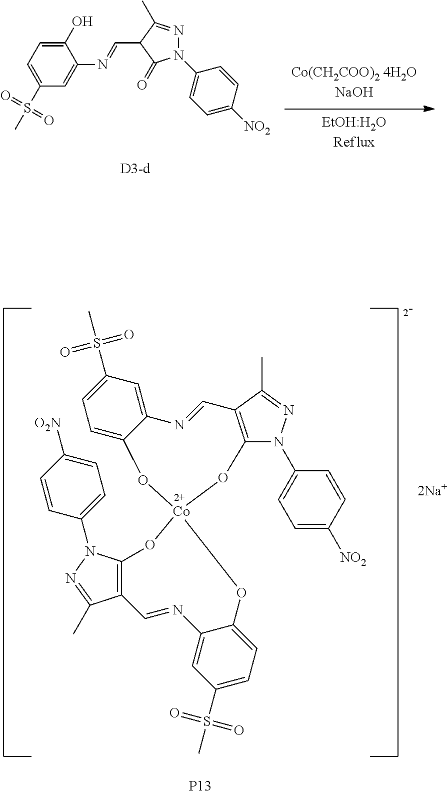

Compound P13 (117 mg, yield: 52%) was prepared by conducting a reaction in the same manner as in Preparation Example 1 except that D3-d (200 mg, 1 equivalent) was used instead of D1-a.

HR LC/MS/MS m/z calculated for C₃₆H₂₈CoN₈Na₂O₁₂S₂ (M+): 933.0395; found: 933.0395.

### Preparation Example 14: Synthesis of Compound P14

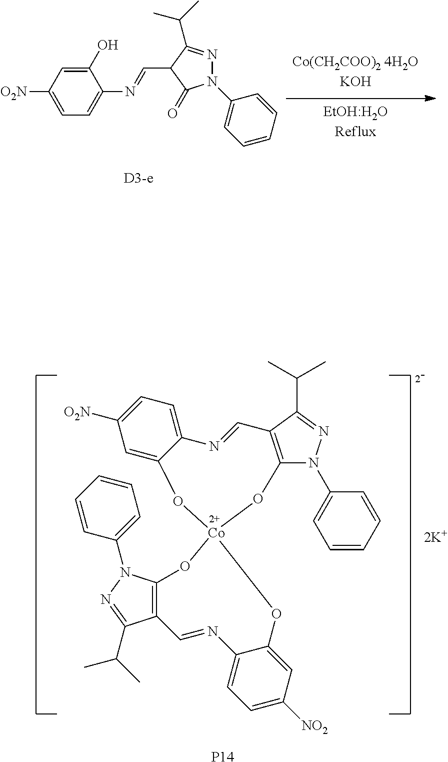

Compound P14 (191 mg, yield: 81%) was prepared by conducting a reaction in the same manner as in Preparation Example 1 except that D3-e (200 mg, 1 equivalent) was used instead of D1-a, and potassium hydroxide (69.8 mg, 2 equivalents) was used instead of sodium hydroxide.

HR LC/MS/MS m/z calculated for C₃₈H₃₂CoK₂N₈O₈ (M+): 865.0949; found: 865.0949.

### Preparation Example 15: Synthesis of Compound P15

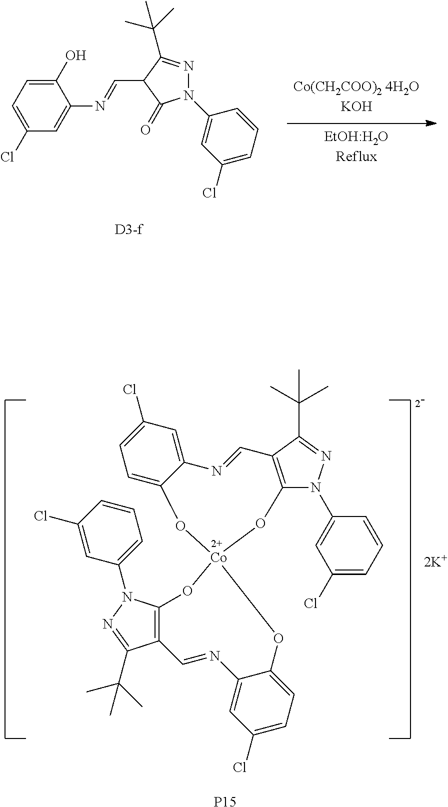

Compound P15 (258 mg, yield: 74%) was prepared by conducting a reaction in the same manner as in Preparation Example 1 except that D3-f (300 mg, 1 equivalent) was used instead of D1-a, and potassium hydroxide (69.8 mg, 2 equivalents) was used instead of sodium hydroxide.

HR LC/MS/MS m/z calculated for C₄₀H₃₄CoK₂N₆O₄ (M+): 940.9972; found: 940.9972.

### Preparation Example 16: Synthesis of Compound P16

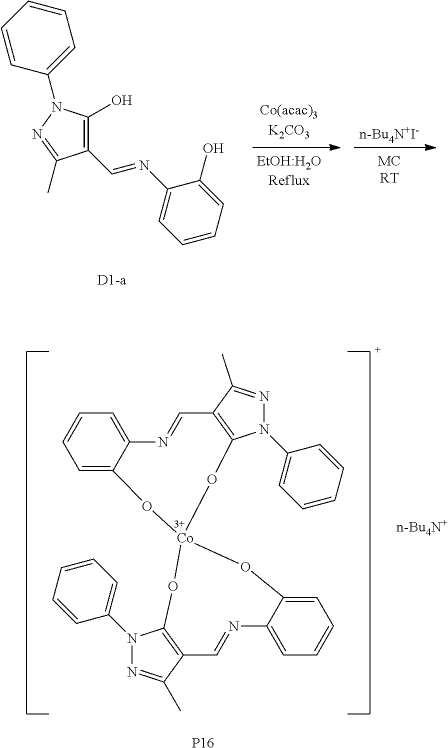

Compound D1-a (500 mg, 1 equivalent) synthesized using the method of Reaction Formula 1 was diluted in ethanol (10 mL), and after adding potassium carbonate (471 mg, 2 equivalents) dissolved in water (10 mL) thereto, the mixture was stirred while heating under nitrogen. After that, tris(acetylacetonato)cobalt (III) (0.304 g, 0.5 equivalents) was added thereto, and the reaction proceeded until the reactant D1-a was not visible on thin layer chromatography (TLC). After concentrating the solvent from the completed reaction material, a dichloromethane solvent (20 mL) was added thereto, then tetra-n-butylammonium iodine (0.378 g, 0.6 equivalents) was introduced thereto, and the result was stirred at room temperature. After the reaction was completed, the result was extracted by adding water thereto. From the organic layer secured therethrough, water was removed using anhydrous magnesium sulfate, and the result was concentrated through vacuum distillation. The concentrated product was diluted in ethanol, and then water was added thereto to obtain Compound P16 (407 mg, yield: 54%).

HR LC/MS/MS m/z calculated for C₅₀H₆₂CoN₇O₄(M+): 883.4195; found: 883.4194.

### Preparation Example 17: Synthesis of Compound P17

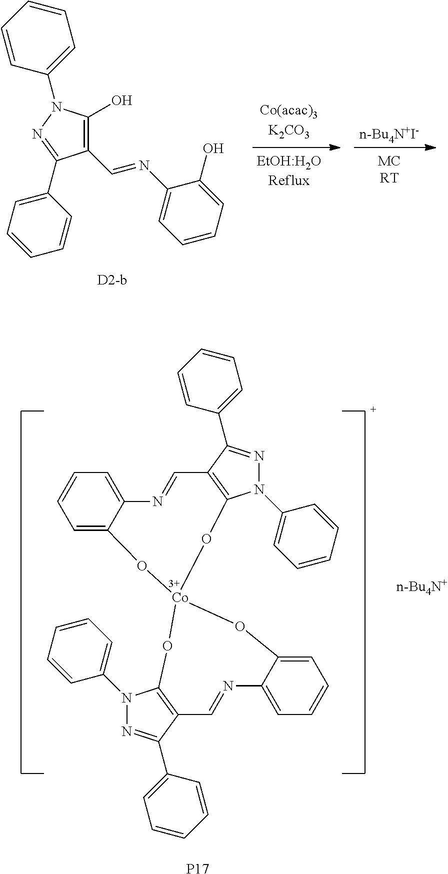

Compound P17 (440 mg, yield: 62%) was prepared by conducting a reaction in the same manner as in Preparation Example 16 except that D2-b (500 mg, 1 equivalent) was used instead of D1-a.

HR LC/MS/MS m/z calculated for C₆₀H₆₆CoN₇O₄(M+): 1007.4508; found: 1007.4508.

### Preparation Example 18: Synthesis of Compound P18

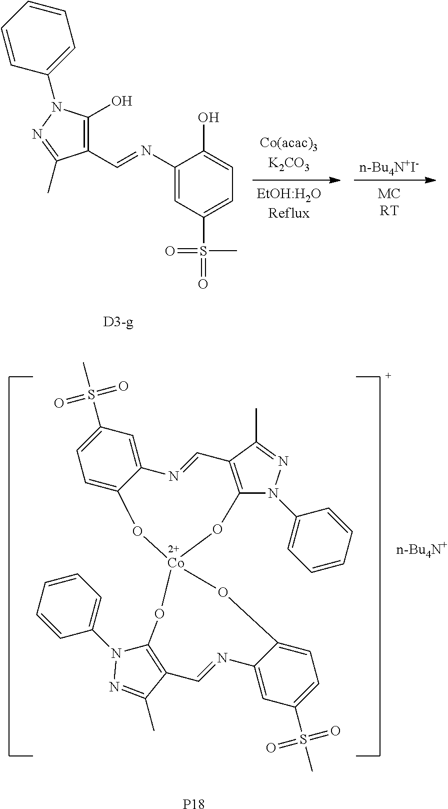

Compound P18 (336 mg, yield: 48%) was prepared by conducting a reaction in the same manner as in Preparation Example 16 except that D3-g (500 mg, 1 equivalent) was used instead of D1-a.

HR LC/MS/MS m/z calculated for C₅₂H₆₆CoN₇O₈S₂ (M+): 1039.3746; found: 1039.3746.

### Preparation Example 19: Synthesis of Compound P19

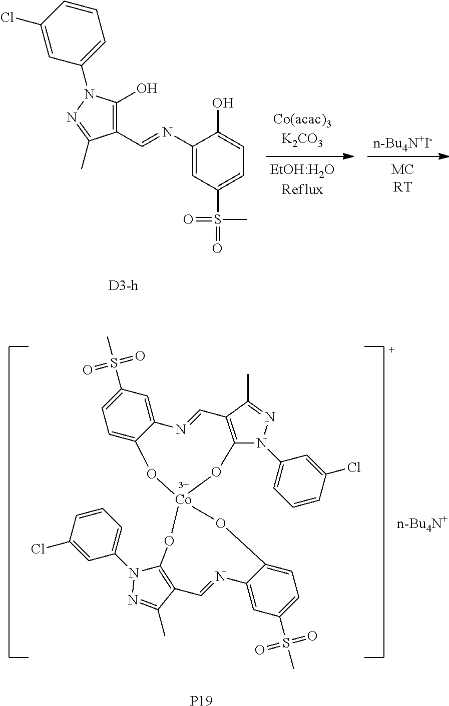

Compound P19 (376 mg, yield: 55%) was prepared by conducting a reaction in the same manner as in Preparation Example 16 except that D3-h (500 mg, 1 equivalent) was used instead of D1-a.

HR LC/MS/MS m/z calculated for C₅₂H₆₄Cl₂CoN₇O₈S₂ (M+): 1107.2967; found: 1107.2967.

### Preparation Example 20: Synthesis of Compound P20

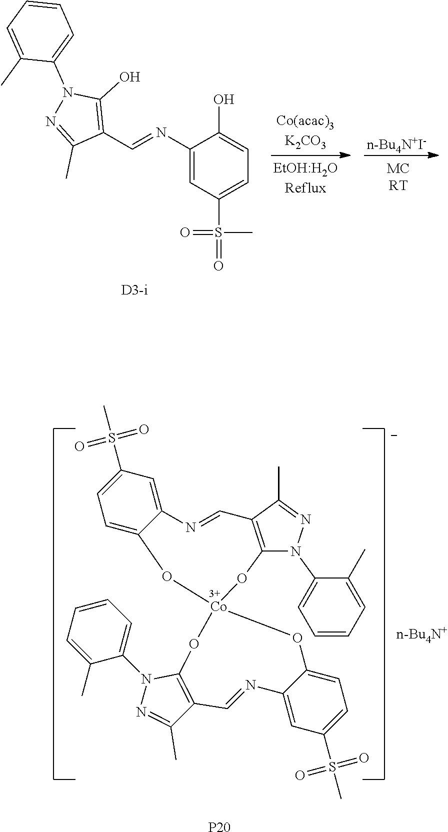

Compound P20 (270 mg, yield: 39%) was prepared by conducting a reaction in the same manner as in Preparation Example 16 except that D3-i (500 mg, 1 equivalent) was used instead of D1-a.

HR LC/MS/MS m/z calculated for C₅₄H₇₀CoN₇O₈S₂ (M+): 1067.4059; found: 1067.4058.

### Preparation Example 21: Synthesis of Compound P21

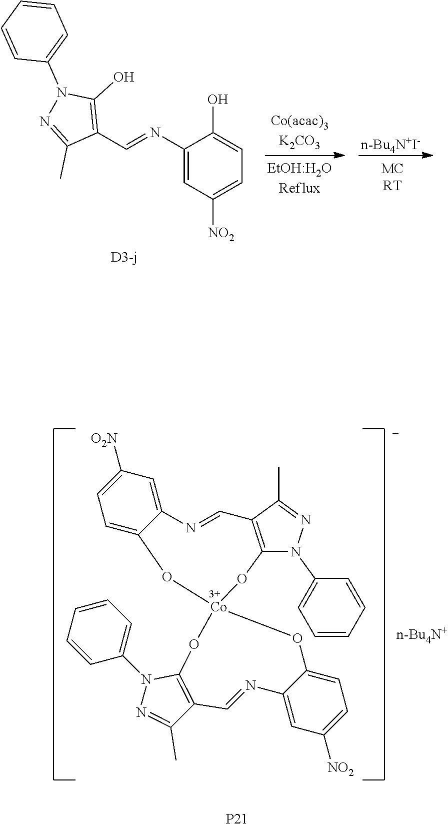

Compound P21 (331 mg, yield: 46%) was prepared by conducting a reaction in the same manner as in Preparation Example 16 except that D3-j (500 mg, 1 equivalent) was used instead of D1-a.

HR LC/MS/MS m/z calculated for C₅₀H₆₀CoN₉O₈(M+): 973.3897; found: 973.3897.

### Preparation Example 22: Synthesis of Compound P22

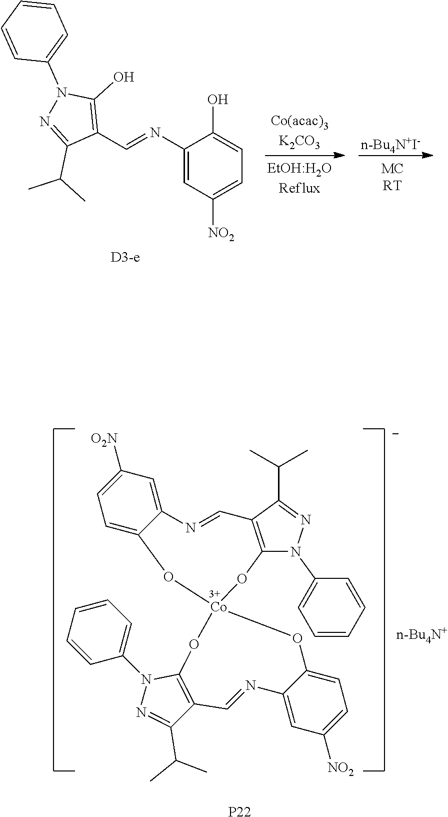

Compound P22 (492 mg, yield: 70%) was prepared by conducting a reaction in the same manner as in Preparation Example 16 except that D3-e (500 mg, 1 equivalent) was used instead of D1-a.

HR LC/MS/MS m/z calculated for C₅₄H₆₈CoN₉O₈ (M+): 1029. 4523; found: 1029.4253.

### Preparation Example 23: Synthesis of Compound P23

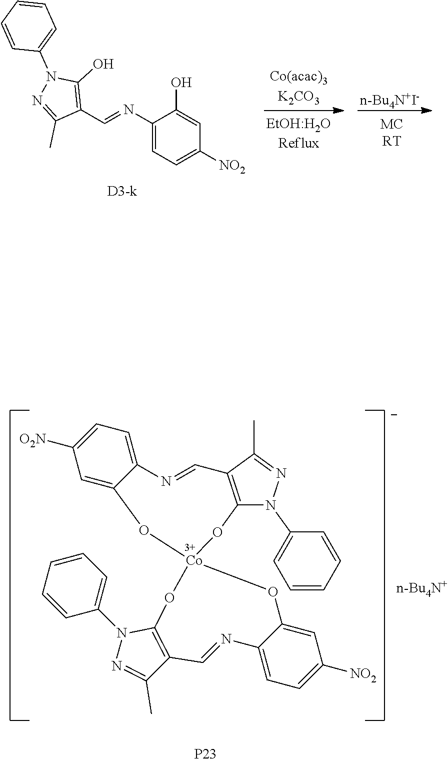

Compound P23 (461 mg, yield: 64%) was prepared by conducting a reaction in the same manner as in Preparation Example 16 except that D3-k (500 mg, 1 equivalent) was used instead of D1-a.

HR LC/MS/MS m/z calculated for C₅₀H₆₀CoN₉O₈ (M+): 973.3897; found: 973.3897.

### Preparation Example 24: Synthesis of Compound P24

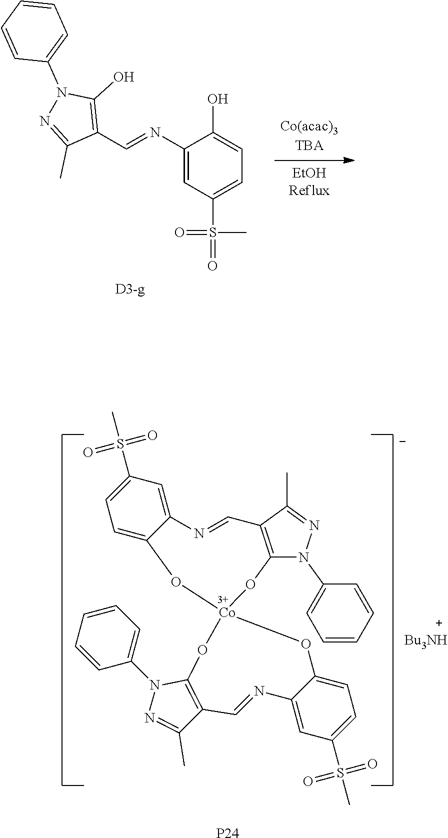

Compound D3-g (400 mg, 1 equivalent) synthesized using the method of Reaction Formula 1 was dissolved in ethanol (10 mL), and after adding tributylamine (0.31 mL, 2 equivalents) and tris(acetylacetonato)cobalt (III) (0.192 g, 0.5 equivalents) thereto, the mixture was stirred while heating under nitrogen. After the reaction was finished, the result was concentrated, and extracted using chloroform and water. From the organic layer secured through extraction, water was removed using anhydrous magnesium sulfate, and the result was concentrated through vacuum distillation. Compound P24 (302 mg, yield: 57%) was obtained from the concentrated product using ethanol and water.

HR LC/MS/MS m/z calculated for C₄₈H₅₈CoN₇O₈S₂ (M+): 983.3120; found: 983.3121.

### Preparation Example 25: Synthesis of Compound P25

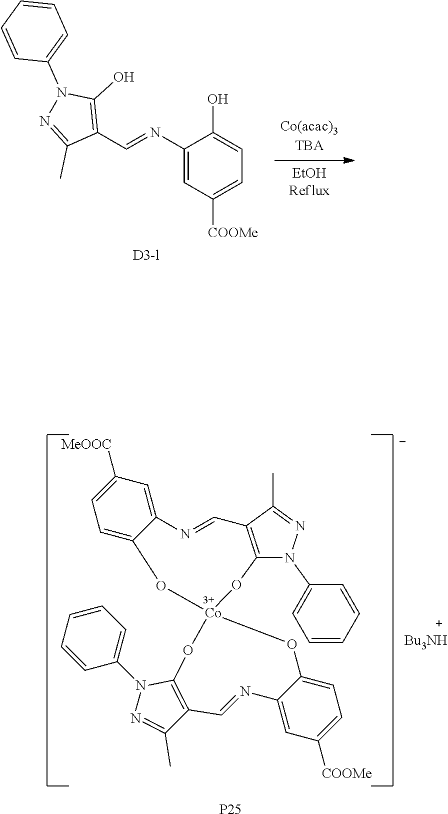

Compound P25 (457 mg, yield: 68%) was prepared by conducting a reaction in the same manner as in Preparation Example 24 except that D3-1 (500 mg, 1 equivalent) was used instead of D3-g.

HR LC/MS/MS m/z calculated for C₅₀H₅₈CoN₇O₈(M+): 943.3680; found: 943.3679.

### Preparation Example 26: Synthesis of Compound P26

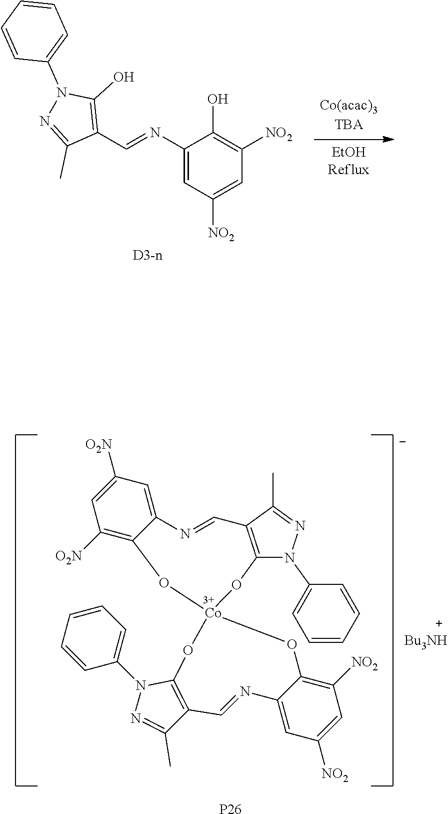

Compound P26 (427 mg, yield: 65%) was prepared by conducting a reaction in the same manner as in Preparation Example 24 except that D3-n (500 mg, 1 equivalent) was used instead of D3-g.

HR LC/MS/MS m/z calculated for C₄₆H₅₀CoN₁₁O₁₂ (M+): 1007.2972; found: 1007.2972.

### Preparation Example 27: Synthesis of Compound P27

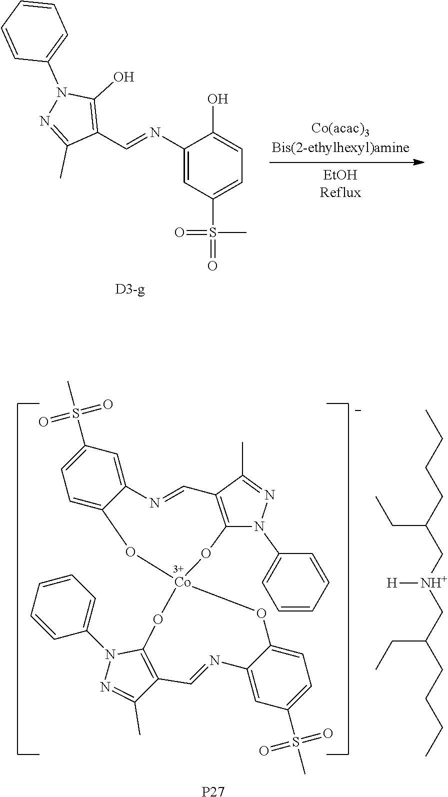

Compound P27 (269 mg, yield: 48%) was prepared by conducting a reaction in the same manner as in Preparation Example 24 except that bis(2-ethylhexyl)amine (0.3 mL, 2 equivalents) was used instead of tributylamine (TBA).

HR LC/MS/MS m/z calculated for C₅₂H₆₆CoN₇O₈S₂ (M+): 1038.3746; found: 1038.3746.

### Preparation Example 28: Synthesis of Compound P28

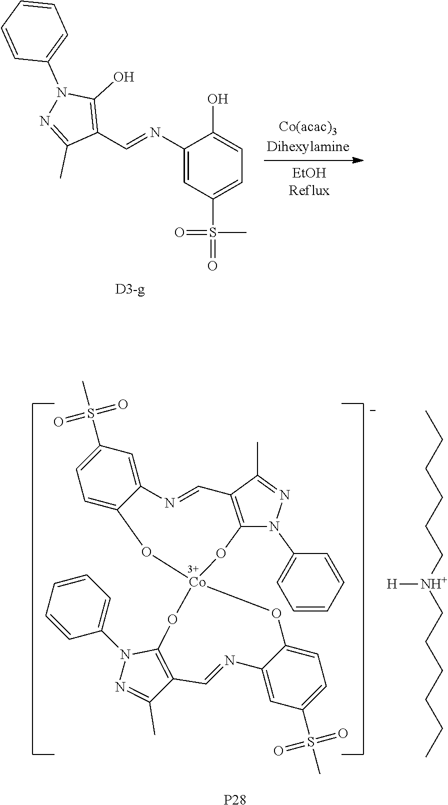

Compound P28 (371 mg, yield: 56%) was prepared by conducting a reaction in the same manner as in Preparation Example 24 except that dihexylamine (0.38 mL, 2 equivalents) was used instead of tributylamine (TBA).

HR LC/MS/MS m/z calculated for C₄₈H₅₈CON₇O₈S₂ (M+): 983.3120; found: 983.3121.

### Preparation Example 29: Synthesis of Compound P29

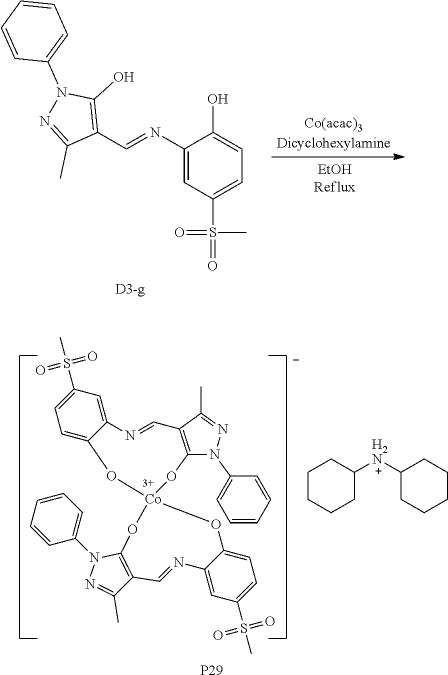

Compound P29 (382 mg, yield: 58%) was prepared by conducting a reaction in the same manner as in Preparation Example 24 except that dicyclohexylamine (490 mg, 2 equivalents) was used instead of tributylamine (TBA).

HR LC/MS/MS m/z calculated for C₄₈H₅₄CoN₇O₈S₂ (M+): 979.2807; found: 979.2807.

### Preparation Example 30: Synthesis of Compound P30

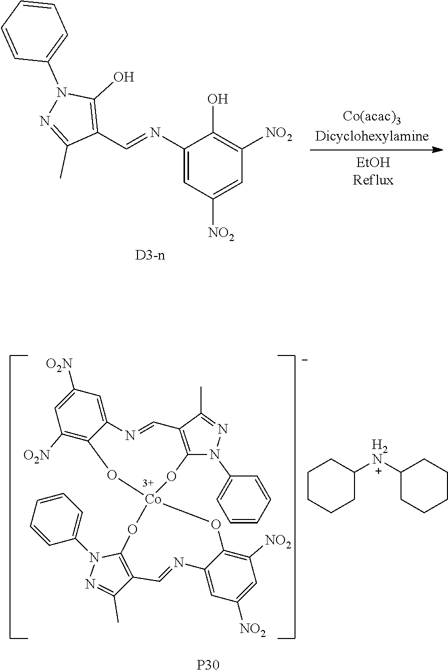

Compound P30 (420 mg, yield: 65%) was prepared by conducting a reaction in the same manner as in Preparation Example 24 except that D3-n (500 mg, 1 equivalent) was used instead of D3-g, and dicyclohexylamine (470 mg, 2 equivalents) was used instead of tributylamine (TBA).

HR LC/MS/MS m/z calculated for C₄₆H₄₆CoN₁₁O₁₂ (M+): 1003.2659; found: 1003.2659.

### Preparation Example 31: Synthesis of Compound P31

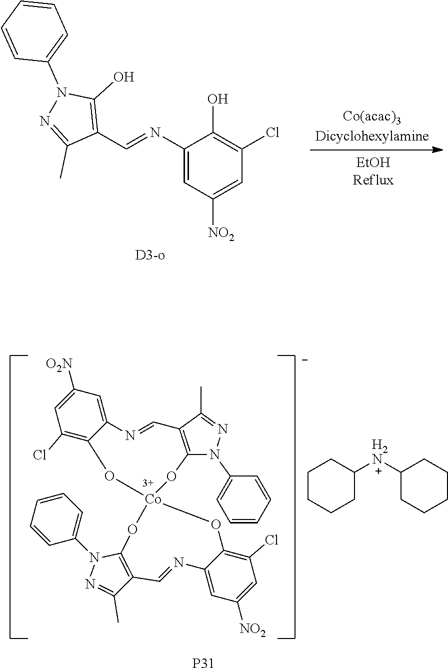

Compound P31 (468 mg, yield: 71%) was prepared by conducting a reaction in the same manner as in Preparation Example 24 except that D3-o (500 mg, 1 equivalent) was used instead of D3-g, and dicyclohexylamine (487 mg, 2 equivalents) was used instead of tributylamine (TBA).

HR LC/MS/MS m/z calculated for C₄₆H₄₆CoN₁₁O₁₂ (M+): 981.2178; found: 981.2179.

### Preparation Example 32: Synthesis of Compound P32

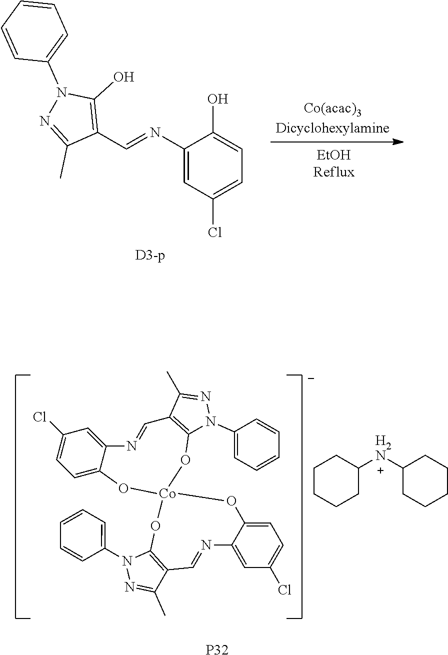

Compound P32 (354 mg, yield: 52%) was prepared by conducting a reaction in the same manner as in Preparation Example 24 except that D3-p (500 mg, 1 equivalent) was used instead of D3-g, and dicyclohexylamine (553 mg, 2 equivalents) was used instead of tributylamine (TBA).

HR LC/MS/MS m/z calculated for C₄₆H₄₆Cl₂CoN₇O₄ (M+): 891.2477; found: 891.2477.

### Preparation Example 33: Synthesis of Compound P33

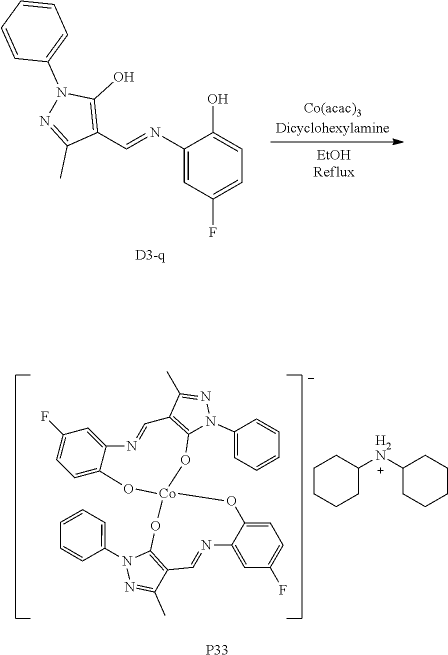

Compound P33 (317 mg, yield: 46%) was prepared by conducting a reaction in the same manner as in Preparation Example 24 except that D3-q (500 mg, 1 equivalent) was used instead of D3-g, and dicyclohexylamine (583 mg, 2 equivalents) was used instead of tributylamine (TBA).

HR LC/MS/MS m/z calculated for C₄₆H₄₈CoF₂N₇O₄ (M+): 859.3068; found: 859.3068.

### Preparation Example 34: Synthesis of Compound P34

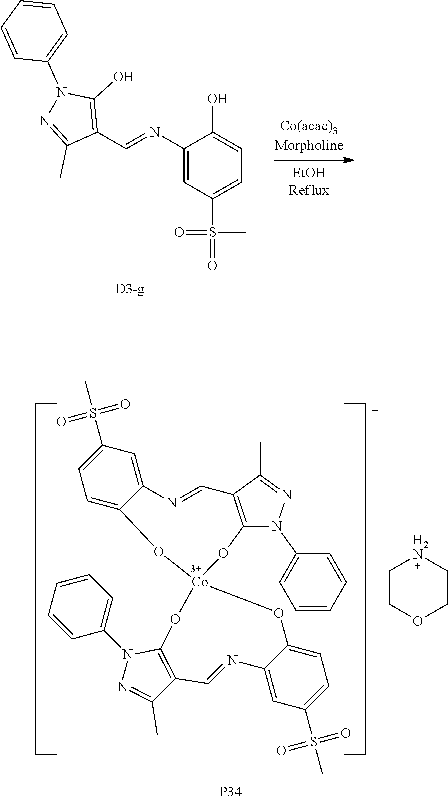

Compound P34 (334 mg, yield: 56%) was prepared by conducting a reaction in the same manner as in Preparation Example 24 except that morpholine (235 mg, 2 equivalents) was used instead of tributylamine (TBA) and D3-g (500 mg, 1 equivalent) used in Preparation Example 24 was used.

HR LC/MS/MS m/z calculated for C₄₀H₄₀CoN₇O₉S₂ (M+): 885.1661; found: 885.1661.

### Preparation Example 35: Synthesis of Compound P35

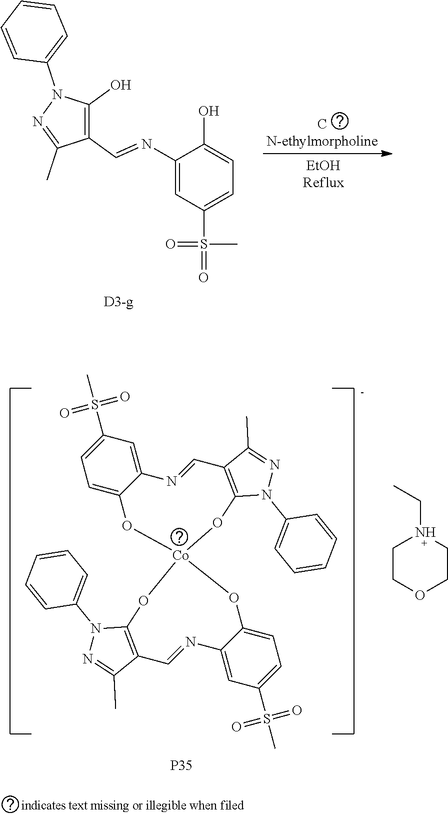

Compound P35 (455 mg, yield: 74%) was prepared by conducting a reaction in the same manner as in Preparation Example 24 except that N-ethyl morpholine (310 mg, 2 equivalents) was used instead of tributylamine (TBA) and D3-g (500 mg, 1 equivalent) used in Preparation Example 24 was used.

HR LC/MS/MS m/z calculated for C₄₂H₄₄CoN₇O₉S₂ (M+): 913.1974; found: 913.1973.

### Preparation Example 36: Synthesis of Compound P36

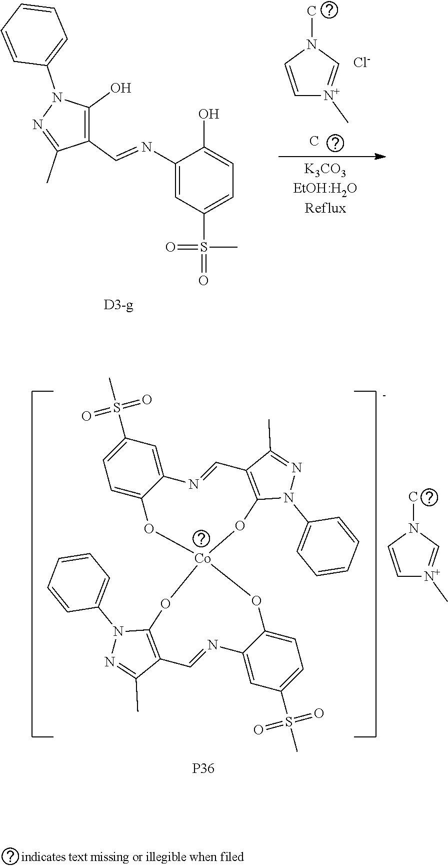

Compound D3-g (400 mg, 1 equivalent) synthesized using the method of Reaction Formula 1 was dissolved in ethanol (10 mL), and potassium carbonate (372 mg, 2 equivalents) dissolved in water (10 mL) was added thereto. After sufficiently stirring the solution, tris(acetylacetonato)cobalt(III) (0.192 mg, 0.5 equivalents) and 1-butyl-3-methylimidazolium chloride (140 mg, 0.6 equivalents) were added thereto, and the mixture was stirred while heating under nitrogen. After the reaction was finished, the result was concentrated, and extracted using chloroform and water. From the organic layer secured through extraction, water was removed using anhydrous magnesium sulfate, then water was removed using anhydrous magnesium sulfate from the organic layer secured through vacuum distillation, and the result was concentrated through vacuum distillation. Compound P36 (183 mg, yield: 29%) was obtained from the concentrated product using ethanol and water.

HR LC/MS/MS m/z calculated for C₄₄H₄₃CON₈O₈S₂ (M+): 934.1972; found: 934.1972.

### Preparation Example 37: Synthesis of Compound P37

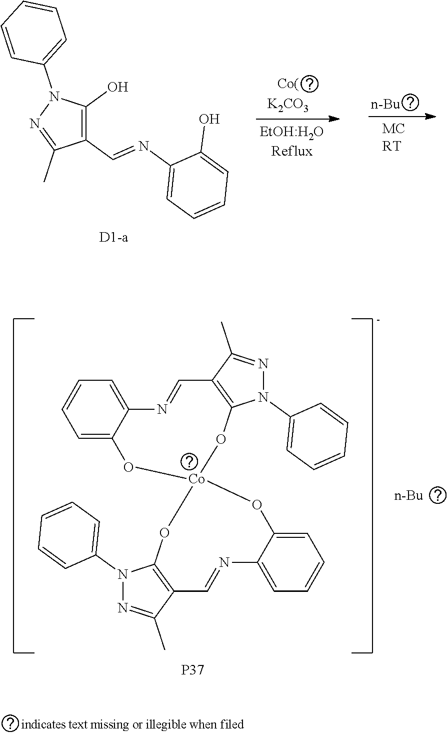

Compound P37 (369 mg, yield: 48%) was prepared by conducting a reaction in the same manner as in Preparation Example 16 except that n-butylphosphine bromide (0.303 mg, 0.6 equivalents) was used instead of tetra-n-butylammonium iodine.

HR LC/MS/MS m/z calculated for C₅₀H₆₂CoN₆O₄P (M+): 900.3902; found: 900.3903.

### Preparation Example 38: Synthesis of Compound P38

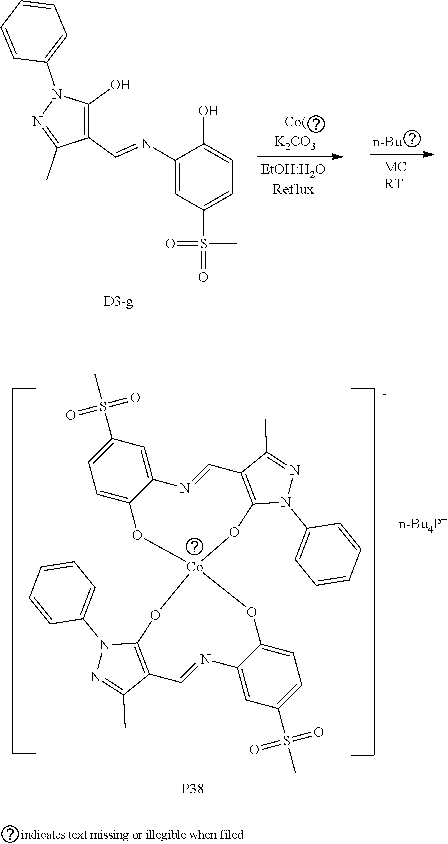

Compound P38 (278 mg, yield: 39%) was prepared by conducting a reaction in the same manner as in Preparation Example 37 except that D3-g (500 mg, 1 equivalent) was used instead of D1-a.

HR LC/MS/MS m/z calculated for C₅₂H₆₆CoN₆O₈PS₂ (M+): 1056.3453; found: 1056.3453.

### Preparation Example 39: Synthesis of Compound P39

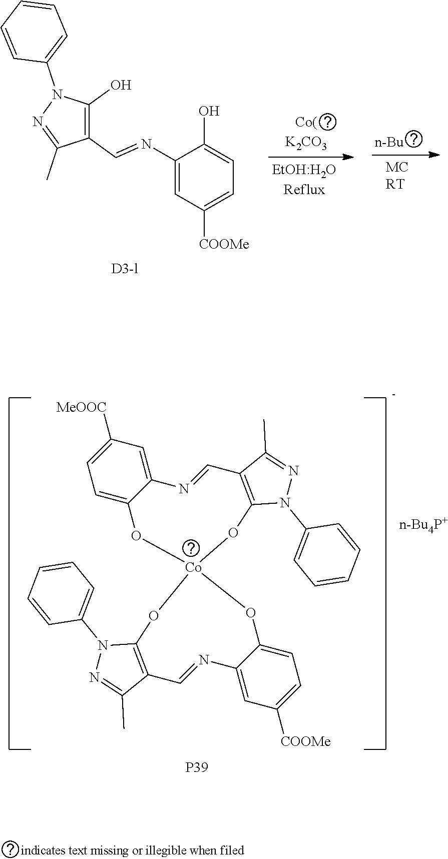

Compound P39 (391 mg, yield: 54%) was prepared by conducting a reaction in the same manner as in Preparation Example 37 except that D3-1 (500 mg, 1 equivalent) was used instead of D1-a.

HR LC/MS/MS m/z calculated for C₅₄H₆₆CoN₆O₈P (M+): 1016.4012; found: 1016.4012.

### Preparation Example 40: Synthesis of Compound P40

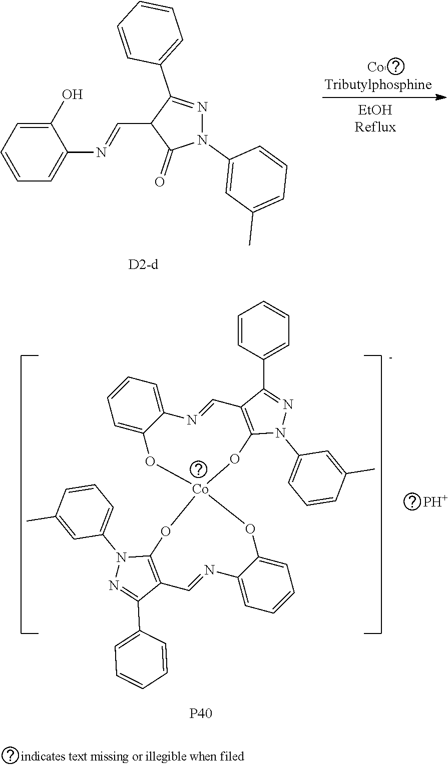

Compound P40 (351 mg, yield: 65%) was prepared by conducting a reaction in the same manner as in Preparation Example 24 except that D2-d (400 mg, 1 equivalent) was used instead of D3-g, and tributylphosphine (0.438 mg, 2 equivalents) was used instead of tributylamine (TBA).

HR LC/MS/MS m/z calculated for C₅₈H₅₈CoN₆O₄P (M+): 996.3902; found: 996.3902.

### Preparation Example 41: Synthesis of Compound P41

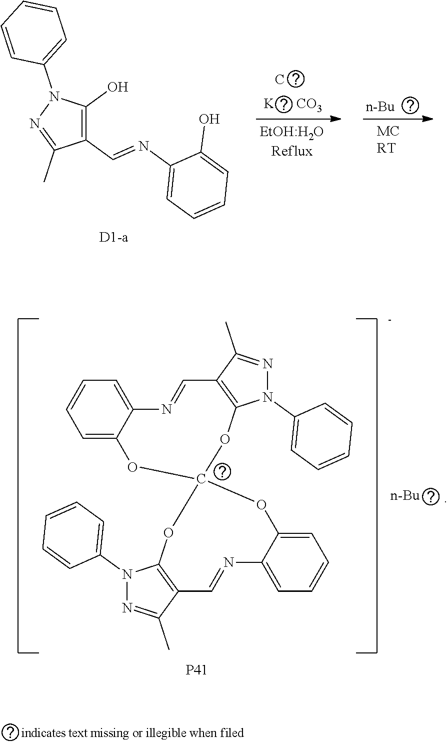

Compound D1-a (500 mg, 1 equivalent) synthesized using the method of Reaction Formula 1 was diluted in ethanol (10 mL), and after adding potassium carbonate (471 mg, 2 equivalents) dissolved in water (10 mL) thereto, the mixture was stirred while heating under nitrogen. After that, chromium acetate hydroxide (193 mg, 0.5 equivalents) was added thereto, and the reaction proceeded until the reactant D1-a was not visible on thin layer chromatography (TLC). After concentrating the solvent from the completed reaction material, a dichloromethane solvent (20 mL) was added thereto, then tetra-n-butylammonium iodine (378 mg, 0.6 equivalents) was introduced thereto, and the result was stirred at room temperature. After the reaction was completed, the result was extracted by adding water thereto, and from the organic layer secured therethrough, water was removed using anhydrous magnesium sulfate, and the result was concentrated through vacuum distillation. The concentrated product was diluted in ethanol, and then water was added thereto to obtain Compound P41 (389 mg, yield: 52%).

HR LC/MS/MS m/z calculated for C₅₀H₆₂CrN₇O₄(M+): 876.4268; found: 876.4268.

### Preparation Example 42: Synthesis of Compound P42

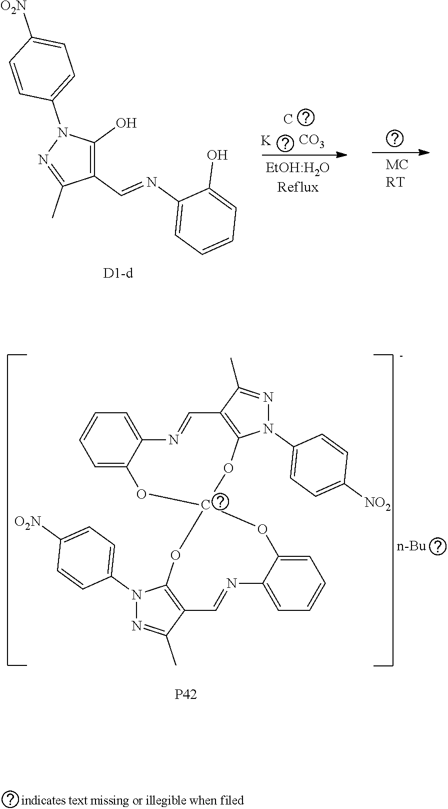

Compound P42 (443 mg, yield: 62%) was prepared by conducting a reaction in the same manner as in Preparation Example 41 except that D1-d (500 mg, 1 equivalent) was used instead of D1-a.

HR LC/MS/MS m/z calculated for C₅₀H₆₀CrN₉O₈(M+): 966.3970; found: 966.3971.

### Preparation Example 43: Synthesis of Compound P43

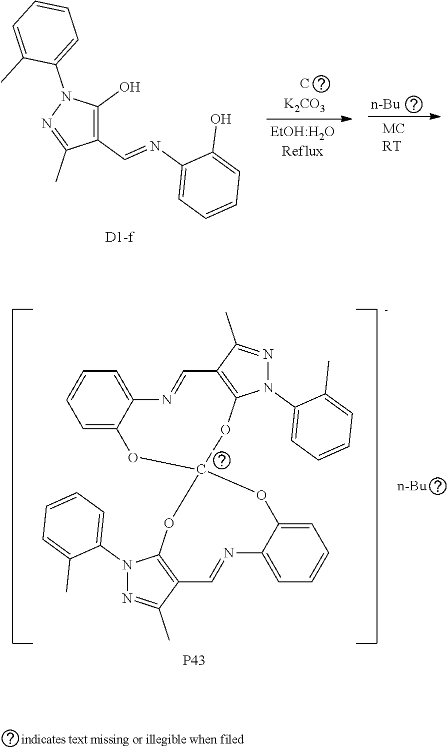

Compound P43 (398 mg, yield: 54%) was prepared by conducting a reaction in the same manner as in Preparation Example 41 except that D1-f (500 mg, 1 equivalent) was used instead of D1-a.

HR LC/MS/MS m/z calculated for C₅₂H₆₆CrN₇O₄(M+): 904.4581; found: 904.4581.

### Preparation Example 44: Synthesis of Compound P44

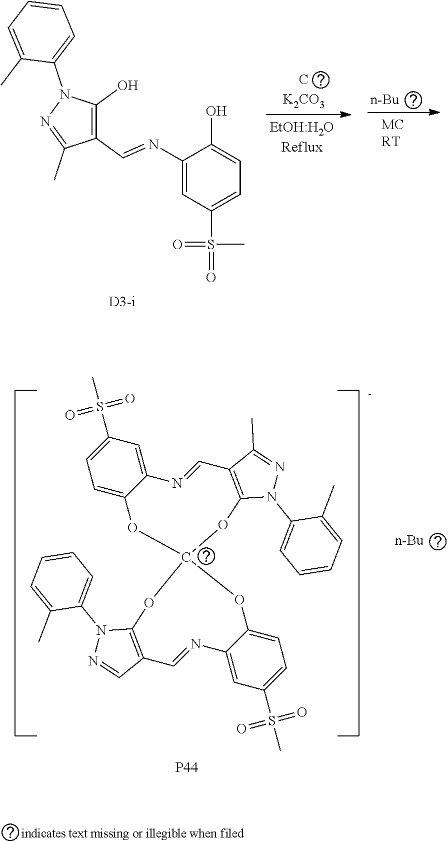

Compound P44 (399 mg, yield: 58%) was prepared by conducting a reaction in the same manner as in Preparation Example 41 except that D3-i (500 mg, 1 equivalent) was used instead of D1-a.

HR LC/MS/MS m/z calculated for C₅₄H₇₀CrN₇O₈S₂ (M+): 1060.4132; found: 1060.4133.

### Preparation Example 45: Synthesis of Compound P45

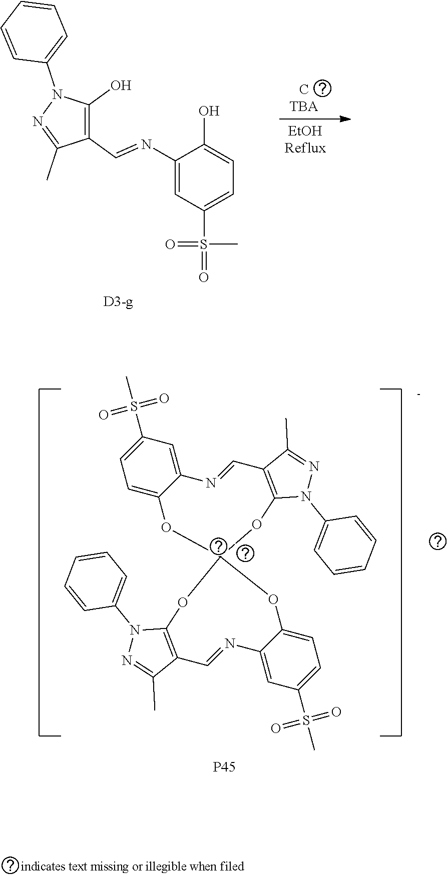

Compound D3-g (400 mg, 1 equivalent) synthesized using the method of Reaction Formula 1 was dissolved in ethanol (10 mL), and tributylamine (499 mg, 2 equivalents) and chromium acetate hydroxide (406 mg, 0.5 equivalents) were added thereto, and the mixture was stirred while heating under nitrogen. After the reaction was finished, the result was concentrated, and extracted using chloroform and water. From the organic layer secured through extraction, water was removed using anhydrous magnesium sulfate, and the result was concentrated through vacuum distillation. Compound P45 (401 mg, yield: 61%) was obtained from the concentrated product using ethanol and water.

HR LC/MS/MS m/z calculated for C₄₈H₅₈CrN₇O₈S₂ (M+): 976.3193; found: 976.3193

### Preparation Example 46: Synthesis of Compound P46

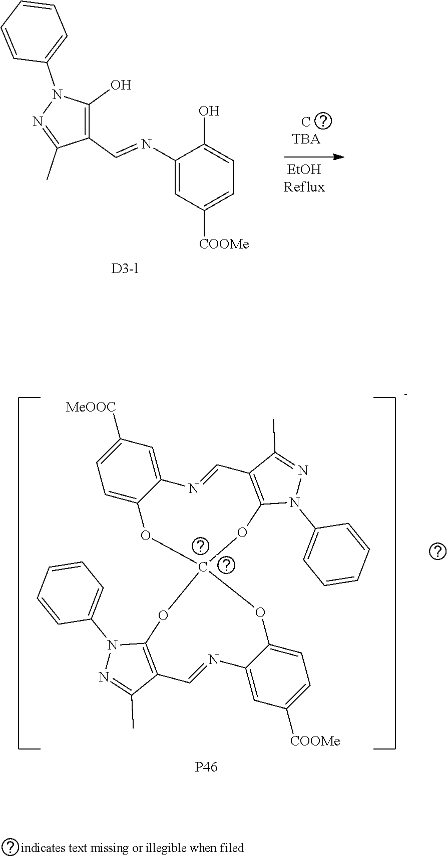

Compound P46 (387 mg, yield: 58%) was prepared by conducting a reaction in the same manner as in Preparation Example 45 except that D3-1 (500 mg, 1 equivalent) was used instead of D3-g.

HR LC/MS/MS m/z calculated for C₅₀H₅₈CrN₇O₈(M+): 936.3752; found: 936.3751.

### Preparation Example 47: Synthesis of Compound P47

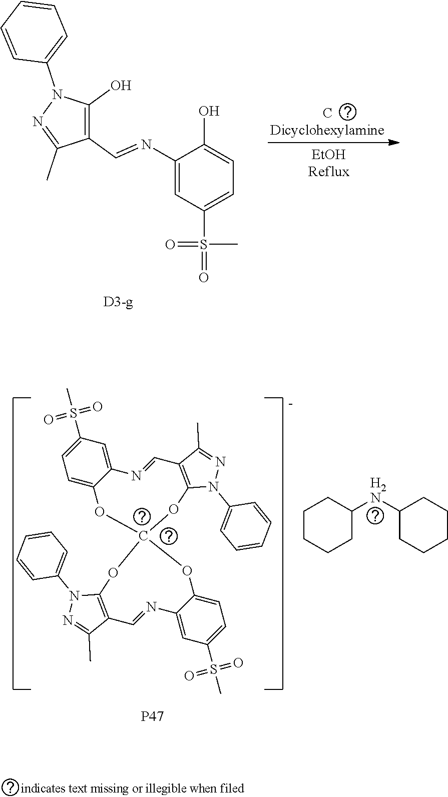

Compound P47 (360 mg, yield: 55%) was obtained by conducting a reaction in the same manner as in Preparation Example 45 except that dicyclohexylamine (488 mg, 2 equivalents) was used instead of tributylamine.

HR LC/MS/MS m/z calculated for C₄₈H₅₄CrN₇O₈S₂ (M+): 972.2880; found: 972.2881.

### Preparation Example 48: Synthesis of Compound P48

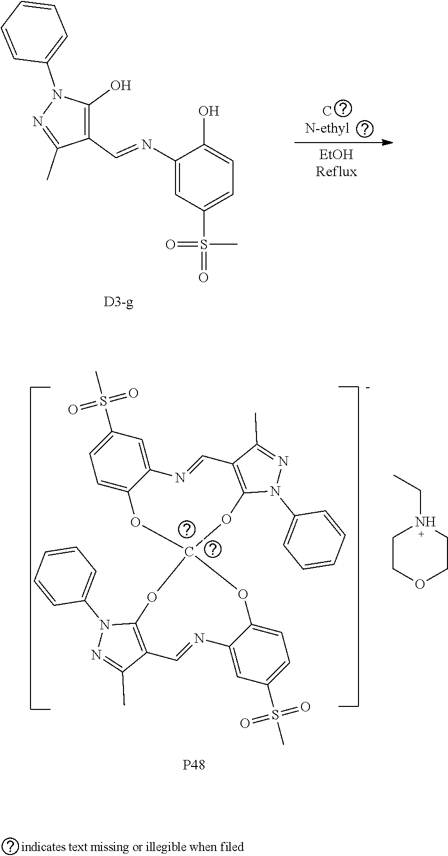

Compound P48 (433 mg, yield: 71%) was prepared by conducting a reaction in the same manner as in Preparation Example 45 except that N-ethyl morpholine (310 mg, 2 equivalents) was used instead of tributylamine.

HR LC/MS/MS m/z calculated for C₄₂H₄₄CrN₇O₉S₂ (M+): 906.2047; found: 906.2047.

### Preparation Example 49: Synthesis of Compound P49

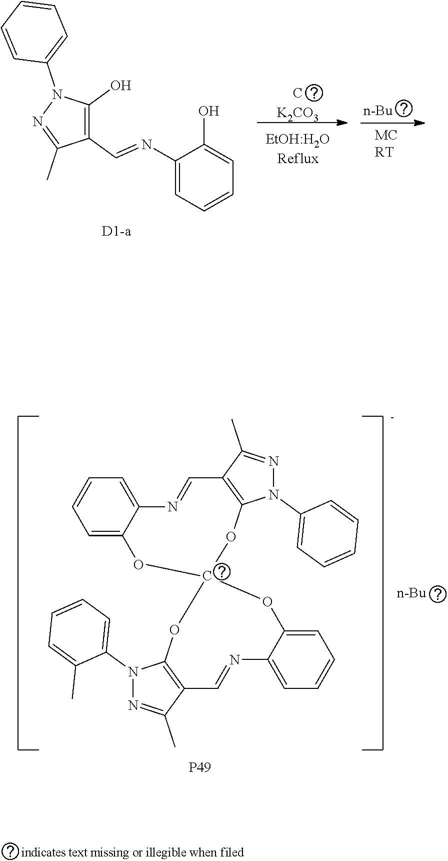

Compound P49 (488 mg, yield: 64%) was prepared by conducting a reaction in the same manner as in Preparation Example 41 except that n-tetrabutylphosphine bromide (471 mg, 0.6 equivalents) was used instead of n-tetrabutylammonium iodine.

HR LC/MS/MS m/z calculated for C₅₀H₆₂CrN₆O₄P (M+): 893.3975; found: 893.3975.

### Preparation Example 50: Synthesis of Compound P50

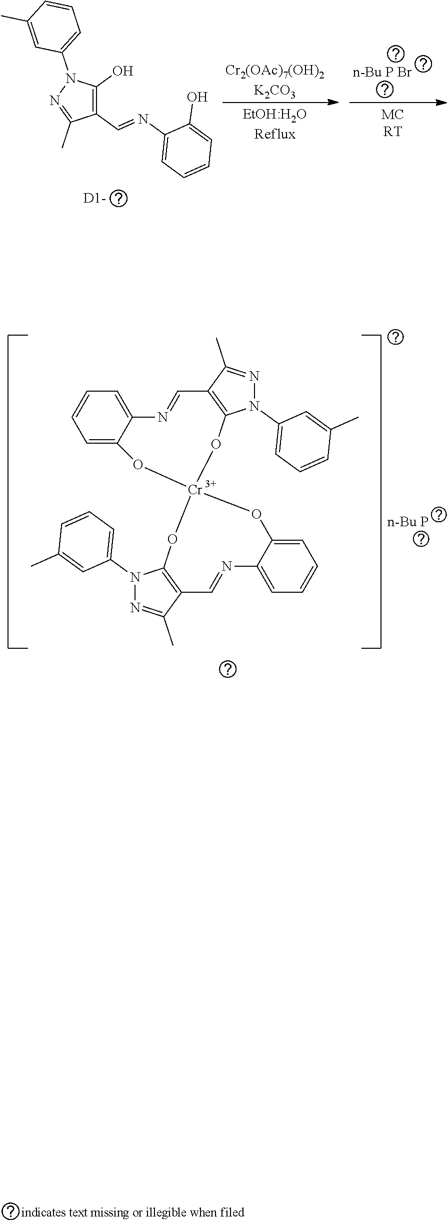

Compound P50 (406 mg, yield: 55%) was prepared by conducting a reaction in the same manner as in Preparation Example 49 except that D1-g (500 mg, 1 equivalent) was used instead of D1-a.

HR LC/MS/MS m/z calculated for C₅₁H₆₃CrN₆O₄P (M+): 906.4054; found: 906.4054.

### Preparation Example 51: Synthesis of Compound P51

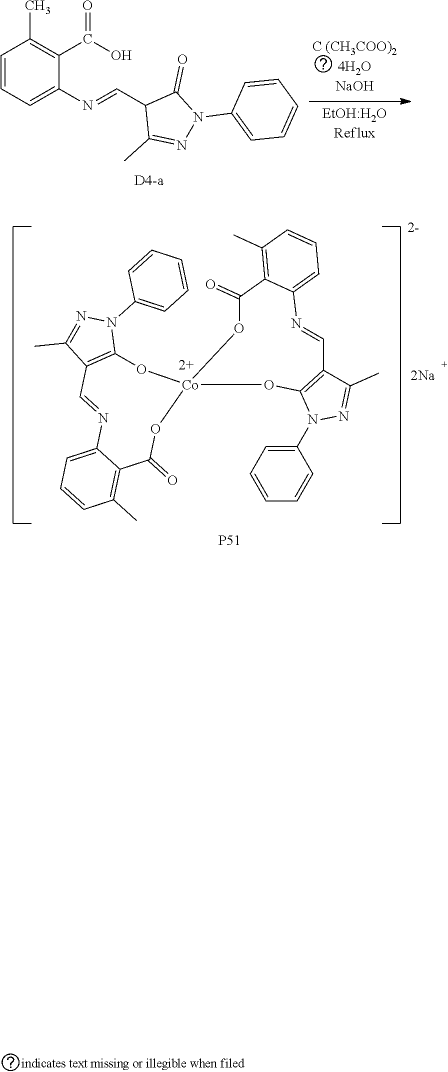

Compound P51 (276 mg, yield: 48%) was prepared by conducting a reaction in the same manner as in Preparation Example 1 except that D4-a (500 mg, 1 equivalent) was used instead of D1-a.

HR LC/MS/MS m/z calculated for C₃₈H₃₀CoN₆Na₂O₆(M+): 771.1354; found: 771.1354.

### Preparation Example 52: Synthesis of Compound P52

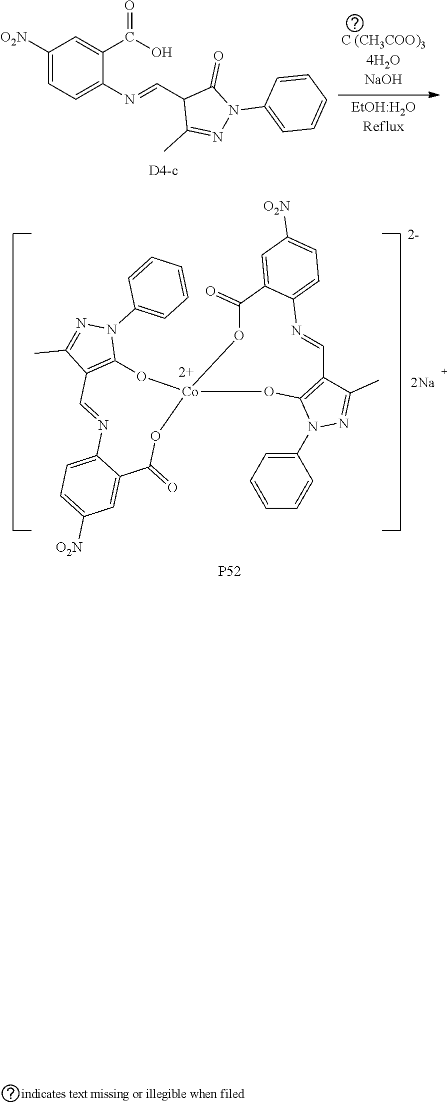

Compound P52 (296 mg, yield: 52%) was prepared by conducting a reaction in the same manner as in Preparation Example 1 except that D4-c (500 mg, 1 equivalent) was used instead of D1-a.

HR LC/MS/MS m/z calculated for C₃₆H₂₄CoN₈Na₂O₁₀ (M+): 833.0743; found: 833.0743.

### Preparation Example 53: Synthesis of Compound P53

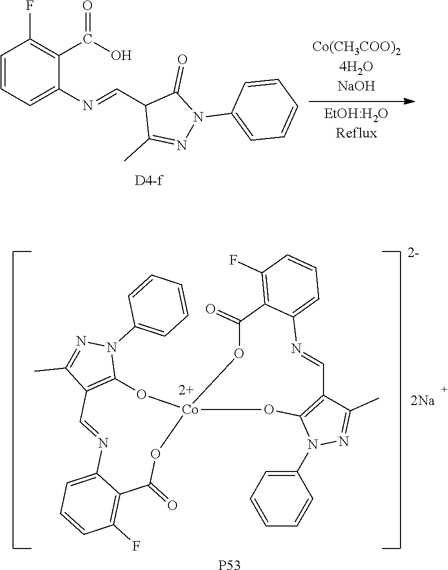

Compound P53 (224 mg, yield: 39%) was prepared by conducting a reaction in the same manner as in Preparation Example 1 except that D4-f (500 mg, 1 equivalent) was used instead of D1-a.

HR LC/MS/MS m/z calculated for C₃₆H₂₄CoF₂N₆Na₂O₆(M+): 779.0853; found: 779.0853.

### Preparation Example 54: Synthesis of Compound P54

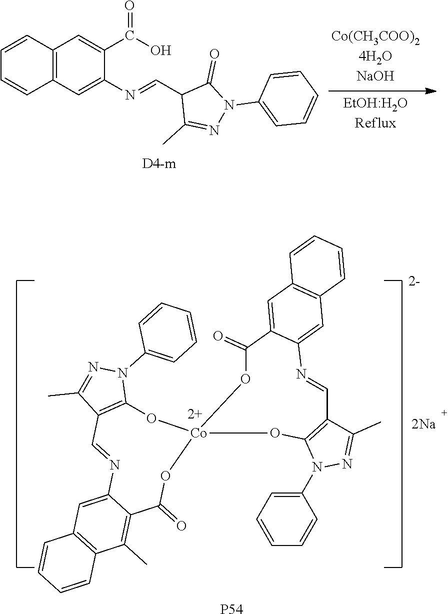

Compound P54 (339 mg, yield: 62%) was prepared by conducting a reaction in the same manner as in Preparation Example 1 except that D4-m (500 mg, 1 equivalent) was used instead of D1-a.

HR LC/MS/MS m/z calculated for C₄₄H₃₀CoN₆Na₂O₆(M+): 843.1354; found: 843.1354.

### Preparation Example 55: Synthesis of Compound P55

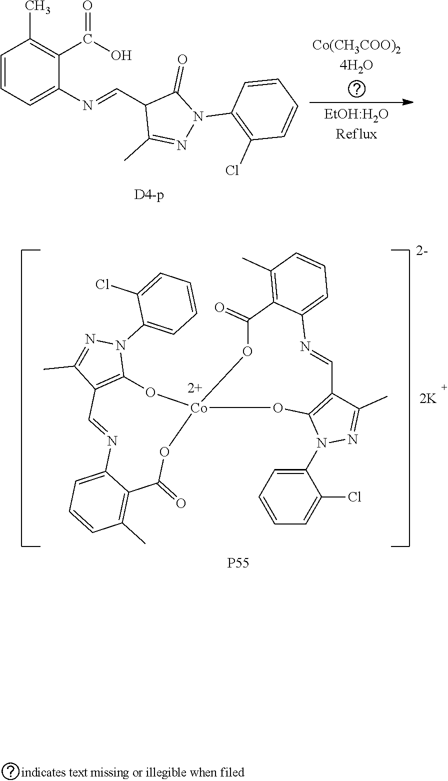

Compound P55 (342 mg, yield: 58%) was prepared by conducting a reaction in the same manner as in Preparation Example 1 except that D4-p (500 mg, 1 equivalent) was used instead of D1-a, and potassium hydroxide (151 mg, 2 equivalents) was used instead of sodium hydroxide (NaOH).

HR LC/MS/MS m/z calculated for C₃₈H₂₈Cl₂CoK₂N₆O₆ (M+): 871.0054; found: 871.0054.

### Preparation Example 56: Synthesis of Compound P56

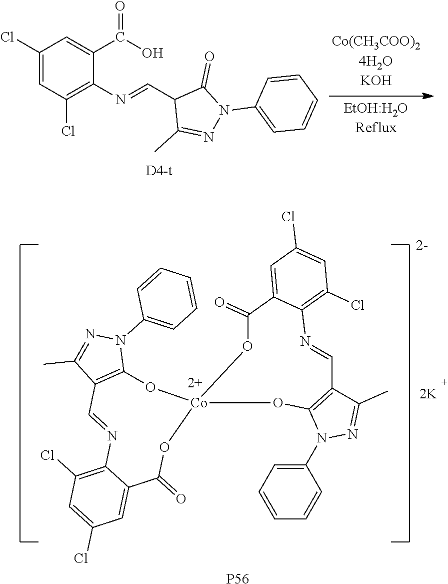

Compound P56 (259 mg, yield: 49%) was prepared by conducting a reaction in the same manner as in Preparation Example 1 except that D4-t (500 mg, 1 equivalent) was used instead of D1-a, and potassium hydroxide (130 mg, 2 equivalents) was used instead of sodium hydroxide (NaOH).

HR LC/MS/MS m/z calculated for C₃₆H₂₂Cl₄CoK₂N₆O₆ (M+): 910.8961; found: 910.8960.

### Preparation Example 57: Synthesis of Compound P57

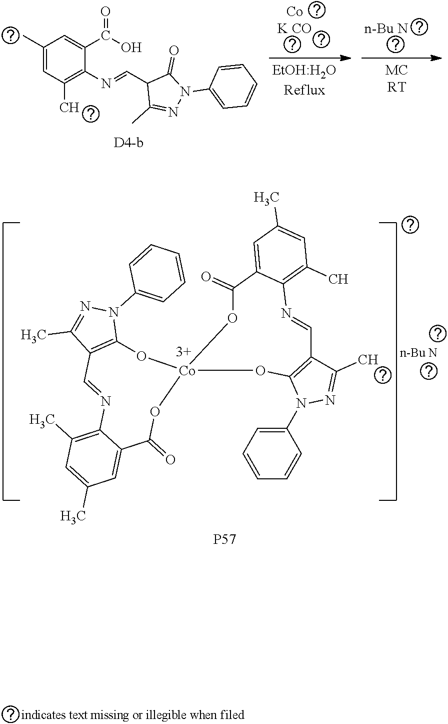

Compound P57 (385 mg, yield: 54%) was prepared by conducting a reaction in the same manner as in Preparation Example 16 except that D4-b (500 mg, 1 equivalent) was used instead of D1-a.

HR LC/MS/MS m/z calculated for C₅₆H₇₀CoN₇O₆(M+): 995.4714; found: 995.4714.

### Preparation Example 58: Synthesis of Compound P58

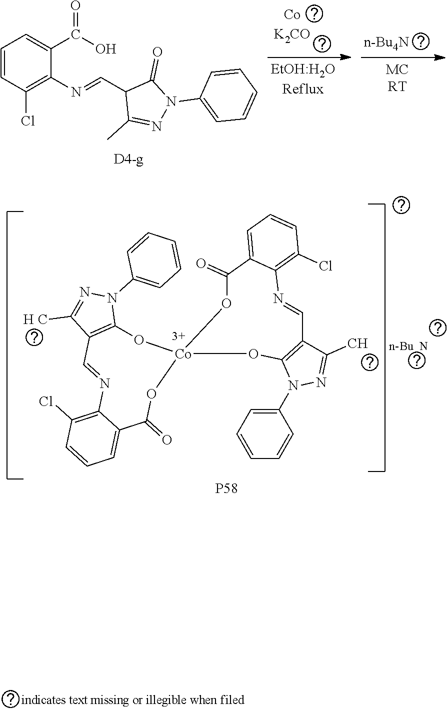

Compound P58 (440 mg, yield: 62%) was prepared by conducting a reaction in the same manner as in Preparation Example 16 except that D4-g (500 mg, 1 equivalent) was used instead of D1-a.

HR LC/MS/MS m/z calculated for C₅₂H₆₀Cl₂CoN₇O₆(M+): 1007.3309; found: 1007.3308.

### Preparation Example 59: Synthesis of Compound P59

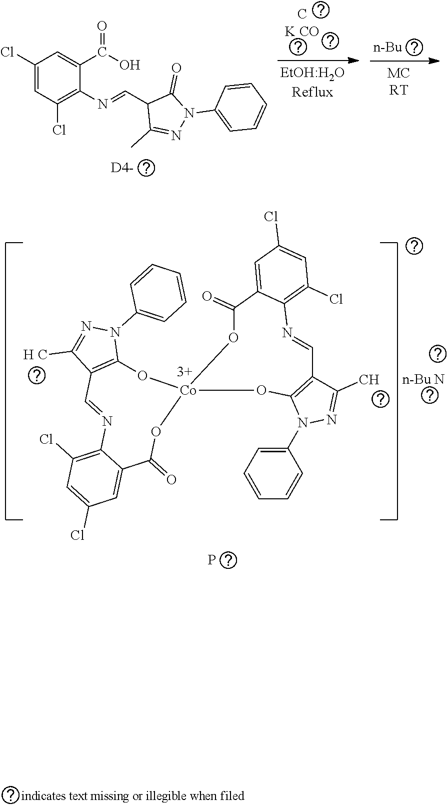

Compound P59 (359 mg, yield: 52%) was prepared by conducting a reaction in the same manner as in Preparation Example 16 except that D4-h (500 mg, 1 equivalent) was used instead of D1-a.

HR LC/MS/MS m/z calculated for C₅₂H₅₈Cl₄CoN₇O₆(M+): 1077.2500; found: 1077.2501.

### Preparation Example 60: Synthesis of Compound P60

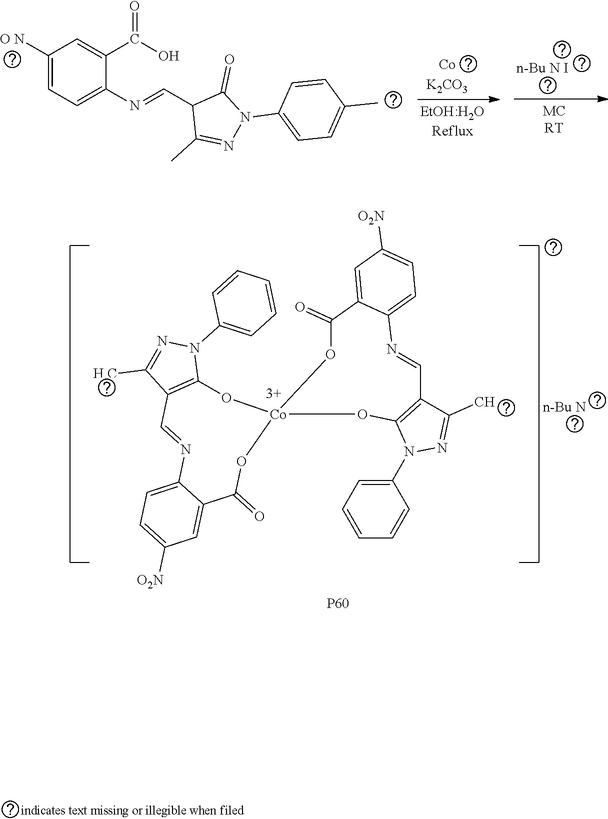

Compound P60 (251 mg, yield: 37%) was prepared by conducting a reaction in the same manner as in Preparation Example 16 except that D4-1 (500 mg, 1 equivalent) was used instead of D1-a.

HR LC/MS/MS m/z calculated for C₅₂H₆₀CoN₉O₁₀ (M+): 1029.3790; found: 1029.3789.

### Preparation Example 61: Synthesis of Compound P61

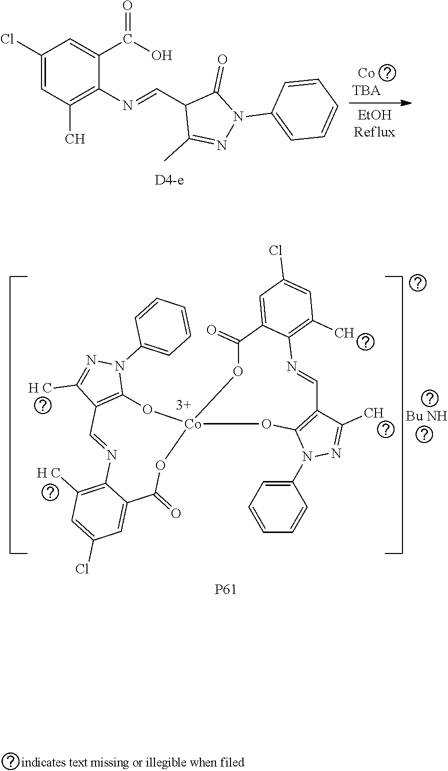

Compound P61 (298 mg, yield: 45%) was prepared by conducting a reaction in the same manner as in Preparation Example 24 except that D4-e (500 mg, 1 equivalent) was used instead of D3-g.

HR LC/MS/MS m/z calculated for C₅₀H₅₅Cl₂CoN₇O₆(M+): 979.2996; found: 979.2996.

### Preparation Example 62: Synthesis of Compound P62

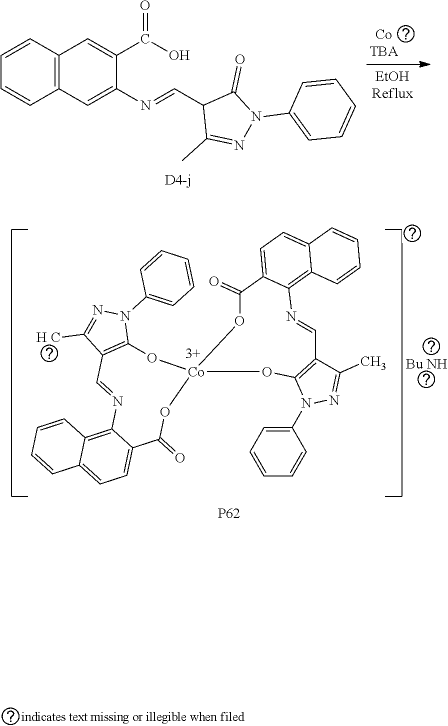

Compound P62 (450 mg, yield: 68%) was prepared by conducting a reaction in the same manner as in Preparation Example 24 except that D4-j (500 mg, 1 equivalent) was used instead of D3-g.

HR LC/MS/MS m/z calculated for C₅₆H₅₈CoN₇O₆(M+): 983.3775; found: 983.3774.

### Preparation Example 63: Synthesis of Compound P63

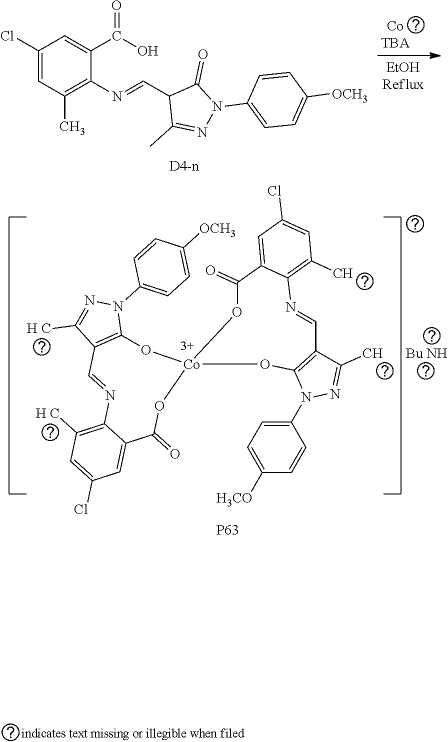

Compound P63 (469 mg, yield: 72%) was prepared by conducting a reaction in the same manner as in Preparation Example 24 except that D4-n (500 mg, 1 equivalent) was used instead of D3-g.

HR LC/MS/MS m/z calculated for C₅₂H₆₀Cl₂CoN₇O₈(M+): 1039.3207; found: 1039.3207.

### Preparation Example 64: Synthesis of Compound P64

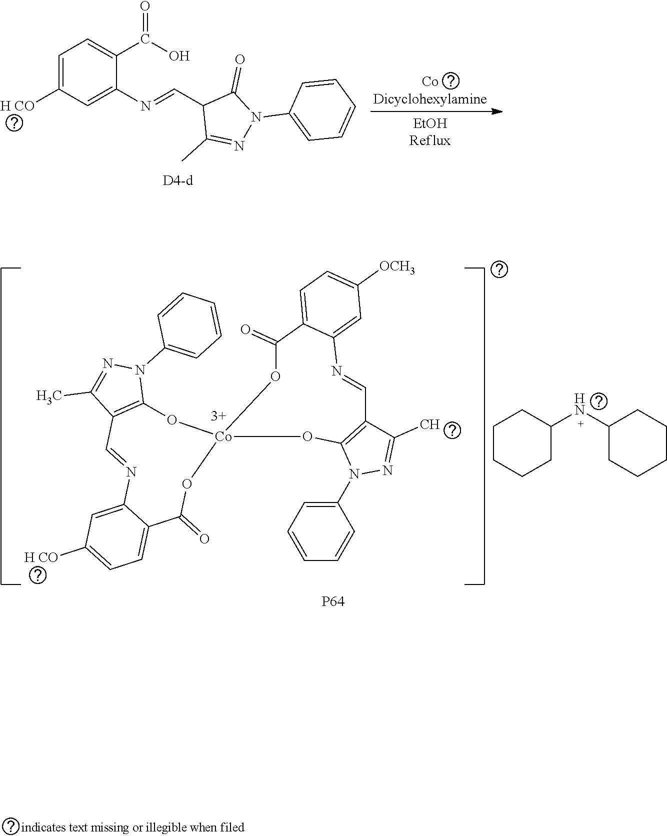

Compound P64 (388 mg, yield: 58%) was prepared by conducting a reaction in the same manner as in Preparation Example 24 except that D4-d (500 mg, 1 equivalent) was used instead of D3-g, and dicyclohexylamine (516 mg, 2 equivalents) was used instead of tributylamine (TBA).

HR LC/MS/MS m/z calculated for C₅₀H₅₄CoN₇O₈(M+): 939.3360; found: 939.3360.

### Preparation Example 65: Synthesis of Compound P65

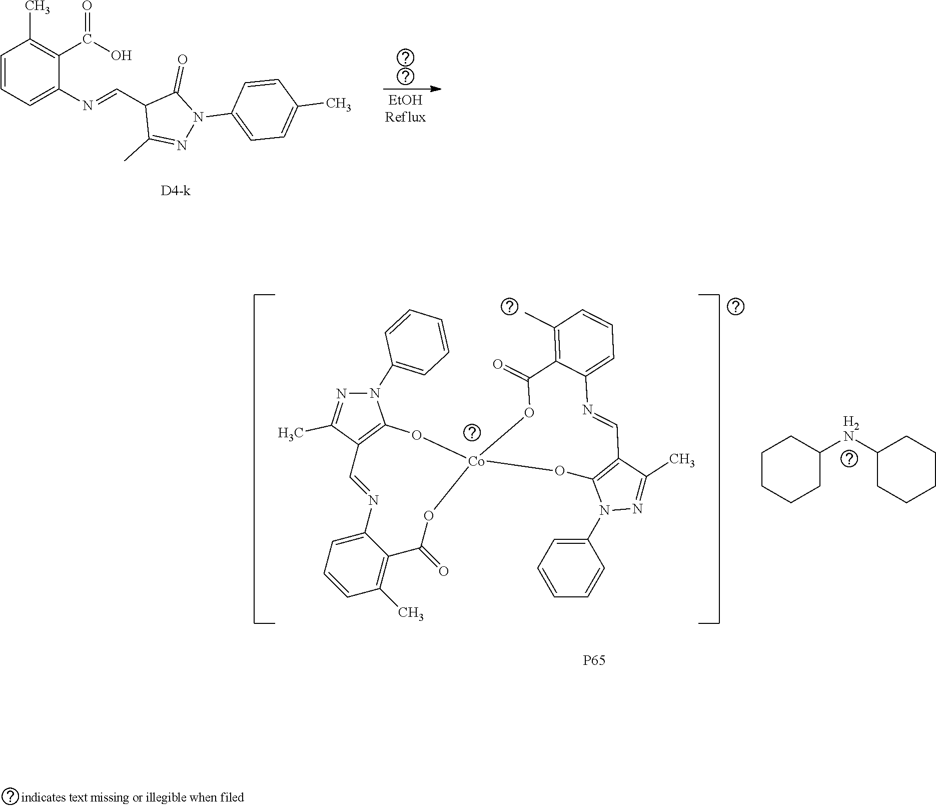

Compound P65 (403 mg, yield: 62%) was prepared by conducting a reaction in the same manner as in Preparation Example 24 except that D4-k (500 mg, 1 equivalent) was used instead of D3-g, and dicyclohexylamine (519 mg, 2 equivalents) was used instead of tributylamine (TBA).

HR LC/MS/MS m/z calculated for C₅₀H₅₄CoN₇O₆ (M+): 907.3462; found: 907.3462.

### Preparation Example 66: Synthesis of Compound P66

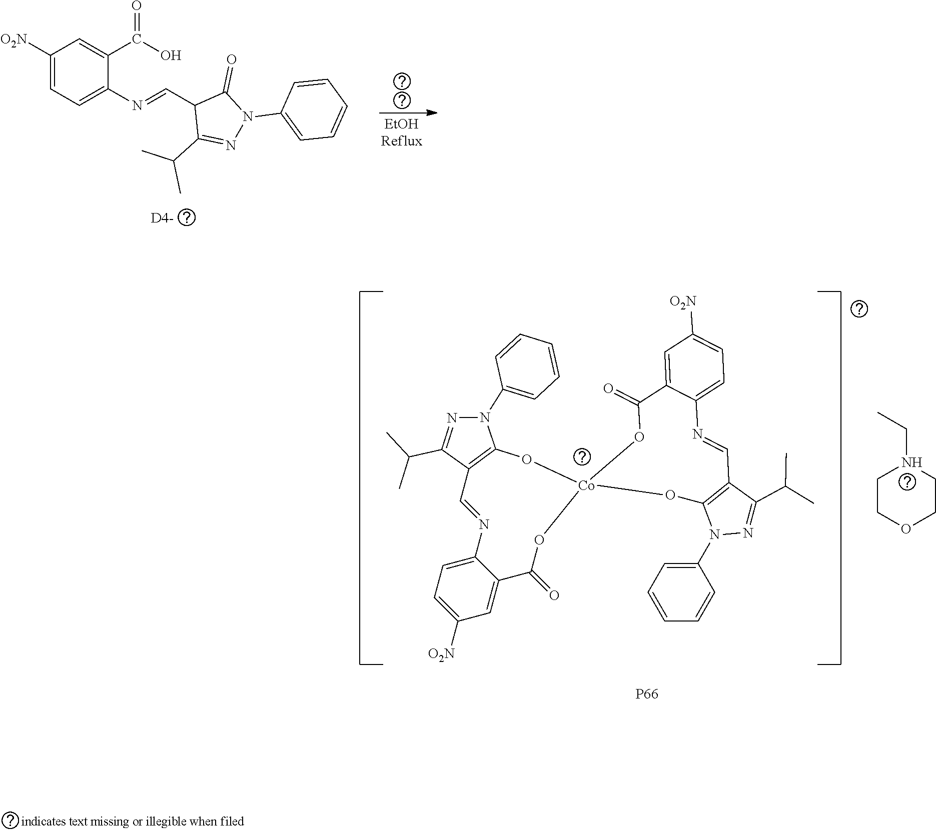

Compound P66 (268 mg, yield: 44%) was prepared by conducting a reaction in the same manner as in Preparation Example 24 except that D4-u (500 mg, 1 equivalent) was used instead of D3-g, and N-ethyl morpholine (292 mg, 2 equivalents) was used instead of tributylamine (TBA).

HR LC/MS/MS m/z calculated for C₄₆H₄₆CoN₉O₁₁ (M+): 959.2600; found: 959.2601.

### Preparation Example 67: Synthesis of Compound P67

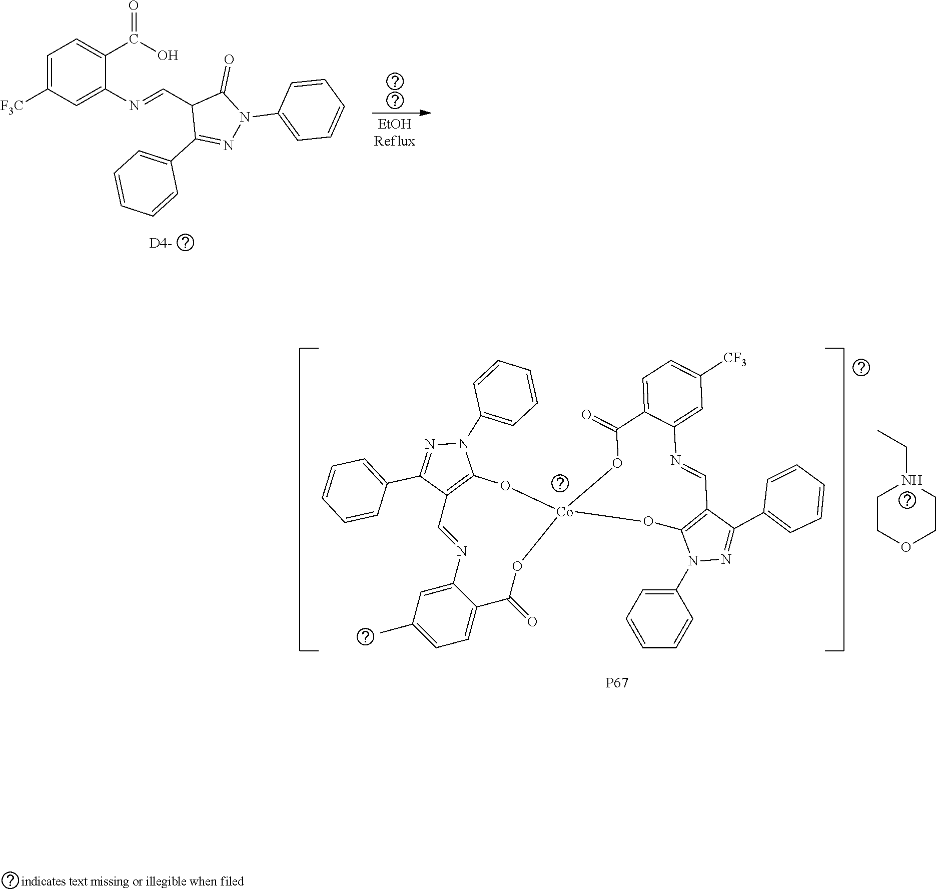

Compound P67 (351 mg, yield: 59%) was prepared by conducting a reaction in the same manner as in Preparation Example 24 except that D4-v (500 mg, 1 equivalent) was used instead of D3-g, and N-ethyl morpholine (255 mg, 2 equivalents) was used instead of tributylamine (TBA).

HR LC/MS/MS m/z calculated for C₅₄H₄₂CoF₆N₇O₇ (M+): 1073.2376; found: 1073.2376.

### Preparation Example 68: Synthesis of Compound P68

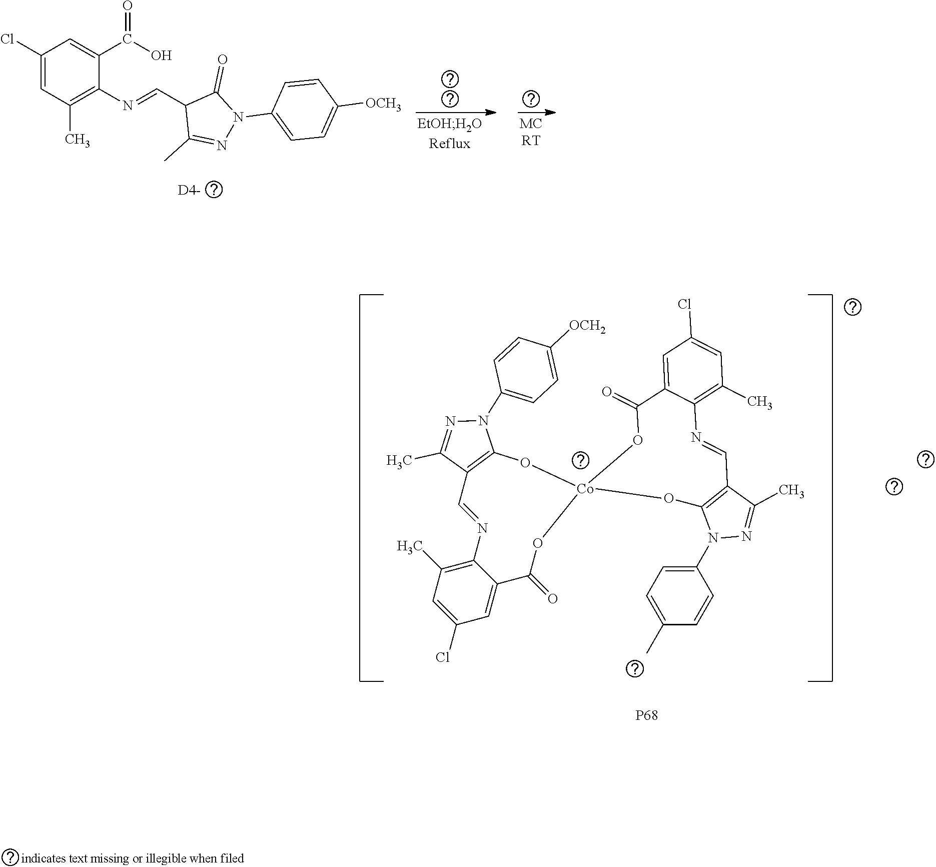

Compound P68 (502 mg, yield: 72%) was prepared by conducting a reaction in the same manner as in Preparation Example 16 except that D4-n (500 mg, 1 equivalent) was used instead of D1-a, and n-tetrabutylphosphine bromide (130 mg, 0.6 equivalents) was used instead of n-tetrabutylammonium iodine.

HR LC/MS/MS m/z calculated for C₅₆H₆₈Cl₂CoN₆O₈P (M+): 1112.3540; found: 1112.3541.

### Preparation Example 69: Synthesis of Compound P69

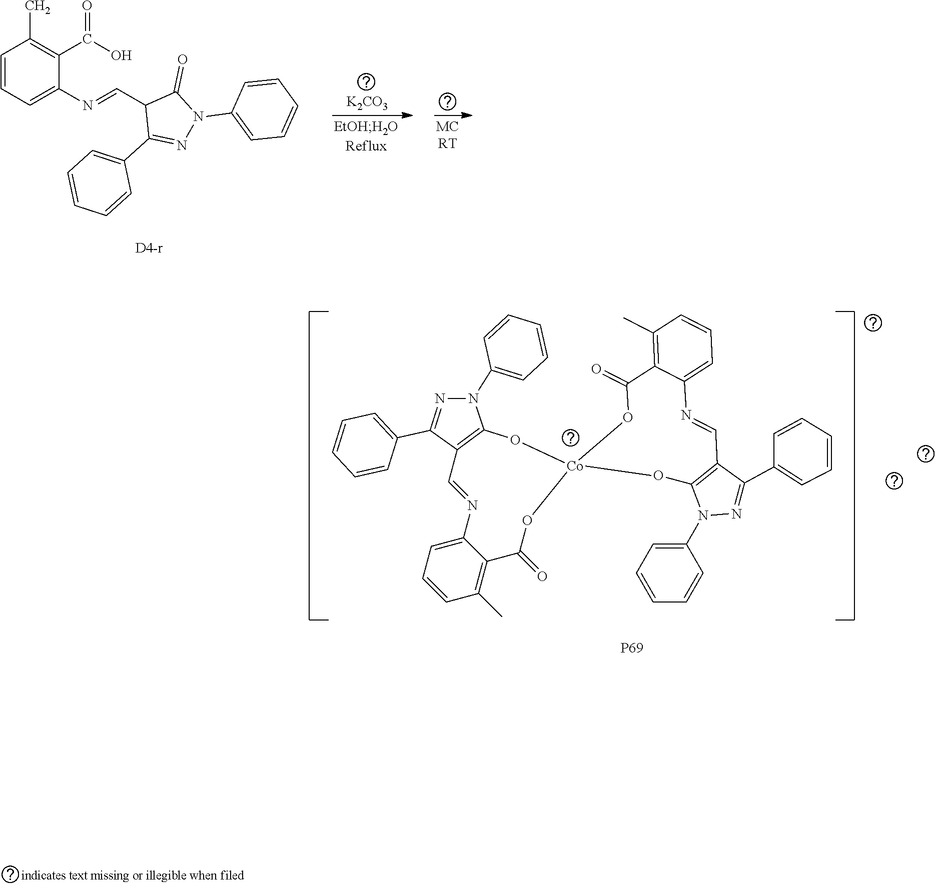

Compound P69 (405 mg, yield: 72%) was prepared by conducting a reaction in the same manner as in Preparation Example 16 except that D4-r (500 mg, 1 equivalent) was used instead of D1-a, and n-tetrabutylphosphine bromide (130 mg, 0.6 equivalents) was used instead of n-tetrabutylammonium iodine.

HR LC/MS/MS m/z calculated for C₆₄H₇₀CoN₆O₆ (M+): 1108.4421; found: 1108.4421.

### Preparation Example 70: Synthesis of Compound P70

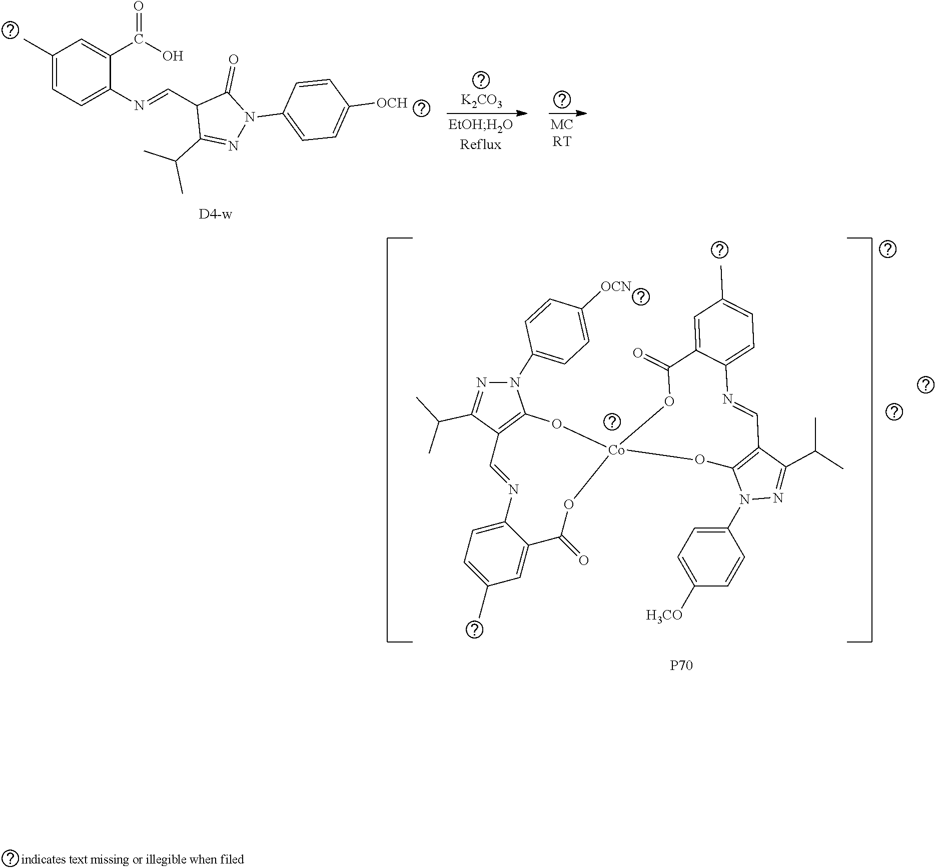

Compound P70 (267 mg, yield: 39%) was prepared by conducting a reaction in the same manner as in Preparation Example 16 except that D4-w (500 mg, 1 equivalent) was used instead of D1-a, and n-tetrabutylphosphine bromide (122 mg, 0.6 equivalents) was used instead of n-tetrabutylammonium iodine.

HR LC/MS/MS m/z calculated for C₅₈H₇₂CoN₈O₁₂P (M+): 1162.4334; found: 1162.4334.

### Preparation Example 71: Synthesis of Compound P71

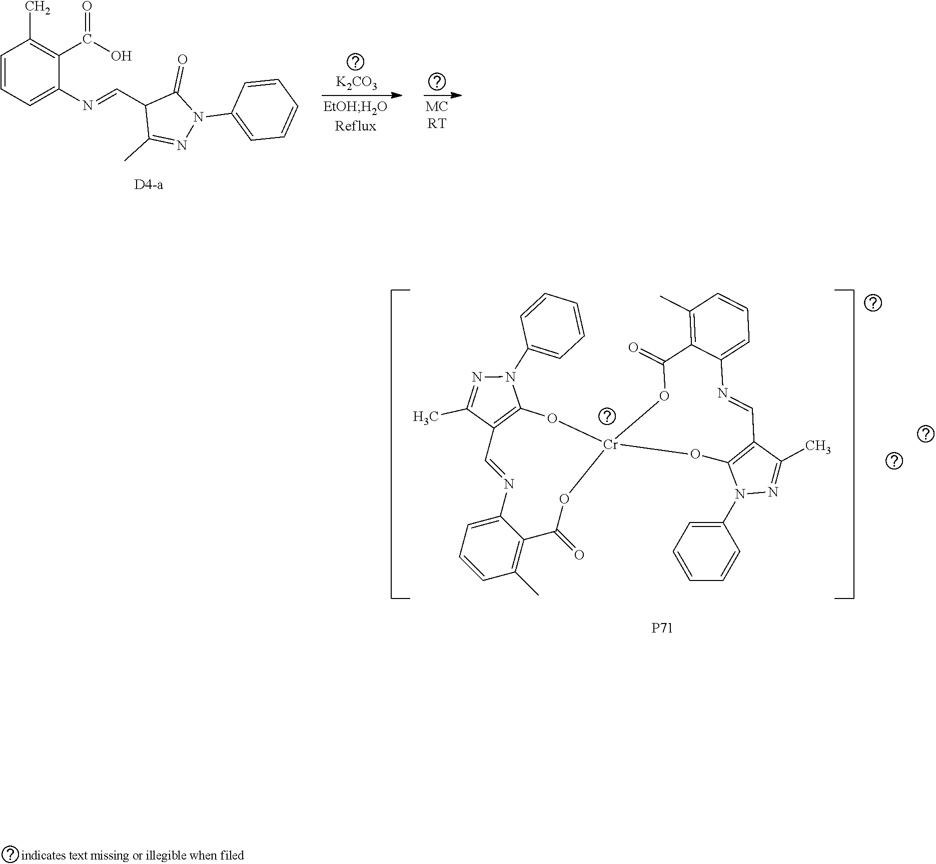

Compound P71 (322 mg, yield: 45%) was prepared by conducting a reaction in the same manner as in Preparation Example 41 except that D4-a (500 mg, 1 equivalent) was used instead of D1-a.

HR LC/MS/MS m/z calculated for C₅₄H₆₆CrN₇O₆(M+): 960.4474; found: 960.4474.

### Preparation Example 72: Synthesis of Compound P72

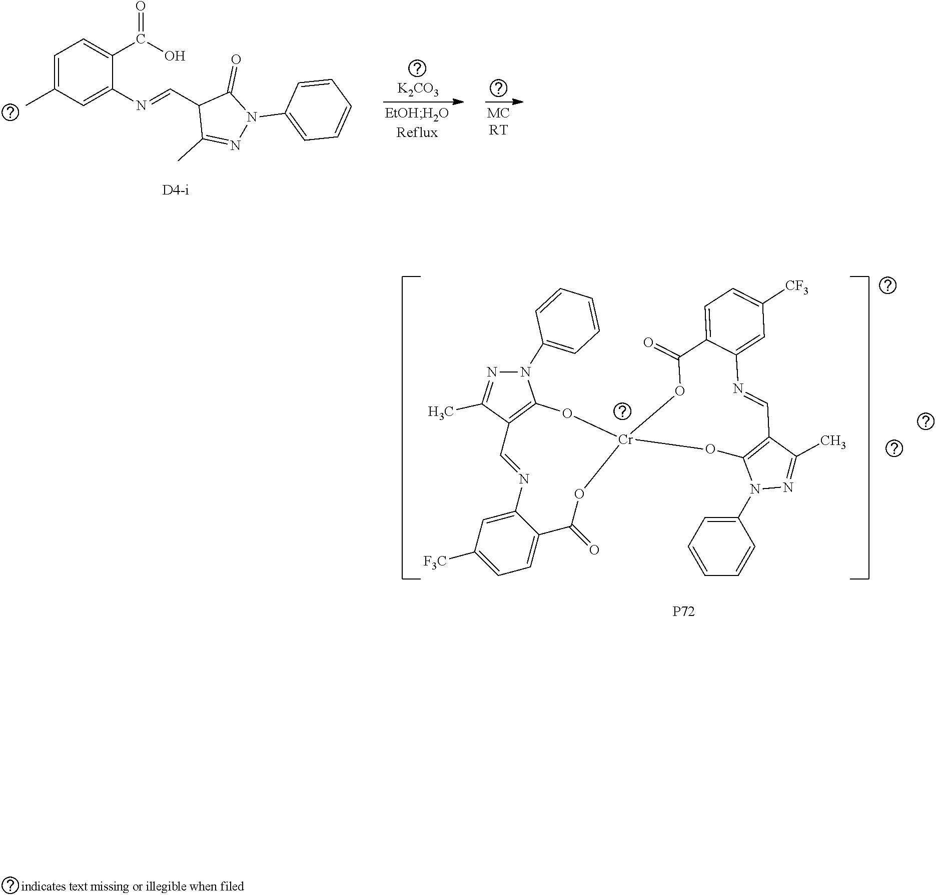

Compound P72 (378 mg, yield: 55%) was prepared by conducting a reaction in the same manner as in Preparation Example 41 except that D4-i (500 mg, 1 equivalent) was used instead of D1-a.

HR LC/MS/MS m/z calculated for C₅₄H₆₀CrF₆N₇O₆ (M+): 1068.3909; found: 1068.3909.

### Preparation Example 73: Synthesis of Compound P73

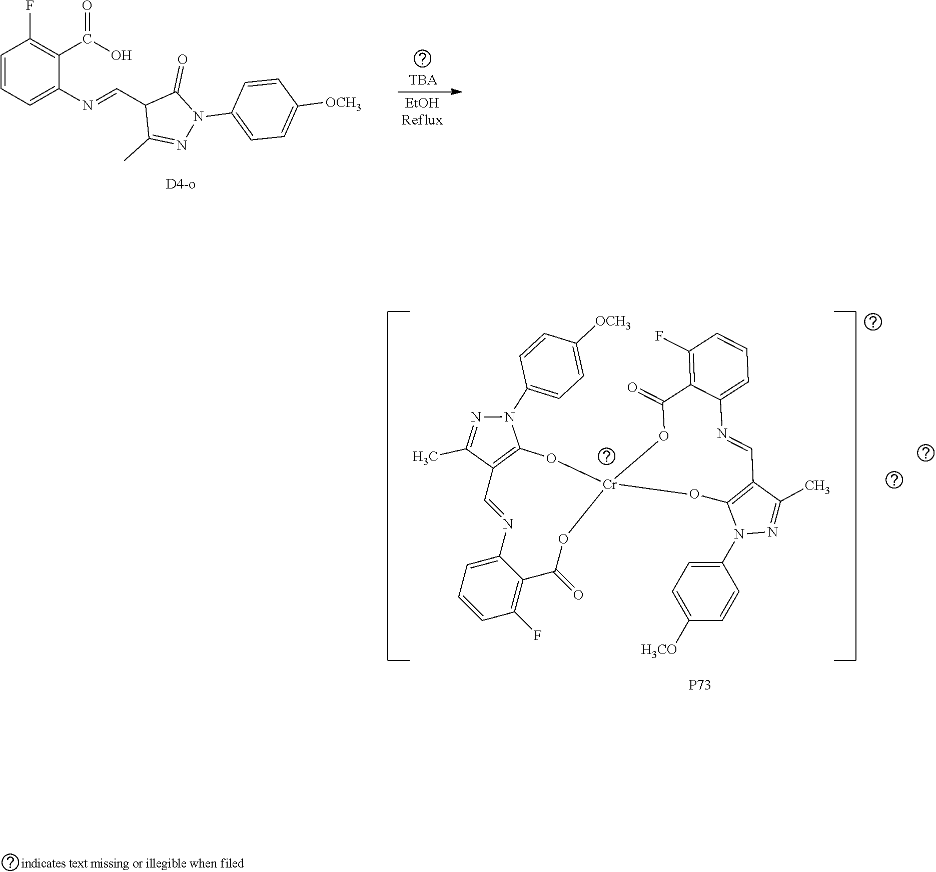

Compound P73 (402 mg, yield: 61%) was prepared by conducting a reaction in the same manner as in Preparation Example 45 except that D4-o (500 mg, 1 equivalent) was used instead of D3-g.

HR LC/MS/MS m/z calculated for C₅₀H₅₆CrF₂N₇O₈(M+): 972.3558; found: 972.3558.

### Preparation Example 74: Synthesis of Compound P74

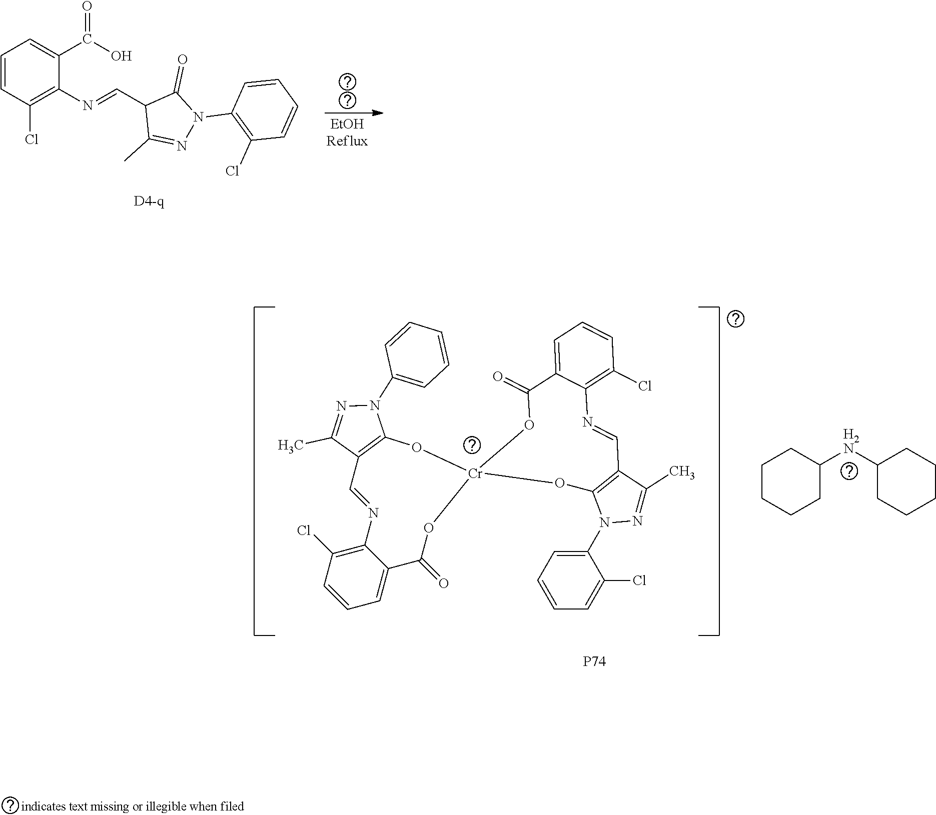

Compound P74 (330 mg, yield: 51%) was prepared by conducting a reaction in the same manner as in Preparation Example 45 except that D4-q (500 mg, 1 equivalent) was used instead of D3-g, and dicyclohexylamine (465 mg, 2 equivalents) was used instead of tributylamine.

HR LC/MS/MS m/z calculated for C₆₆H₇₄CrN₆O₆P (M+): 1129.4807; found: 1129.4807.

### Preparation Example 75: Synthesis of Compound P75

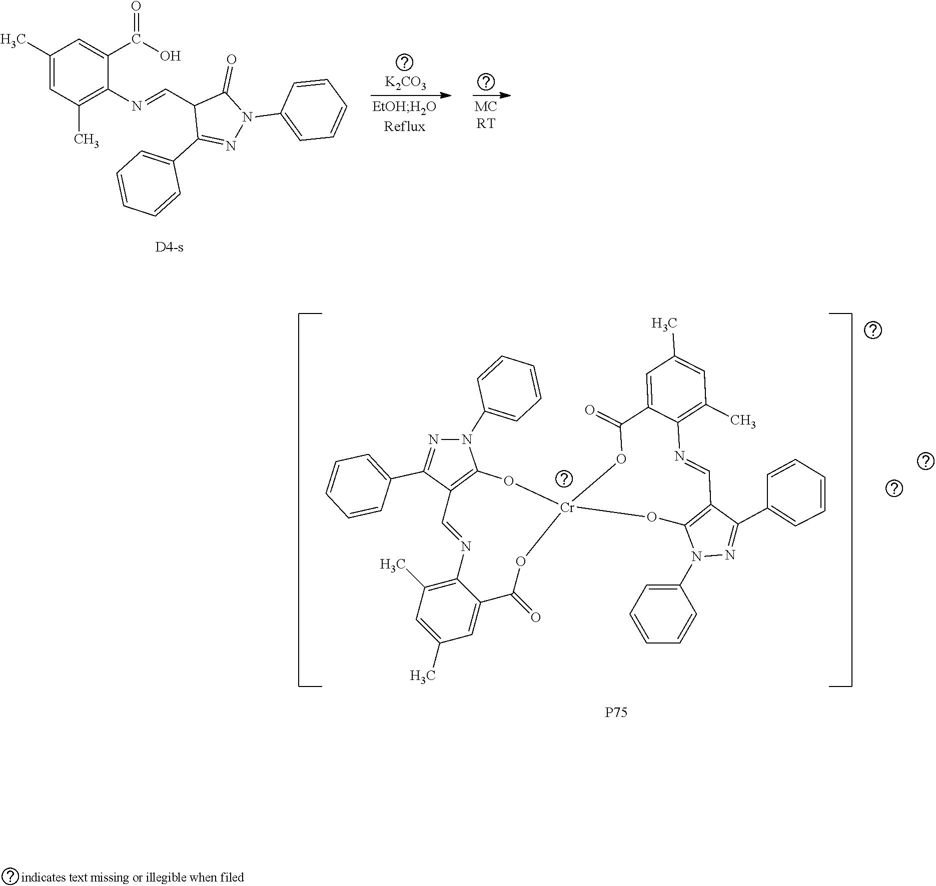

Compound P75 (261 mg, yield: 38%) was prepared by conducting a reaction in the same manner as in Preparation Example 41 except that D4-s (500 mg, 1 equivalent) was used instead of D1-a, and n-tetrabutylphosphine bromide (269 mg, 0.6 equivalents) was used instead of n-tetrabutylammonium iodine.

HR LC/MS/MS m/z calculated for C₆₆H₇₄CrN₆O₆P (M+): 1129.4807; found: 1129.4807.

### Preparation Example 76: Synthesis of Comparative Compound P76

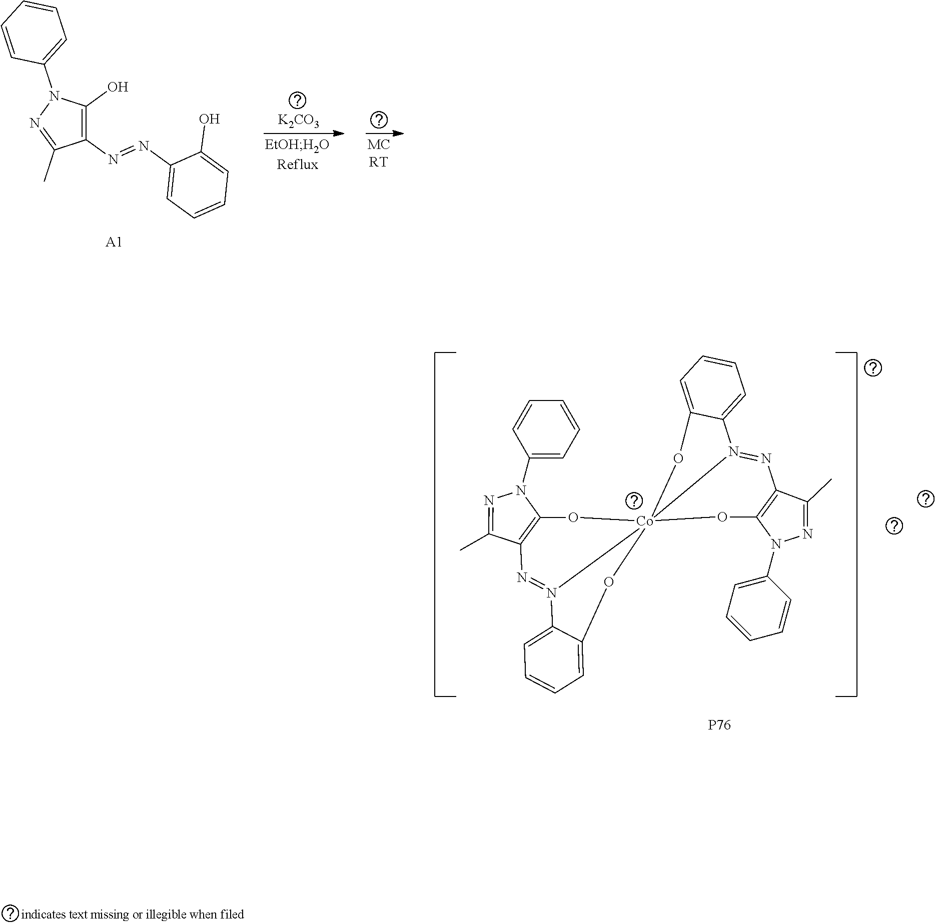

Comparative Compound P76 (565 mg, yield: 75%) was prepared by conducting a reaction in the same manner as in Preparation Example 16 except that A1 (500 mg, 1 equivalent) was used instead of D1-a.

HR LC/MS/MS m/z calculated for C₄₈H₄₆CoN₉O₄(M+): 885.4100; found: 885.4100.

### Preparation Example 77: Synthesis of Comparative Compound P77

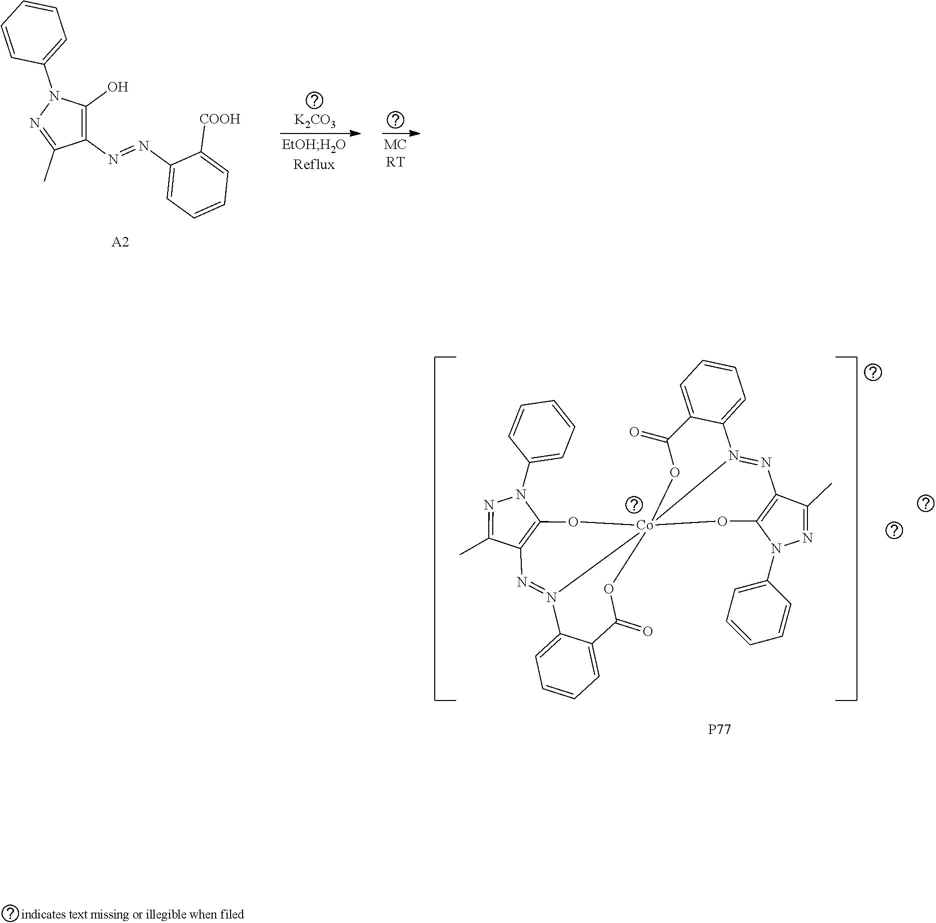

Comparative Compound P77 (482 mg, yield: 66%) was prepared by conducting a reaction in the same manner as in Preparation Example 16 except that A2 (500 mg, 1 equivalent) was used instead of D1-a.

HR LC/MS/MS m/z calculated for C₅₀H₆₀CoN₉O₆(M+): 941.3999; found: 941.3999.

### Preparation Example 78: Synthesis of Comparative Compound P78

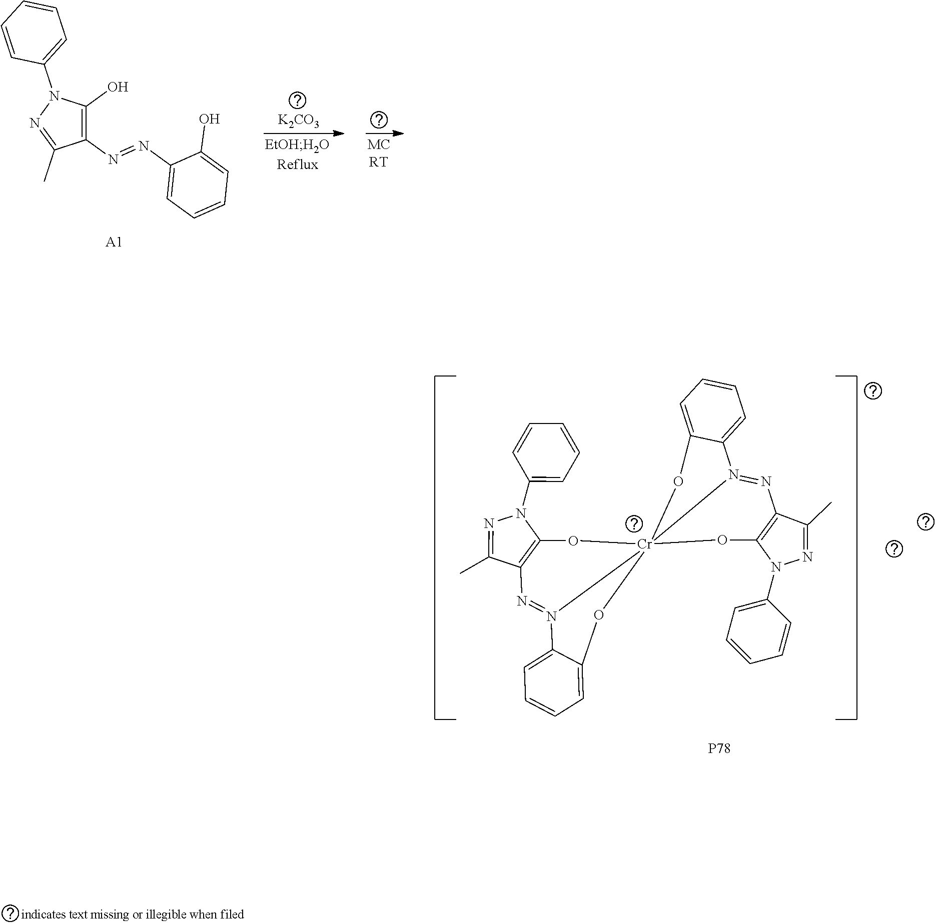

Comparative Compound P78 (336 mg, yield: 45%) was prepared by conducting a reaction in the same manner as in Preparation Example 41 except that A1 was used instead of D1-a.

HR LC/MS/MS m/z calculated for C₄₈H₆₀CrN₉O₄(M+): 878.4173; found: 878.4173.

### Preparation Example 79: Synthesis of Comparative Compound P79

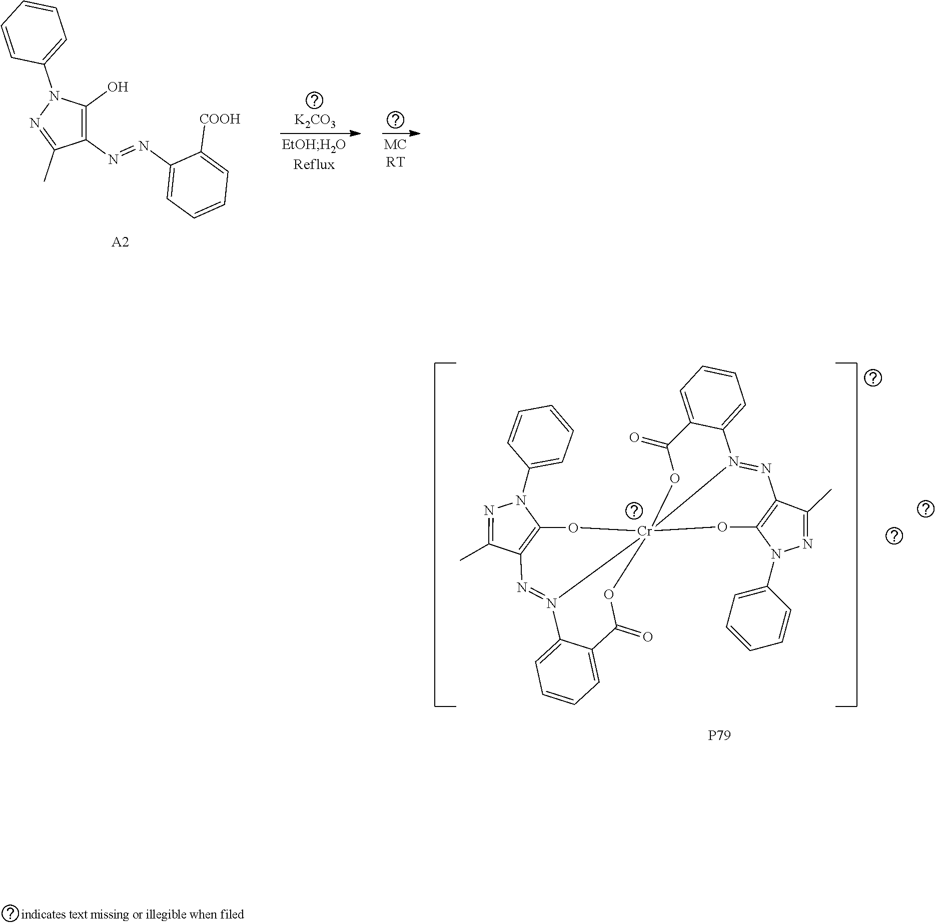

Compound P79 (370 mg, yield: 51%) was prepared by conducting a reaction in the same manner as in Preparation Example 41 except that A2 was used instead of D1-a.

HR LC/MS/MS m/z calculated for C₅₀H₆₀CrN₉O₆(M+): 934.4072; found: 934.4072.

### EXPERIMENTAL EXAMPLE

### Example 1

Based on 100 parts by weight of a solid content excluding a solvent in an adhesive composition, 95.4 parts by weight of an adhesive material (AD-701 solid content, LG Chem.), 0.29 parts by weight of Compound P1, 0.1 parts by weight of an isocyanate-based crosslinking agent (T39M, Soken Chemical & Engineering Co., Ltd.), 0.2 parts by weight of a silane-based coupling agent (T-789J, Soken Chemical & Engineering Co., Ltd.), 0.5 parts by weight of an antioxidant (Kinox-80, Hannong Chemicals Inc.), 1.5 parts by weight of an antistatic agent (FC-4400, 3M Corporation), 2 parts by weight of a hindered amine light stabilizer (Tinuvin 123, BASF Corporation) and 0.001 parts by weight a catalyst (dibutyltin dilaurate, Sigma-Aldrich) were mixed, and a solvent (methyl ethyl ketone, MEK) was added in 25 parts by weight based on the total parts by weight of the adhesive composition. The adhesive composition obtained by mixing these components using a shaker (SKC 6100, JEIO Tech.) was coated on a release layer (PET) to a thickness of 22 μm to 23 μm using a knife bar coating apparatus (KP-3000, Kipae E&T Co., Ltd.) to prepare an adhesive film. After the coating, the release layer was removed, and the adhesive film and a binder resin film (TAC: cellulose triacetate) were consecutively laminated on glass through a lamination process to prepare a sample. The binder resin film (TAC: triacetate cellulose) was used to prepare the sample using the adhesive film including the adhesive composition including Compound P1, and does not affect measured properties of the adhesive film.

Maximum absorption wavelengths Amax (nm) of the adhesive composition and the adhesive film were each measured using a UV-vis apparatus (Shimazu UV-3600). In addition, suitability as a light absorber was identified by subdividing the maximum absorption wavelength range, and the results are described in Table 1.

[Wavelength Suitability as Light Absorber]

Maximum absorption wavelength of 380 nm to 420 nm: ◯

Maximum absorption wavelength of 421 nm to 450 nm: Δ

Maximum absorption wavelength of greater than 450 nm: x

### Example 2

An experiment was conducted under the same conditions as in Example 1 except that Compound P5 was used instead of Compound P1, and the results are described in Table 1.

### Example 3

An experiment was conducted under the same conditions as in Example 1 except that Compound P6 was used instead of Compound P1, and the results are described in Table 1

### Example 4

An experiment was conducted under the same conditions as in Example 1 except that Compound P7 was used instead of Compound P1, and the results are described in Table 1.

### Example 5

An experiment was conducted under the same conditions as in Example 1 except that Compound P10 was used instead of Compound P1, and the results are described in Table 1.

### Example 6

An experiment was conducted under the same conditions as in Example 1 except that Compound P16 was used instead of Compound P1, and the results are described in Table 1.

### Example 7

An experiment was conducted under the same condition as in Example 1 except that Compound P17 was used instead of Compound P1, and the results are described in the following Table 1.

### Example 8

An experiment was conducted under the same conditions as in Example 1 except that Compound P18 was used instead of Compound P1, and the results are described in Table 1.

### Example 9

An experiment was conducted under the same conditions as in Example 1 except that Compound P19 was used instead of Compound P1, and the results are described in Table 1.

### Example 10

An experiment was conducted under the same conditions as in Example 1 except that Compound P20 was used instead of Compound P1, and the results are described in Table 1.

### Example 11

An experiment was conducted under the same conditions as in Example 1 except that Compound P21 was used instead of Compound P1, and the results are described in Table 1.

### Example 12

An experiment was conducted under the same conditions as in Example 1 except that Compound P22 was used instead of Compound P1, and the results are described in Table 1.

### Example 13

An experiment was conducted under the same conditions as in Example 1 except that Compound P24 was used instead of Compound P1, and the results are described in Table 1.

### Example 14

An experiment was conducted under the same conditions as in Example 1 except that Compound P25 was used instead of Compound P1, and the results are described in Table 1.

### Example 15

An experiment was conducted under the same conditions as in Example 1 except that Compound P26 was used instead of Compound P1, and the results are described in Table 1.

### Example 16

An experiment was conducted under the same conditions as in Example 1 except that Compound P27 was used instead of Compound P1, and the results are described in Table 1.

### Example 17

An experiment was conducted under the same conditions as in Example 1 except that Compound P28 was used instead of Compound P1, and the results are described in Table 1.

### Example 18

An experiment was conducted under the same conditions as in Example 1 except that Compound P29 was used instead of Compound P1, and the results are described in Table 1.

### Example 19

An experiment was conducted under the same conditions as in Example 1 except that Compound P30 was used instead of Compound P1, and the results are described in Table 1.

### Example 20

An experiment was conducted under the same conditions as in Example 1 except that Compound P31 was used instead of Compound P1, and the results are described in Table 1.

### Example 21

An experiment was conducted under the same conditions as in Example 1 except that Compound P32 was used instead of Compound P1, and the results are described in Table 1.

### Example 22

An experiment was conducted under the same conditions as in Example 1 except that Compound P33 was used instead of Compound P1, and the results are described in Table 1.

### Example 23

An experiment was conducted under the same conditions as in Example 1 except that Compound P34 was used instead of Compound P1, and the results are described in Table 1.

### Example 24

An experiment was conducted under the same conditions as in Example 1 except that Compound P35 was used instead of Compound P1, and the results are described in Table 1.

### Example 25

An experiment was conducted under the same conditions as in Example 1 except that Compound P36 was used instead of Compound P1, and the results are described in Table 1.

### Example 26

An experiment was conducted under the same conditions as in Example 1 except that Compound P37 was used instead of Compound P1, and the results are described in Table 1.

### Example 27

An experiment was conducted under the same conditions as in Example 1 except that Compound P38 was used instead of Compound P1, and the results are described in Table 1.

### Example 28

An experiment was conducted under the same conditions as in Example 1 except that Compound P41 was used instead of Compound P1, and the results are described in Table 1.

### Example 29

An experiment was conducted under the same conditions as in Example 1 except that Compound P43 was used instead of Compound P1, and the results are described in Table 1.

### Example 30

An experiment was conducted under the same conditions as in Example 1 except that Compound P44 was used instead of Compound P1, and the results are described in Table 1.

### Example 31

An experiment was conducted under the same conditions as in Example 1 except that Compound P45 was used instead of Compound P1, and the results are described in Table 1.

### Example 32

An experiment was conducted under the same conditions as in Example 1 except that Compound P46 was used instead of Compound P1, and the results are described in Table 1.

### Example 33

An experiment was conducted under the same conditions as in Example 1 except that Compound P49 was used instead of Compound P1, and the results are described in Table 1.

### Example 34

An experiment was conducted under the same conditions as in Example 1 except that Compound P52 was used instead of Compound P1, and the results are described in Table 1.

### Example 35

An experiment was conducted under the same conditions as in Example 1 except that Compound P54 was used instead of Compound P1, and the results are described in Table 1.

### Example 36

An experiment was conducted under the same conditions as in Example 1 except that Compound P59 was used instead of Compound P1, and the results are described in Table 1.

### Example 37

An experiment was conducted under the same conditions as in Example 1 except that Compound P60 was used instead of Compound P1, and the results are described in Table 1.

### Example 38

An experiment was conducted under the same conditions as in Example 1 except that Compound P71 was used instead of Compound P1, and the results are described in Table 1.

### Comparative Experimental Example 1

An experiment was conducted under the same conditions as in Example 1 except that Comparative Compound P76 was used instead of Compound P1, and the results are described in Table 1.

### Comparative Experimental Example 2

An experiment was conducted under the same conditions as in Example 1 except that Comparative Compound P77 was used instead of Compound P1, and the results are described in Table 1.

### Comparative Experimental Example 3

An experiment was conducted under the same conditions as in Example 1 except that Comparative Compound P78 was used instead of Compound P1, and the results are described in Table 1.

### Comparative Experimental Example 4

An experiment was conducted under the same conditions as in Example 1 except that Comparative Compound P79 was used instead of Compound P1, and the results are described in Table 1.

TABLE 1

| | Preparation | Solution | Film | Wavelength |
|---|---|---|---|---|
| Example | Example | λmax (nm) | λmax (nm) | Suitability |
| Example 1 | P1 | 382 | 382 | ∘ |
| Example 2 | P5 | 380 | 381 | ∘ |
| Example 3 | P6 | 383 | 383 | ∘ |
| Example 4 | P7 | 383 | 382 | ∘ |
| Example 5 | P10 | 384 | 386 | ∘ |
| Example 6 | P16 | 416 | 415 | ∘ |
| Example 7 | P17 | 417 | 418 | ∘ |
| Example 8 | P18 | 411 | 412 | ∘ |
| Example 9 | P19 | 411 | 411 | ∘ |
| Example 10 | P20 | 409 | 408 | ∘ |
| Example 11 | P21 | 427 | 429 | Δ |
| Example 12 | P22 | 426 | 427 | Δ |
| Example 13 | P24 | 408 | 409 | ∘ |
| Example 14 | P25 | 429 | 428 | Δ |
| Example 15 | P26 | 435 | 437 | Δ |
| Example 16 | P27 | 407 | 410 | ∘ |
| Example 17 | P28 | 408 | 409 | ∘ |
| Example 18 | P29 | 409 | 409 | ∘ |
| Example 19 | P30 | 436 | 438 | Δ |
| Example 20 | P31 | 421 | 422 | Δ |
| Example 21 | P32 | 418 | 418 | ∘ |
| Example 22 | P33 | 418 | 419 | ∘ |
| Example 23 | P34 | 406 | 405 | ∘ |
| Example 24 | P35 | 404 | 404 | ∘ |
| Example 25 | P36 | 409 | 409 | ∘ |
| Example 26 | P37 | 415 | 416 | ∘ |
| Example 27 | P38 | 413 | 412 | ∘ |
| Example 28 | P41 | 446 | 446 | Δ |
| Example 29 | P43 | 435 | 436 | Δ |
| Example 30 | P44 | 428 | 429 | Δ |
| Example 31 | P45 | 425 | 426 | Δ |
| Example 32 | P46 | 433 | 432 | Δ |
| Example 33 | P49 | 447 | 446 | Δ |
| Example 34 | P52 | 401 | 402 | ∘ |
| Example 35 | P54 | 397 | 399 | ∘ |
| Example 36 | P59 | 394 | 393 | ∘ |
| Example 37 | P60 | 396 | 397 | ∘ |
| Example 38 | P71 | 384 | 384 | ∘ |
| Comparative | P76 | 492 | 495 | x |
| Experimental | | | | |
| Example 1 | | | | |
| Comparative | P77 | 473 | 471 | x |
| Experimental | | | | |
| Example 2 | | | | |
| Comparative | P78 | 504 | 504 | x |
| Experimental | | | | |
| Example 3 | | | | |
| Comparative | P79 | 471 | 472 | x |
| Experimental | | | | |
| Example 4 | | | | |

From the experimental results of Table 1, it is identified that the adhesive films including the coordination compound according to one embodiment of the present disclosure have excellent wavelength suitability compared to the films that do not include the coordination compound. Specifically, when the adhesive films according to the examples of the present disclosure have a maximum absorption wavelength (Amax) in a range of 380 nm to 450 nm unlike the adhesive films according to the comparative examples having a maximum absorption wavelength (Amax) of greater than 470 nm, a decrease in the blue luminance may be minimized by absorbing a longer wavelength region compared to an existing blue light blocking film, and efficiency may be maximized.

1. A coordination compound, wherein the coordination compound is represented by Aⁿ⁻, wherein A is represented by Chemical Formula A, and n is 1 or 2: 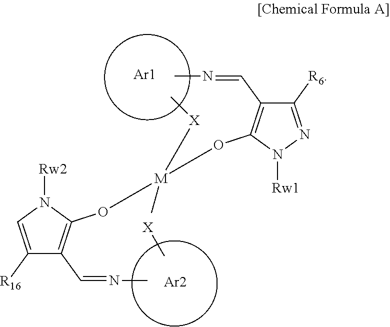
wherein in Chemical Formula A,
M is a Cr ion or a Co ion;
X is O or O—C═O;
Rw1 and Rw2 are the same as or different from each other, and each independently is a substituted or unsubstituted alkyl group; a substituted or unsubstituted alkenyl group; a substituted or unsubstituted cycloalkyl group; a substituted or unsubstituted aryl group; or a substituted or unsubstituted heteroaryl group;
Ar1 and Ar2 are the same as or different from each other, and each independently is a substituted or unsubstituted arylene group; or a substituted or unsubstituted heteroarylene group;
R₆ and R₁₆ are the same as or different from each other, and each independently is hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; a substituted or unsubstituted styryl group; —OR₁₀₀; —CO₂R₁₀₁; —COR₁₀₂; —OCOR₁₀₃; —CONR₁₀₄R₁₀₅; or —SO₂R₁₀₆; and
R₁₀₀ to R₁₀₆ are the same as or different from each other, and each independently is hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

2. The coordination compound of claim 1, wherein A is represented by Chemical Formula A-1: 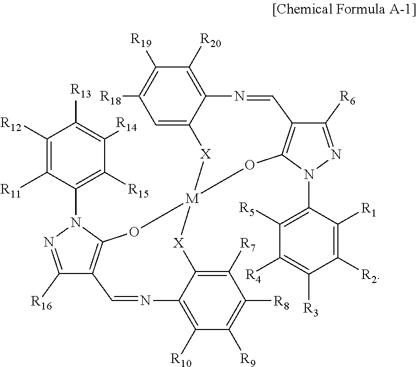
wherein in Chemical Formula A-1,
M, X, R₆ and R₁₆ have the same definitions as in Chemical Formula A;
R₁ to R₅, R₇ to R₁₅ and R₁₇ to R₂₀ are the same as or different from each other, and each independently is hydrogen; deuterium; a nitrile group; a nitro group; a hydroxyl group; —COOH; a halogen group; an imide group; a substituted or unsubstituted alkyl group; a substituted or unsubstituted cycloalkyl group; a substituted or unsubstituted heterocyclic group; a substituted or unsubstituted aryl group; a substituted or unsubstituted heteroaryl group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted arylphosphine group; a substituted or unsubstituted phosphine oxide group; —OR₁₀₀; —CO₂R₁₀₁; —COR₁₀₂; —OCOR₁₀₃; —CONR₁₀₄R₁₀₅; or —SO₂R₁₀₆, or adjacent substituents selected from R₁ to R₅, R₇ to R₁₅ and R₁₇ to R₂₀ bond to each other to form a substituted or unsubstituted aromatic ring or a substituted or unsubstituted aliphatic ring; and
R₁₀₀ to R₁₀₆ are the same as or different from each other, and each independently is hydrogen; a substituted or unsubstituted alkyl group; a substituted or unsubstituted cycloalkyl group; a substituted or unsubstituted aryl group; a substituted or unsubstituted heteroaryl group; or a substituted or unsubstituted amine group.

3. The coordination compound of claim 2, wherein R₁ to R₅ and R₁₁ to R₁₅ are the same as or different from each other, and each independently is hydrogen; deuterium; a halogen group; a nitrile group; a nitro group; —COOH; a substituted or unsubstituted alkyl group having 1 to 10 carbon atoms; a substituted or unsubstituted cycloalkyl group having 3 to 10 carbon atoms; an unsubstituted aryl group having 6 to 10 carbon atoms or an aryl group having 6 to 10 carbon atoms substituted with an alkyl group; a substituted or unsubstituted heteroaryl group having 2 to 10 carbon atoms; —OR₁₀₀; or —CO₂R₁₀₁; and
R₁₀₀ and R₁₀₁ are the same as or different from each other, and each independently is a substituted or unsubstituted alkyl group having 1 to 10 carbon atoms.

4. The coordination compound of claim 2, wherein R₇ to R₁₀ and R₁₇ to R₂₀ are the same as or different from each other, and each independently is hydrogen; deuterium; a halogen group; a nitrile group; a nitro group; a substituted or unsubstituted alkyl group having 1 to 10 carbon atoms; a substituted or unsubstituted cycloalkyl group having 3 to 10 carbon atoms; an unsubstituted aryl group having 6 to 10 carbon atoms or an aryl group having 6 to 10 carbon atoms substituted with —OR₁₀₀ or an alkyl group; a substituted or unsubstituted heteroaryl group having 2 to 10 carbon atoms; —OR₁₀₀; —CO₂R₁₀₁; —COR₁₀₂; or —SO₂R₁₀₆, or adjacent substituents selected from R₇ to R₁₀ and R₁₇ to R₂₀ bond to each other to form a substituted or unsubstituted aromatic ring;
R₁₀₀, R₁₀₁ and R₁₀₂ are the same as or different from each other, and each independently is a substituted or unsubstituted alkyl group having 1 to 10 carbon atoms; and
R₁₀₆ is a substituted or unsubstituted alkyl group having 1 to 10 carbon atoms; or —NH₂.

5. The coordination compound of claim 1, wherein R₆ and R₁₆ are the same as or different from each other, and each independently is a nitrile group; an unsubstituted methyl group or a methyl group substituted with a fluoro group; a substituted or unsubstituted ethyl group; a substituted or unsubstituted isopropyl group; an unsubstituted phenyl group or a phenyl group substituted with one or more substituents selected from the group consisting of a halogen group, a nitrile group, a nitro group, —COOH, an alkyl group, —OR₁₀₀ and —CO₂R₁₀₁; a substituted or unsubstituted benzoxazole group; a substituted or unsubstituted pyridine group; an unsubstituted styryl group or a styryl group substituted with an alkyl group or a nitro group; —OR₁₀₀; or —CO₂R₁₀₁; and
R₁₀₁ and R₁₀₂ are the same as or different from each other, and each independently is a substituted or unsubstituted methyl group; or a substituted or unsubstituted ethyl group.

6. The coordination compound of claim 1, wherein M is Co²⁺; Co³⁺; Cr²⁺; or Cr³⁺.

7. The coordination compound of claim 1, wherein Chemical Formula A is any one selected from the group consisting of: 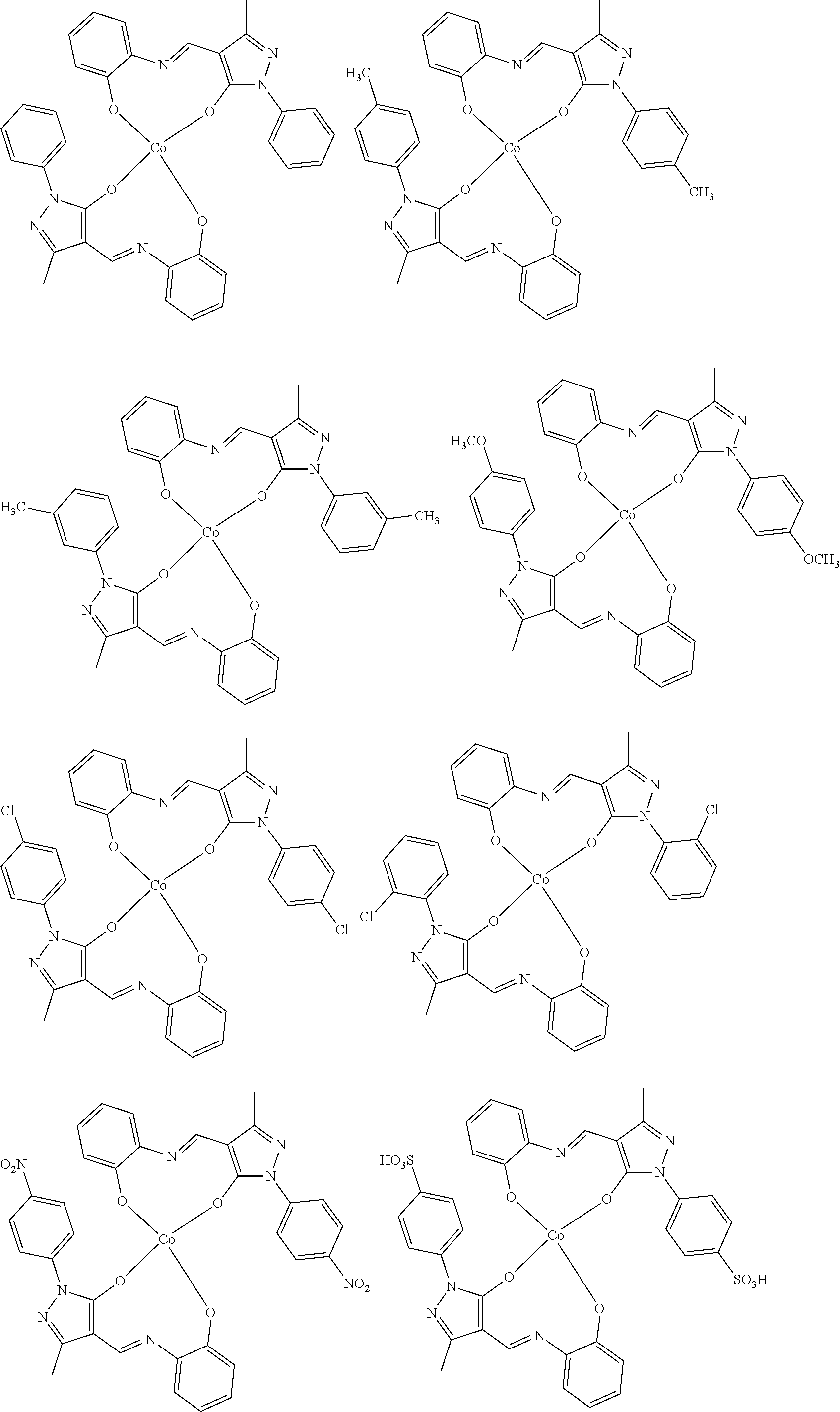 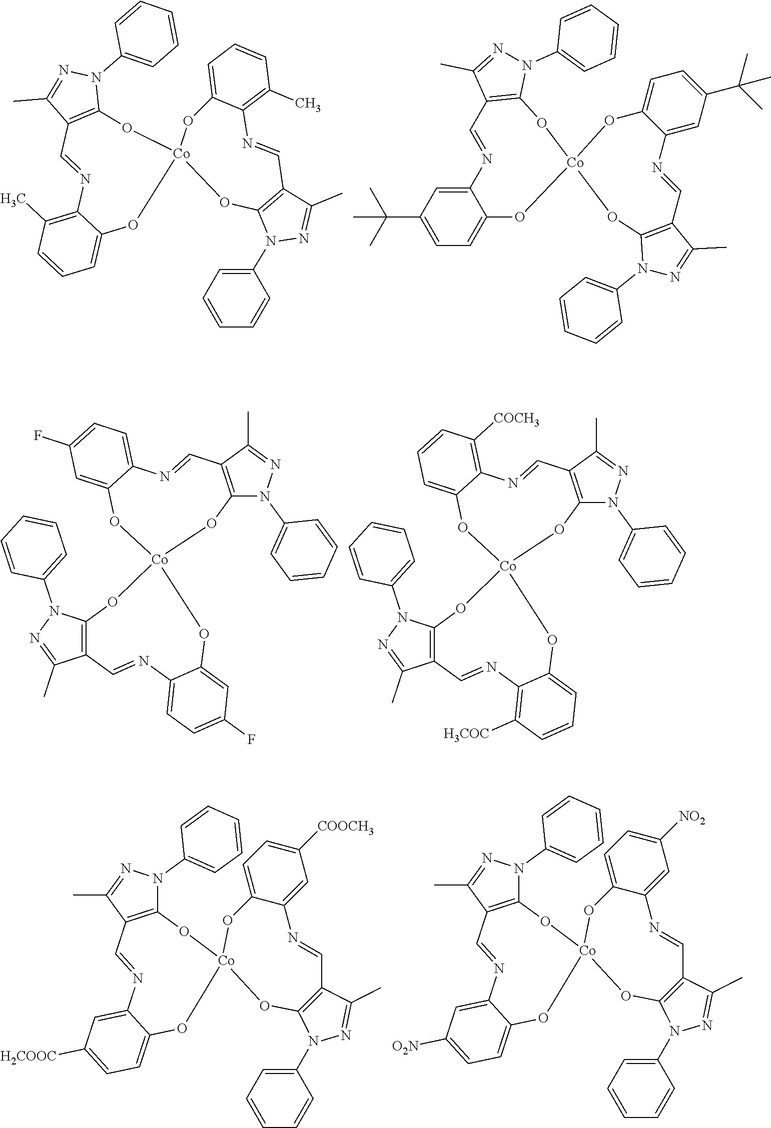 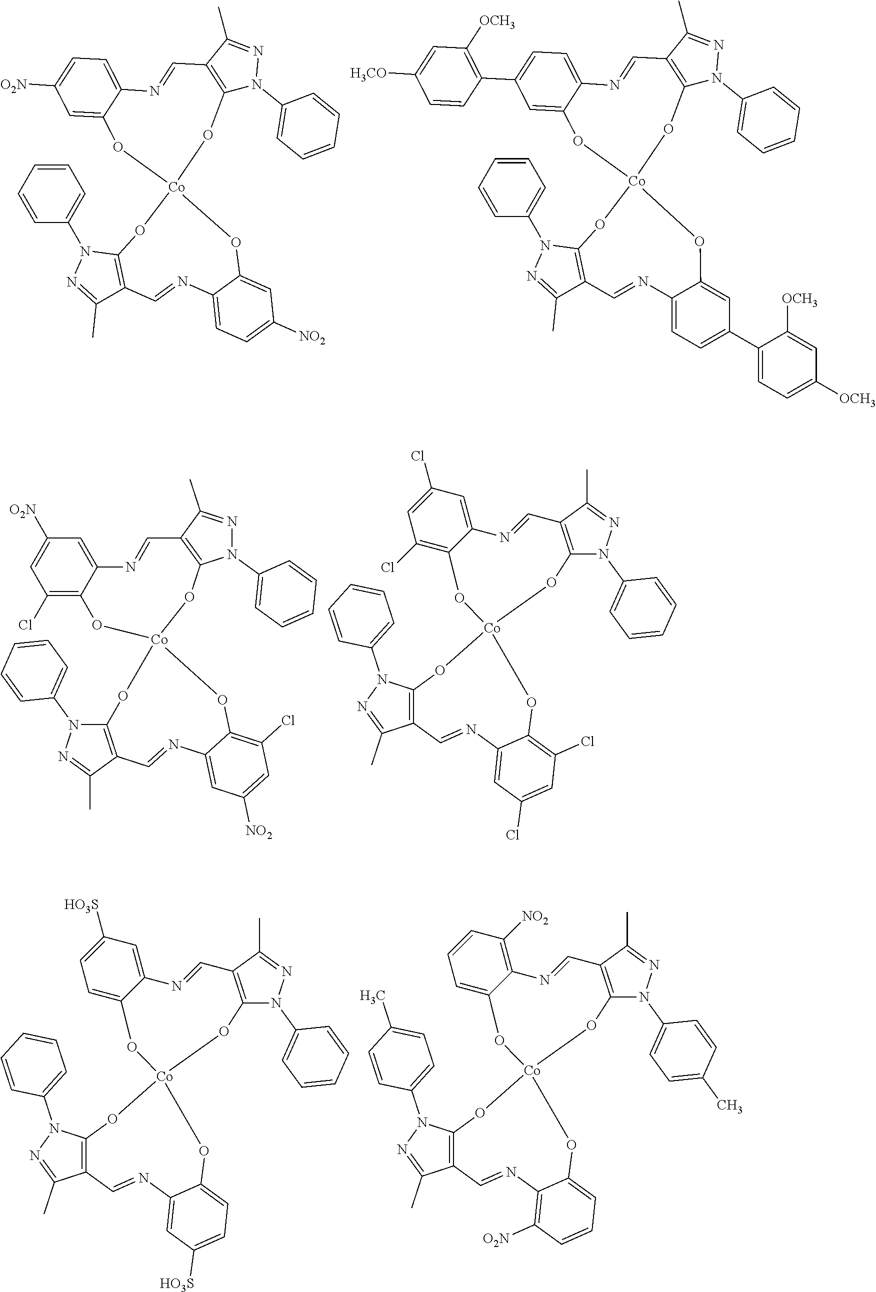 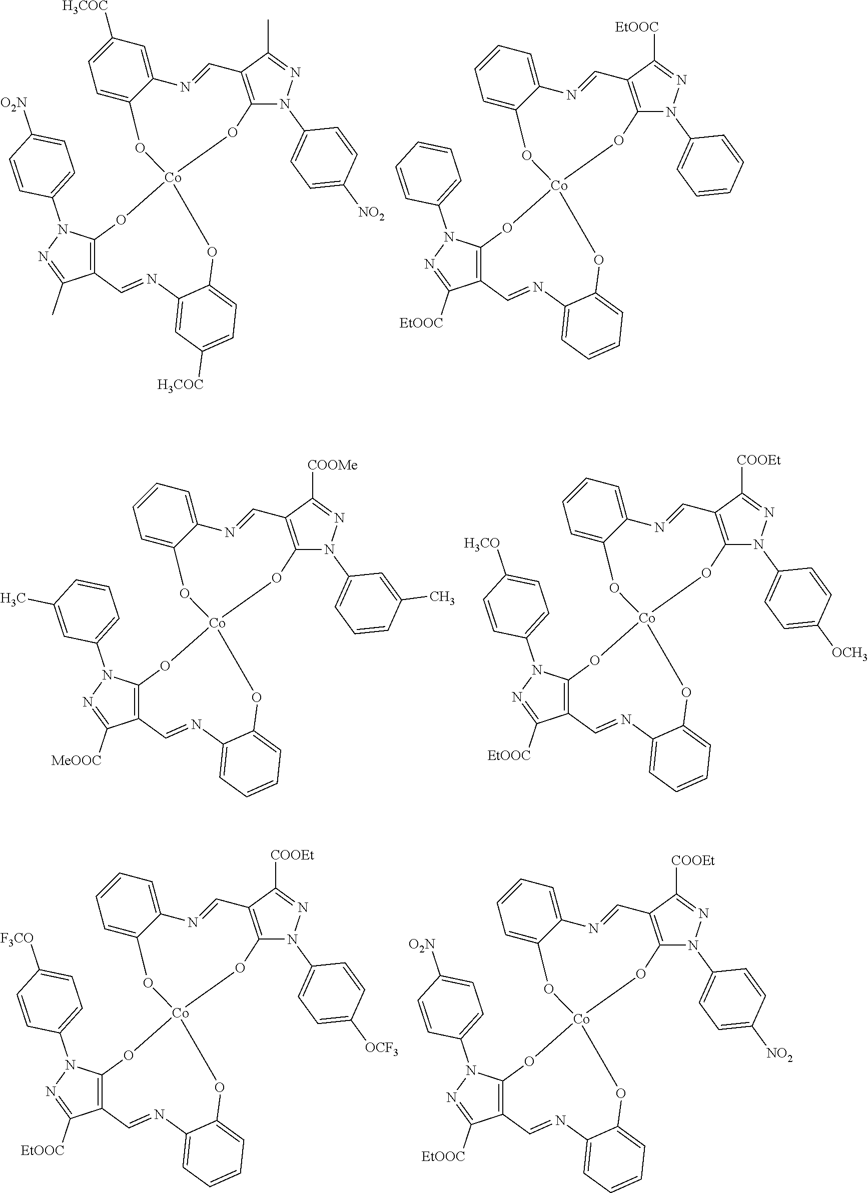 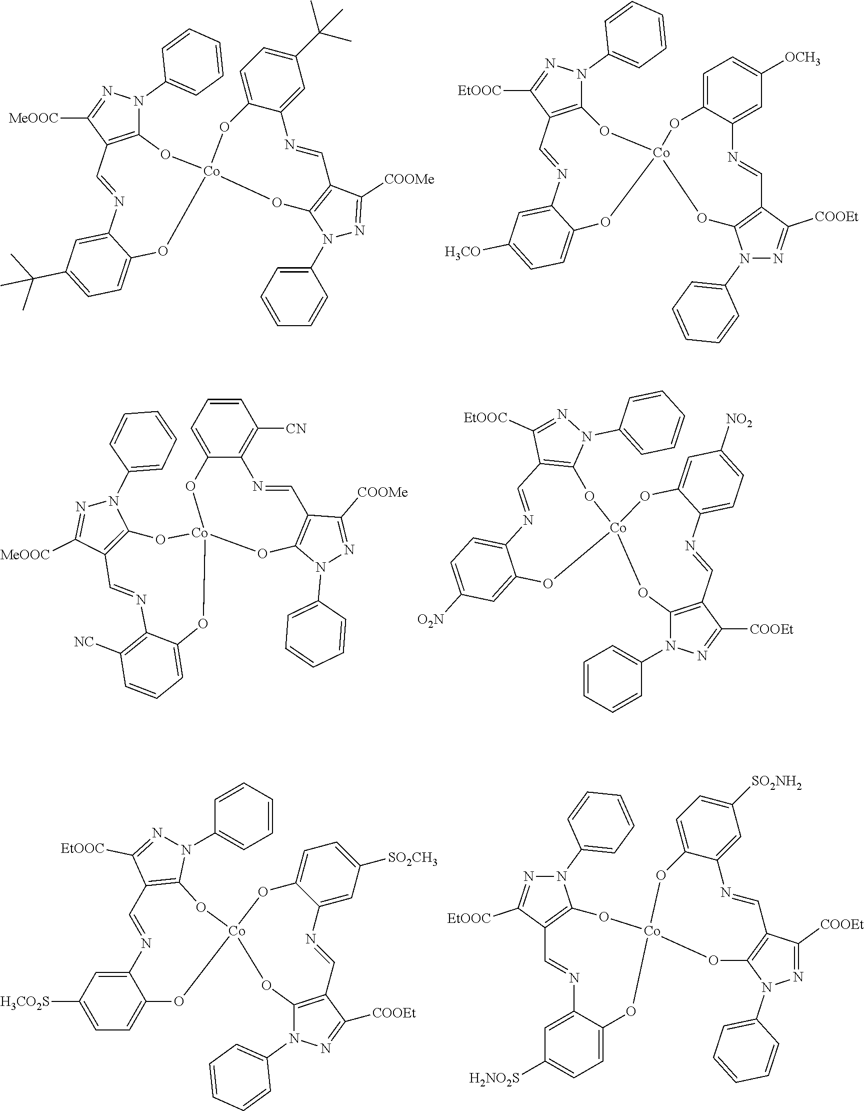 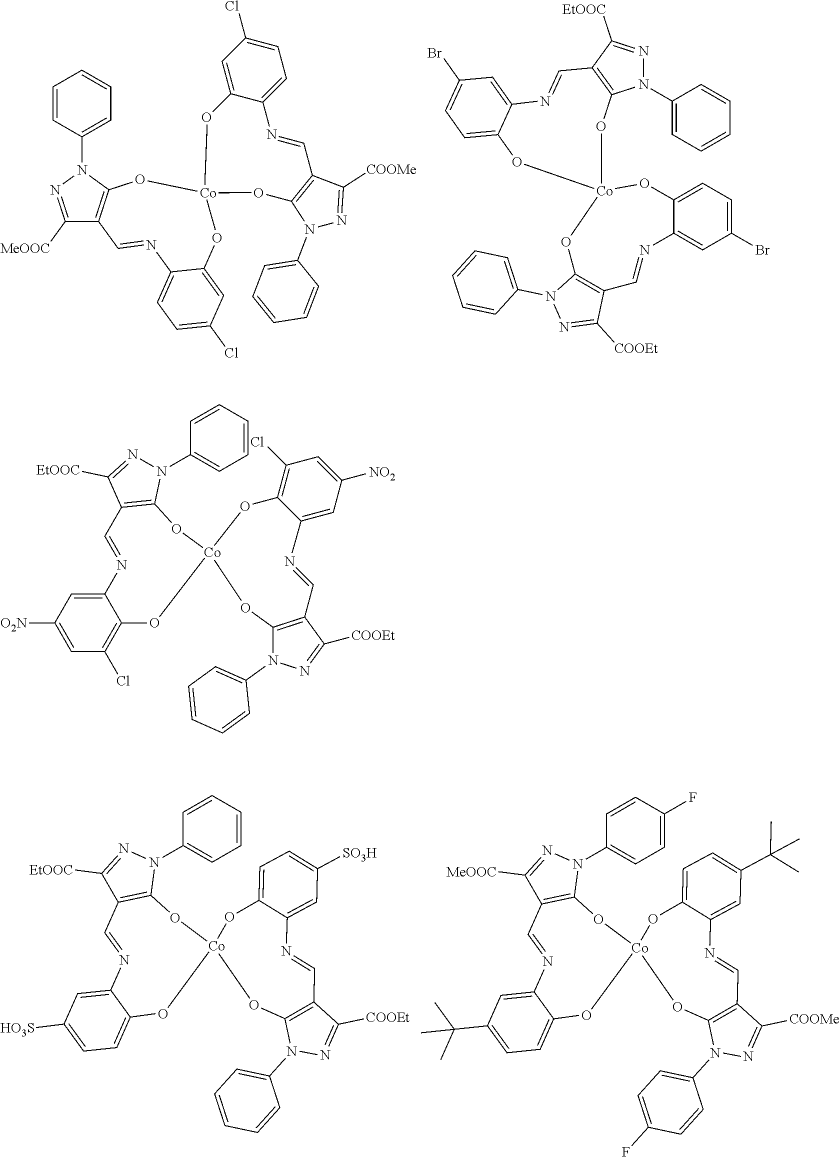 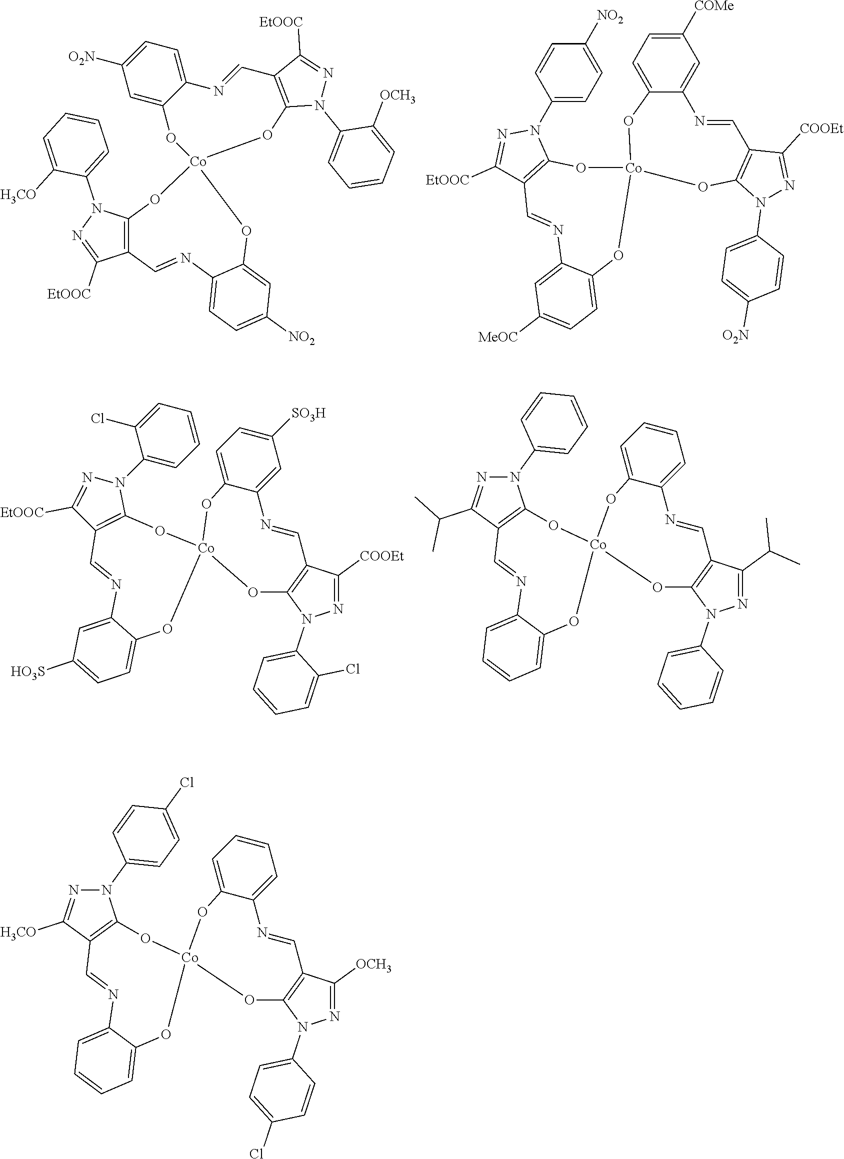 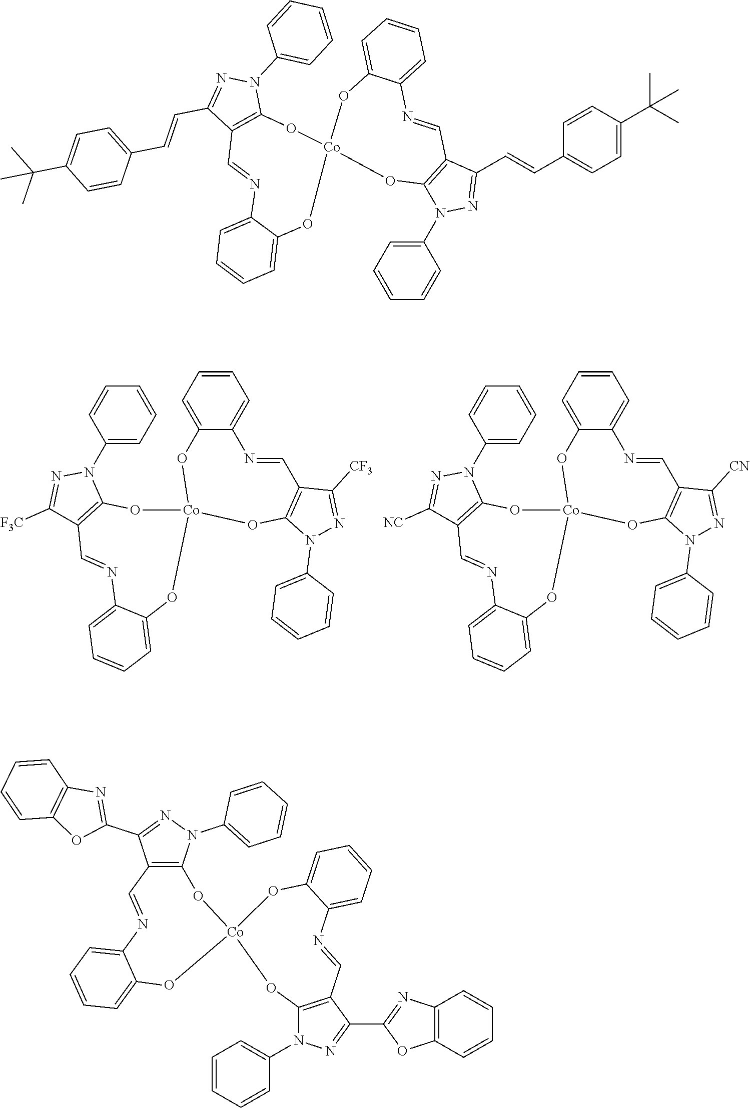 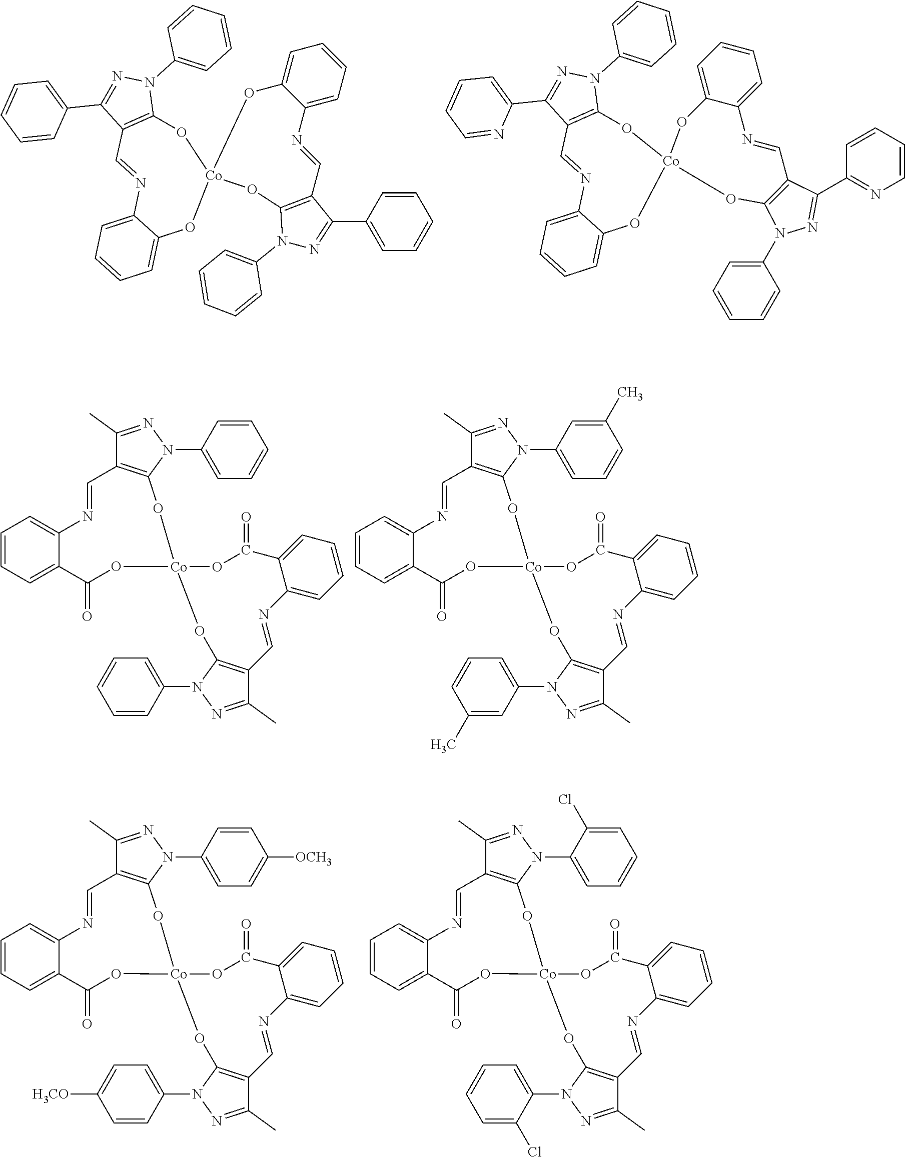 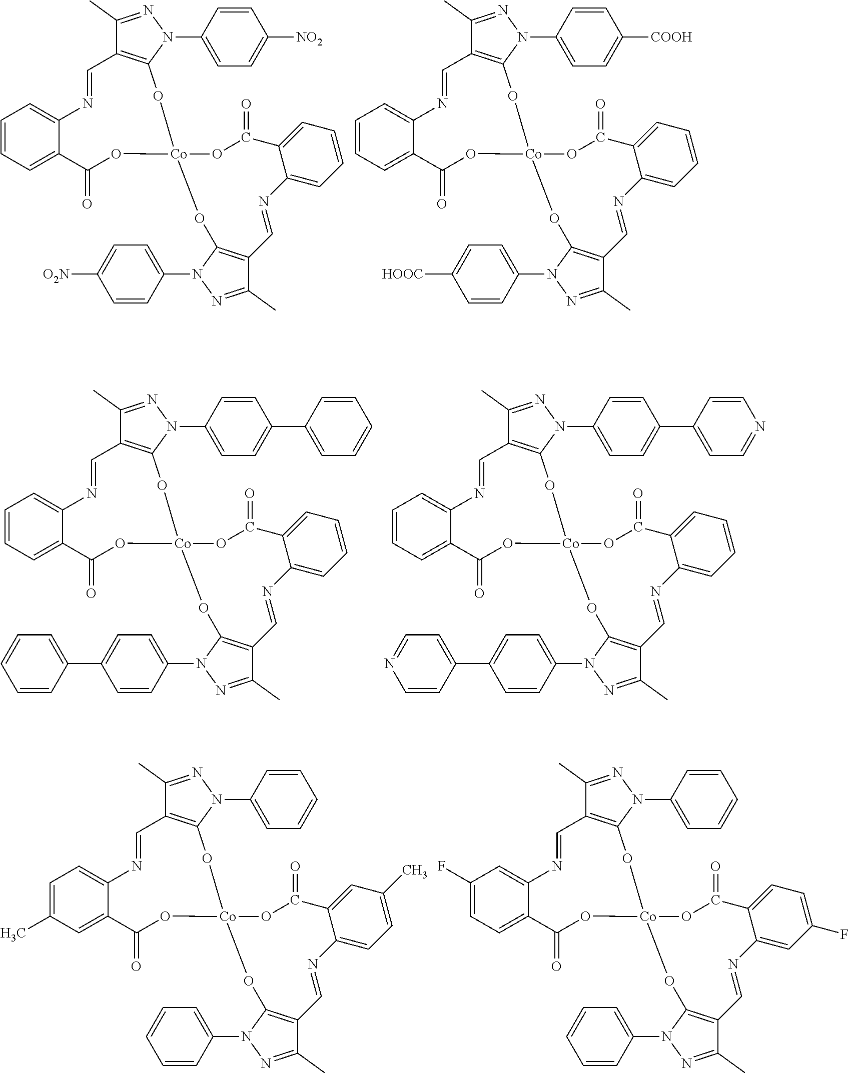 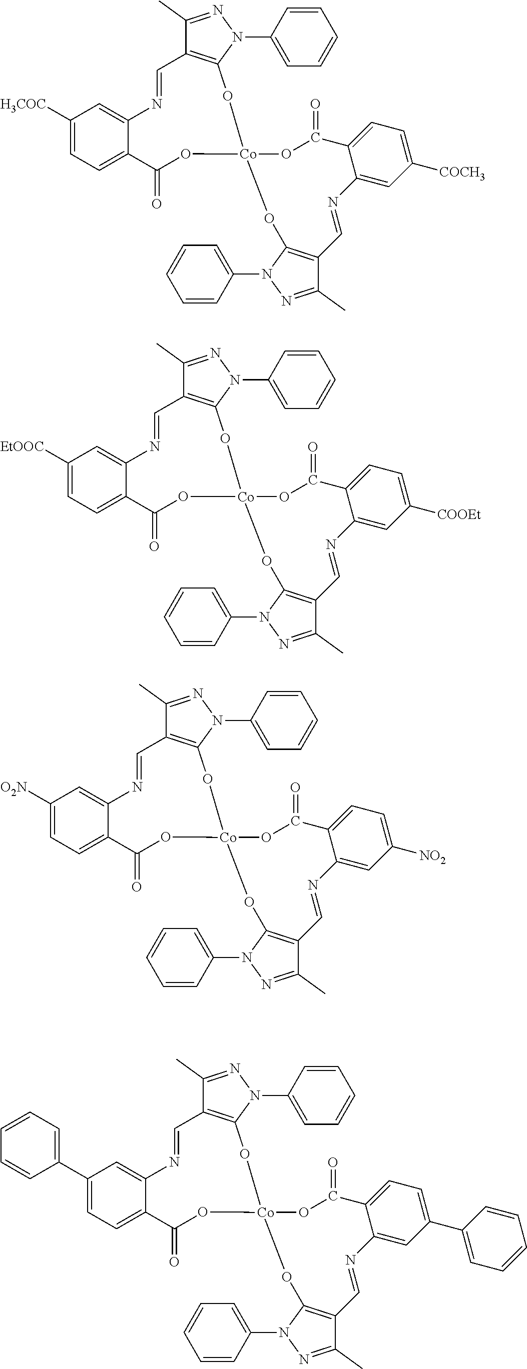 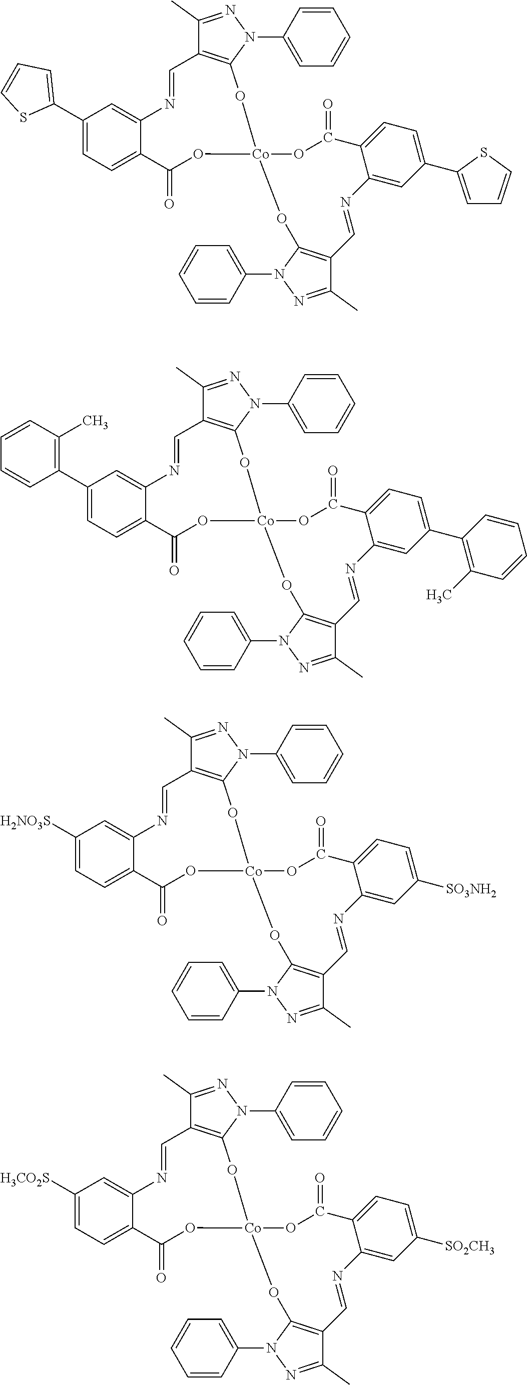 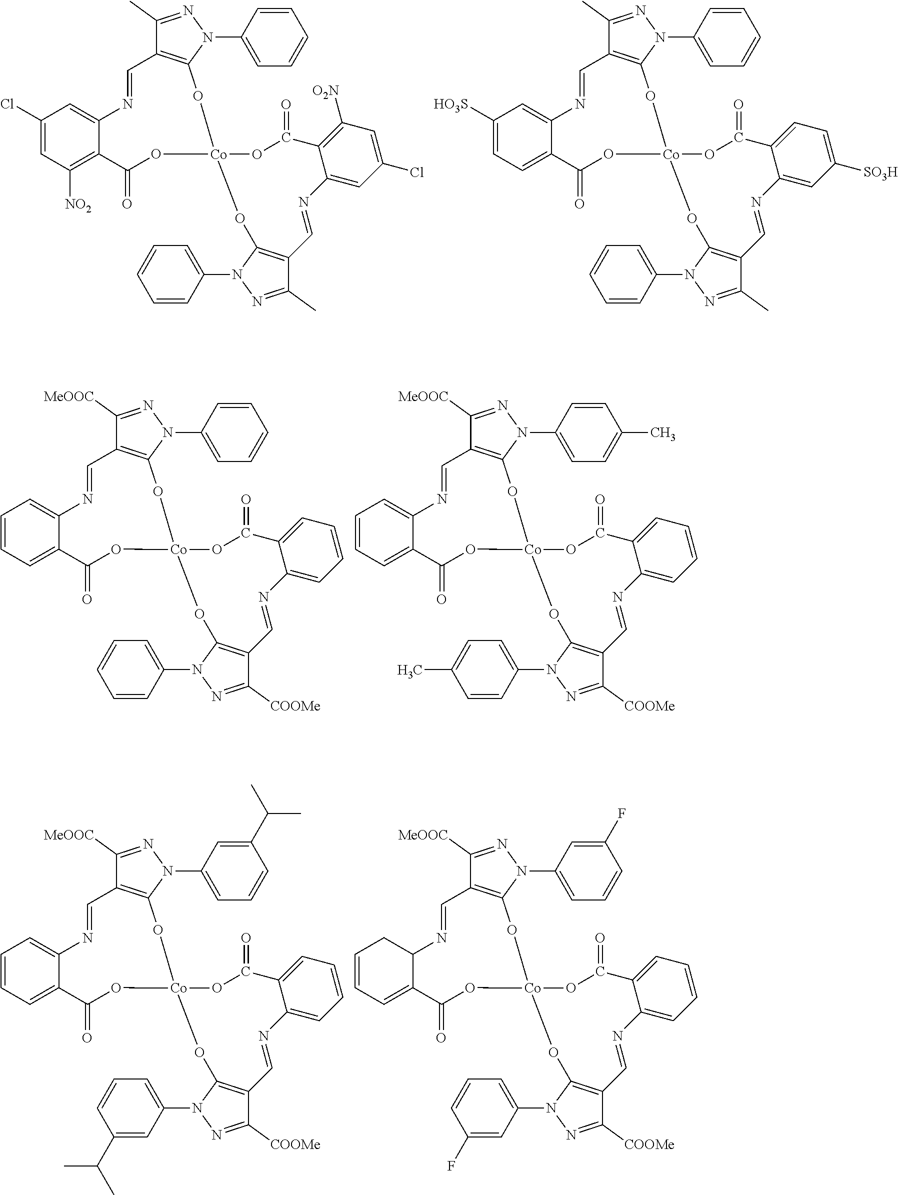 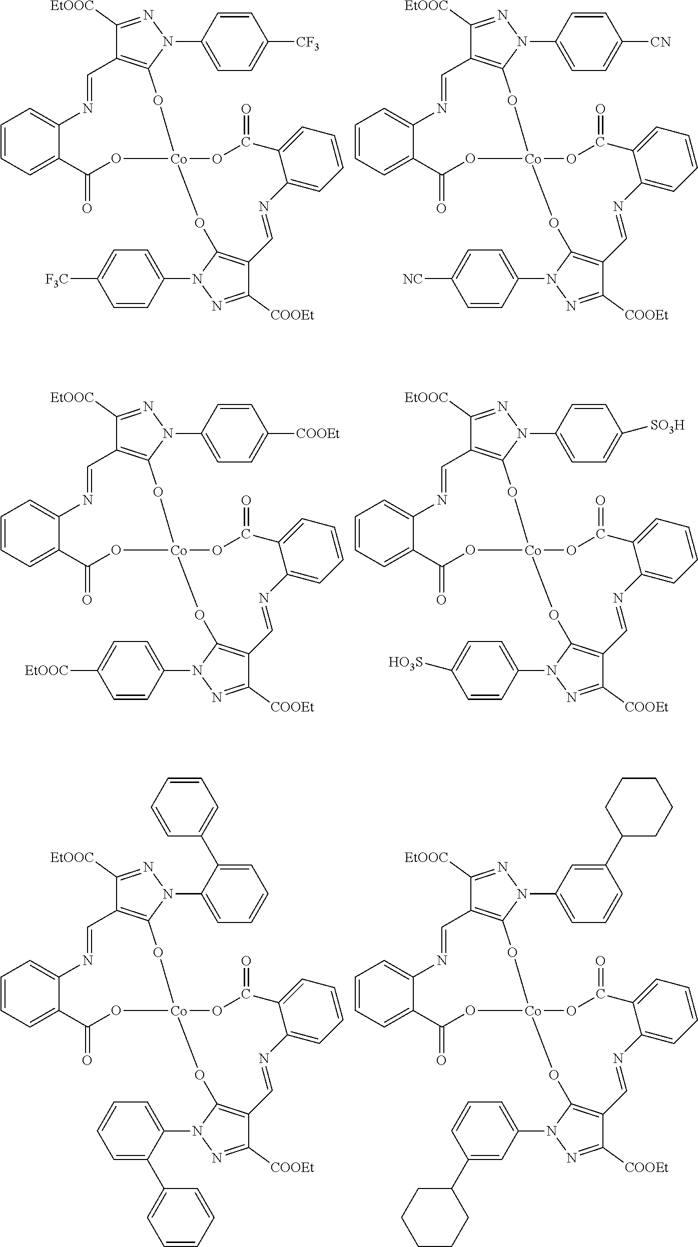 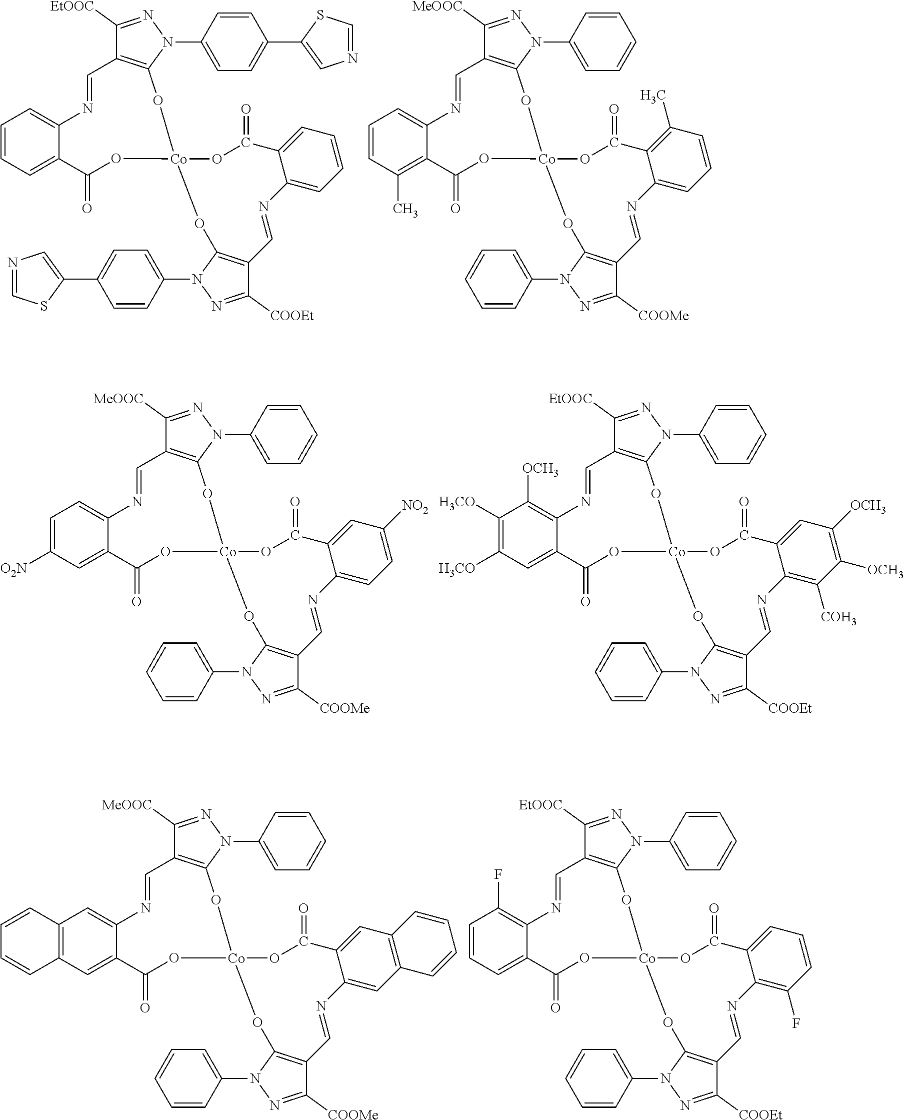 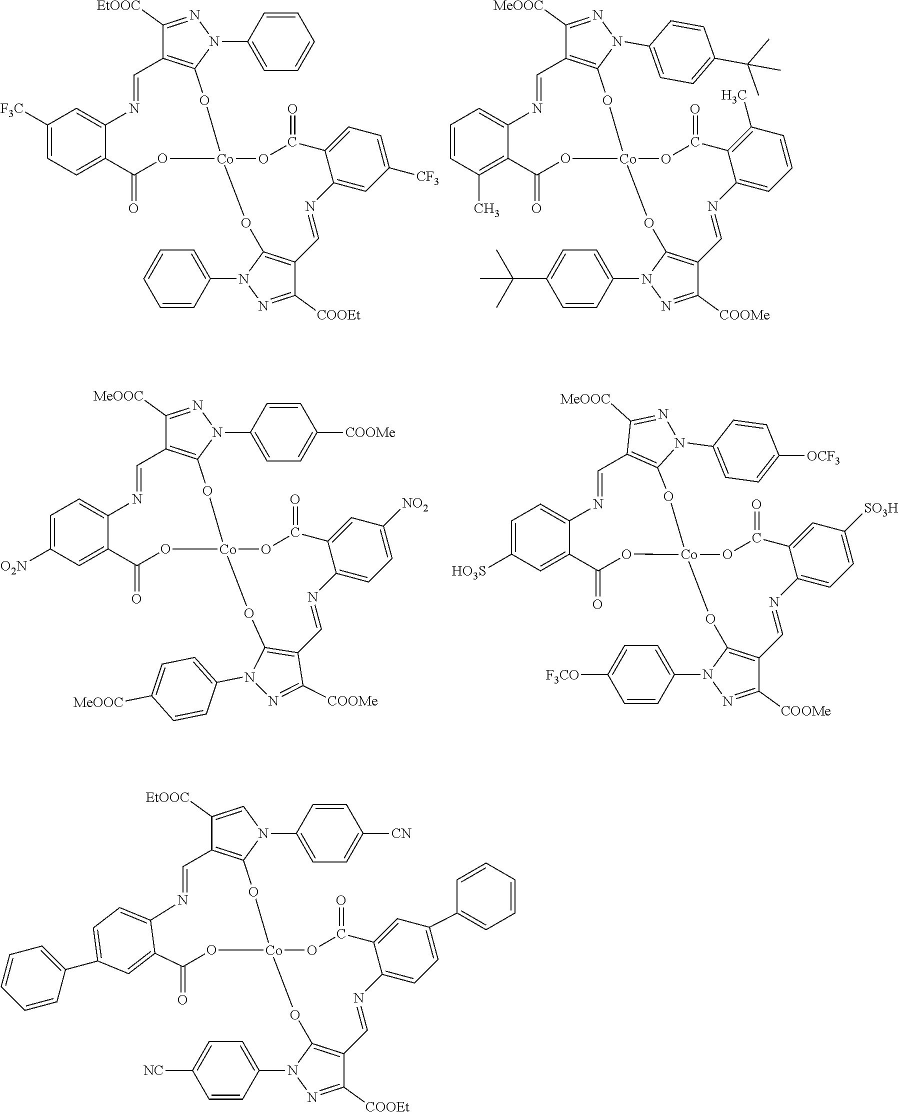 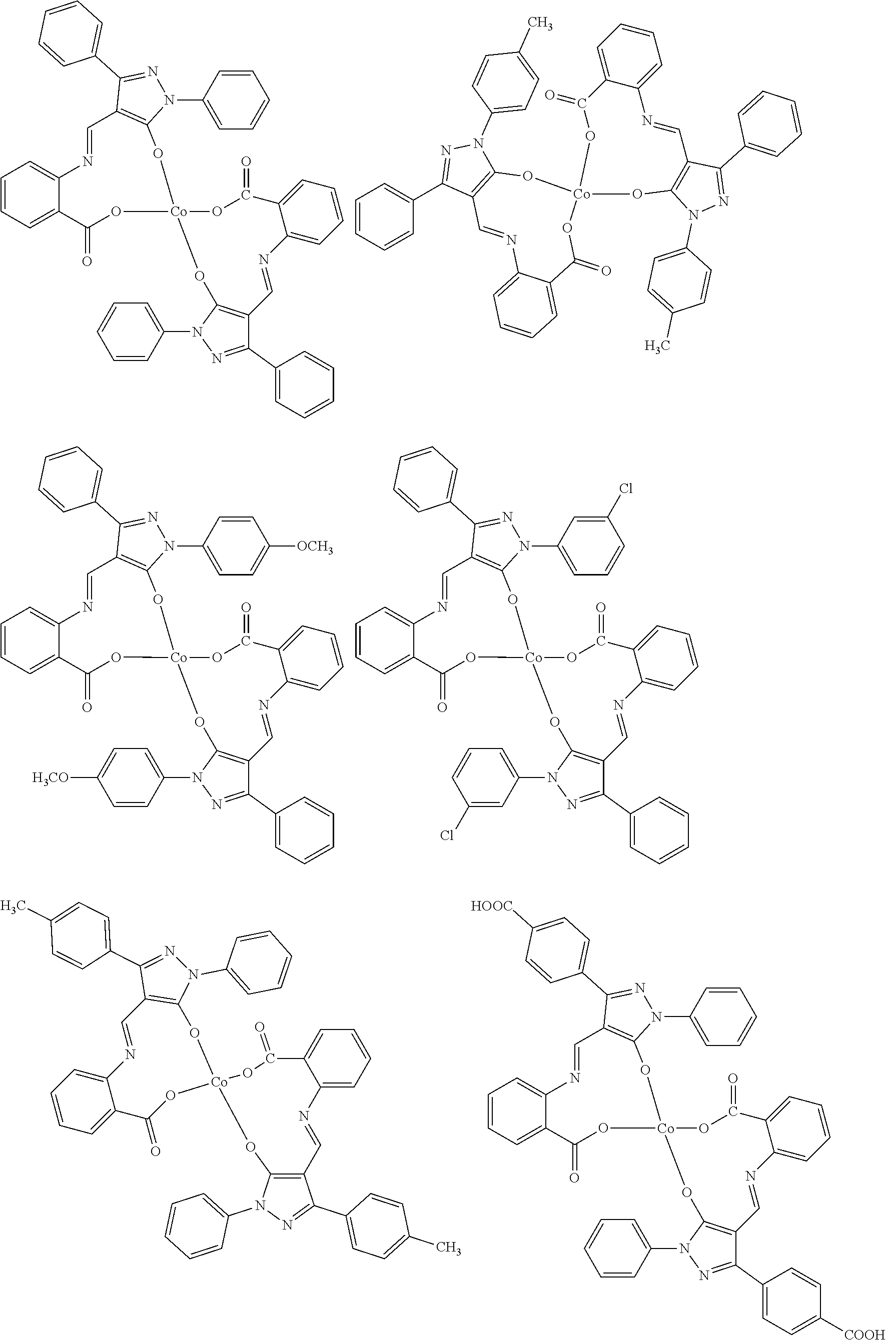 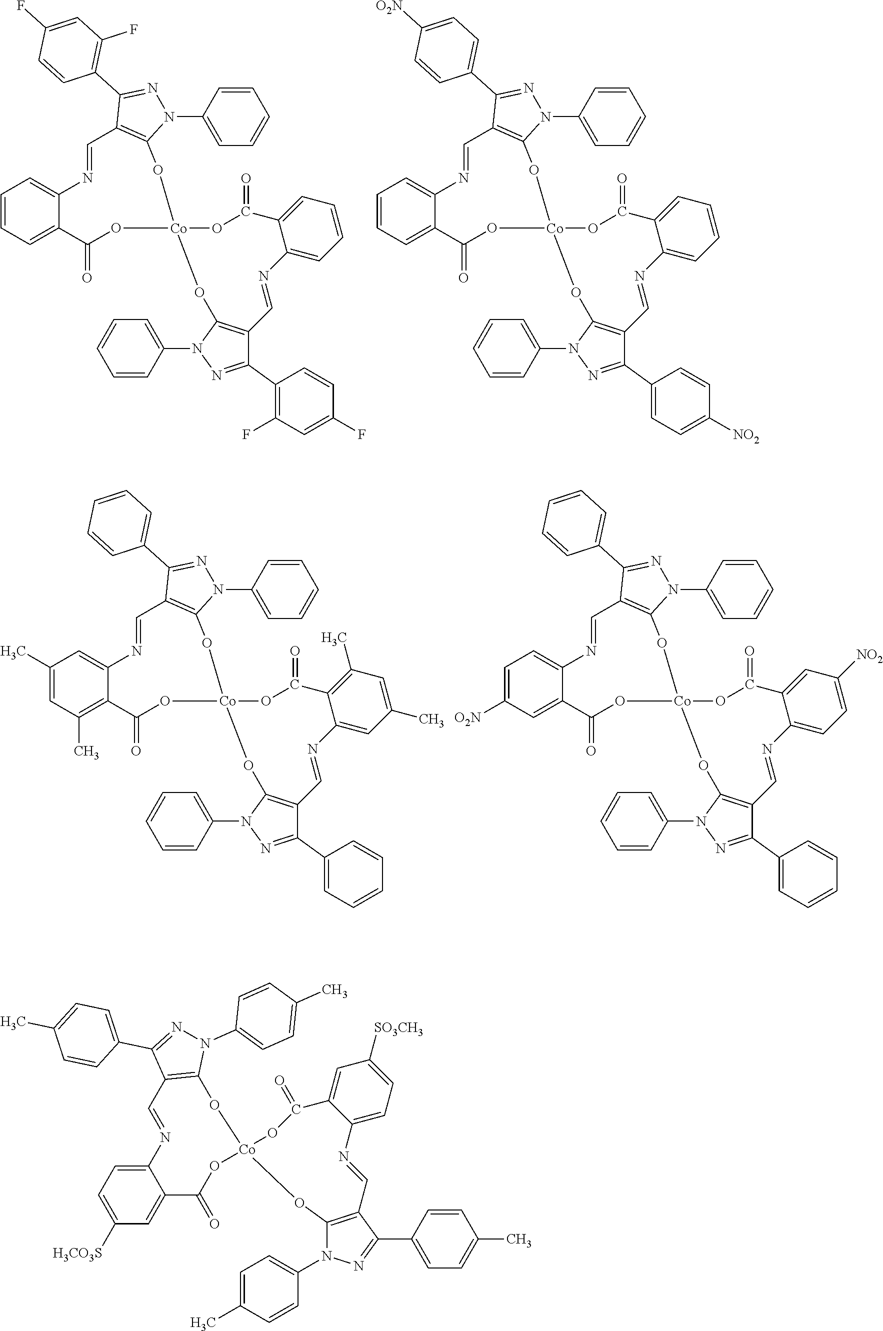 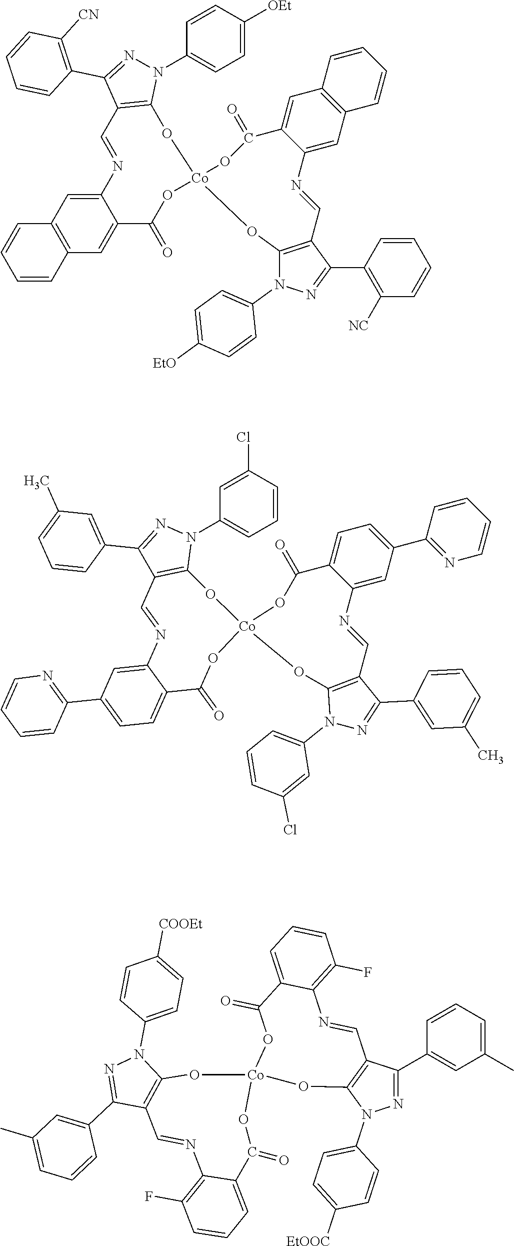 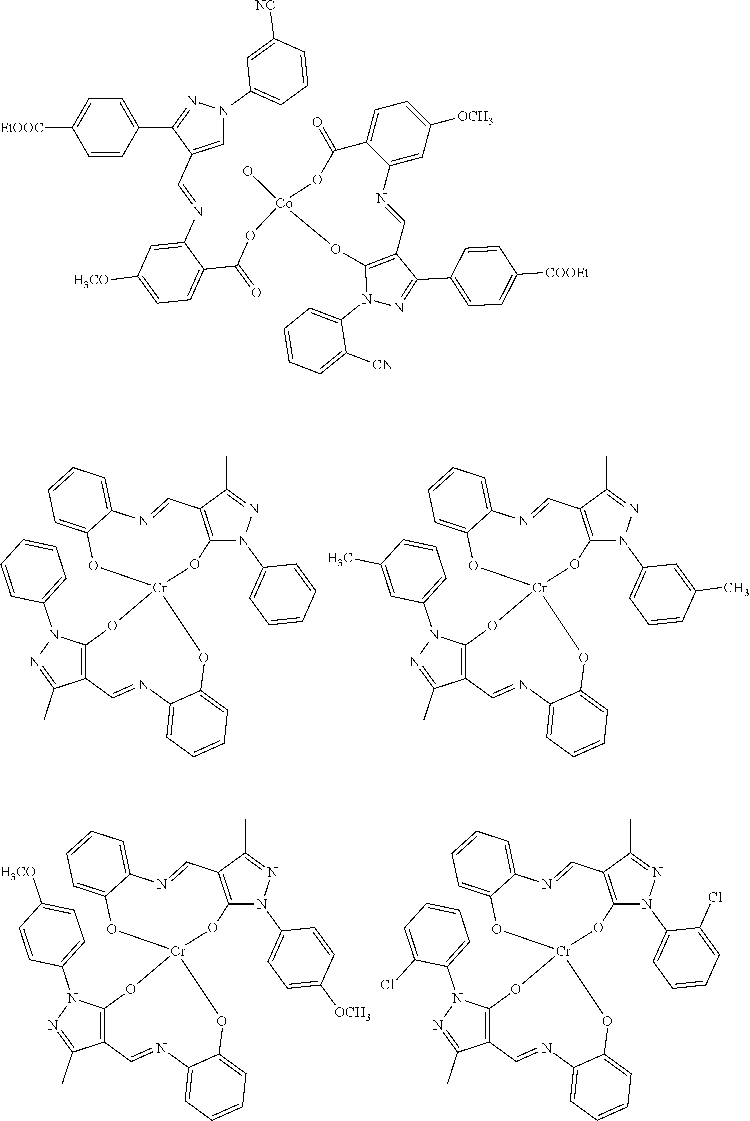 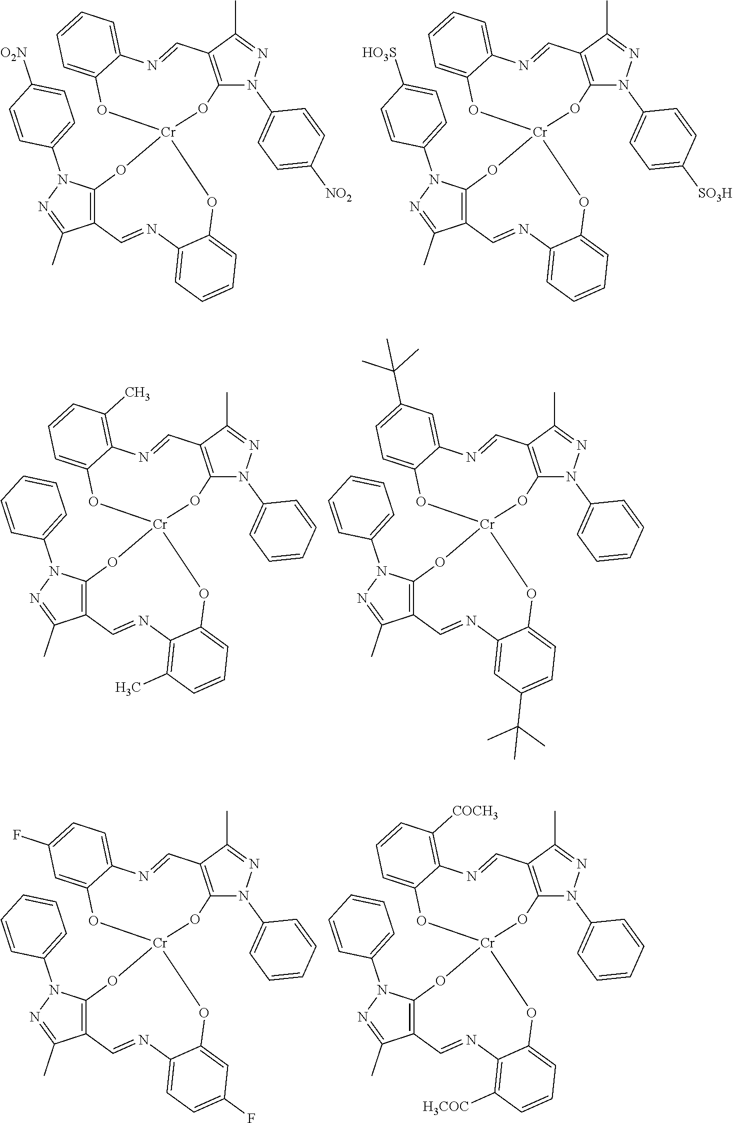 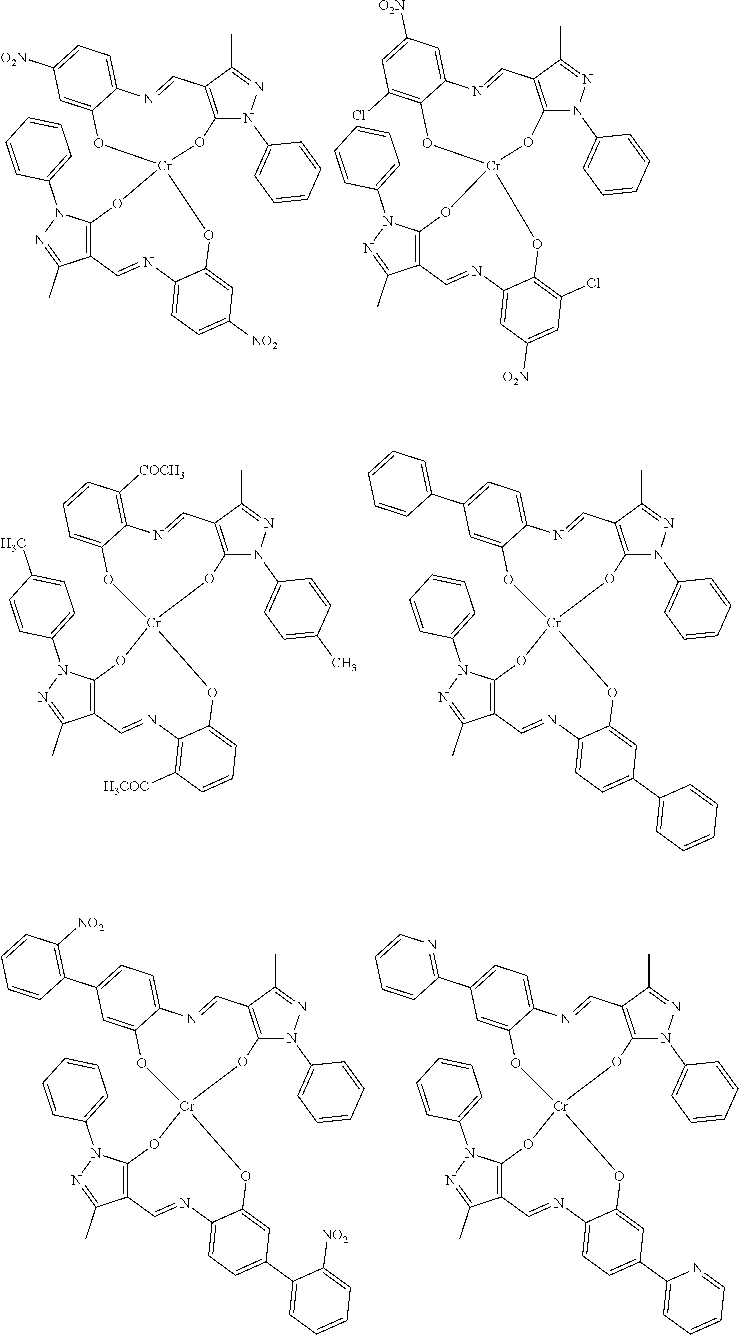 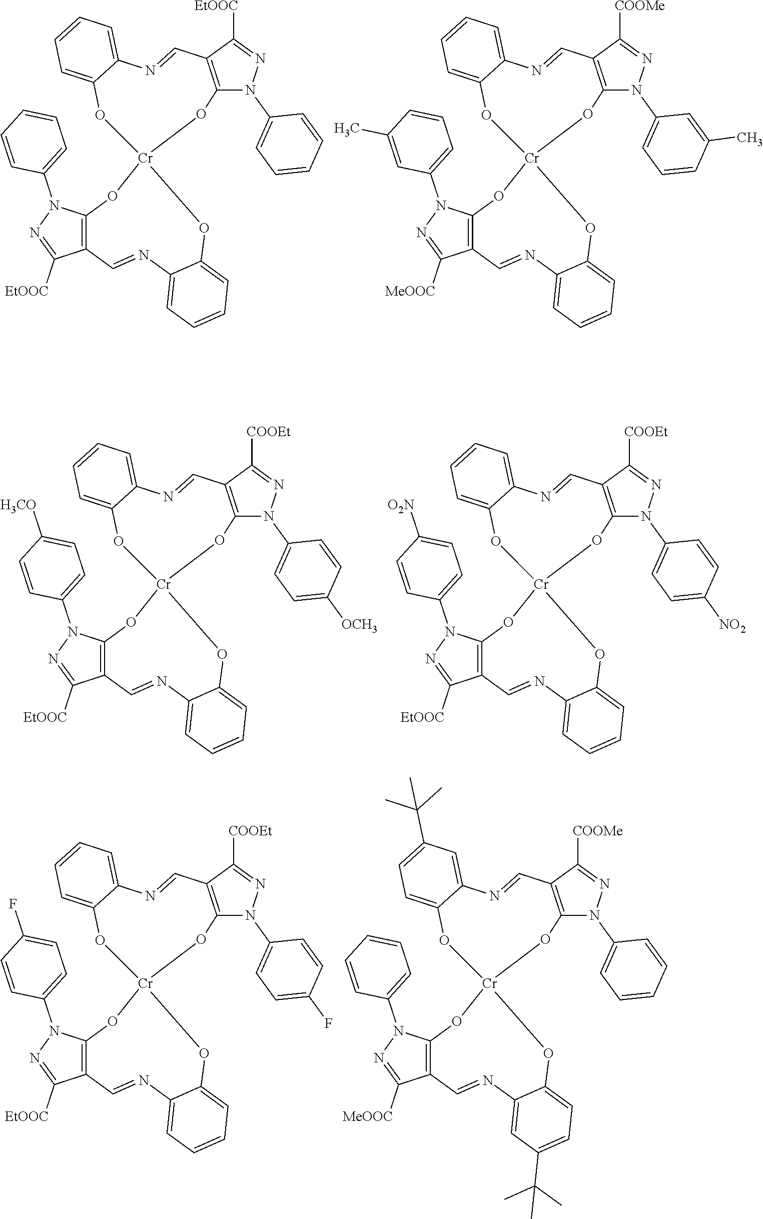 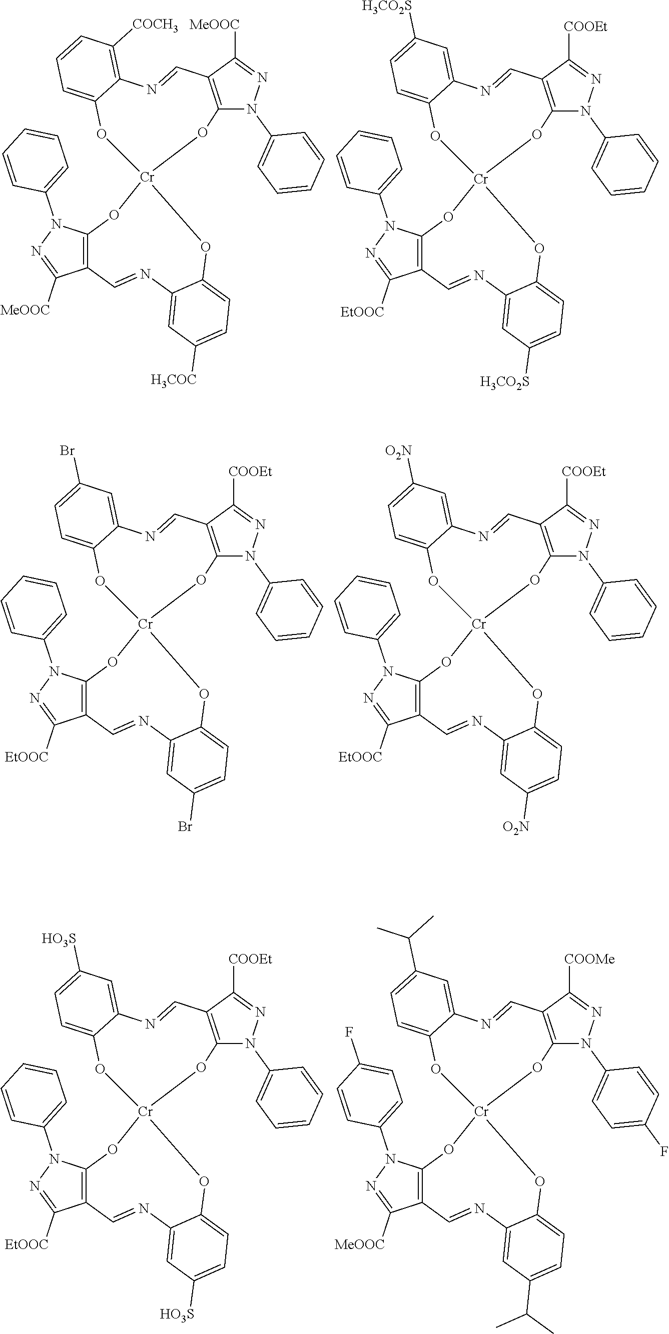 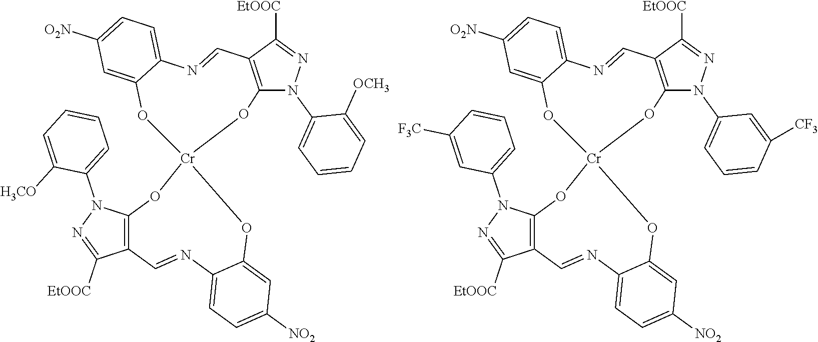 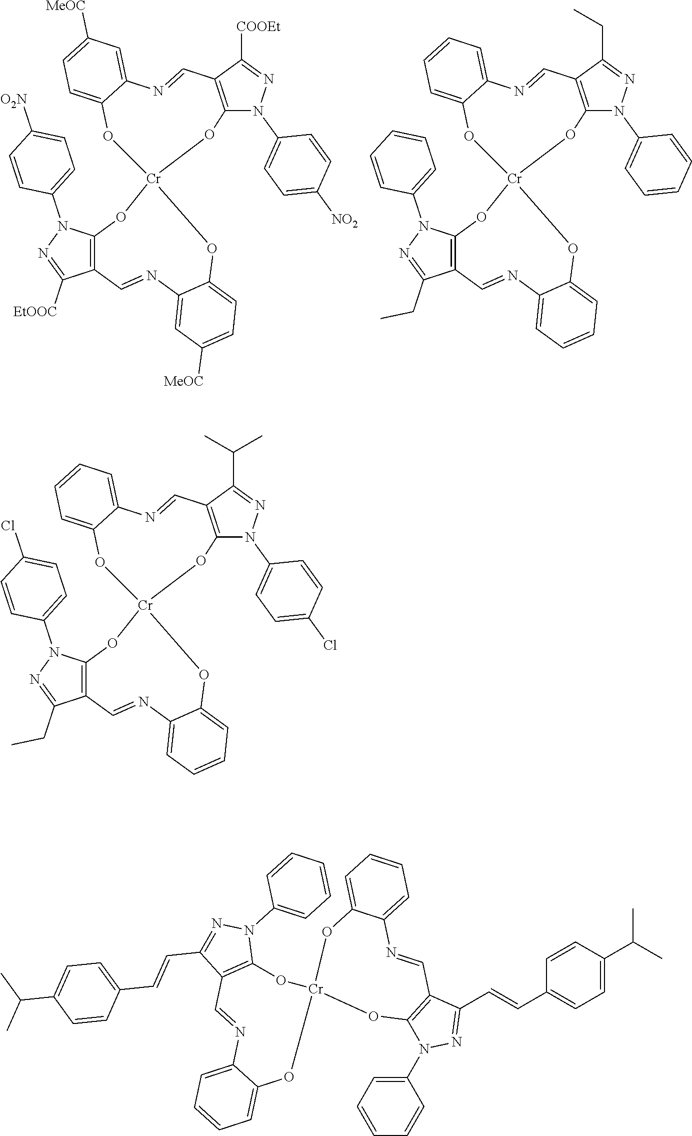 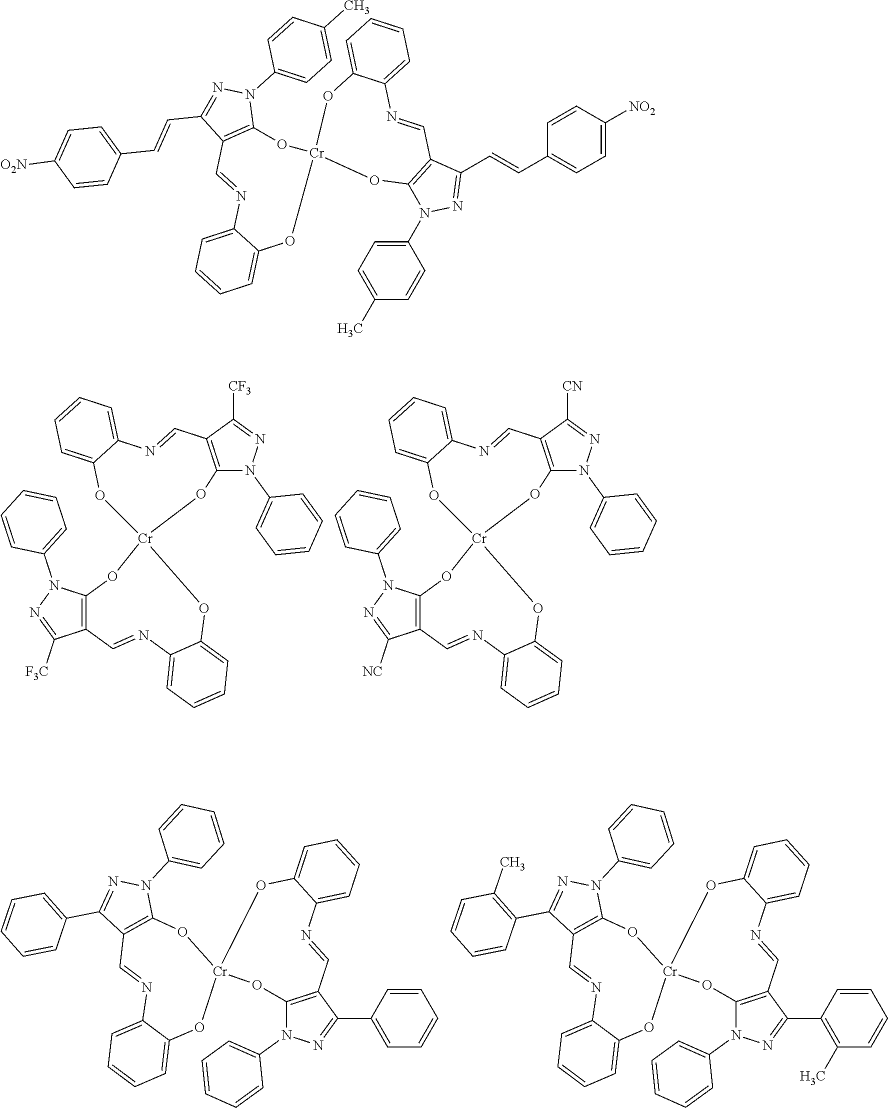 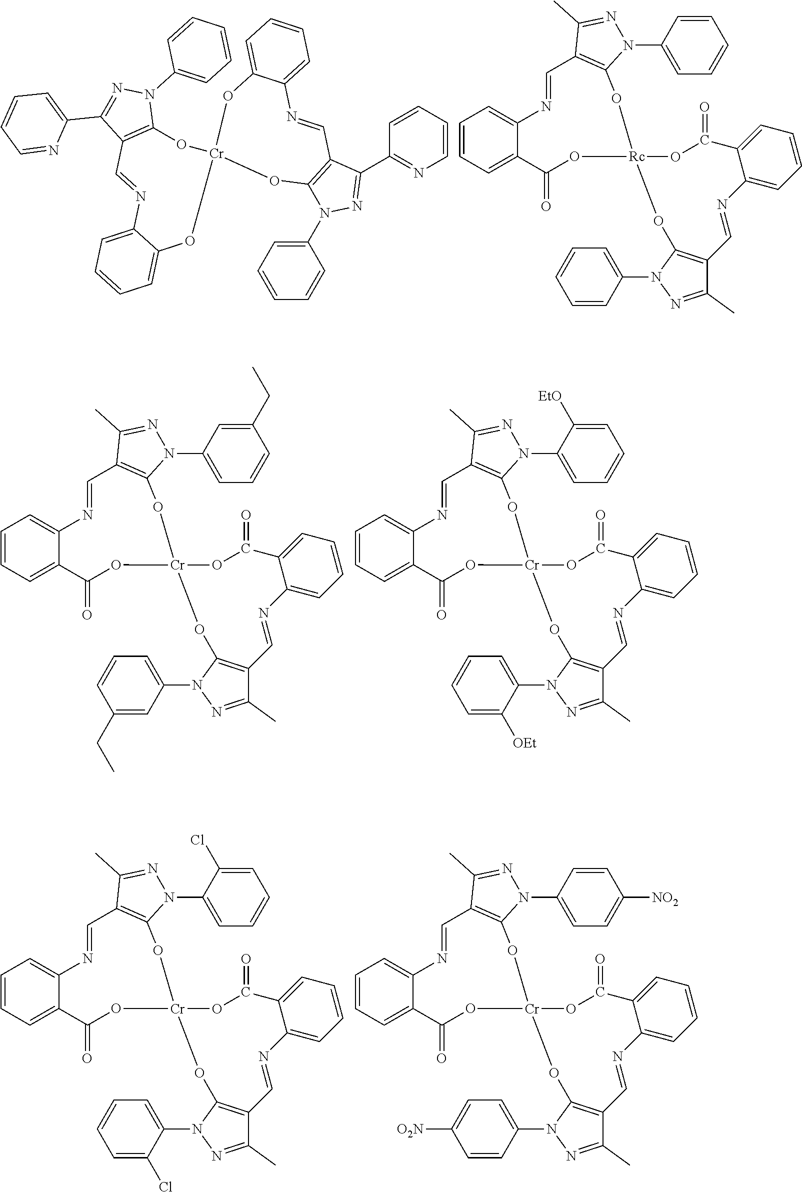 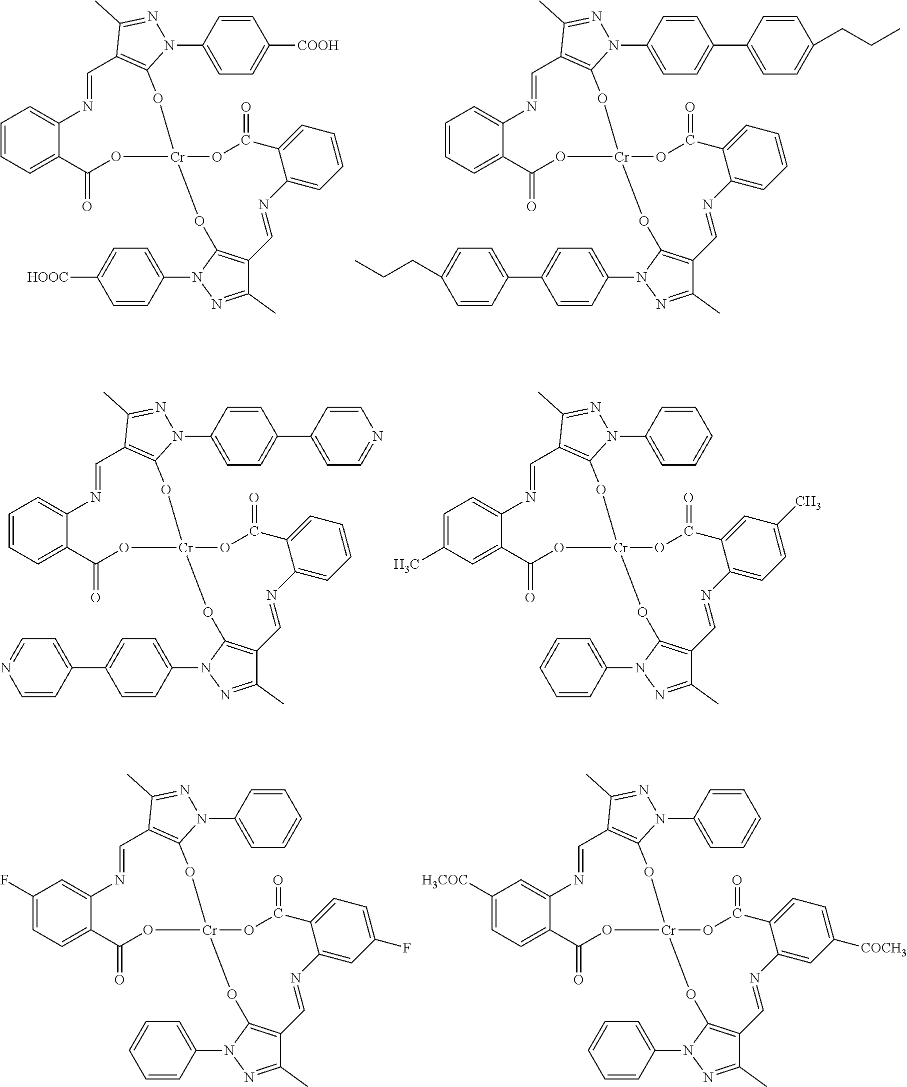 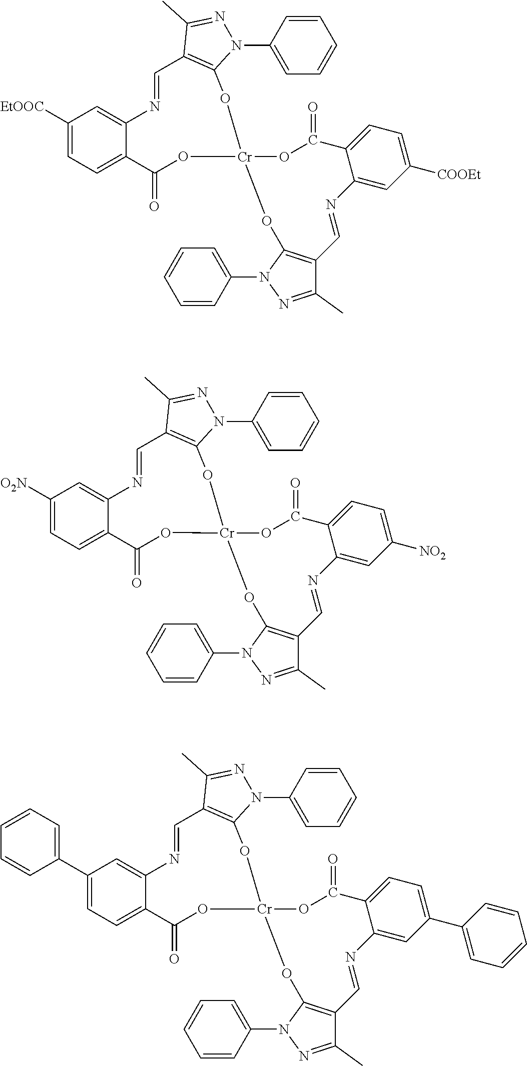 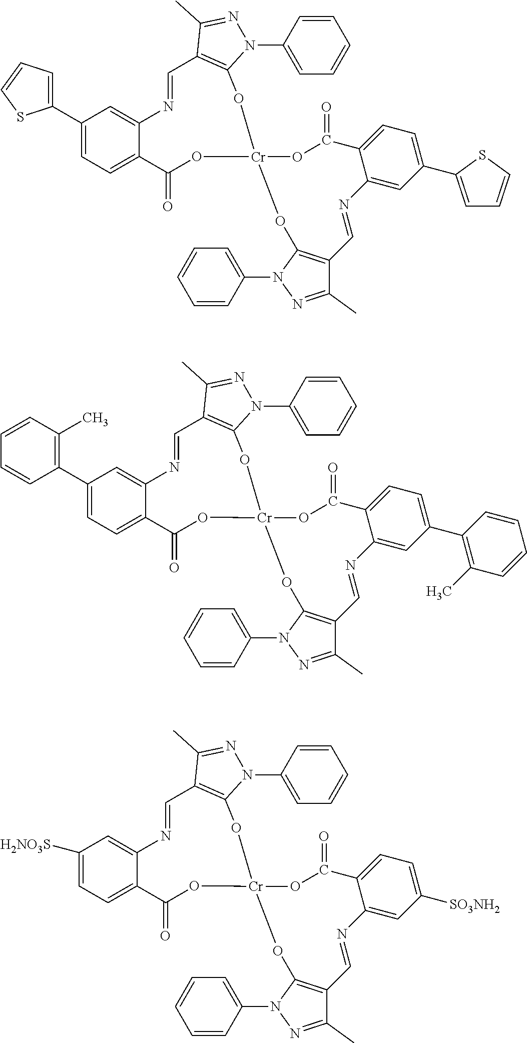 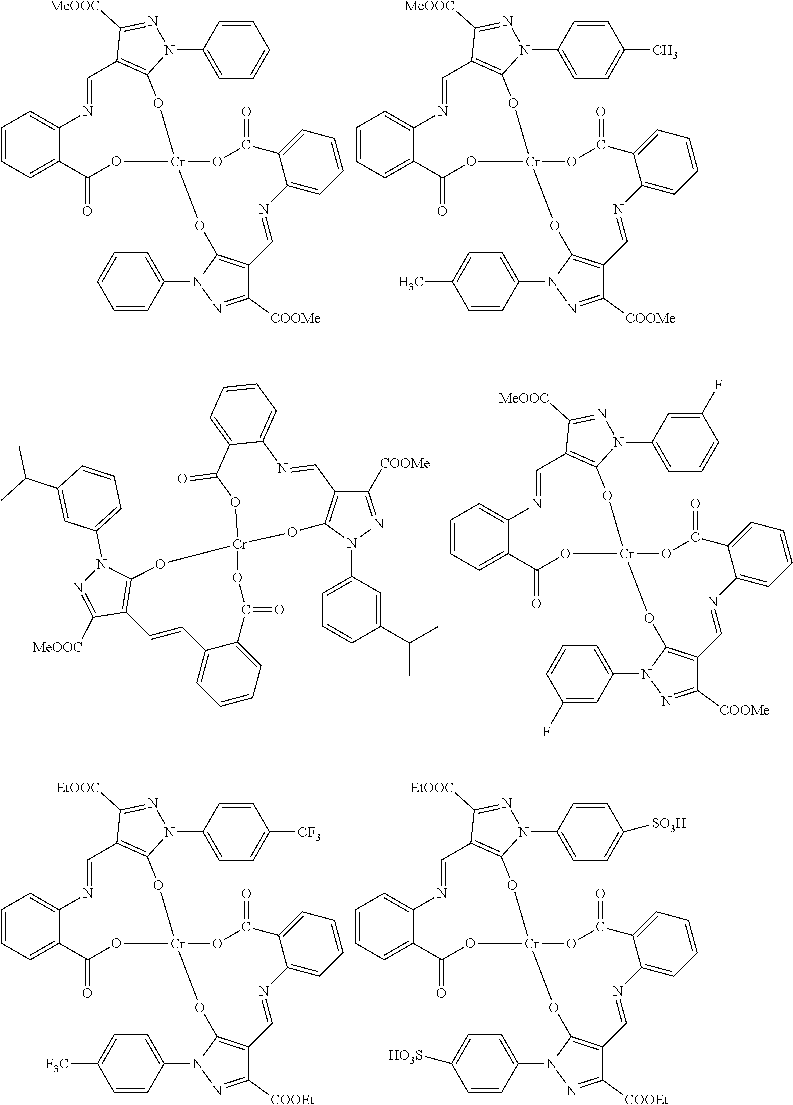 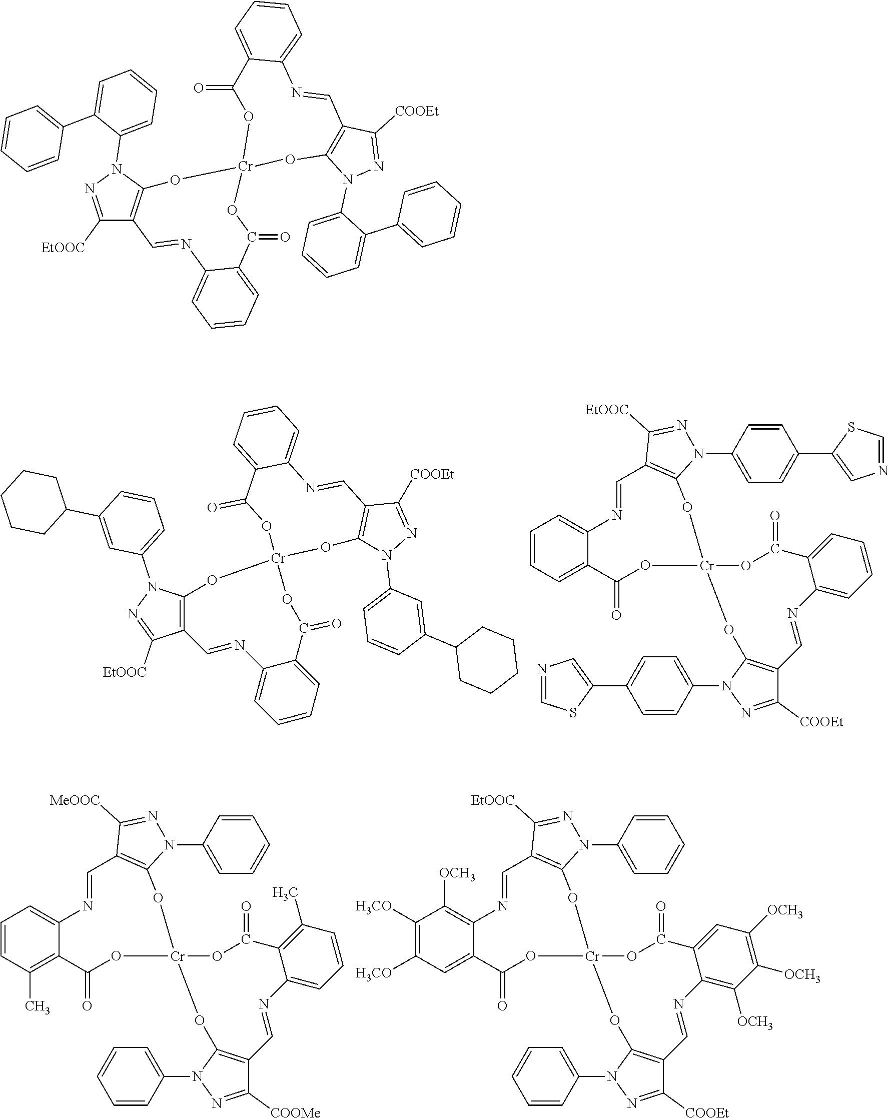 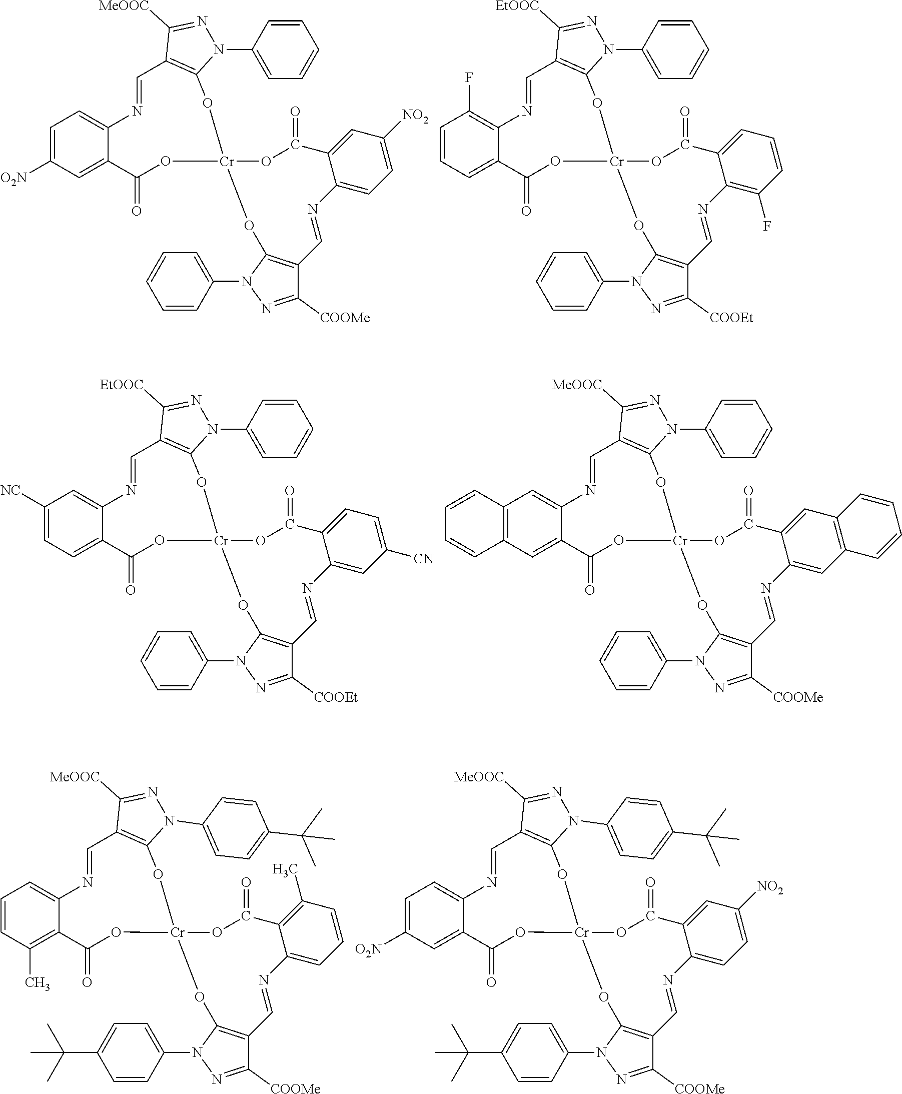 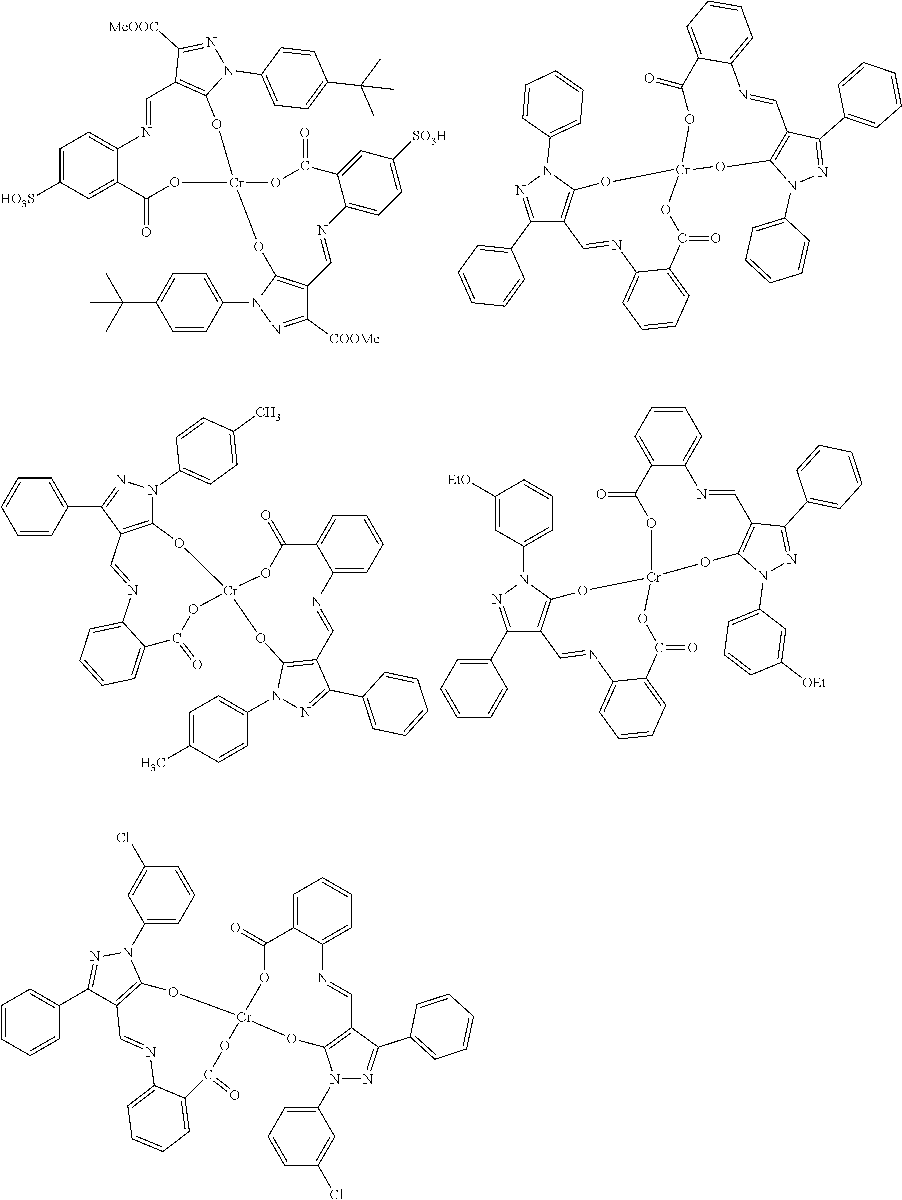 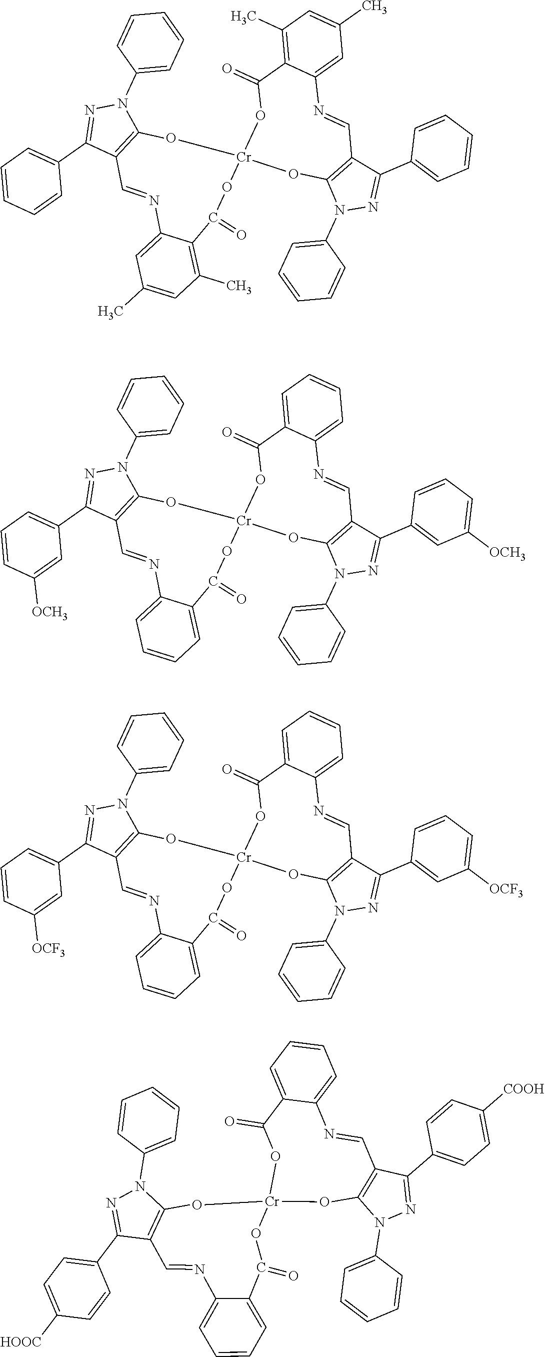 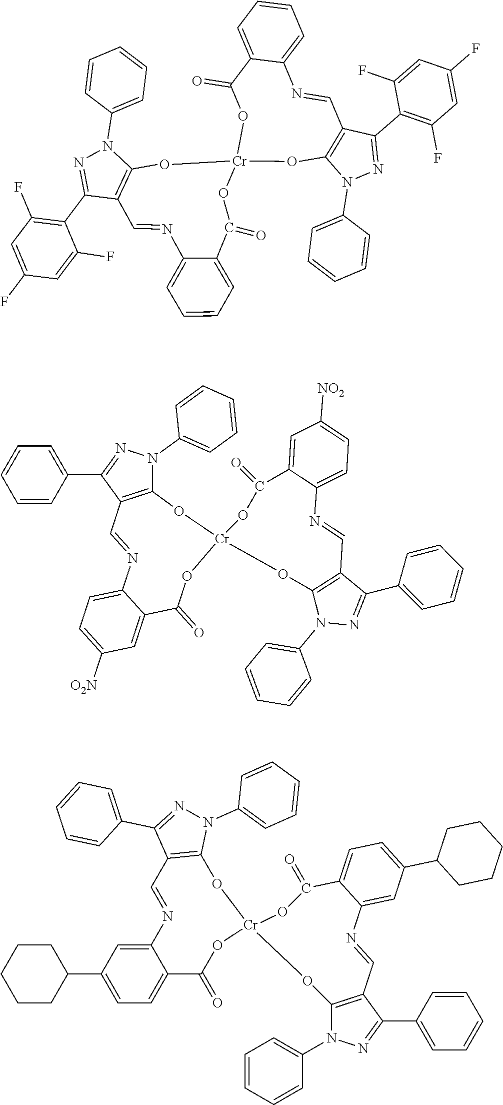 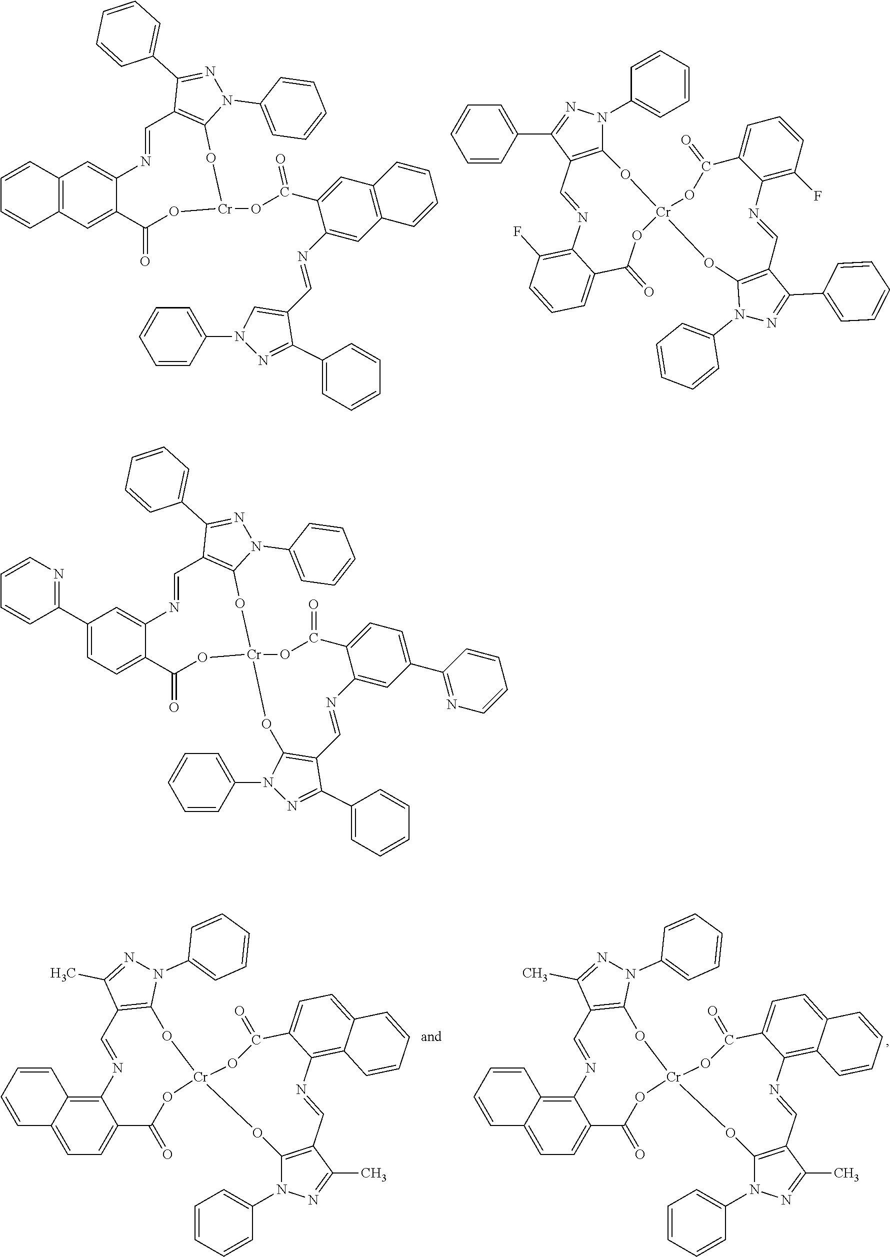
wherein
Me is a methyl group, Et is an ethyl group, and Co is Co²⁺ or Co³⁺, and Cr is Cr²⁺ or Cr³⁺.

8. The coordination compound of claim 1, further comprising a cation (B⁺)m,
wherein B⁺ is H⁺; Na⁺; K⁺; a phosphonium cation; an ammonium cation; a sulfonium cation; or a cationic heteroring, and m is an integer of 1 or 2.

9. The coordination compound of claim 8, wherein B⁺ is H⁺; Na⁺; K⁺; P⁺ReRfRgRh; N⁺RiRjRkRl; S⁺RmRnRo; or a cationic heteroring; and
wherein Re to Ro are the same as or different from each other, and each independently is hydrogen; a substituted or unsubstituted alkyl group; or a substituted or unsubstituted cycloalkyl group.

10. The coordination compound of claim 8, wherein the cationic heteroring is any one selected from the group consisting of Formulae B1 to B4: 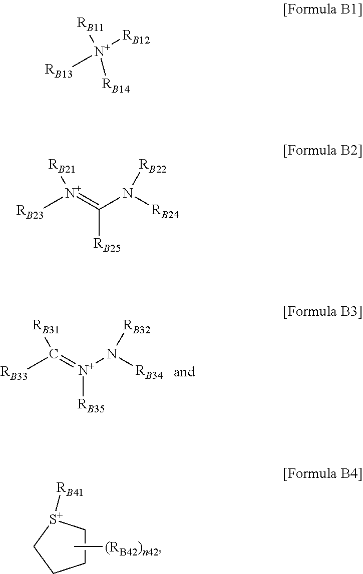
wherein in Formulae B1 to B4,
R_{B11} and R_{B12} bond to each other to form a substituted or unsubstituted heteroring having 4 to 30 carbon atoms, and R_{B13} and R_{B14} are the same as or different from each other and each independently is hydrogen; a hydrocarbon group having 1 to 30 carbon atoms; or a substituent including one, two or more heteroatoms selected from the group consisting of N, O, P, S, Si and Se;
R_{B21} and R_{B22} bond to each other to form a substituted or unsubstituted heteroring having 2 to 30 carbon atoms, and R_{B23} to R_{B25} are the same as or different from each other and each independently is hydrogen; a hydrocarbon group having 1 to 30 carbon atoms; or a substituent including one, two or more heteroatoms selected from the group consisting of N, O, P, S, Si and Se;
R_{B31} and R_{B32} bond to each other to form a substituted or unsubstituted heteroring having 2 to 30 carbon atoms, and R_{B33} to R_{B35} are the same as or different from each other and each independently is hydrogen; a hydrocarbon group having 1 to 30 carbon atoms; or a substituent including one, two or more heteroatoms selected from the group consisting of N, O, P, S, Si and Se; and
R_{B41} is hydrogen; a hydrocarbon group having 1 to 30 carbon atoms; or a substituent including one, two or more heteroatoms selected from the group consisting of N, O, P, S, Si and Se, R_{B42} is hydrogen; a hydrocarbon group having 1 to 30 carbon atoms; or a substituent including one, two or more heteroatoms selected from the group consisting of N, O, P, S, Si and Se, and n42 is an integer of 1 to 8.

11. The coordination compound of claim 8, wherein the compound further including (B)m is any one selected from the group consisting of:

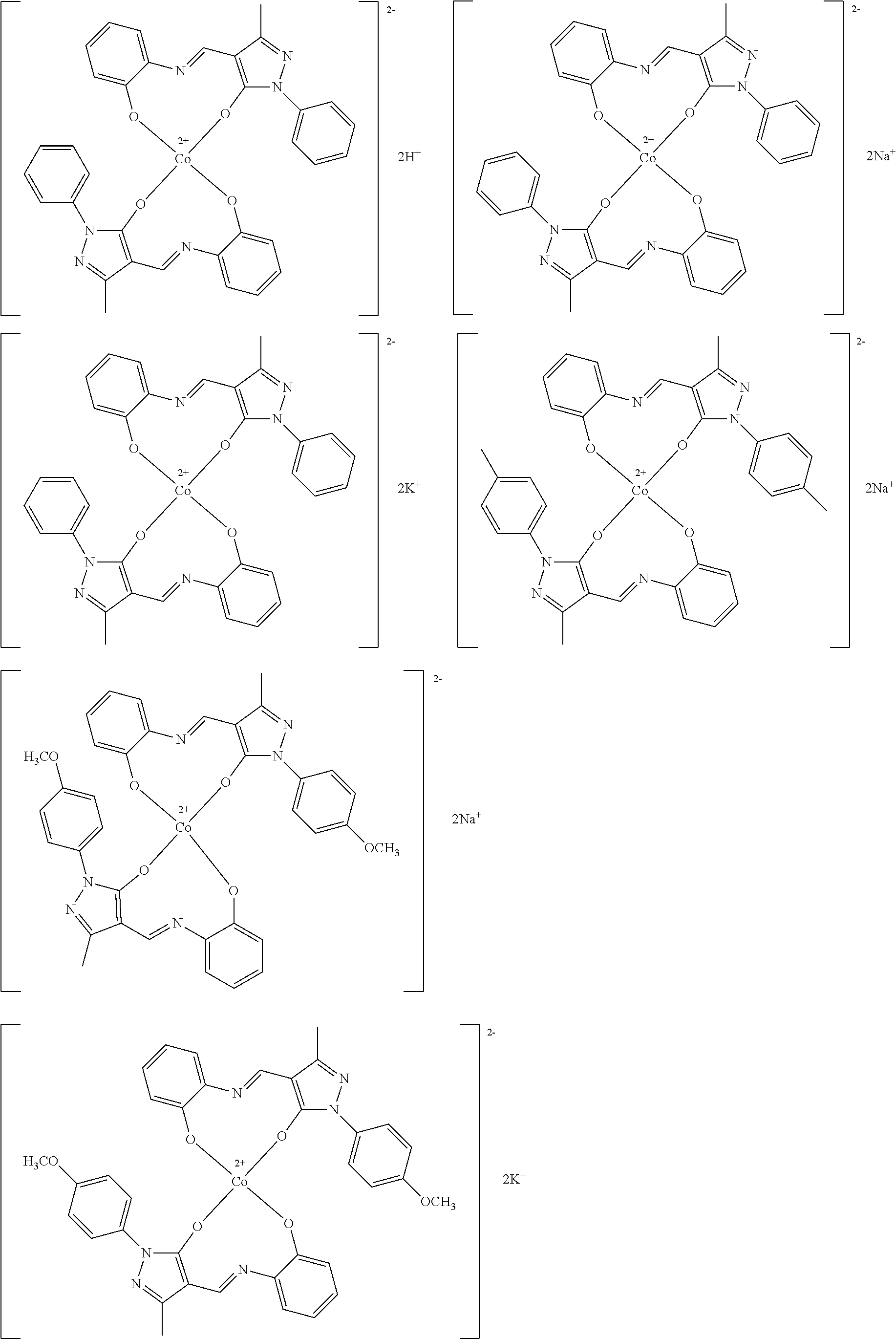

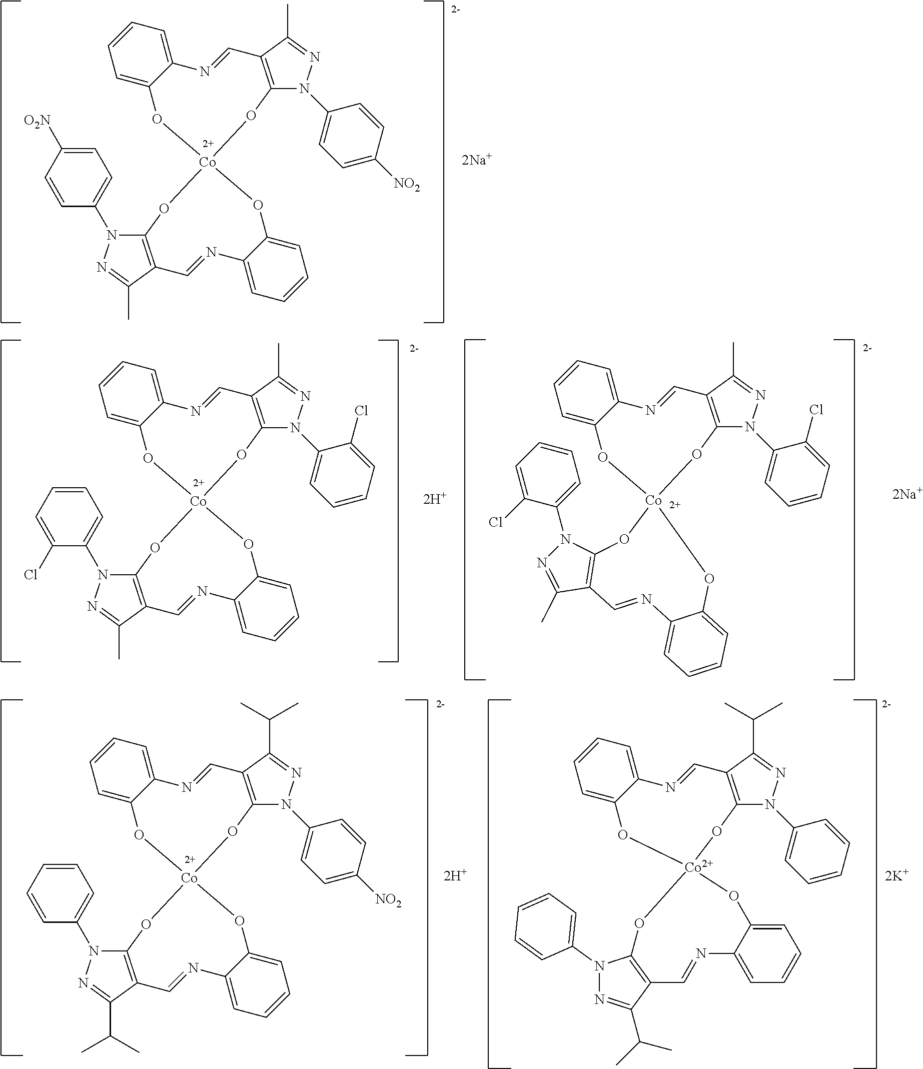

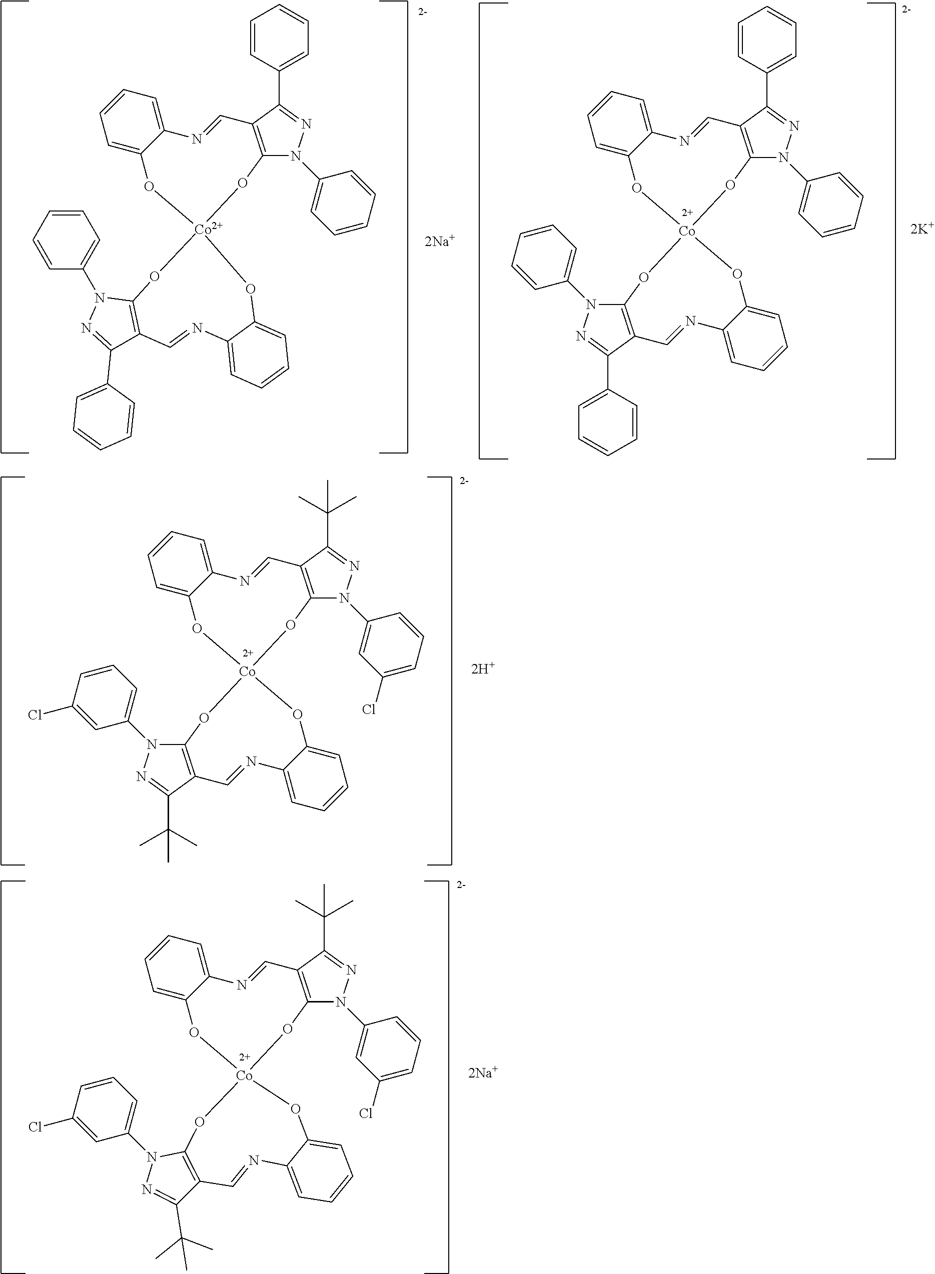

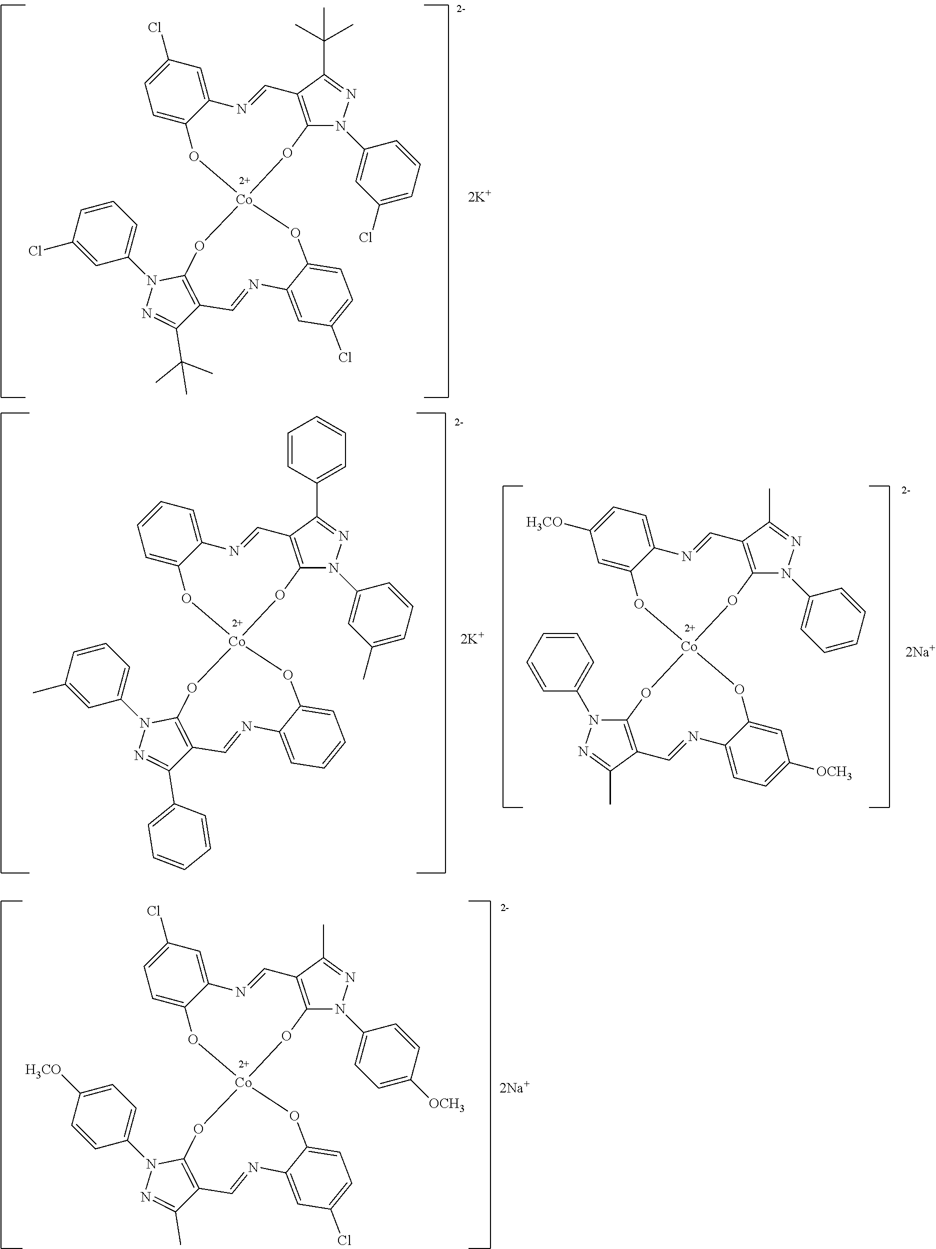

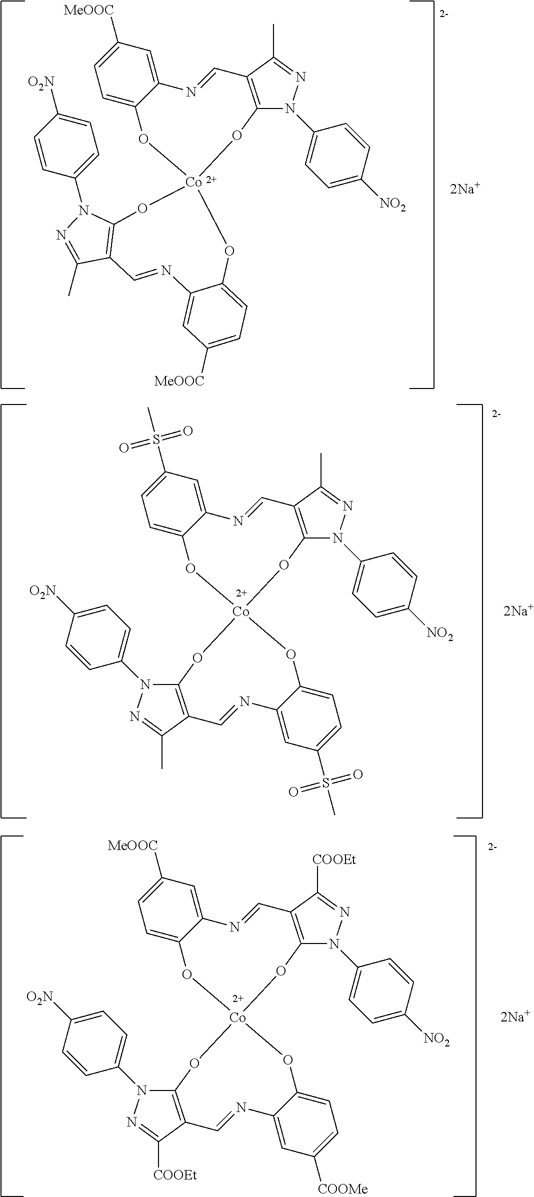

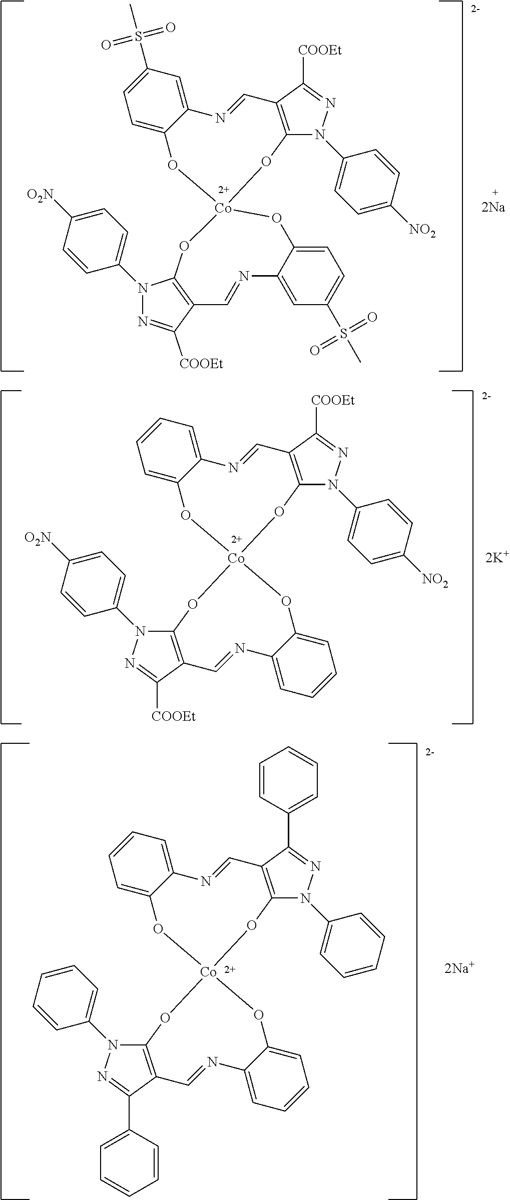

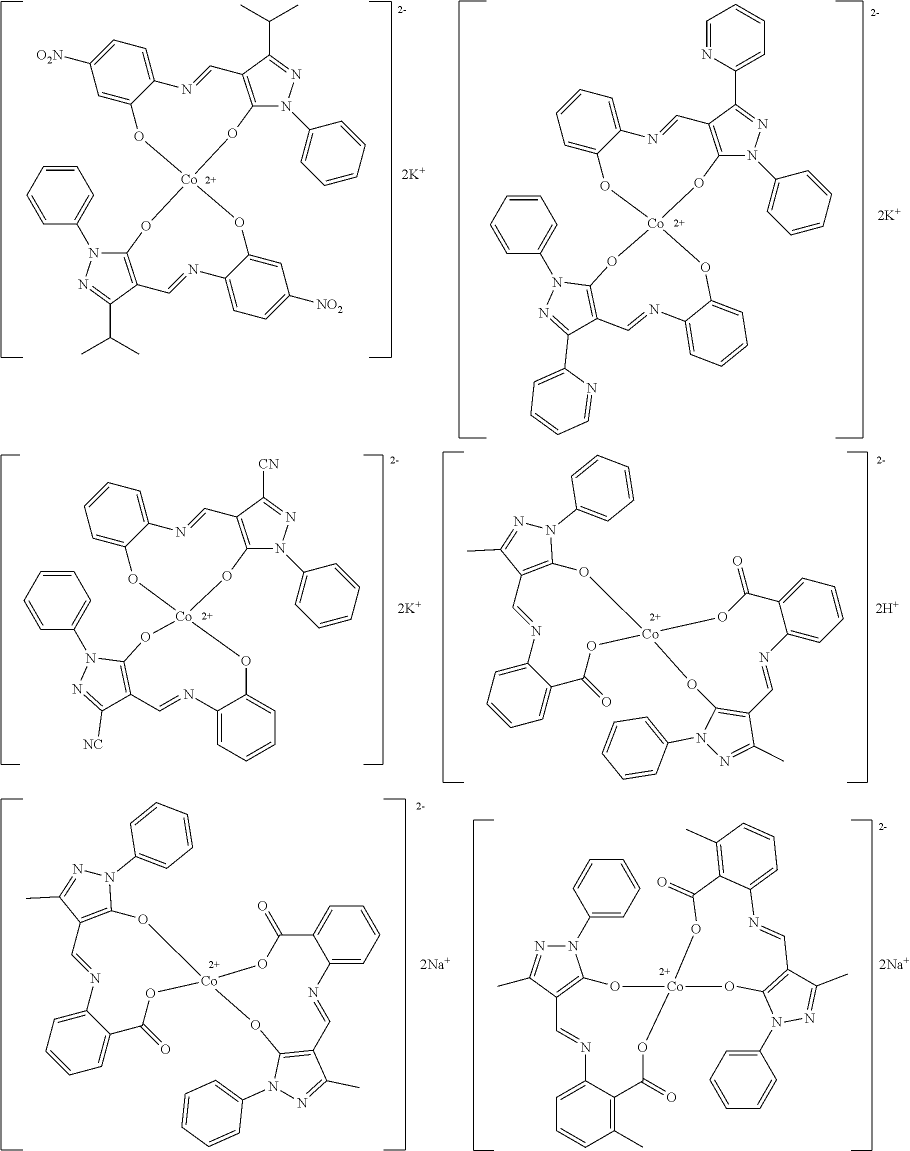

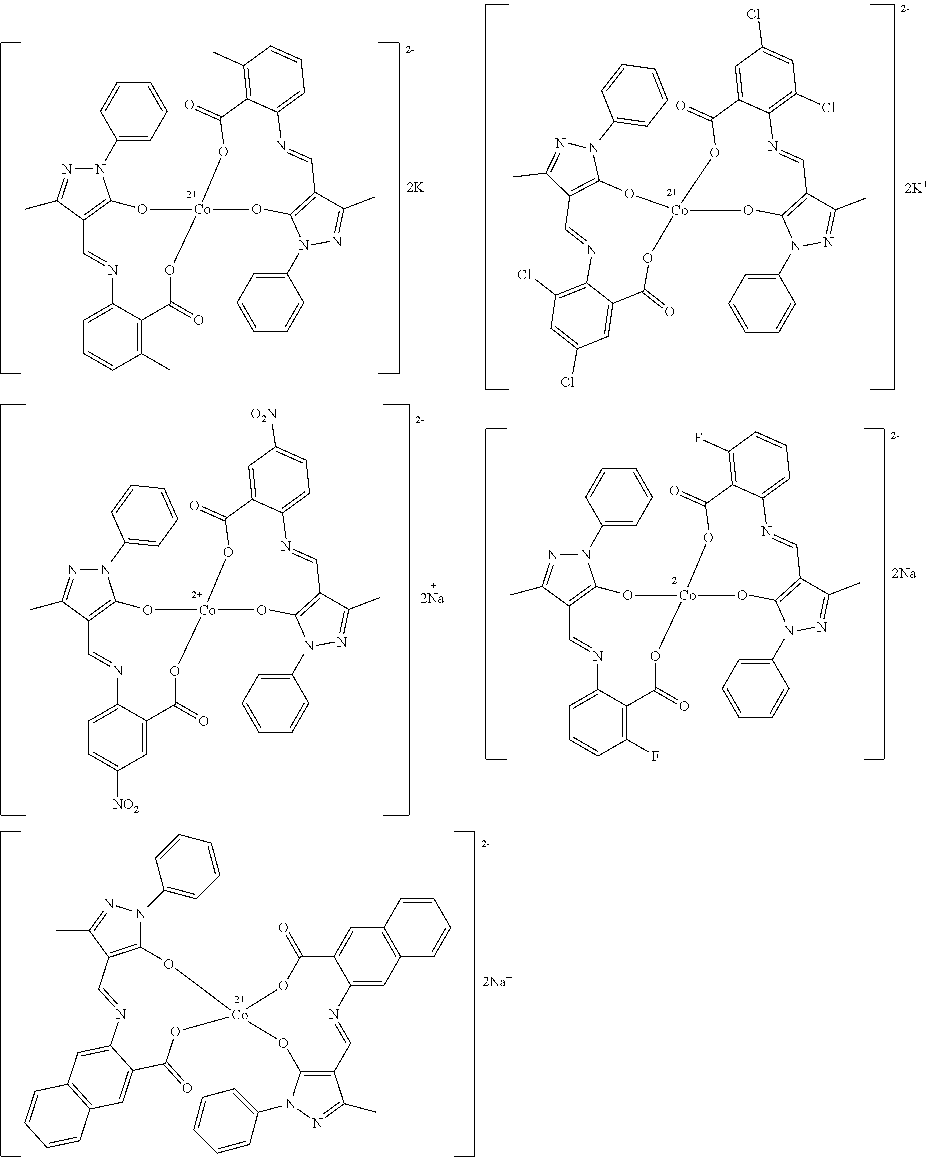

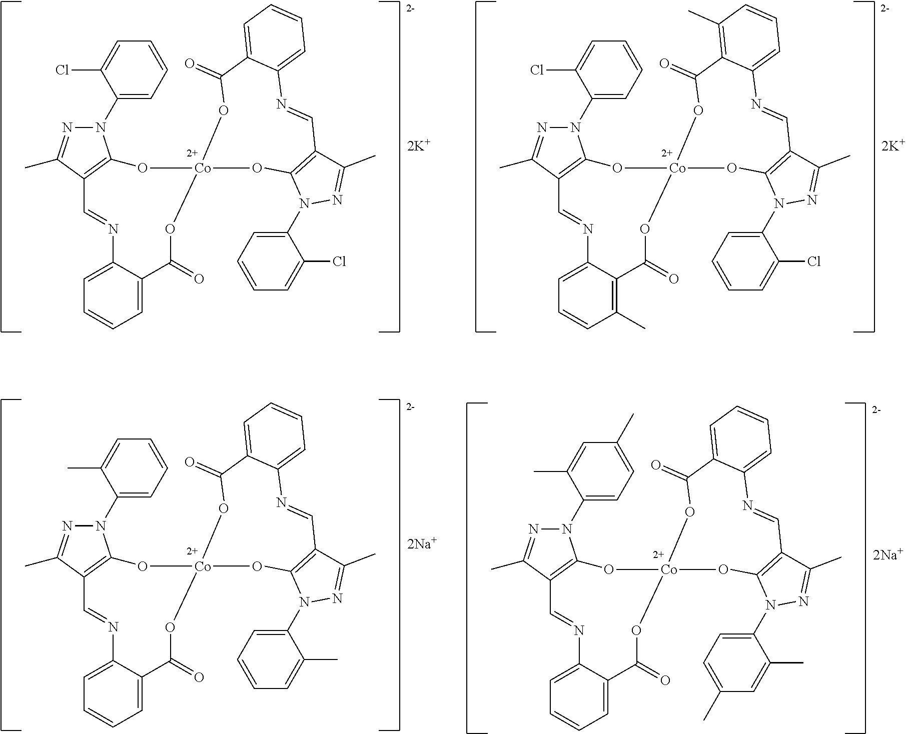

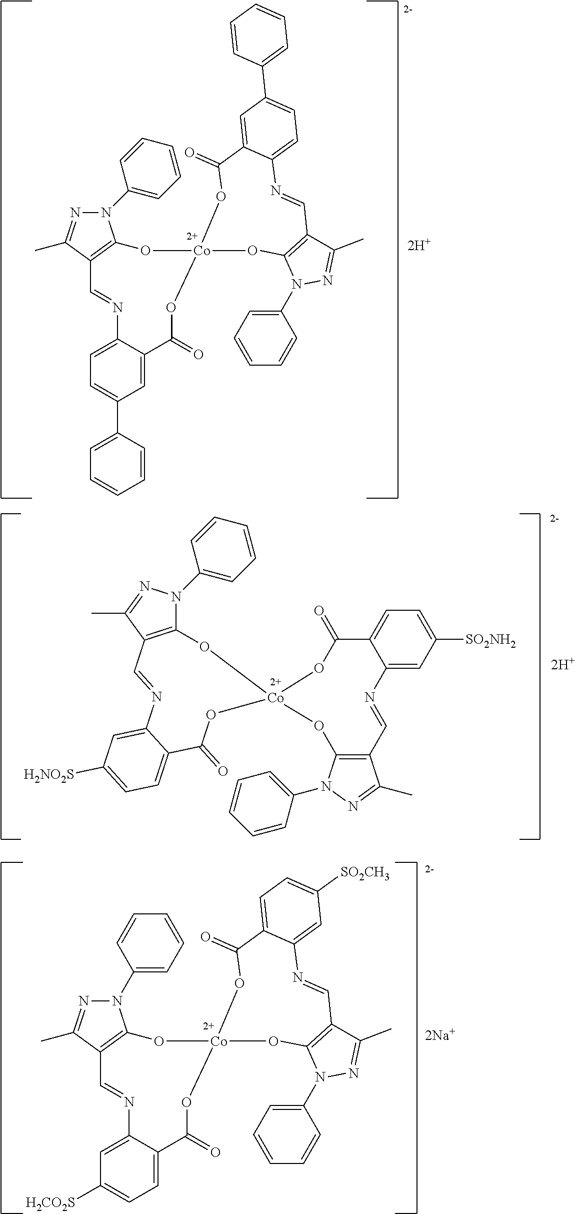

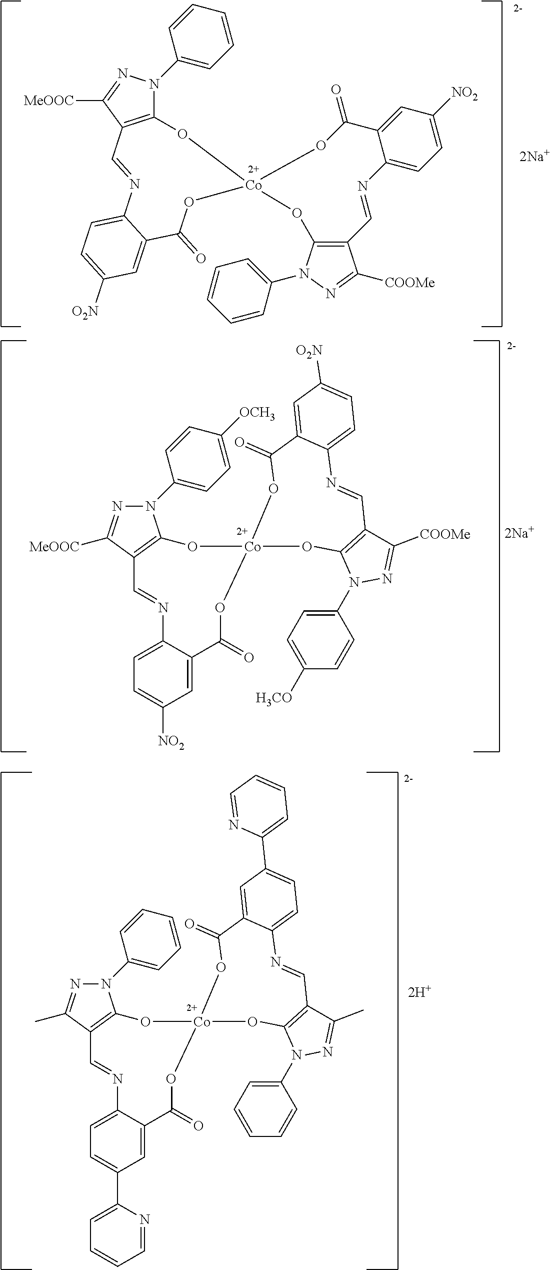

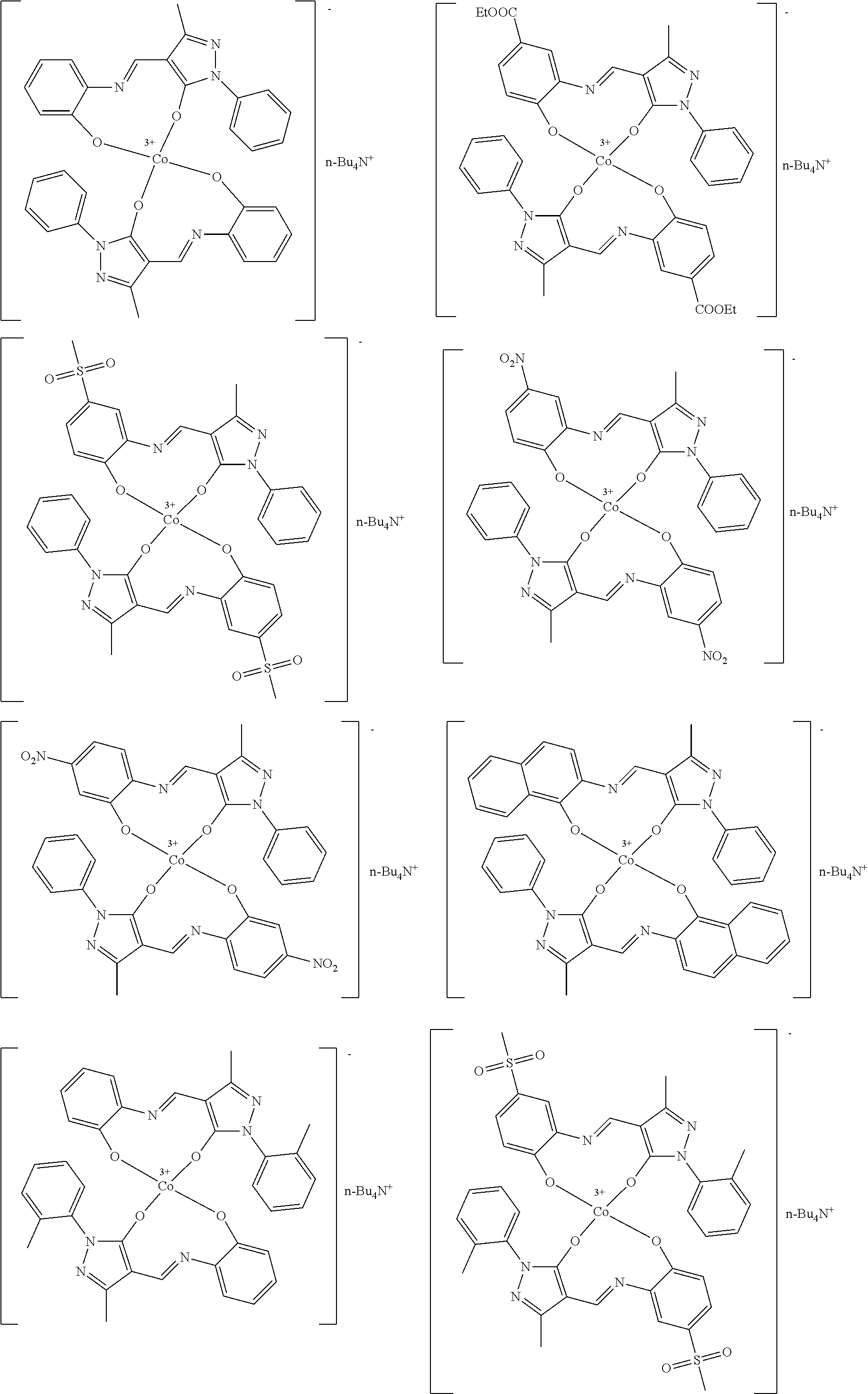

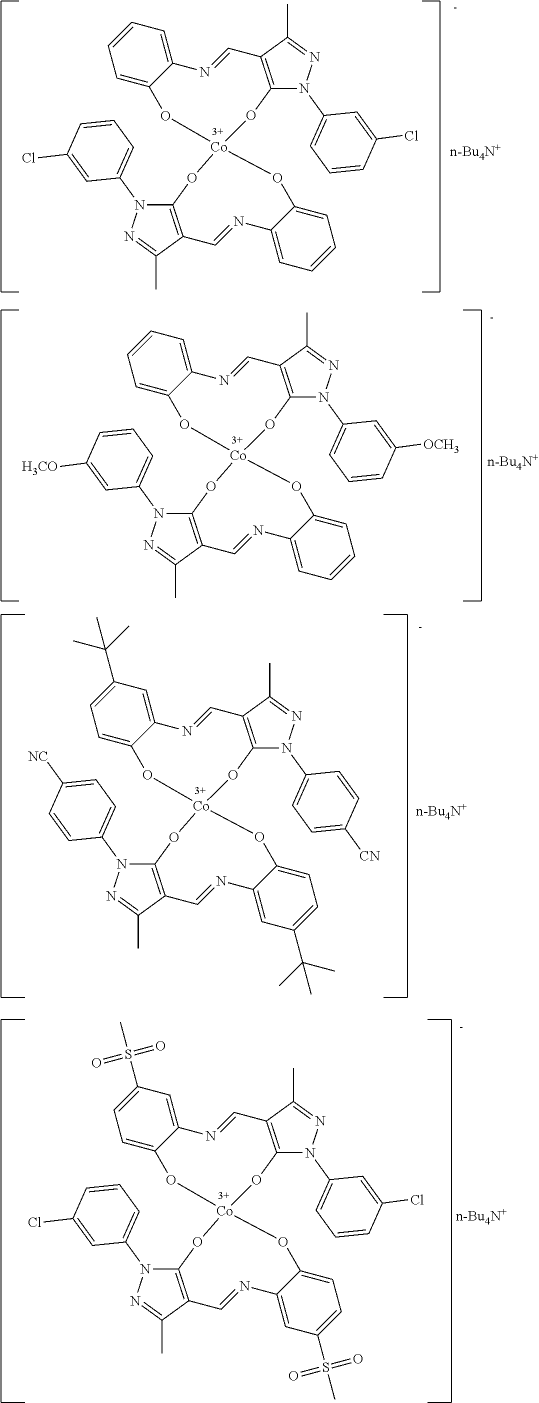

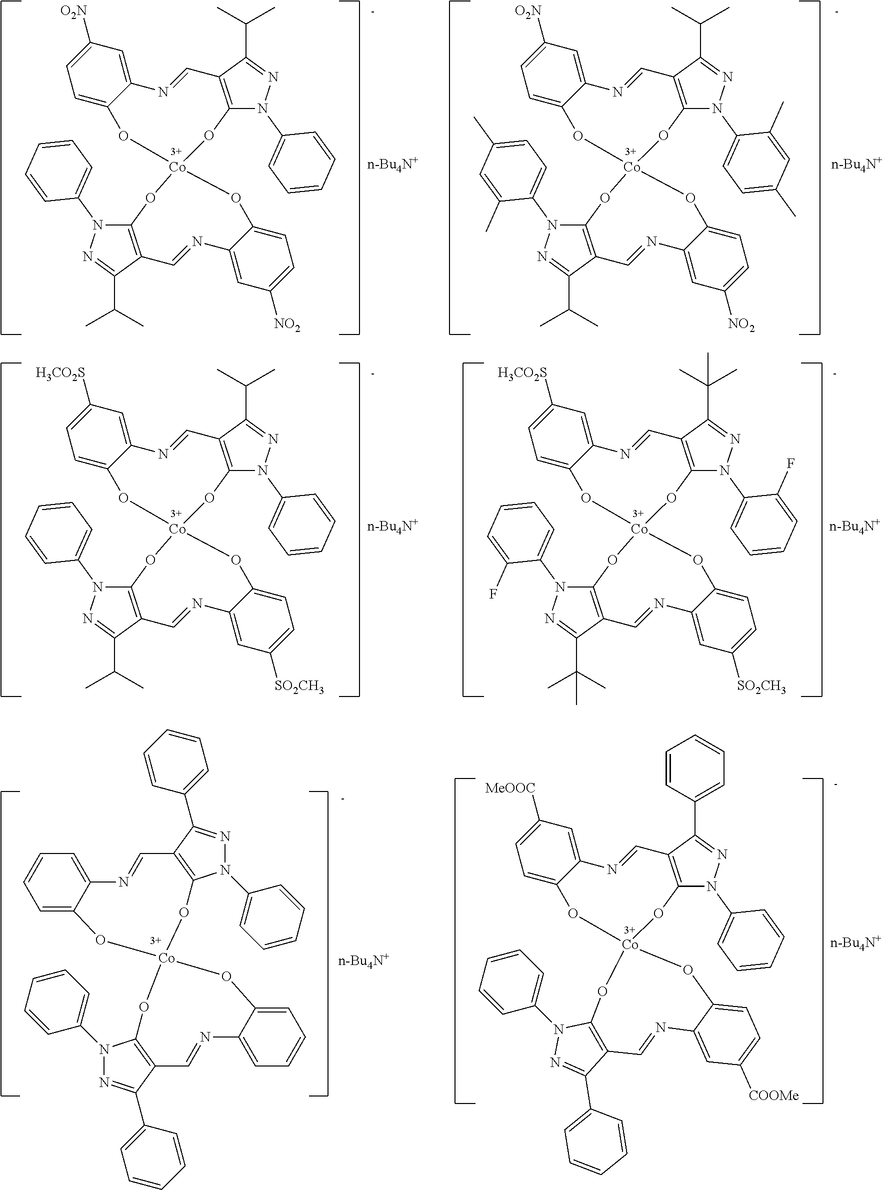

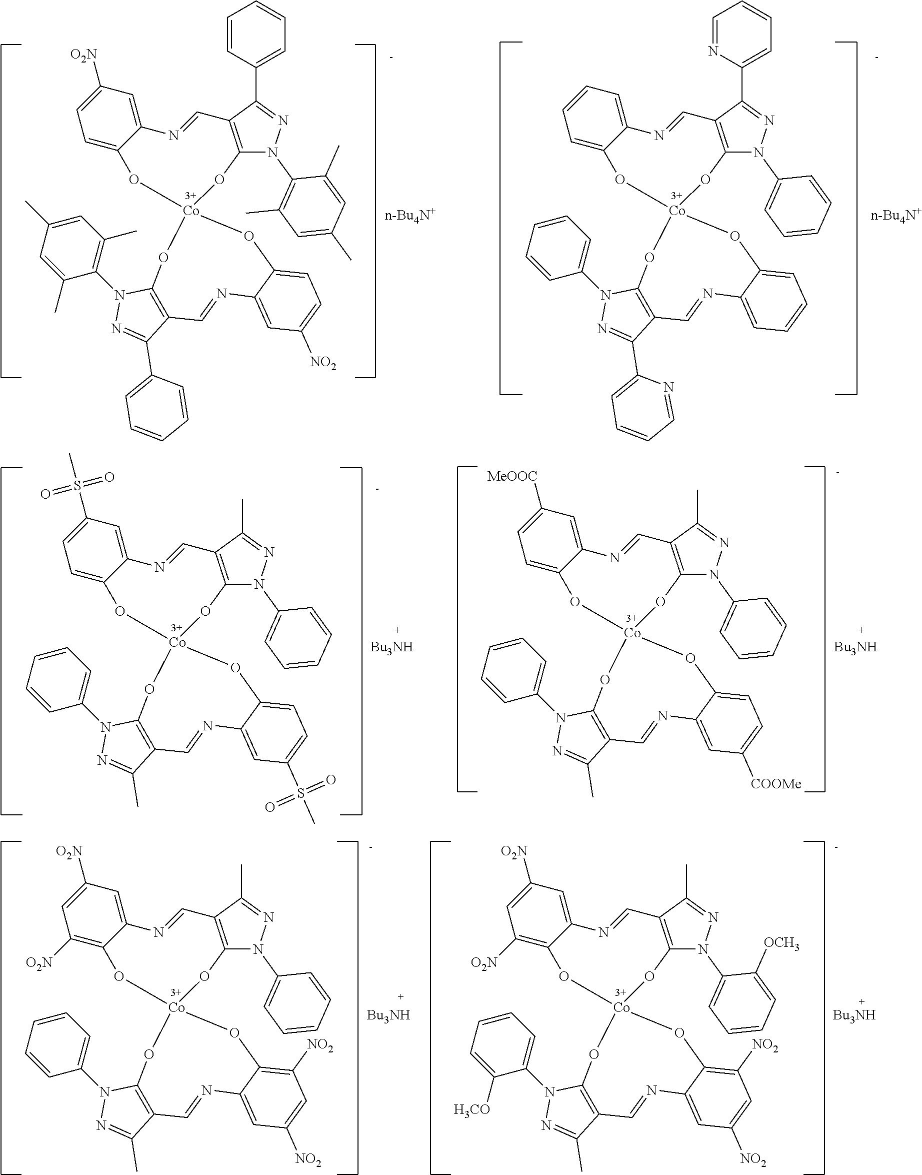

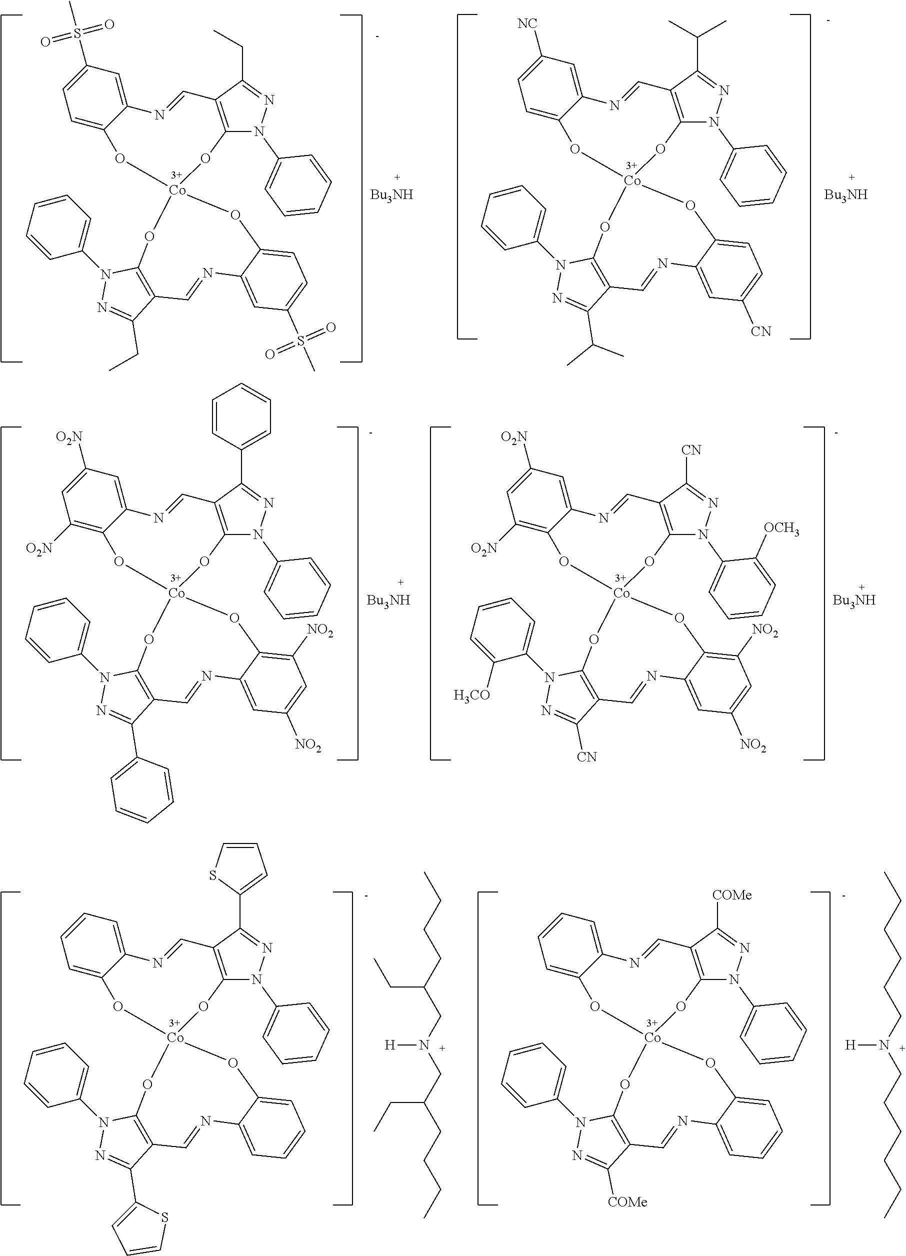

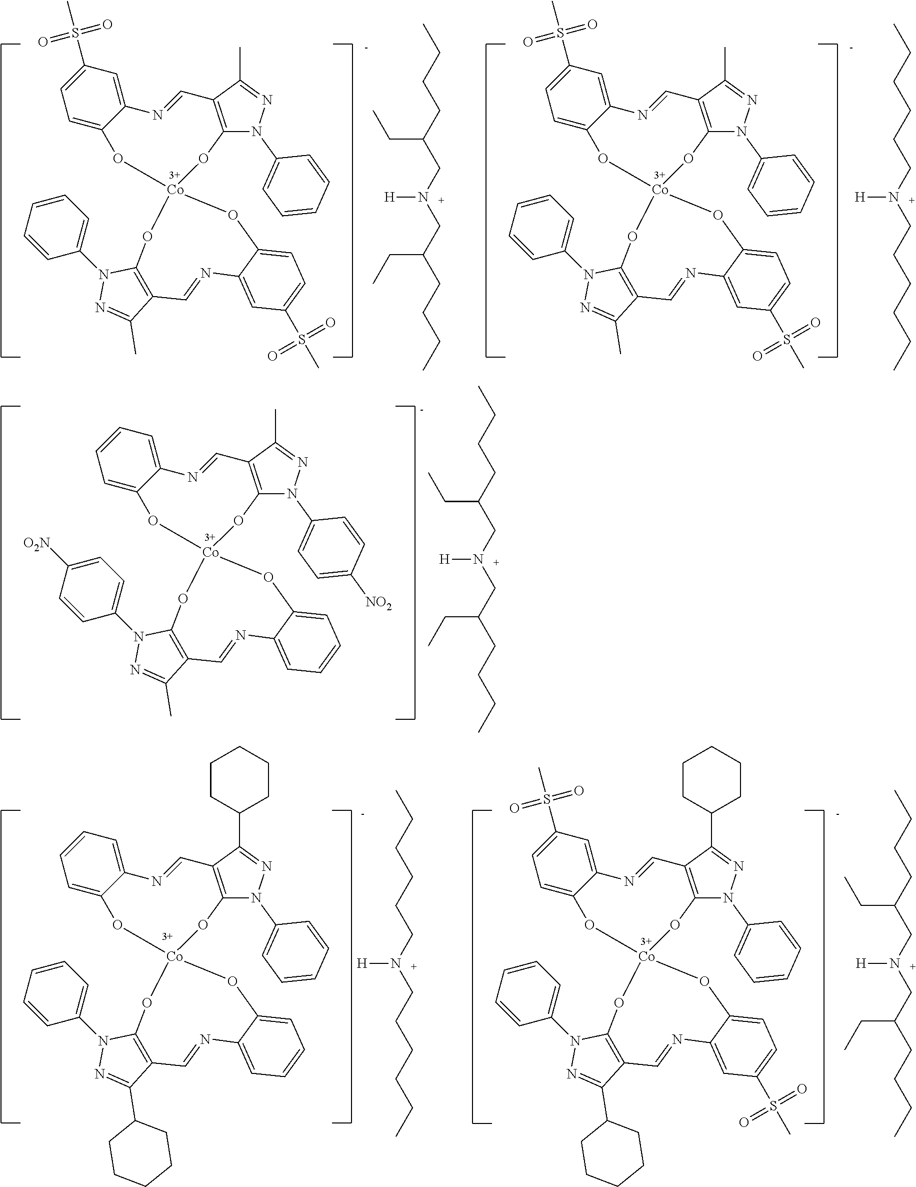

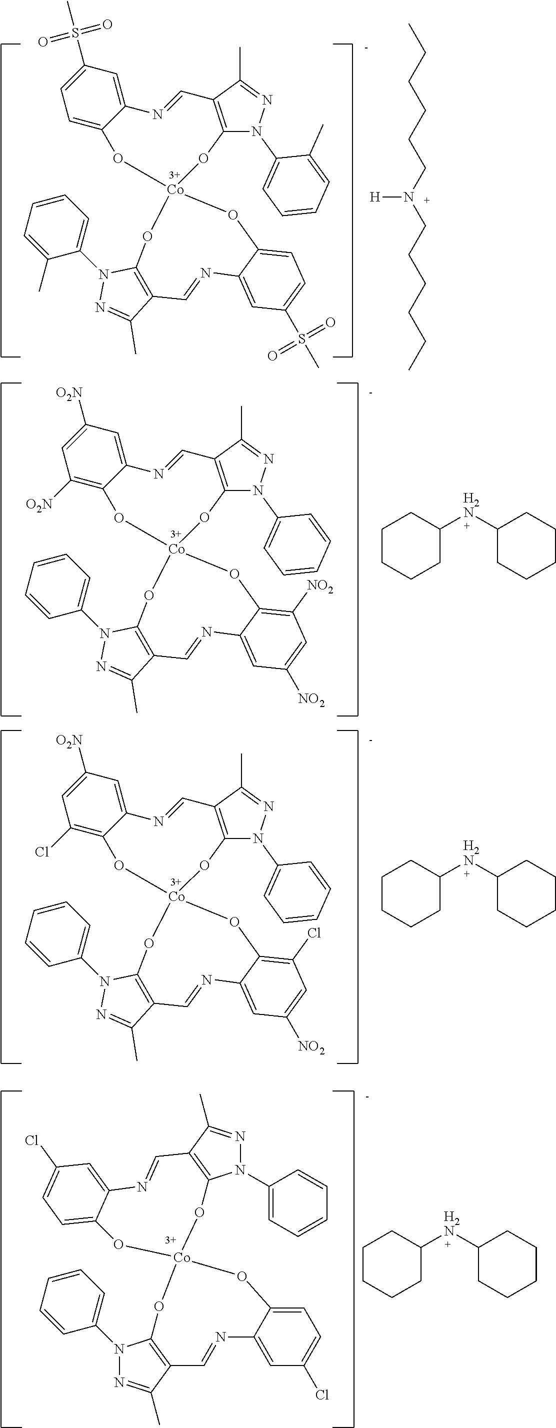

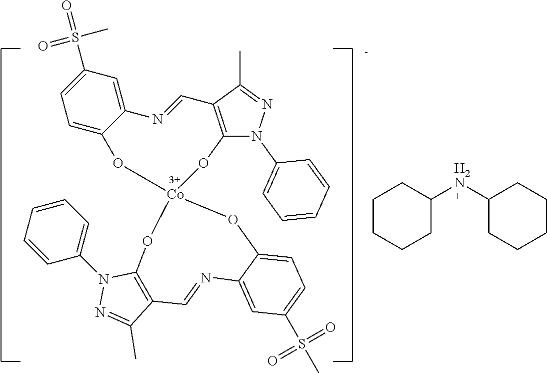

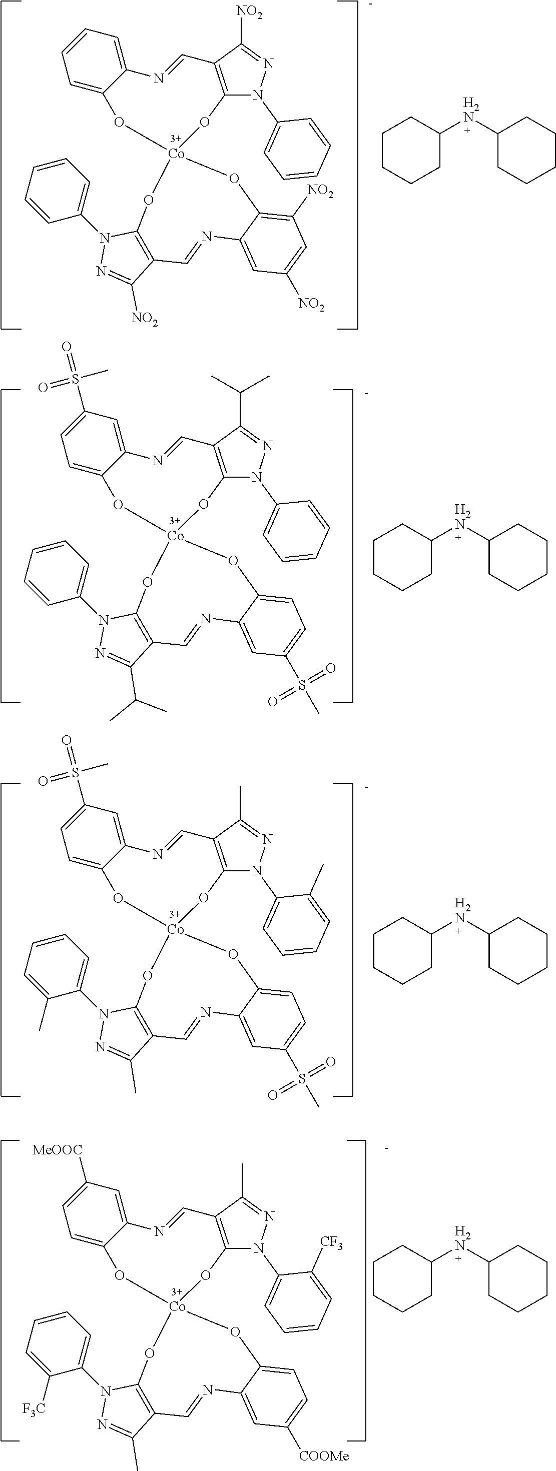

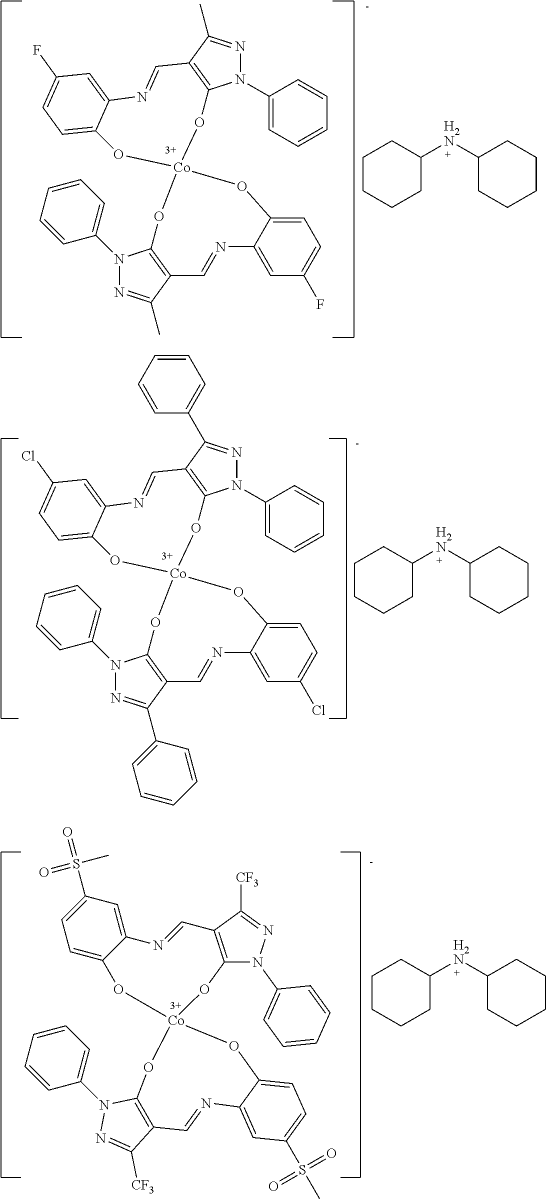

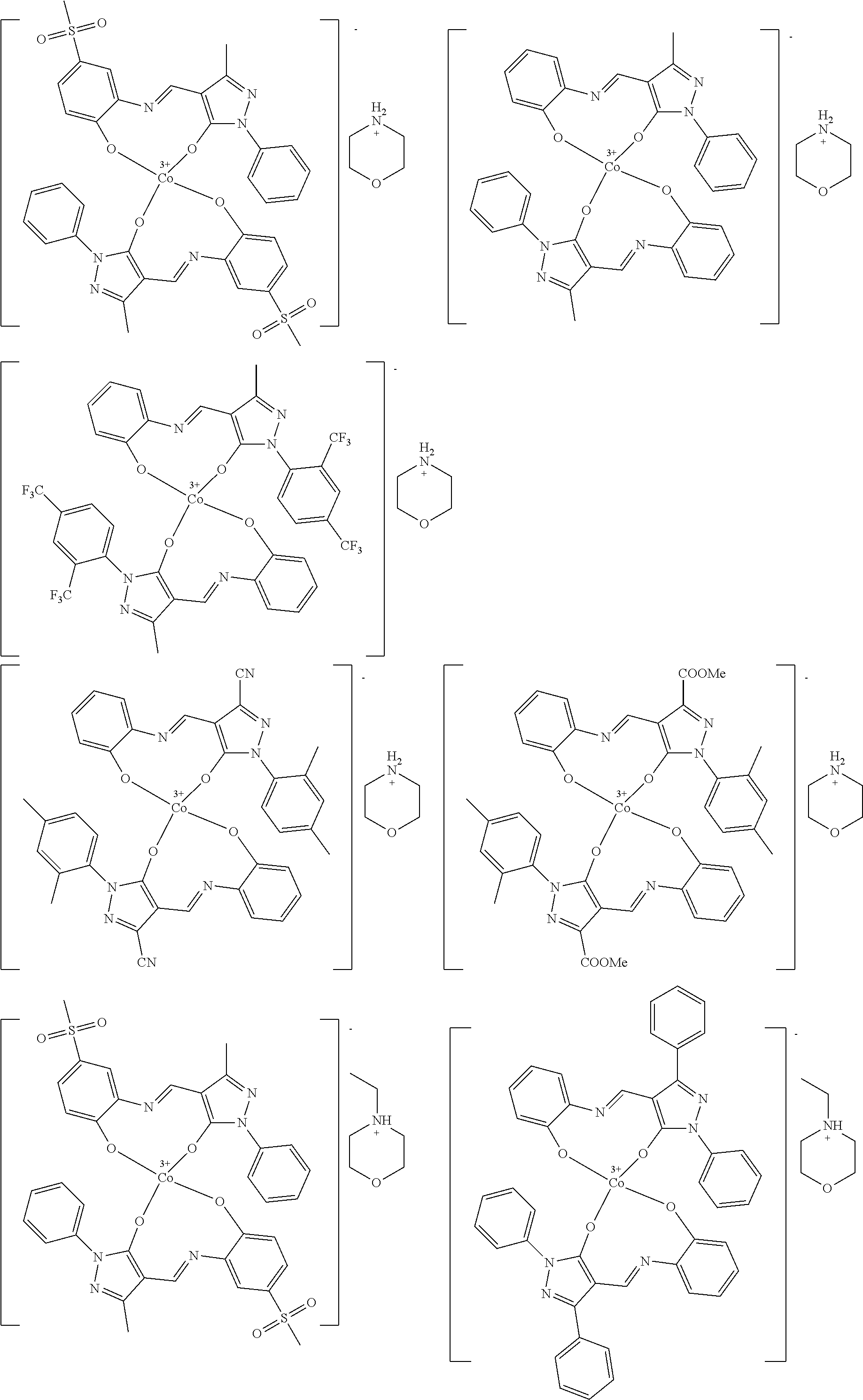

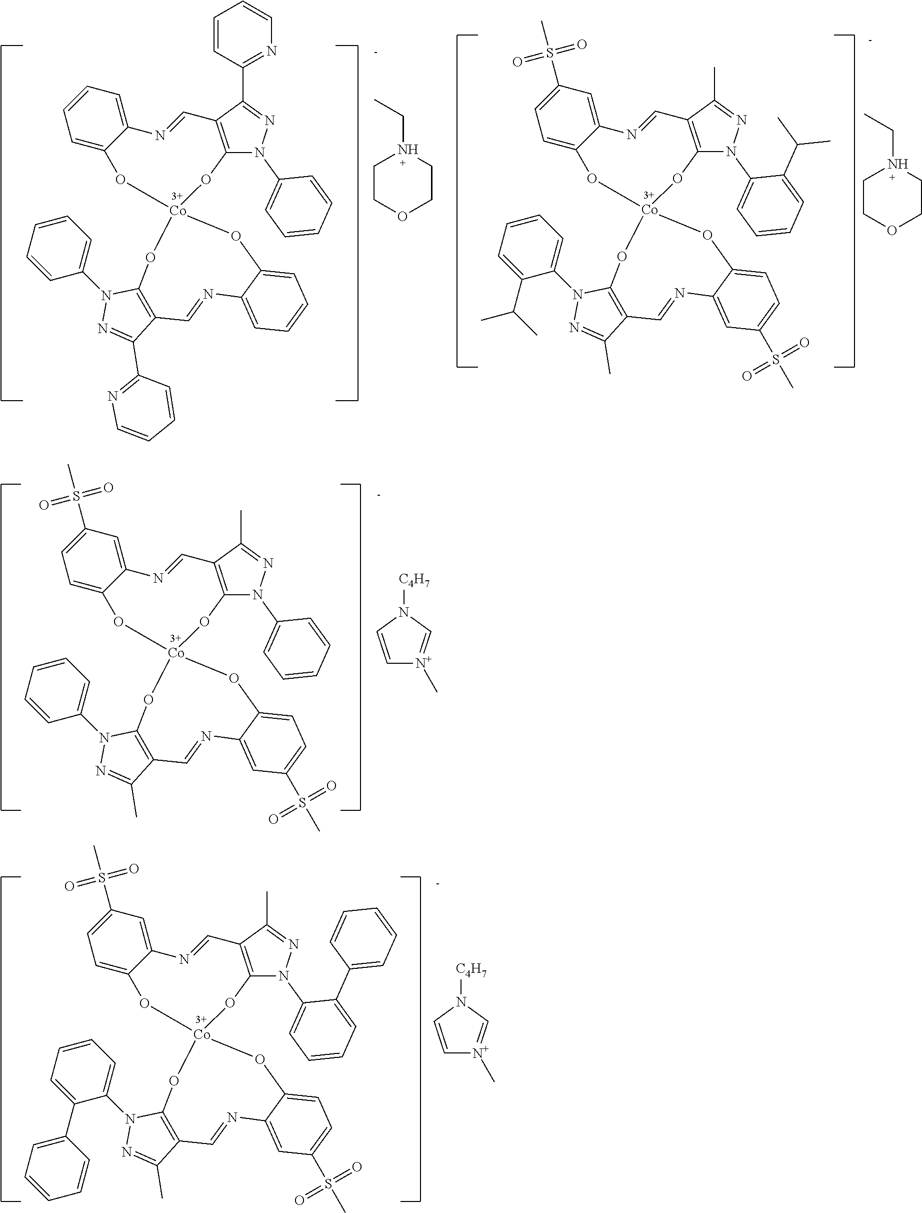

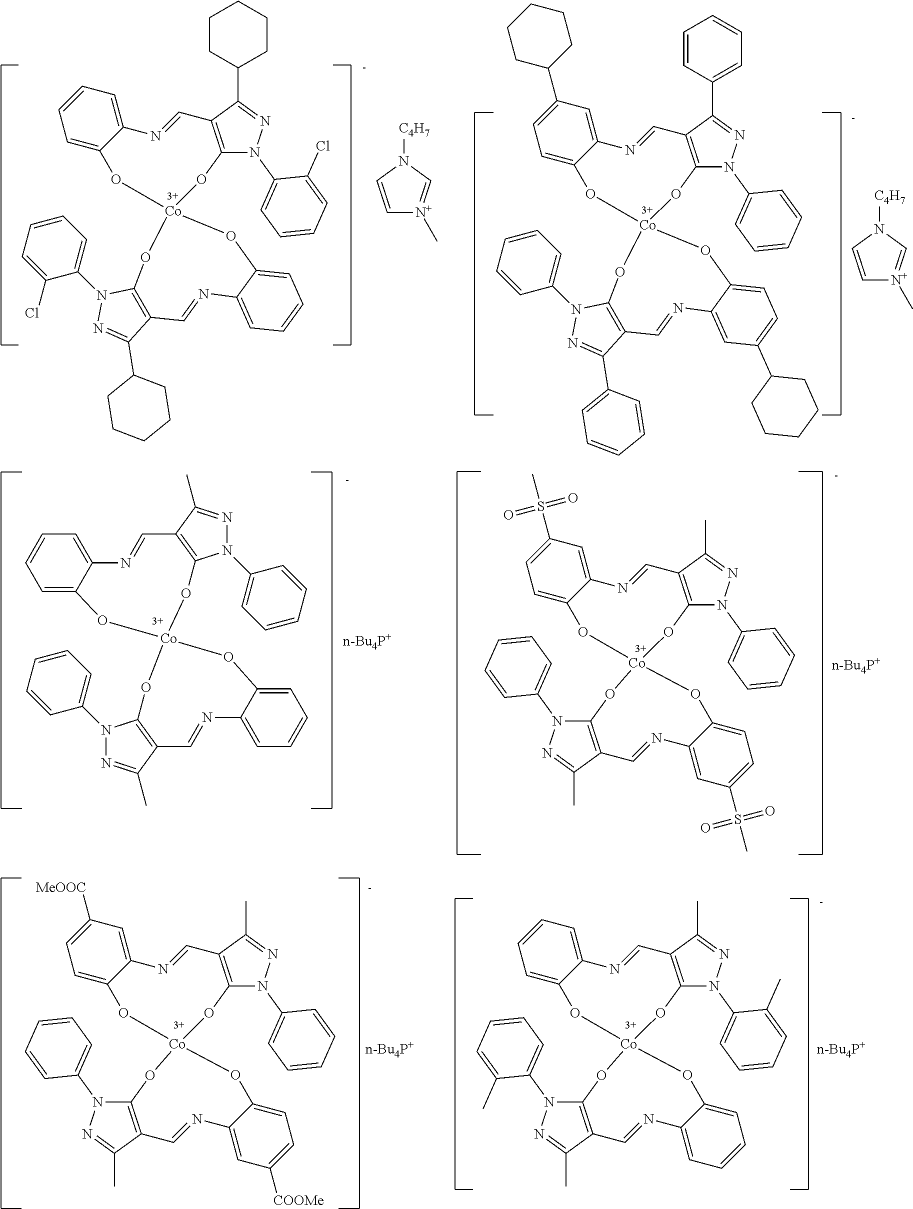

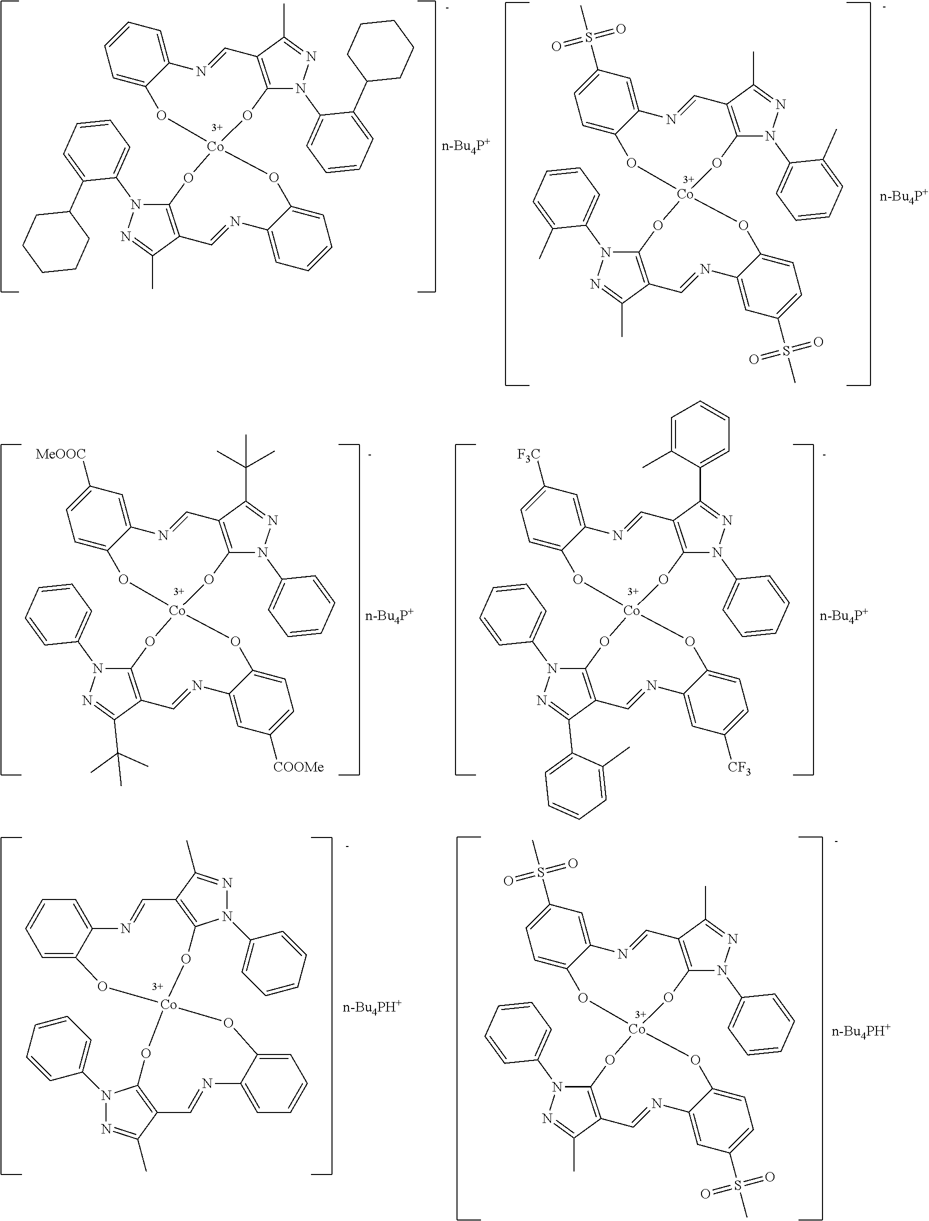

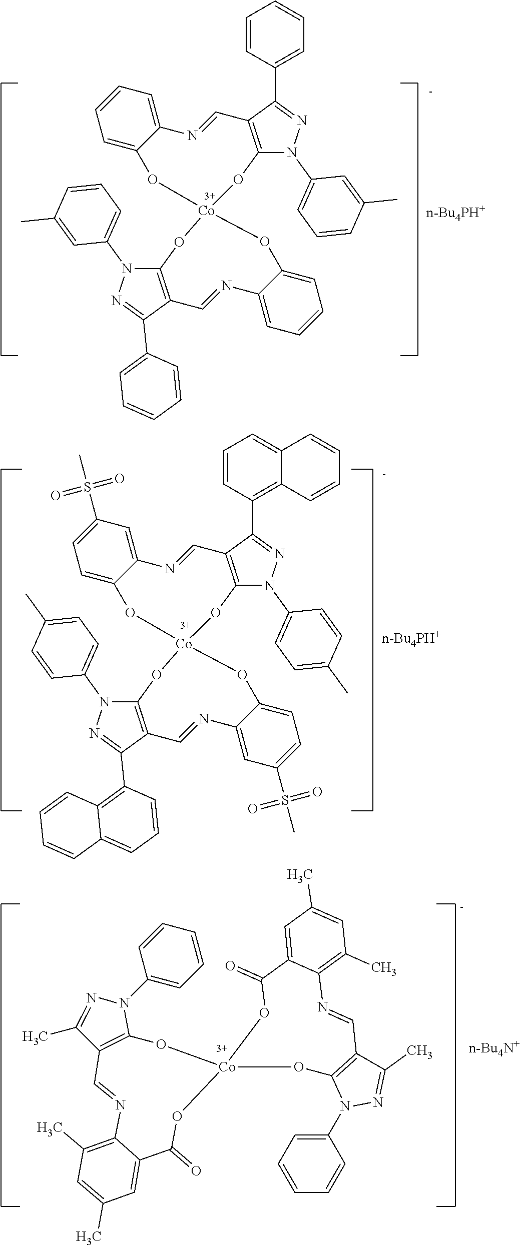

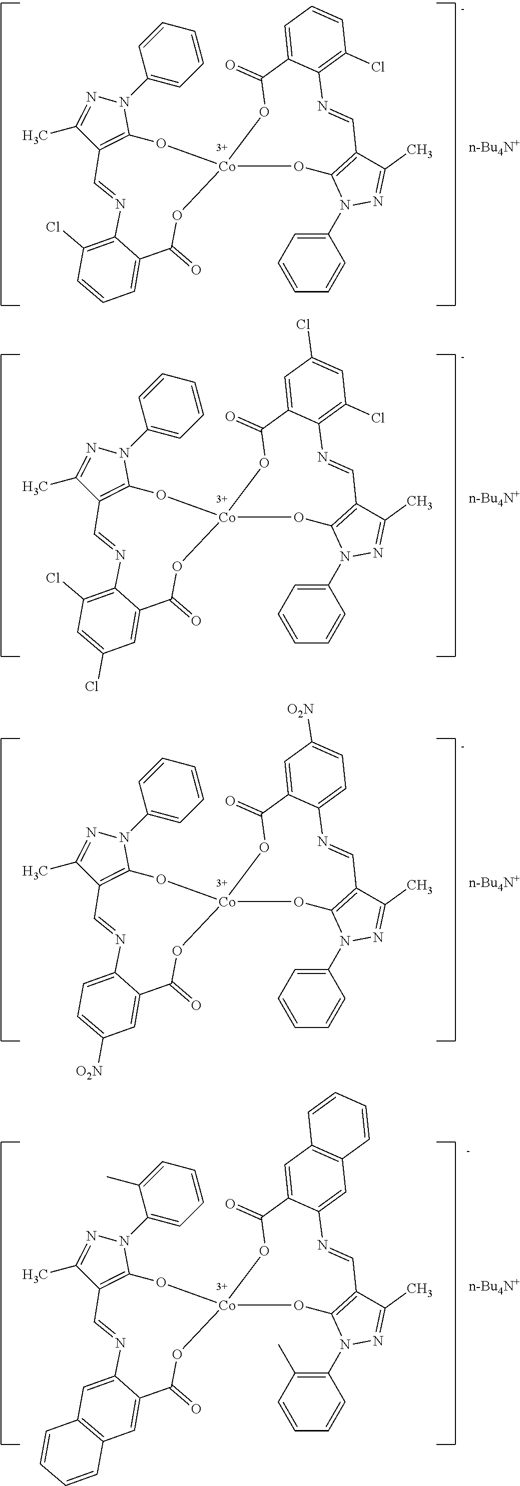

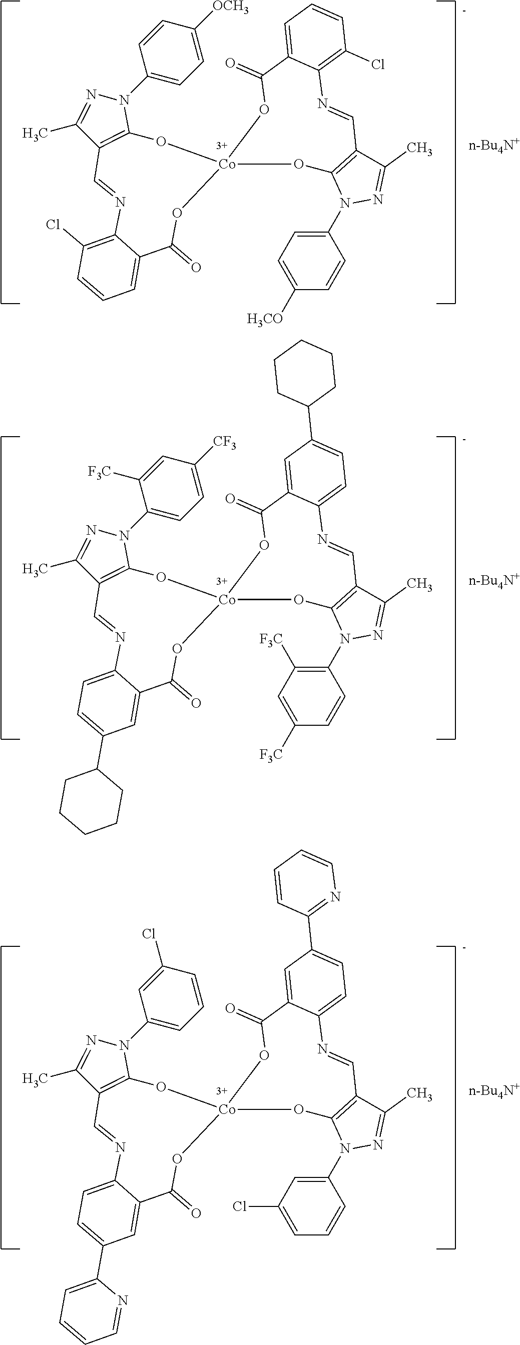

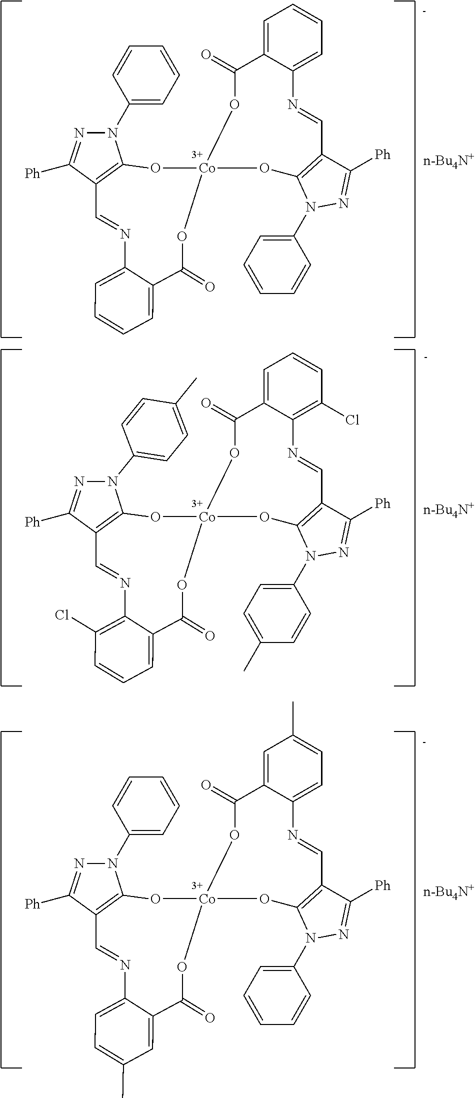

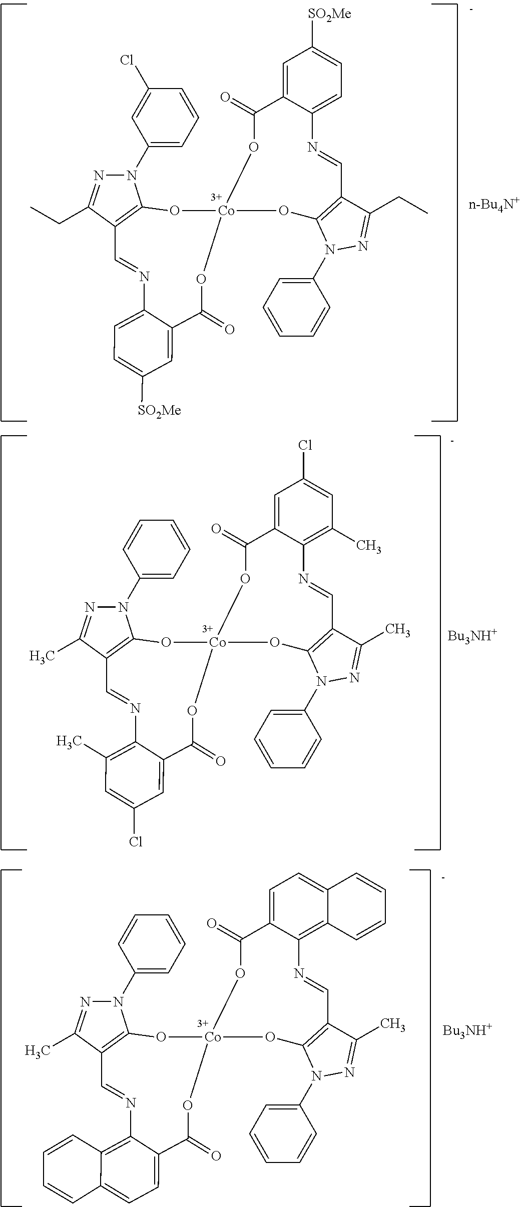

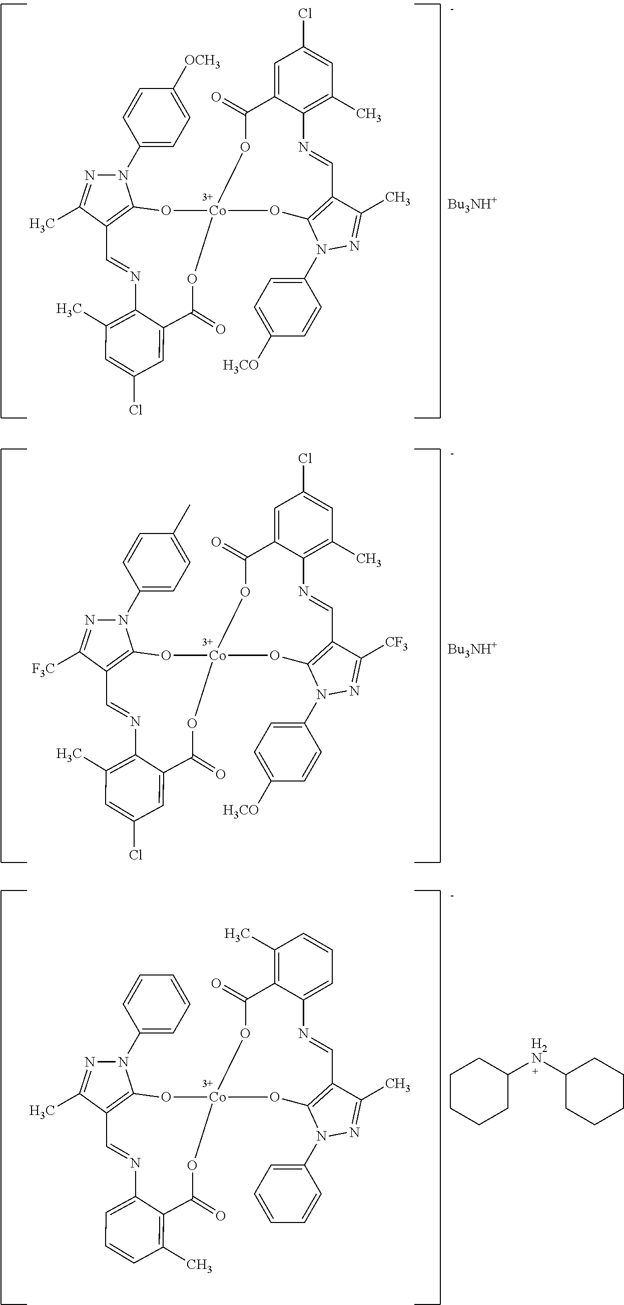

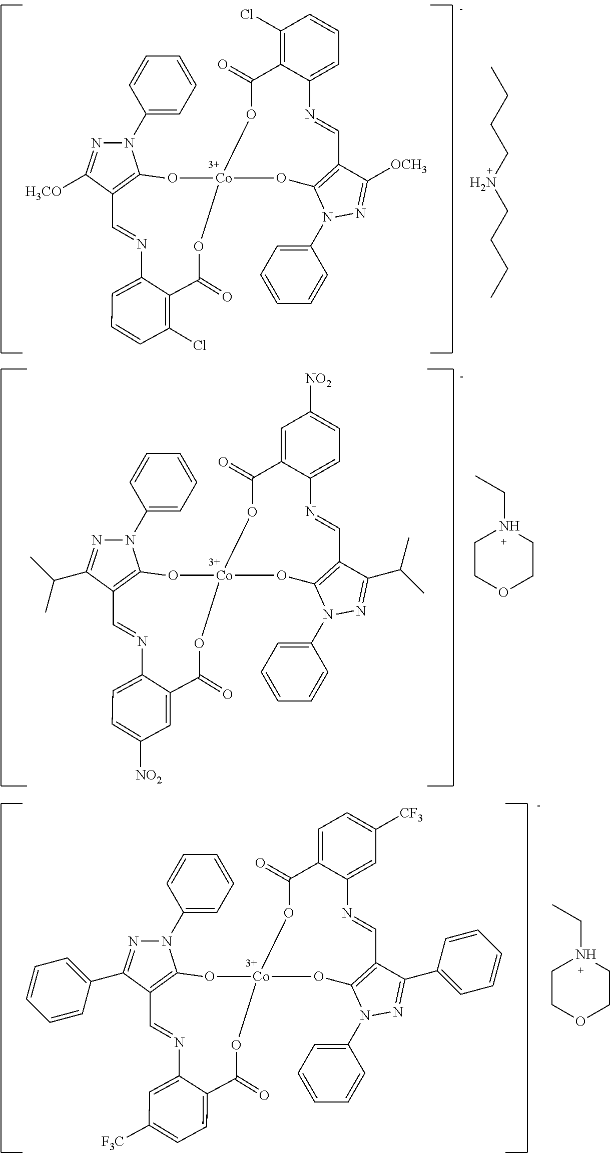

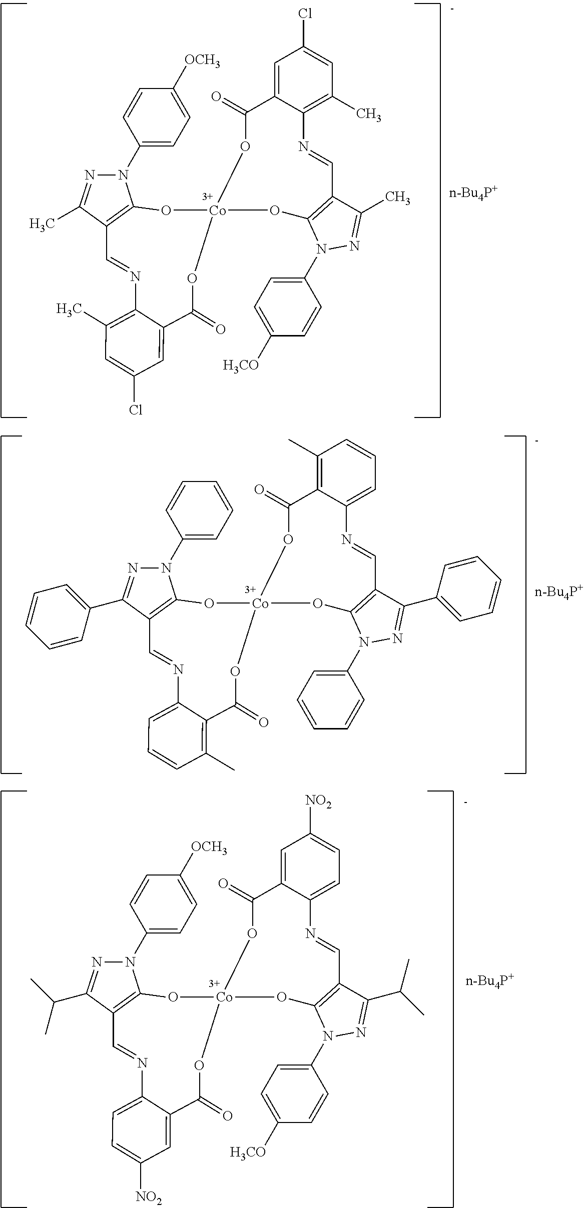

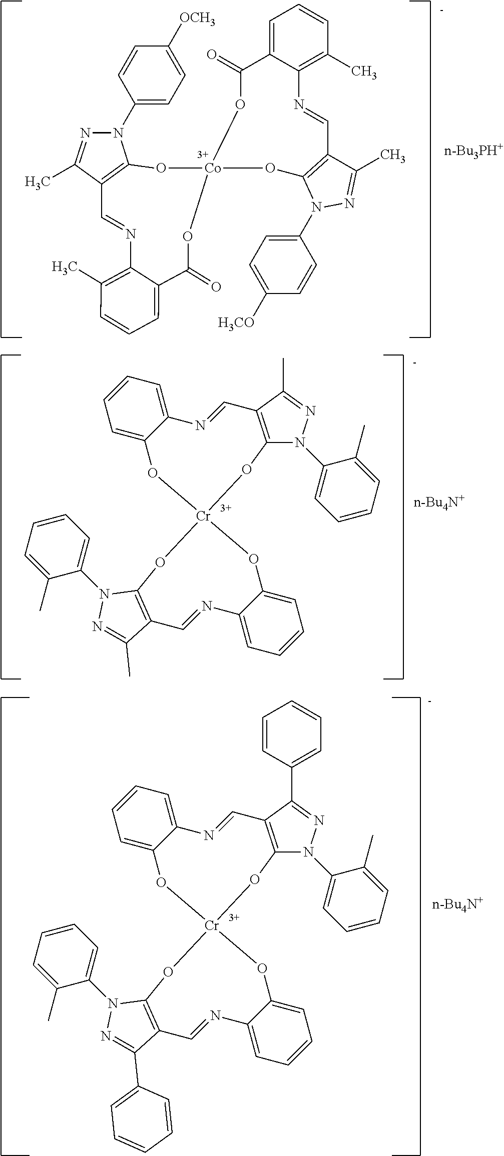

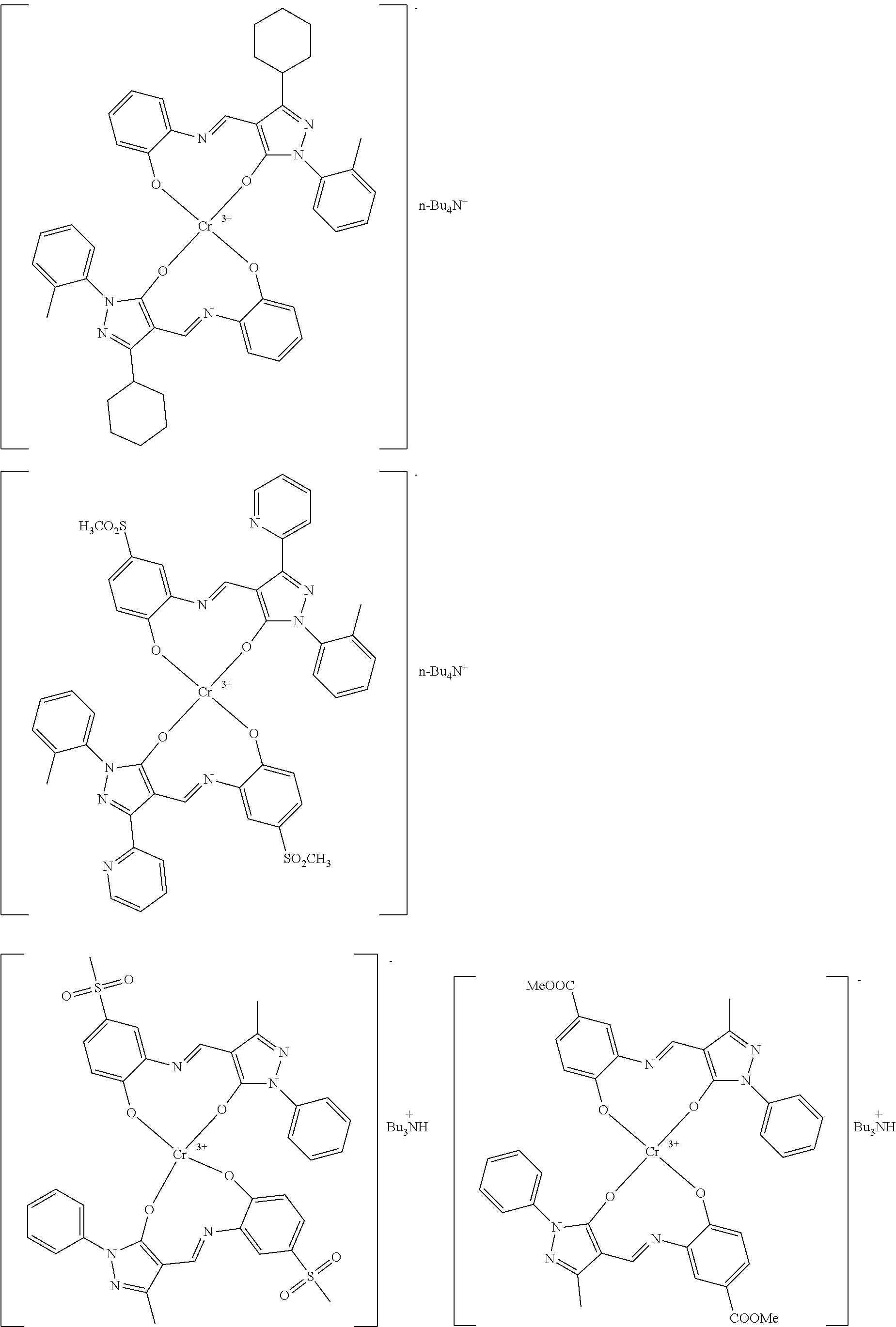

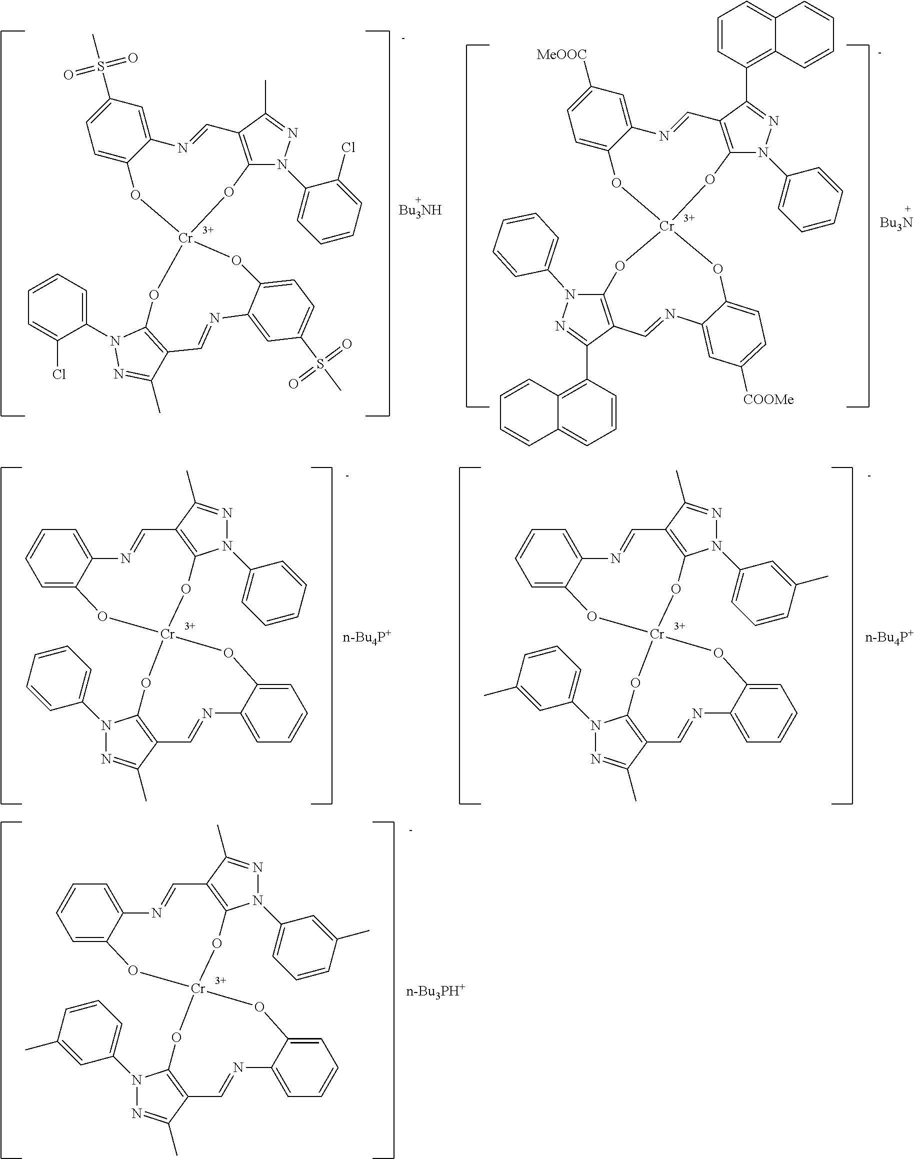

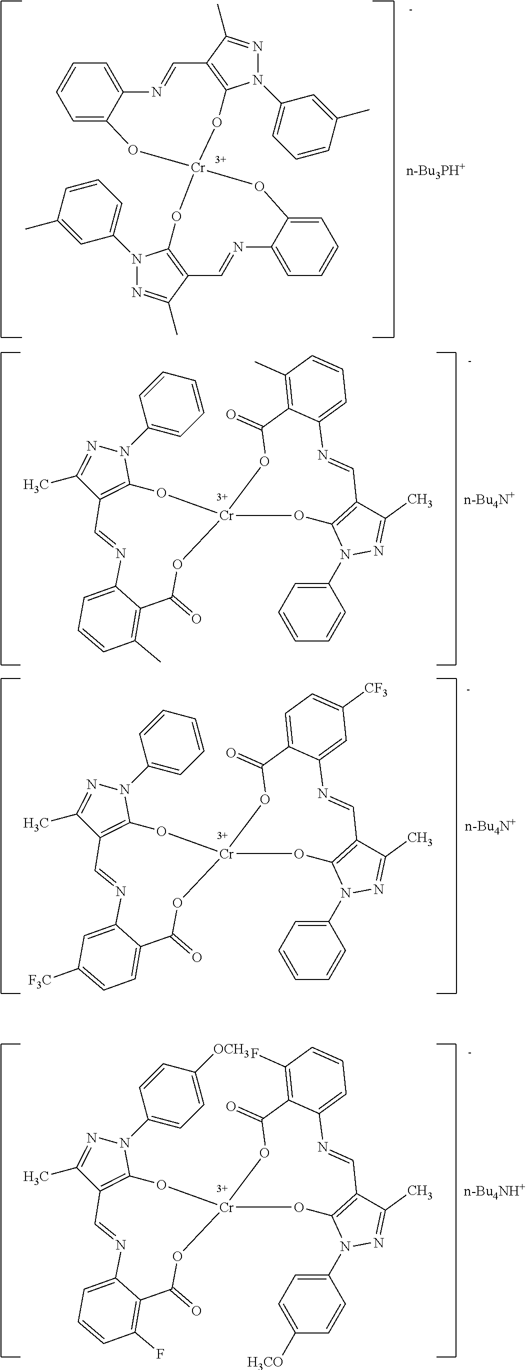

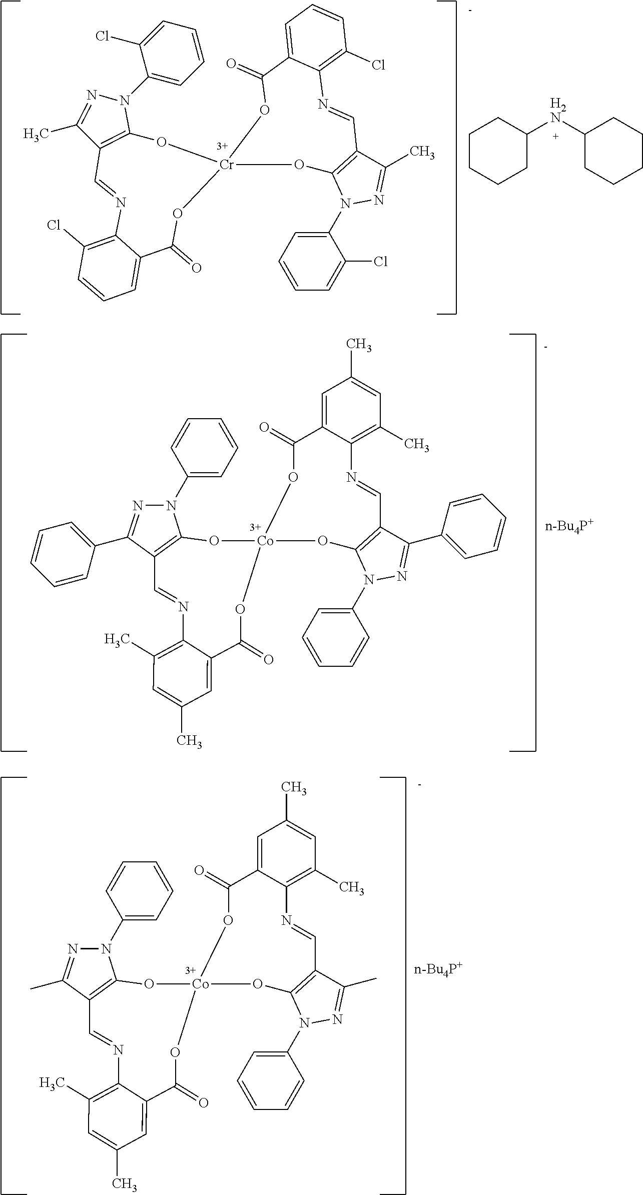

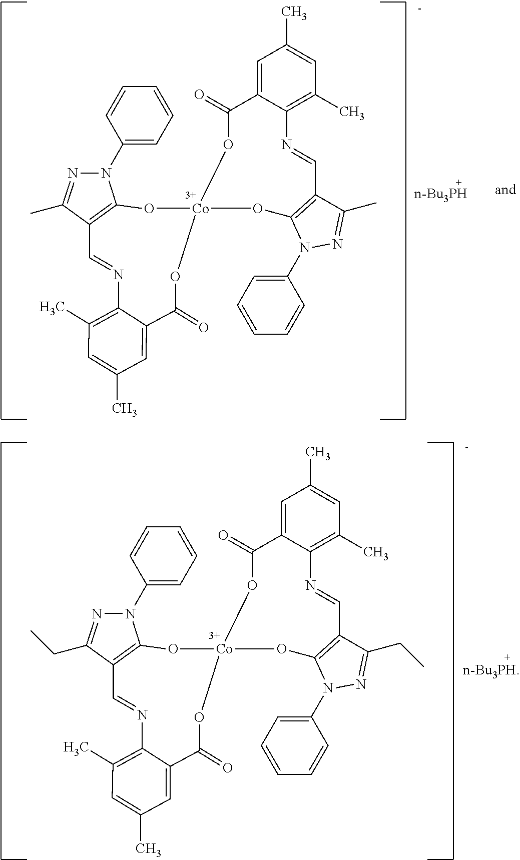

12. A light absorber comprising the coordination compound of claim 1.

13. The light absorber of claim 12, which absorbs one or more wavelengths selected from among 350 nm to 450 nm.

14. An adhesive film comprising the light absorber of claim 12.

15. An optical film comprising the light absorber of claim 12.

16. The optical film claim 15, comprising:
an adhesive film; and
a binder resin film,
wherein the adhesive film includes the light absorber.

17. An electronic device comprising the light absorber of claim 12.

18. The electronic device of claim 17, comprising an adhesive film, wherein the adhesive film includes the light absorber.
